(19) United States
(12) Patent Application Publication
(10) Pub. No.: US 2026/0265829 A1
(43) Pub. Date: Sep. 10, 2026

(54) Diagnostic, Prognostic And Therapeutic Uses Of Long Noncoding RNAs For Heart Disease And Regenerative Medicine

(71) Applicant: UNIVERSITE DE LAUSANNE, Lausanne (CH)

(72) Inventors: Samir OUNZAIN, Lausanne (CH); Thierry PEDRAZZINI, Le Mont-sur-Lausanne (CH)

(21) Appl. No.: 19/671,417

(22) Filed: May 8, 2026

Related U.S. Application Data
(62) Division of application No. 17/505,153, filed on Oct. 19, 2021, now Pat. No. 12,649,948.
Application No. 17/505,153 is a continuation of application No. 15/105,319, filed on Dec. 19, 2014, now Pat. No. 11,193,171.
(60) Provisional application No. 61/964,591, filed on Dec. 20, 2013.

(51) Int. Cl. C12Q 1/6883

(52) U.S. Cl. CPC C12Q 1/6883; C12Q 2600/158; C12Q 2600/178

(57) ABSTRACT

Provided herein are lncRNAs and methods for diagnosing cardiac pathologies in a subject. Also provided are methods for treating a cardiac pathology in a subject comprising administering to said subject an effective amount of a modulator of one or more lncRNAs.

### CROSS REFERENCE TO RELATED APPLICATIONS

This patent application is a divisional of U.S. application Ser. No. 17/505,153 filed on 19 Oct. 2021, which is a continuation of U.S. patent application Ser. No. 15/105,319 filed on 16 Jun. 2016, now U.S. Pat. No. 11,193,171, which is a U.S. national stage filing under 35 U.S.C. § 371 of International Patent Application No. PCT/EP2014/078868 filed on 19 Dec. 2014, which claims the benefit of U.S. Provisional Patent Application No. 61/964,591 filed on 20 Dec. 2013. The entire disclosures of each of the above recited applications are incorporated herein by reference.

### REFERENCE TO AN ELECTRONIC SEQUENCE LISTING

This application contains references to nucleic acid sequences and/or amino acid sequences which have been submitted concurrently herewith as the sequence listing .XML file “6170-000096-US-DVB_2026-05-04_ST26” file size 521,421 Bytes (B), created on 4 May 2026. The aforementioned sequence listing is hereby incorporated by reference in its entirety pursuant to 37 C.F.R. § 1.52 (e) (5).

### FIELD OF THE INVENTION

This invention generally relates to lncRNAs and methods for diagnosing cardiac pathologies in a subject. The invention also provides methods for treating a cardiac pathology in a subject comprising administering to said subject an effective amount of a modulator of one or more lncRNAs of the invention.

### BACKGROUND OF THE INVENTION

The recent statistics on heart disease from the American Heart Association reports that one in nine death certificates in the United States mentioned heart failure as the cause of death in 2010. The burden of cardiovascular disease remains particularly high, with an overall rate of death attributable to cardiovascular disease of 235 per 100,000. Coronary artery disease is the most frequent cardiovascular disorder and typically leads to acute myocardial infarction and ultimately heart failure (HF). Despite continued advances, no approach currently exists to reverse the loss of functional myocardium, and HF is thus rapidly evolving into a major global epidemic requiring novel therapeutic approaches. In light of this, the elucidation of novel pathways and mechanisms involved in HF pathogenesis holds the promise of identifying new avenues and targets for this prevalent and deadly disease. In the adult heart, stress-dependant pathological hemodynamic and neurohormonal signals induce a maladaptive remodeling response, a process characterized by increased cardiomyocyte size (cellular hypertrophy), interstitial fibrosis and ultimately cellular dysfunction resulting in contractile and functional failure. At the molecular level, these signals activate a network of interacting cardiac signal transduction cascades that converge on a defined set of evolutionary conserved cardiac transcription factors (TFs). These core cardiac TFs (SRF, Nkx2.5, Mef2c, Gata4, TBox) interact in a combinatorial manner to elicit specific temporal and spatial gene expression programs. This integrated modulation of protein coding gene expression is ultimately responsible for cellular fate and is integral to the pathological remodeling process.

The notion of gene regulatory networks (GRNs) being primarily protein-based regulatory systems has been somewhat premature. A number of recent studies have demonstrated that GRN activity is under the control of a myriad of interleaved networks of non-coding RNAs (ncRNAs). Non-coding RNAs control every aspect of GRN activity including transcriptional control, post-transcriptional processing and epigenetic targeting (Ounzain et al., 2013). The best-characterized ncRNAs in the heart are the small microRNAs (miRNAs). Cardiovascular miRNAs adjust entire functional networks of mRNAs via post-transcriptional gene silencing, implicating miRNAs as important stress-dependant modulators. In addition to small ncRNAs, global transcriptional screens have identified other functional classes of transcripts, which are larger than 200 nucleotides, collectively known as long non-coding RNAs (lncRNAs). The functions of most lncRNAs remain unknown, however many have been shown to exert non-redundant roles in a diverse array of biological processes including X inactivation, imprinting, splicing and transcriptional regulation. In particular lncRNAs appear to be important for the global modulation of cell-specific epigenomic states via directing chromatin modification complexes to their sites of action (. Furthermore, mammalian lncRNAs appear to be expressed in a highly cell-type and context-specific manner Considering the functionality of these transcripts, this raises the possibility that lncRNAs are an important class of regulatory mediators of cardiogenic lineage-specific developmental or specialized cellular functions. The majority of lncRNAs functionally characterized to date regulate developmental processes. However, their potential role controlling mature tissue homeostasis and adaptation to stress remains largely unexplored.

Identification of novel regulatory molecules and/or pathways that participate in the adaptation of the heart to stress is an important step towards the development of new therapeutic strategies aimed at preventing the progression to heart failure. Importantly, the hallmark of pathological remodeling in the adult heart is a global transcriptional reprogramming, resulting in the reactivation of a “fetal” cardiac gene program. The intrinsic-cis and -trans activating and epigenomic orchestrating properties of lncRNAs warrants the need to explore and generate catalogues of cardiac-specific lncRNAs in diseased adult tissues.

### SUMMARY OF THE INVENTION

The Invention relates to a method for diagnosing a cardiac pathology in a subject, the method comprising: a) measuring the level of a plurality of biomarkers in a biological sample derived from the subject, wherein the plurality of biomarkers comprises one or more lncRNAs selected from the group comprising one or more lncRNAs selected from the group comprising SEQ ID No 1, SEQ ID No 2, SEQ ID No 3, SEQ ID No 4, SEQ ID No 5, SEQ ID No 6, SEQ ID No 7, SEQ ID No 8, SEQ ID No 9, SEQ ID No 10, SEQ ID No 11, SEQ ID No 12, SEQ ID No 13, SEQ ID No 14, SEQ ID No 15, SEQ ID No 16, SEQ ID No 17, SEQ ID No 18, SEQ ID No 19, SEQ ID No 20, SEQ ID No 21, SEQ ID No 22, SEQ ID No 23, SEQ ID No 24, SEQ ID No 25, SEQ ID No 26, SEQ ID No 27, SEQ ID No 28, SEQ ID No 29, SEQ ID No 30, SEQ ID No 31, SEQ ID No 32, SEQ ID No 33, SEQ ID No 34, SEQ ID No 35, SEQ ID No 36, SEQ ID No 37, SEQ ID No 38, SEQ ID No 39, SEQ ID No 40, SEQ ID No 41, SEQ ID No 42, SEQ ID No 43, SEQ ID No 44, SEQ ID No 45, SEQ ID No 46, SEQ ID No 47, SEQ ID No 48, SEQ ID No 49, SEQ ID No 50, SEQ ID No 51, SEQ ID No 52, SEQ ID No 53, SEQ ID No 54, SEQ ID No 55, SEQ ID No 56, SEQ ID No 57, SEQ ID No 58, SEQ ID No 59, SEQ ID No 60, SEQ ID No 61, SEQ ID No 62, SEQ ID No 63, SEQ ID No 64, SEQ ID No 65, SEQ ID No 66, SEQ ID No 67, SEQ ID No 68, SEQ ID No 69, SEQ ID No 70, SEQ ID No 71, SEQ ID No 72, SEQ ID No 73, SEQ ID No 74, SEQ ID No 75, SEQ ID No 76, SEQ ID No 77, SEQ ID No 78, SEQ ID No 79, SEQ ID No 80, SEQ ID No 81, SEQ ID No 82, SEQ ID No 83, SEQ ID No 84, SEQ ID No 85, SEQ ID No 86, SEQ ID No 87, SEQ ID No 88, SEQ ID No 89, SEQ ID No 90, SEQ ID No 91, SEQ ID No 92, SEQ ID No 93, SEQ ID No 94, SEQ ID No 95, SEQ ID No 96, SEQ ID No 97, SEQ ID No 98, SEQ ID No 99, SEQ ID No 100, SEQ ID No 101, SEQ ID No 102, SEQ ID No103, and SEQ ID No 104, fragments thereof, isoforms thereof and variants sharing at least 80% nucleotide sequence identity thereto; and b) analyzing the levels of the biomarkers, fragments thereof, isoforms thereof and variants sharing at least 80% nucleotide sequence identity thereto, in conjunction with respective reference value ranges for said plurality of biomarkers, wherein differential expression of one or more biomarkers in the biological sample compared to one or more biomarkers in a control sample from a normal subject indicates that the subject has a cardiac pathology.

A further object of the present invention is to provide a method for treating a cardiac pathology in a subject comprising administering to said subject an effective amount of a modulator of one or more lncRNAs of the invention, fragments thereof, isoforms thereof and variants sharing at least 80% nucleotide sequence identity thereto.

The invention also contemplates a composition comprising a modulator of one or more lncRNAs of the invention wherein wherein the modulator is selected from the group comprising a chemical agent, an antibody, an engineered protease, a CRISPR based technology and enzymatically active RNA.

A further object of the invention is to provide a method for modulating one or more lncRNAs of the invention wherein the modulator is selected from the group comprising a miRNA, a siRNA, a piRNA, a snRNA and an antisense oligonucleotide.

Another object of the invention is to provide a pharmaceutical composition comprising an effective amount of a modulator of one or more lncRNAs wherein the modulator is selected from the group comprising a chemical agent, an antibody, an engineered protease, and enzymatically active RNA, optionally in combination with pharmaceutically acceptable carriers, diluents and/or adjuvants.

Also comtemplated is a kit comprising the compositions of the invention.

Other objects of the invention concern a method for diagnosing a cardiac pathology in a subject, a method for monitoring the effects of a treatment on cardiac tissue in a subject, a method for monitoring the efficacy of surgical and/or pharmacological cardiac therapies in a subject, a method for measuring cardiac tissue regeneration in a subject, a method for monitoring in vitro cardiogenic cell differentiation, a method for monitoring in vivo cardiogenic cell differentiation, and a method for monitoring efficacy of agents and/or small molecules that can induce cardiac reprogramming.

The invention also concerns a microarray comprising a plurality of probes that hybridize to one or more lncRNAs of the invention, fragments thereof, isoforms thereof and variants sharing at least 80% nucleotide sequence identity thereto.

### BRIEF DESCRIPTION OF THE FIGURES

FIGS. 1A-F show that the cardiac coding and noncoding transcriptome is highly correlated with cardiac physiological traits. FIG. 1A shows that mRNAs are highly clustered in their correlations with cardiac physiological traits and that such clusters are associated with genes implicated in expected processes. FIG. 1B shows that for specific traits, each cluster globally either correlates or does not correlate with each individual trait. FIG. 1C shows that novel lncRNAs are highly clustered in their correlations with physiological traits and that the closest coding genes to lncRNAs in each cluster are associated with specific biological processes. FIG. 1D shows that identified novel lncRNA clusters correlate specifically with individual physiological traits. FIG. 1E shows the heart specificity of mRNAs in clusters 1 to 4. FIG. 1F shows the heart specificity of novel lncRNAs in clusters 1 to 4.

FIGS. 2A-G show the validation and manipulation of selected novel lncRNAs in vivo and in vitro. FIG. 5A shows the validation of expression via quantitative RT-PCR of novel lncRNAs in the border and remote zones of infracted hearts one and seven days post myocardial infarction. FIG. 2B (left) shows the relative expression of candidate novel lncRNAs in isolated mouse cardiomyocytes and fibroblasts. FIG. 2C (right) shows the nuclear and cytoplasmic enrichment of candidate novel lncRNAs in cardiomyoctes and fibroblasts. FIG. 2D shows the correlation of candidate novel lncRNAs with physiological traits. FIG. 2E shows the expression of candidate lncRNAs in mouse embryonic stem cells undergoing cardiogenic differentiation. FIG. 2F shows the chromatin state patterns observed at novel lncRNA promoters during cardiogenic differentiation of mouse embryonic stem cells. FIG. 2G shows that modified antisense oligonucleotide mediated knock-down of specific novel lncRNA (Novlnc6) results in specific modulation of a key cardiac transcription factor, Nkx2-5 in isolated mouse cardiomyocytes.

FIGS. 3A-E show the characterisation and validation of human orthologs in cardiac pathology. FIG. 3A shows an example of a transmapped human lncRNA orthogous sequence (Novlnc6). FIG. 3B shows the expression of novel orthologous human lncRNAs in patients suffering with dilated cardiomyopathy (DCM) or aortic stenosis (AOS). FIG. 3C shows expression of validated cardiac biomarkers, ANF and Colla2, in DCM and AOS patients. FIG. 3D shows ejection fraction percentage (EF %), wall thickness, left ventricular (LV) mass index, and end-diastolic dimension (EDD). FIG. 3E shows the relative expression of Hs NovInc6, Hs NovInc23, Hs NovInc44, Nkx2.5, Nppa, and Colla2.

FIGS. 4A-D show in vitro cell-specific lncRNA expression, and effects of lncRNA downregulation in cardiac fibroblasts and cardiomyocytes. FIG. 4A shows the relative expression of specific lncRNAs in isolated cardiomyocytes versus cardiac fibroblasts. FIG. 4B shows the relative expression of Lnc-019010 in fibroblasts derived form the heart, the lung and the tail. FIG. 4C shows the impact of Lnc-019010 loss-of-function using modified antisense oligonucleotides in cardiac fibroblasts on a panel of coding genes that control the fibrotic response. FIG. 4D shows the impact of Lnc-033521 loss-of-function on predicted target genes in isolated cardiomyocytes.

FIGS. 5A-B show in vivo LncRNAs downregulation in 12 weeks old BL6/C7 mice that received one intraperitoneal injection of GapmeR (20 mg/kg). FIG. 5A shows Inc-019010 loss-of-function impact on cardiac functional parameters as assessed by echocardiography in vivo. FIG. 5B shows lnc-033521 losss-of-function impact on cardiac conduction parameters as assessed by electrocardiogram in vivo.

### DETAILED DESCRIPTION OF THE INVENTION

The Inventors set out to characterize the cardiac long non-coding transcriptome and in particular the dynamically modulated fraction post myocardial infarction. The Inventors coupled deep RNA-sequencing with ab initio transcript reconstruction, and integrated genome-wide data sets to systematically identify and annotate novel heart-specific lncRNAs.

Surprisingly, they showed that the lncRNAs of the invention are highly cardiac and context specific, correlating with cardiac physiology, suggesting a role as modulators of the pathological response and critical for physiological homeostasis. Using functional inference based on developmental chromatin state transitions, the Inventors functionally annotated these novel lncRNAs demonstrating that they are predominantly implicated with cardiac developmental, structural and functional gene programs. In particular, novel lncRNAs are predominantly associated with active enhancer states. The Inventors validated several novel lncRNAs in developmental and pathological models in vitro and in vivo and identified hundreds of predicted human orthologs and validated their expression in human samples. A number of these validated human orthologs were differentially expressed in human pathological cardiac states, supporting conserved roles in cardiac remodeling. Collectively, the Inventors have described a novel class of mammalian heart-specific lncRNAs with unique regulatory and functional characteristics, relevant to maladaptive pathological remodeling, cardiac function and potentially regeneration.

Accordingly, the present invention relates to a method for diagnosing a cardiac pathology in a subject, the method comprising:
a) measuring, directly or indirectly, the level of a plurality of biomarkers in a biological sample derived from the subject, wherein the plurality of biomarkers comprises one or more lncRNA having a cDNA sequence selected from the group comprising SEQ ID No 1, SEQ ID No 2, SEQ ID No 3, SEQ ID No 4, SEQ ID No 5, SEQ ID No 6, SEQ ID No 7, SEQ ID No 8, SEQ ID No 9, SEQ ID No 10, SEQ ID No 11, SEQ ID No 12, SEQ ID No 13, SEQ ID No 14, SEQ ID No 15, SEQ ID No 16, SEQ ID No 17, SEQ ID No 18, SEQ ID No 19, SEQ ID No 20, SEQ ID No 21, SEQ ID No 22, SEQ ID No 23, SEQ ID No 24, SEQ ID No 25, SEQ ID No 26, SEQ ID No 27, SEQ ID No 28, SEQ ID No 29, SEQ ID No 30, SEQ ID No 31, SEQ ID No 32, SEQ ID No 33, SEQ ID No 34, SEQ ID No 35, SEQ ID No 36, SEQ ID No 37, SEQ ID No 38, SEQ ID No 39, SEQ ID No 40, SEQ ID No 41, SEQ ID No 42, SEQ ID No 43, SEQ ID No 44, SEQ ID No 45, SEQ ID No 46, SEQ ID No 47, SEQ ID No 48, SEQ ID No 49, SEQ ID No 50, SEQ ID No 51, SEQ ID No 52, SEQ ID No 53, SEQ ID No 54, SEQ ID No 55, SEQ ID No 56, SEQ ID No 57, SEQ ID No 58, SEQ ID No 59, SEQ ID No 60, SEQ ID No 61, SEQ ID No 62, SEQ ID No 63, SEQ ID No 64, SEQ ID No 65, SEQ ID No 66, SEQ ID No 67, SEQ ID No 68, SEQ ID No 69, SEQ ID No 70, SEQ ID No 71, SEQ ID No 72, SEQ ID No 73, SEQ ID No 74, SEQ ID No 75, SEQ ID No 76, SEQ ID No 77, SEQ ID No 78, SEQ ID No 79, SEQ ID No 80, SEQ ID No 81, SEQ ID No 82, SEQ ID No 83, SEQ ID No 84, SEQ ID No 85, SEQ ID No 86, SEQ ID No 87, SEQ ID No 88, SEQ ID No 89, SEQ ID No 90, SEQ ID No 91, SEQ ID No 92, SEQ ID No 93, SEQ ID No 94, SEQ ID No 95, SEQ ID No 96, SEQ ID No 97, SEQ ID No 98, SEQ ID No 99, SEQ ID No 100, SEQ ID No 101, SEQ ID No 102, SEQ ID No103, and SEQ ID No 104, fragments thereof, isoforms thereof and variants sharing at least 80% nucleotide sequence identity thereto; and
b) analyzing the levels of said biomarkers, fragments thereof, isoforms thereof and variants sharing at least 80% nucleotide sequence identity thereto, in conjunction with respective reference value ranges for said plurality of biomarkers, wherein differential expression of one or more biomarkers in the biological sample compared to one or more biomarkers in a control sample from a normal subject indicates that the subject has a cardiac pathology.

Preferably, the biological sample derived from the subject is selected from the group comprising whole blood, serum, plasma, semen, saliva, tears, urine, fecal material, sweat, buccal smears, skin, cardiac tissue, liver, brain tissue, amniotic fluid, nerve tissue and hair. More preferably, the biological sample is cardiac tissue.

Usually, the cardiac pathology is selected from the group comprising Interventricular Septal Thickness (IVS), heart failure, EF, LVID, MI, HFrEF, viral myocarditis, brachycardia, arrhythmia, congenital heart defects, diabetic cardiomyopathy, idiopathic and dilated cardiomyopathy, pathologies characterized by malformation such as congenital heart disease and inherited heart disease; by tissue remodeling such as hypertrophic cardiomyopathies, dilated cardiomyopathies, hypertensive cardiomyopathies, ischemic heart disease, coronary heart disease, myocardial infarction and cardiac fibrosis; by affected function such as systolic dysfunction, diastolic dysfunction, heart failure with reduced ejection fraction and heart failure with preserved ejection fraction; by disorders of the right heart such as right ventricular heart failure, pulmonary hypertension and pulmonary embolism; by arrhythmias such as cardiac arrhythmias, fibrillation, channelopathies, syncope and sudden death; by valvular dysfunction such as valvular heart disease, valvular stenosis and valvular regurgitation; by inflammation such as viral, bacterial, protozoal and metazoal infection of the heart, myocarditis, pericarditis, endocarditis, cardiac disease associated to HIV infection, Chagas' disease and restrictive infiltrative cardiomyopathies; by intoxication such as toxin-induced cardiac disease, drug-induced cardiac disease, alcohol-induced cardiac disease, pharmaceutical-induced cardiac disease, chemical-induced cardiac disease, cardiac disease following exposure to heavy metals and cardiac complications of anti-cancer therapies; by cancer such as neoplastic infiltrative cardiomyopathies, carcinoid heart disease and primary tumors of the heart; by neurologic disorders such as muscular dystrophies; by autonomic disorders; by emotional stress such as cardiac disease associated to acute and chronic psychological stress; by metabolic disease such as diabetic cardiomyopathy, heart disease associated to endocrine disorders and mitochondrial disorders; by trauma such as traumatic heart disease, consequences of cardiac surgery and angioplasty; by a change in hemodynamic such as renal disease, thrombosis and rheumatic disease. Most preferably, the heart failure is heart failure with preserved or reduced ejection fraction.

The present invention also relates to a method for treating a cardiac pathology in a subject comprising administering to said subject an effective amount of a modulator of one or more lncRNAs having a cDNA sequence selected from the group comprising SEQ ID No 1, SEQ ID No 2, SEQ ID No 3, SEQ ID No 4, SEQ ID No 5, SEQ ID No 6, SEQ ID No 7, SEQ ID No 8, SEQ ID No 9, SEQ ID No 10, SEQ ID No 11, SEQ ID No 12, SEQ ID No 13, SEQ ID No 14, SEQ ID No 15, SEQ ID No 16, SEQ ID No 17, SEQ ID No 18, SEQ ID No 19, SEQ ID No 20, SEQ ID No 21, SEQ ID No 22, SEQ ID No 23, SEQ ID No 24, SEQ ID No 25, SEQ ID No 26, SEQ ID No 27, SEQ ID No 28, SEQ ID No 29, SEQ ID No 30, SEQ ID No 31, SEQ ID No 32, SEQ ID No 33, SEQ ID No 34, SEQ ID No 35, SEQ ID No 36, SEQ ID No 37, SEQ ID No 38, SEQ ID No 39, SEQ ID No 40, SEQ ID No 41, SEQ ID No 42, SEQ ID No 43, SEQ ID No 44, SEQ ID No 45, SEQ ID No 46, SEQ ID No 47, SEQ ID No 48, SEQ ID No 49, SEQ ID No 50, SEQ ID No 51, SEQ ID No 52, SEQ ID No 53, SEQ ID No 54, SEQ ID No 55, SEQ ID No 56, SEQ ID No 57, SEQ ID No 58, SEQ ID No 59, SEQ ID No 60, SEQ ID No 61, SEQ ID No 62, SEQ ID No 63, SEQ ID No 64, SEQ ID No 65, SEQ ID No 66, SEQ ID No 67, SEQ ID No 68, SEQ ID No 69, SEQ ID No 70, SEQ ID No 71, SEQ ID No 72, SEQ ID No 73, SEQ ID No 74, SEQ ID No 75, SEQ ID No 76, SEQ ID No 77, SEQ ID No 78, SEQ ID No 79, SEQ ID No 80, SEQ ID No 81, SEQ ID No 82, SEQ ID No 83, SEQ ID No 84, SEQ ID No 85, SEQ ID No 86, SEQ ID No 87, SEQ ID No 88, SEQ ID No 89, SEQ ID No 90, SEQ ID No 91, SEQ ID No 92, SEQ ID No 93, SEQ ID No 94, SEQ ID No 95, SEQ ID No 96, SEQ ID No 97, SEQ ID No 98, SEQ ID No 99, SEQ ID No 100, SEQ ID No 101, SEQ ID No 102, SEQ ID No103, and SEQ ID No 104, fragments thereof, isoforms thereof and variants sharing at least 80% nucleotide sequence identity thereto.

Usually, the modulator of one or more lncRNAs is selected from the group comprising a chemical agent, a RNA mimic, an antibody, an engineered protease, a CRISPR based technology and enzymatically active RNA.

Most preferably, the enzymatically active RNA is selected from the group comprising a miRNA, a siRNA, a piRNA, a hnRNA, a snRNA, esiRNA, shRNA, decoys, RNA aptamers and an antisense oligonucleotide. One will appreciate that any compound with different formulations capable to inhibit one or more physiological actions effected by lncRNA is encompassed by the present invention.

The siRNA of the invention may, e.g., comprise a nucleotide sequence as set forth in SEQ ID No 105, fragments thereof, isoforms thereof and variants sharing at least 80% nucleotide sequence identity thereto.

Another aspect of the present invention relates to a composition comprising a modulator of one or more lncRNAs wherein the modulator is selected from the group comprising a chemical agent, an antibody, an engineered protease, and enzymatically active RNA.

A method for modulating one or more lncRNAs wherein the modulator is selected from the group comprising the modulator of one or more lncRNAs is selected from the group comprising a chemical agent, a RNA mimic, an antibody, an engineered protease, and enzymatically active RNA is also part of the invention.

Preferably, the modulator modulates cardiac fibrosis, myopathy, hypertrophy, apoptosis, inflammation, extracellular remodeling, cardiac regeneration, CM and CF cell cycle and activation of endogenous CPCs, direct reprogramming of CF, ECs, in vitro reprogramming and differention of cell types for generation of cardiac cells for cell therapy, Cardiac epigenomic targeting of ubiquitous chromatin remodeling complexes, cardiac physiology and heart rate.

A further aspect of the present invention relates to a pharmaceutical composition comprising an effective amount of a modulator of one or more lncRNAs wherein the modulator is selected from the group comprising the modulator of one or more lncRNAs is selected from the group comprising a chemical agent, a RNA mimic, an antibody, an engineered protease, and enzymatically active RNA, optionally in combination with pharmaceutically acceptable carriers, diluents and/or adjuvants.

Another aspect of the invention relates to a kit comprising i) a composition comprising a modulator of one or more lncRNAs wherein the modulator is selected from the group comprising a chemical agent, a RNA mimic, an antibody, an engineered protease, and enzymatically active RNA or ii) a pharmaceutical composition comprising an effective amount of a modulator of one or more lncRNAs wherein the modulator is selected from the group comprising the modulator of one or more lncRNAs is selected from the group comprising a chemical agent, a RNA mimic, an antibody, an engineered protease, and enzymatically active RNA, optionally in combination with pharmaceutically acceptable carriers, diluents and/or adjuvants.

Also encompassed in the present invention is a method for diagnosing a cardiac pathology in a subject, the method comprising:
a) measuring, directly or indirectly, the level of a plurality of biomarkers in a biological sample derived from the subject, wherein the plurality of biomarkers comprises one or more lncRNAs selected from the group comprising SEQ ID No 1, SEQ ID No 2, SEQ ID No 3, SEQ ID No 4, SEQ ID No 5, SEQ ID No 6, SEQ ID No 7, SEQ ID No 8, SEQ ID No 9, SEQ ID No 10, SEQ ID No 11, SEQ ID No 12, SEQ ID No 13, SEQ ID No 14, SEQ ID No 15, SEQ ID No 16, SEQ ID No 17, SEQ ID No 18, SEQ ID No 19, SEQ ID No 20, SEQ ID No 21, SEQ ID No 22, SEQ ID No 23, SEQ ID No 24, SEQ ID No 25, SEQ ID No 26, SEQ ID No 27, SEQ ID No 28, SEQ ID No 29, SEQ ID No 30, SEQ ID No 31, SEQ ID No 32, SEQ ID No 33, SEQ ID No 34, SEQ ID No 35, SEQ ID No 36, SEQ ID No 37, SEQ ID No 38, SEQ ID No 39, SEQ ID No 40, SEQ ID No 41, SEQ ID No 42, SEQ ID No 43, SEQ ID No 44, SEQ ID No 45, SEQ ID No 46, SEQ ID No 47, SEQ ID No 48, SEQ ID No 49, SEQ ID No 50, SEQ ID No 51, SEQ ID No 52, SEQ ID No 53, SEQ ID No 54, SEQ ID No 55, SEQ ID No 56, SEQ ID No 57, SEQ ID No 58, SEQ ID No 59, SEQ ID No 60, SEQ ID No 61, SEQ ID No 62, SEQ ID No 63, SEQ ID No 64, SEQ ID No 65, SEQ ID No 66, SEQ ID No 67, SEQ ID No 68, SEQ ID No 69, SEQ ID No 70, SEQ ID No 71, SEQ ID No 72, SEQ ID No 73, SEQ ID No 74, SEQ ID No 75, SEQ ID No 76, SEQ ID No 77, SEQ ID No 78, SEQ ID No 79, SEQ ID No 80, SEQ ID No 81, SEQ ID No 82, SEQ ID No 83, SEQ ID No 84, SEQ ID No 85, SEQ ID No 86, SEQ ID No 87, SEQ ID No 88, SEQ ID No 89, SEQ ID No 90, SEQ ID No 91, SEQ ID No 92, SEQ ID No 93, SEQ ID No 94, SEQ ID No 95, SEQ ID No 96, SEQ ID No 97, SEQ ID No 98, SEQ ID No 99, SEQ ID No 100, SEQ ID No 101, SEQ ID No 102, SEQ ID No103, and SEQ ID No 104, fragments thereof, isoforms thereof and variants sharing at least 80% nucleotide sequence identity thereto; and
b) analyzing the levels of said biomarkers, fragments thereof, isoforms thereof and variants sharing at least 80% nucleotide sequence identity thereto, in conjunction with respective reference value ranges for said plurality of biomarkers, wherein differential expression of one or more biomarkers in the biological sample compared to one or more biomarkers in a control sample from a normal subject indicates that the subject has a cardiac pathology and/or a cardiac pathology thereby alleviating the need to execute echocardiography.

This invention also concerns a method for monitoring the effects of a treatment on cardiac tissue in a subject, the method comprising:
a) measuring, directly or indirectly, the level of a plurality of biomarkers in a biological sample derived from the subject, wherein the plurality of biomarkers comprises one or more lncRNAs selected from the group comprising SEQ ID No 1, SEQ ID No 2, SEQ ID No 3, SEQ ID No 4, SEQ ID No 5, SEQ ID No 6, SEQ ID No 7, SEQ ID No 8, SEQ ID No 9, SEQ ID No 10, SEQ ID No 11, SEQ ID No 12, SEQ ID No 13, SEQ ID No 14, SEQ ID No 15, SEQ ID No 16, SEQ ID No 17, SEQ ID No 18, SEQ ID No 19, SEQ ID No 20, SEQ ID No 21, SEQ ID No 22, SEQ ID No 23, SEQ ID No 24, SEQ ID No 25, SEQ ID No 26, SEQ ID No 27, SEQ ID No 28, SEQ ID No 29, SEQ ID No 30, SEQ ID No 31, SEQ ID No 32, SEQ ID No 33, SEQ ID No 34, SEQ ID No 35, SEQ ID No 36, SEQ ID No 37, SEQ ID No 38, SEQ ID No 39, SEQ ID No 40, SEQ ID No 41, SEQ ID No 42, SEQ ID No 43, SEQ ID No 44, SEQ ID No 45, SEQ ID No 46, SEQ ID No 47, SEQ ID No 48, SEQ ID No 49, SEQ ID No 50, SEQ ID No 51, SEQ ID No 52, SEQ ID No 53, SEQ ID No 54, SEQ ID No 55, SEQ ID No 56, SEQ ID No 57, SEQ ID No 58, SEQ ID No 59, SEQ ID No 60, SEQ ID No 61, SEQ ID No 62, SEQ ID No 63, SEQ ID No 64, SEQ ID No 65, SEQ ID No 66, SEQ ID No 67, SEQ ID No 68, SEQ ID No 69, SEQ ID No 70, SEQ ID No 71, SEQ ID No 72, SEQ ID No 73, SEQ ID No 74, SEQ ID No 75, SEQ ID No 76, SEQ ID No 77, SEQ ID No 78, SEQ ID No 79, SEQ ID No 80, SEQ ID No 81, SEQ ID No 82, SEQ ID No 83, SEQ ID No 84, SEQ ID No 85, SEQ ID No 86, SEQ ID No 87, SEQ ID No 88, SEQ ID No 89, SEQ ID No 90, SEQ ID No 91, SEQ ID No 92, SEQ ID No 93, SEQ ID No 94, SEQ ID No 95, SEQ ID No 96, SEQ ID No 97, SEQ ID No 98, SEQ ID No 99, SEQ ID No 100, SEQ ID No 101, SEQ ID No 102, SEQ ID No103, and SEQ ID No 104, fragments thereof, isoforms thereof and variants sharing at least 80% nucleotide sequence identity thereto; and
b) analyzing the levels of said biomarkers, fragments thereof, isoforms thereof and variants sharing at least 80% nucleotide sequence identity thereto, in conjunction with respective reference value ranges for said plurality of biomarkers, wherein differential expression of one or more biomarkers in the biological sample compared to one or more biomarkers in a control sample from a normal subject gives an indication on the effects of a treatment on cardiac tissue.

Further encompassed is a method for monitoring the efficacy of surgical and/or pharmacological cardiac therapies in a subject, the method comprising:
a) measuring, directly or indirectly, the level of a plurality of biomarkers in a biological sample derived from the subject, wherein the plurality of biomarkers comprises one or more lncRNAs selected from the group comprising SEQ ID No 1, SEQ ID No 2, SEQ ID No 3, SEQ ID No 4, SEQ ID No 5, SEQ ID No 6, SEQ ID No 7, SEQ ID No 8, SEQ ID No 9, SEQ ID No 10, SEQ ID No 11, SEQ ID No 12, SEQ ID No 13, SEQ ID No 14, SEQ ID No 15, SEQ ID No 16, SEQ ID No 17, SEQ ID No 18, SEQ ID No 19, SEQ ID No 20, SEQ ID No 21, SEQ ID No 22, SEQ ID No 23, SEQ ID No 24, SEQ ID No 25, SEQ ID No 26, SEQ ID No 27, SEQ ID No 28, SEQ ID No 29, SEQ ID No 30, SEQ ID No 31, SEQ ID No 32, SEQ ID No 33, SEQ ID No 34, SEQ ID No 35, SEQ ID No 36, SEQ ID No 37, SEQ ID No 38, SEQ ID No 39, SEQ ID No 40, SEQ ID No 41, SEQ ID No 42, SEQ ID No 43, SEQ ID No 44, SEQ ID No 45, SEQ ID No 46, SEQ ID No 47, SEQ ID No 48, SEQ ID No 49, SEQ ID No 50, SEQ ID No 51, SEQ ID No 52, SEQ ID No 53, SEQ ID No 54, SEQ ID No 55, SEQ ID No 56, SEQ ID No 57, SEQ ID No 58, SEQ ID No 59, SEQ ID No 60, SEQ ID No 61, SEQ ID No 62, SEQ ID No 63, SEQ ID No 64, SEQ ID No 65, SEQ ID No 66, SEQ ID No 67, SEQ ID No 68, SEQ ID No 69, SEQ ID No 70, SEQ ID No 71, SEQ ID No 72, SEQ ID No 73, SEQ ID No 74, SEQ ID No 75, SEQ ID No 76, SEQ ID No 77, SEQ ID No 78, SEQ ID No 79, SEQ ID No 80, SEQ ID No 81, SEQ ID No 82, SEQ ID No 83, SEQ ID No 84, SEQ ID No 85, SEQ ID No 86, SEQ ID No 87, SEQ ID No 88, SEQ ID No 89, SEQ ID No 90, SEQ ID No 91, SEQ ID No 92, SEQ ID No 93, SEQ ID No 94, SEQ ID No 95, SEQ ID No 96, SEQ ID No 97, SEQ ID No 98, SEQ ID No 99, SEQ ID No 100, SEQ ID No 101, SEQ ID No 102, SEQ ID No103, and SEQ ID No 104, fragments thereof, isoforms thereof and variants sharing at least 80% nucleotide sequence identity thereto; and
b) analyzing the levels of said biomarkers, fragments thereof, isoforms thereof and variants sharing at least 80% nucleotide sequence identity thereto, in conjunction with respective reference value ranges for said plurality of biomarkers, wherein differential expression of one or more biomarkers in the biological sample compared to one or more biomarkers in a control sample from a normal subject gives an indication on the efficacy of surgical and/or pharmacological cardiac therapies.

Also comprised in the present invention is a method for measuring cardiac tissue regeneration in a subject, the method comprising: a) measuring, directly or indirectly, the level of a plurality of biomarkers in a biological sample derived from the subject, wherein the plurality of biomarkers comprises one or more lncRNAs selected from the group comprising SEQ ID No 1, SEQ ID No 2, SEQ ID No 3, SEQ ID No 4, SEQ ID No 5, SEQ ID No 6, SEQ ID No 7, SEQ ID No 8, SEQ ID No 9, SEQ ID No 10, SEQ ID No 11, SEQ ID No 12, SEQ ID No 13, SEQ ID No 14, SEQ ID No 15, SEQ ID No 16, SEQ ID No 17, SEQ ID No 18, SEQ ID No 19, SEQ ID No 20, SEQ ID No 21, SEQ ID No 22, SEQ ID No 23, SEQ ID No 24, SEQ ID No 25, SEQ ID No 26, SEQ ID No 27, SEQ ID No 28, SEQ ID No 29, SEQ ID No 30, SEQ ID No 31, SEQ ID No 32, SEQ ID No 33, SEQ ID No 34, SEQ ID No 35, SEQ ID No 36, SEQ ID No 37, SEQ ID No 38, SEQ ID No 39, SEQ ID No 40, SEQ ID No 41, SEQ ID No 42, SEQ ID No 43, SEQ ID No 44, SEQ ID No 45, SEQ ID No 46, SEQ ID No 47, SEQ ID No 48, SEQ ID No 49, SEQ ID No 50, SEQ ID No 51, SEQ ID No 52, SEQ ID No 53, SEQ ID No 54, SEQ ID No 55, SEQ ID No 56, SEQ ID No 57, SEQ ID No 58, SEQ ID No 59, SEQ ID No 60, SEQ ID No 61, SEQ ID No 62, SEQ ID No 63, SEQ ID No 64, SEQ ID No 65, SEQ ID No 66, SEQ ID No 67, SEQ ID No 68, SEQ ID No 69, SEQ ID No 70, SEQ ID No 71, SEQ ID No 72, SEQ ID No 73, SEQ ID No 74, SEQ ID No 75, SEQ ID No 76, SEQ ID No 77, SEQ ID No 78, SEQ ID No 79, SEQ ID No 80, SEQ ID No 81, SEQ ID No 82, SEQ ID No 83, SEQ ID No 84, SEQ ID No 85, SEQ ID No 86, SEQ ID No 87, SEQ ID No 88, SEQ ID No 89, SEQ ID No 90, SEQ ID No 91, SEQ ID No 92, SEQ ID No 93, SEQ ID No 94, SEQ ID No 95, SEQ ID No 96, SEQ ID No 97, SEQ ID No 98, SEQ ID No 99, SEQ ID No 100, SEQ ID No 101, SEQ ID No 102, SEQ ID No103, and SEQ ID No 104, fragments thereof, isoforms thereof and variants sharing at least 80% nucleotide sequence identity thereto; and
b) analyzing the levels of said biomarkers, fragments thereof, isoforms thereof and variants sharing at least 80% nucleotide sequence identity thereto, in conjunction with respective reference value ranges for said plurality of biomarkers, wherein differential expression of one or more biomarkers in the biological sample compared to one or more biomarkers in a control sample from a normal subject gives an indication on cardiac tissue regeneration.

The present invention also comprises a method for monitoring in vitro cardiogenic cell differentiation, the method comprising:
a) measuring, directly or indirectly, the level of a plurality of biomarkers in a biological sample derived from a cell in culture, wherein the plurality of biomarkers comprises one or more lncRNAs selected from the group comprising SEQ ID No 1, SEQ ID No 2, SEQ ID No 3, SEQ ID No 4, SEQ ID No 5, SEQ ID No 6, SEQ ID No 7, SEQ ID No 8, SEQ ID No 9, SEQ ID No 10, SEQ ID No 11, SEQ ID No 12, SEQ ID No 13, SEQ ID No 14, SEQ ID No 15, SEQ ID No 16, SEQ ID No 17, SEQ ID No 18, SEQ ID No 19, SEQ ID No 20, SEQ ID No 21, SEQ ID No 22, SEQ ID No 23, SEQ ID No 24, SEQ ID No 25, SEQ ID No 26, SEQ ID No 27, SEQ ID No 28, SEQ ID No 29, SEQ ID No 30, SEQ ID No 31, SEQ ID No 32, SEQ ID No 33, SEQ ID No 34, SEQ ID No 35, SEQ ID No 36, SEQ ID No 37, SEQ ID No 38, SEQ ID No 39, SEQ ID No 40, SEQ ID No 41, SEQ ID No 42, SEQ ID No 43, SEQ ID No 44, SEQ ID No 45, SEQ ID No 46, SEQ ID No 47, SEQ ID No 48, SEQ ID No 49, SEQ ID No 50, SEQ ID No 51, SEQ ID No 52, SEQ ID No 53, SEQ ID No 54, SEQ ID No 55, SEQ ID No 56, SEQ ID No 57, SEQ ID No 58, SEQ ID No 59, SEQ ID No 60, SEQ ID No 61, SEQ ID No 62, SEQ ID No 63, SEQ ID No 64, SEQ ID No 65, SEQ ID No 66, SEQ ID No 67, SEQ ID No 68, SEQ ID No 69, SEQ ID No 70, SEQ ID No 71, SEQ ID No 72, SEQ ID No 73, SEQ ID No 74, SEQ ID No 75, SEQ ID No 76, SEQ ID No 77, SEQ ID No 78, SEQ ID No 79, SEQ ID No 80, SEQ ID No 81, SEQ ID No 82, SEQ ID No 83, SEQ ID No 84, SEQ ID No 85, SEQ ID No 86, SEQ ID No 87, SEQ ID No 88, SEQ ID No 89, SEQ ID No 90, SEQ ID No 91, SEQ ID No 92, SEQ ID No 93, SEQ ID No 94, SEQ ID No 95, SEQ ID No 96, SEQ ID No 97, SEQ ID No 98, SEQ ID No 99, SEQ ID No 100, SEQ ID No 101, SEQ ID No 102, SEQ ID No103, and SEQ ID No 104, fragments thereof, isoforms thereof and variants sharing at least 80% nucleotide sequence identity thereto; and
b) analyzing the levels of said biomarkers, fragments thereof, isoforms thereof and variants sharing at least 80% nucleotide sequence identity thereto, in conjunction with respective reference value ranges for said plurality of biomarkers, wherein differential expression of one or more biomarkers in the biological sample compared to one or more biomarkers in a control sample from a normal subject gives an indication on cardiogenic cell differentiation.

Another aspect of the invention concerns a method for monitoring in vitro cardiogenic cell differentiation, the method comprising:
a) measuring, directly or indirectly, the level of a plurality of biomarkers in a biological sample derived from a cell in culture, wherein the plurality of biomarkers comprises one or more lncRNAs selected from the group comprising SEQ ID No 1, SEQ ID No 2, SEQ ID No 3, SEQ ID No 4, SEQ ID No 5, SEQ ID No 6, SEQ ID No 7, SEQ ID No 8, SEQ ID No 9, SEQ ID No 10, SEQ ID No 11, SEQ ID No 12, SEQ ID No 13, SEQ ID No 14, SEQ ID No 15, SEQ ID No 16, SEQ ID No 17, SEQ ID No 18, SEQ ID No 19, SEQ ID No 20, SEQ ID No 21, SEQ ID No 22, SEQ ID No 23, SEQ ID No 24, SEQ ID No 25, SEQ ID No 26, SEQ ID No 27, SEQ ID No 28, SEQ ID No 29, SEQ ID No 30, SEQ ID No 31, SEQ ID No 32, SEQ ID No 33, SEQ ID No 34, SEQ ID No 35, SEQ ID No 36, SEQ ID No 37, SEQ ID No 38, SEQ ID No 39, SEQ ID No 40, SEQ ID No 41, SEQ ID No 42, SEQ ID No 43, SEQ ID No 44, SEQ ID No 45, SEQ ID No 46, SEQ ID No 47, SEQ ID No 48, SEQ ID No 49, SEQ ID No 50, SEQ ID No 51, SEQ ID No 52, SEQ ID No 53, SEQ ID No 54, SEQ ID No 55, SEQ ID No 56, SEQ ID No 57, SEQ ID No 58, SEQ ID No 59, SEQ ID No 60, SEQ ID No 61, SEQ ID No 62, SEQ ID No 63, SEQ ID No 64, SEQ ID No 65, SEQ ID No 66, SEQ ID No 67, SEQ ID No 68, SEQ ID No 69, SEQ ID No 70, SEQ ID No 71, SEQ ID No 72, SEQ ID No 73, SEQ ID No 74, SEQ ID No 75, SEQ ID No 76, SEQ ID No 77, SEQ ID No 78, SEQ ID No 79, SEQ ID No 80, SEQ ID No 81, SEQ ID No 82, SEQ ID No 83, SEQ ID No 84, SEQ ID No 85, SEQ ID No 86, SEQ ID No 87, SEQ ID No 88, SEQ ID No 89, SEQ ID No 90, SEQ ID No 91, SEQ ID No 92, SEQ ID No 93, SEQ ID No 94, SEQ ID No 95, SEQ ID No 96, SEQ ID No 97, SEQ ID No 98, SEQ ID No 99, SEQ ID No 100, SEQ ID No 101, SEQ ID No 102, SEQ ID No103, and SEQ ID No 104, fragments thereof, isoforms thereof and variants sharing at least 80% nucleotide sequence identity thereto; and
b) analyzing the levels of said biomarkers, fragments thereof, isoforms thereof and variants sharing at least 80% nucleotide sequence identity thereto, in conjunction with respective reference value ranges for said plurality of biomarkers, wherein differential expression of one or more biomarkers in the biological sample compared to one or more biomarkers in a control sample from a normal subject gives an indication on cardiogenic cell differentiation.

Another aspect of the invention concerns a method for monitoring efficacy of agents and/or small molecules that can induce cell reprogramming, the method comprising:
a) measuring, directly or indirectly, the level of a plurality of biomarkers in a biological sample derived from a subject or a cell in culture, wherein the plurality of biomarkers comprises one or more lncRNAs selected from the group comprising SEQ ID No 1, SEQ ID No 2, SEQ ID No 3, SEQ ID No 4, SEQ ID No 5, SEQ ID No 6, SEQ ID No 7, SEQ ID No 8, SEQ ID No 9, SEQ ID No 10, SEQ ID No 11, SEQ ID No 12, SEQ ID No 13, SEQ ID No 14, SEQ ID No 15, SEQ ID No 16, SEQ ID No 17, SEQ ID No 18, SEQ ID No 19, SEQ ID No 20, SEQ ID No 21, SEQ ID No 22, SEQ ID No 23, SEQ ID No 24, SEQ ID No 25, SEQ ID No 26, SEQ ID No 27, SEQ ID No 28, SEQ ID No 29, SEQ ID No 30, SEQ ID No 31, SEQ ID No 32, SEQ ID No 33, SEQ ID No 34, SEQ ID No 35, SEQ ID No 36, SEQ ID No 37, SEQ ID No 38, SEQ ID No 39, SEQ ID No 40, SEQ ID No 41, SEQ ID No 42, SEQ ID No 43, SEQ ID No 44, SEQ ID No 45, SEQ ID No 46, SEQ ID No 47, SEQ ID No 48, SEQ ID No 49, SEQ ID No 50, SEQ ID No 51, SEQ ID No 52, SEQ ID No 53, SEQ ID No 54, SEQ ID No 55, SEQ ID No 56, SEQ ID No 57, SEQ ID No 58, SEQ ID No 59, SEQ ID No 60, SEQ ID No 61, SEQ ID No 62, SEQ ID No 63, SEQ ID No 64, SEQ ID No 65, SEQ ID No 66, SEQ ID No 67, SEQ ID No 68, SEQ ID No 69, SEQ ID No 70, SEQ ID No 71, SEQ ID No 72, SEQ ID No 73, SEQ ID No 74, SEQ ID No 75, SEQ ID No 76, SEQ ID No 77, SEQ ID No 78, SEQ ID No 79, SEQ ID No 80, SEQ ID No 81, SEQ ID No 82, SEQ ID No 83, SEQ ID No 84, SEQ ID No 85, SEQ ID No 86, SEQ ID No 87, SEQ ID No 88, SEQ ID No 89, SEQ ID No 90, SEQ ID No 91, SEQ ID No 92, SEQ ID No 93, SEQ ID No 94, SEQ ID No 95, SEQ ID No 96, SEQ ID No 97, SEQ ID No 98, SEQ ID No 99, SEQ ID No 100, SEQ ID No 101, SEQ ID No 102, SEQ ID No103, and SEQ ID No 104, fragments thereof, isoforms thereof and variants sharing at least 80% nucleotide sequence identity thereto; and
b) analyzing the levels of said biomarkers, fragments thereof, isoforms thereof and variants sharing at least 80% nucleotide sequence identity thereto, in conjunction with respective reference value ranges for said plurality of biomarkers, wherein differential expression of one or more biomarkers in the biological sample compared to one or more biomarkers in a control sample from a normal subject gives an indication on efficacy of agents and/or small molecules that can induce cardiac reprogramming.

Another aspect of the invention concerns a microarray comprising a plurality of probes that hybridize to one or more lncRNAs selected from the group comprising SEQ ID No 1, SEQ ID No 2, SEQ ID No 3, SEQ ID No 4, SEQ ID No 5, SEQ ID No 6, SEQ ID No 7, SEQ ID No 8, SEQ ID No 9, SEQ ID No 10, SEQ ID No 11, SEQ ID No 12, SEQ ID No 13, SEQ ID No 14, SEQ ID No 15, SEQ ID No 16, SEQ ID No 17, SEQ ID No 18, SEQ ID No 19, SEQ ID No 20, SEQ ID No 21, SEQ ID No 22, SEQ ID No 23, SEQ ID No 24, SEQ ID No 25, SEQ ID No 26, SEQ ID No 27, SEQ ID No 28, SEQ ID No 29, SEQ ID No 30, SEQ ID No 31, SEQ ID No 32, SEQ ID No 33, SEQ ID No 34, SEQ ID No 35, SEQ ID No 36, SEQ ID No 37, SEQ ID No 38, SEQ ID No 39, SEQ ID No 40, SEQ ID No 41, SEQ ID No 42, SEQ ID No 43, SEQ ID No 44, SEQ ID No 45, SEQ ID No 46, SEQ ID No 47, SEQ ID No 48, SEQ ID No 49, SEQ ID No 50, SEQ ID No 51, SEQ ID No 52, SEQ ID No 53, SEQ ID No 54, SEQ ID No 55, SEQ ID No 56, SEQ ID No 57, SEQ ID No 58, SEQ ID No 59, SEQ ID No 60, SEQ ID No 61, SEQ ID No 62, SEQ ID No 63, SEQ ID No 64, SEQ ID No 65, SEQ ID No 66, SEQ ID No 67, SEQ ID No 68, SEQ ID No 69, SEQ ID No 70, SEQ ID No 71, SEQ ID No 72, SEQ ID No 73, SEQ ID No 74, SEQ ID No 75, SEQ ID No 76, SEQ ID No 77, SEQ ID No 78, SEQ ID No 79, SEQ ID No 80, SEQ ID No 81, SEQ ID No 82, SEQ ID No 83, SEQ ID No 84, SEQ ID No 85, SEQ ID No 86, SEQ ID No 87, SEQ ID No 88, SEQ ID No 89, SEQ ID No 90, SEQ ID No 91, SEQ ID No 92, SEQ ID No 93, SEQ ID No 94, SEQ ID No 95, SEQ ID No 96, SEQ ID No 97, SEQ ID No 98, SEQ ID No 99, SEQ ID No 100, SEQ ID No 101, SEQ ID No 102, SEQ ID No103, and SEQ ID No 104, fragments thereof, isoforms thereof and variants sharing at least 80% nucleotide sequence identity thereto.

In describing the present invention, the following terms will be employed, and are intended to be defined as indicated below.

It must be noted that, as used in this specification and the appended claims, the singular forms “a,” “an” and “the” include plural referents unless the content clearly dictates otherwise. Thus, for example, reference to “a lncRNA” includes a mixture of two or more lncRNAs, and the like.

The term “about,” particularly in reference to a given quantity, is meant to encompass deviations of plus or minus ten percent.

The terms “microRNA,” “miRNA,” and MiR” are interchangeable and refer to endogenous or artificial non-coding RNAs that are capable of regulating gene expression. It is believed that miRNAs function via RNA interference. The terms “siRNA” and “short interfering RNA” are interchangeable and refer to single-stranded or double-stranded RNA molecules that are capable of inducing RNA interference. SiRNA molecules typically have a duplex region that is between 18 and 30 base pairs in length.

The terms “piRNA” and “Piwi-interacting RNA” are interchangeable and refer to a class of small RNAs involved in gene silencing. PiRNA molecules typically are between 26 and 31 nucleotides in length.

The terms “snRNA” and “small nuclear RNA” are interchangeable and refer to a class of small RNAs involved in a variety of processes including RNA splicing and regulation of transcription factors. The subclass of small nucleolar RNAs (snoRNAs) is also included. The term is also intended to include artificial snRNAs, such as antisense derivatives of snRNAs comprising antisense sequences directed against one or more lncRNAs.

The terms “polynucleotide,” “oligonucleotide,” “nucleic acid” and “nucleic acid molecule” are used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), any other type of polynucleotide which is an N- or C-glycoside of a purine or pyrimidine base, and other polymers containing nonnucleotidic backbones, for example, polyamide (e.g., peptide nucleic acids (PNAs)) and polymorpholino (commercially available from the Anti-Virals, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA. There is no intended distinction in length between the terms “polynucleotide,” “oligonucleotide,” “nucleic acid” and “nucleic acid molecule,” and these terms will be used interchangeably. Thus, these terms include, for example, 3′-deoxy-2′,5′-DNA, oligodeoxyribonucleotide N3′ P5′ phosphoramidates, 2′-O-alkyl-substituted RNA, double- and single-stranded DNA, as well as double- and single-stranded RNA, microRNA, DNA:RNA hybrids, and hybrids between PNAs and DNA or RNA, and also include known types of modifications, for example, labels which are known in the art, methylation, “caps,” substitution of one or more of the naturally occurring nucleotides with an analog (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide. The term also includes locked nucleic acids (e.g., comprising a ribonucleotide that has a methylene bridge between the 2′-oxygen atom and the 4′-carbon atom). See, for example, Kurreck et al. (2002) Nucleic Acids Res. 30:1911-1918.

The term “complementary” and “complementarity” are interchangeable and refer to the ability of polynucleotides to form base pairs with one another. Base pairs are typically formed by hydrogen bonds between nucleotide units in antiparallel polynucleotide strands or regions. Complementary polynucleotide strands or regions can base pair in the Watson-Crick manner (e.g., A to T, A to U, C to G). 100% complementary refers to the situation in which each nucleotide unit of one polynucleotide strand or region can hydrogen bond with each nucleotide unit of a second polynucleotide strand or region. Less than perfect complementarity refers to the situation in which some, but not all, nucleotide units of two strands or two regions can hydrogen bond with each other and can be expressed as a percentage.

“Administering”, as it applies in the present invention, refers to contact of an effective amount of a modulator of one or more lncRNAs of the invention, to the subject.

Admnistering a nucleic acid, such as a microRNA, siRNA, piRNA, snRNA, antisense nucleic acid, or lncRNA to a cell comprises transducing, transfecting, electroporating, translocating, fusing, phagocytosing, shooting or ballistic methods, etc., i.e., any means by which a nucleic acid can be transported across a cell membrane.

“Pharmaceutically acceptable excipient or carrier” refers to an excipient that may optionally be included in the compositions of the invention and that causes no significant adverse toxicological effects to the patient.

“Pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly salts containing pharmaceutically acceptable cations include, but are not limited to, sodium, potassium, calcium, aluminum, lithium, and ammonium (including substituted ammonium).

An “effective amount” of modulator of one or more lncRNAs of the invention (e.g., microRNA, siRNA, piRNA, snRNA, antisense nucleic acid, ribozyme, or small molecule inhibitor, CRISPRs etc) is an amount sufficient to effect beneficial or desired results, such as an amount that inhibits the activity of a lncRNA, for example by interfering with transcription. An effective amount can be administered in one or more administrations, applications, or dosages.

By “therapeutically effective dose or amount” of a modulator of one or more lncRNAs of the invention is intended an amount that, when administered as described herein, brings about a positive therapeutic response. The exact amount required will vary from subject to subject, depending on the species, age, and general condition of the subject, the severity of the condition being treated, the particular drug or drugs employed, mode of administration, and the like. An appropriate “effective” amount in any individual case may be determined by one of ordinary skill in the art using routine experimentation, based upon the information provided herein.

“Homology” refers to the percent identity between two polynucleotide or two polypeptide moieties. Two nucleic acid, or two polypeptide sequences are “substantially homologous” to each other when the sequences exhibit at least about 50% sequence identity, preferably at least about 75% sequence identity, more preferably at least about 80%-85% sequence identity, more preferably at least about 90% sequence identity, and most preferably at least about 95%-98% sequence identity over a defined length of the molecules. As used herein, substantially homologous also refers to sequences showing complete identity to the specified sequence.

In general, “identity” refers to an exact nucleotide to nucleotide or amino acid to amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100.

Alternatively, homology can be determined by readily available computer programs or by hybridization of polynucleotides under conditions which form stable duplexes between homologous regions, followed by digestion with single stranded specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art.

“Recombinant” as used herein to describe a nucleic acid molecule means a polynucleotide of genomic, cDNA, viral, semisynthetic, or synthetic origin which, by virtue of its origin or manipulation, is not associated with all or a portion of the polynucleotide with which it is associated in nature. The term “recombinant” as used with respect to a protein or polypeptide means a polypeptide produced by expression of a recombinant polynucleotide. In general, the gene of interest is cloned and then expressed in transformed organisms, as described further below. The host organism expresses the foreign gene to produce the protein under expression conditions.

The term “transformation” refers to the insertion of an exogenous polynucleotide into a host cell, irrespective of the method used for the insertion. For example, direct uptake, transduction or f-mating are included. The exogenous polynucleotide may be maintained as a non-integrated vector, for example, a plasmid, or alternatively, may be integrated into the host genome.

“Recombinant host cells”, “host cells,” “cells”, “cell lines,” “cell cultures”, and other such terms denoting microorganisms or higher eukaryotic cell lines cultured as unicellular entities refer to cells which can be, or have been, used as recipients for recombinant vector or other transferred DNA, and include the original progeny of the original cell which has been transfected.

“Purified polynucleotide” refers to a polynucleotide of interest or fragment thereof which is essentially free, e.g., contains less than about 50%, preferably less than about 70%, and more preferably less than about at least 90%, of the protein with which the polynucleotide is naturally associated. Techniques for purifying polynucleotides of interest are well-known in the art and include, for example, disruption of the cell containing the polynucleotide with a chaotropic agent and separation of the polynucleotide(s) and proteins by ion-exchange chromatography, affinity chromatography and sedimentation according to density.

A “vector” is capable of transferring nucleic acid sequences to target cells (e.g., viral vectors, non-viral vectors, particulate carriers, and liposomes). Typically, “vector construct,” “expression vector,” and “gene transfer vector,” mean any nucleic acid construct capable of directing the expression of a nucleic acid of interest and which can transfer nucleic acid sequences to target cells. Thus, the term includes cloning and expression vehicles, as well as viral vectors.

The terms “variant” refers to biologically active derivatives of a biomarker, i.e. one or more lncRNAs. In general, the term “variant” refers to molecules having a native sequence and structure with one or more additions, substitutions (generally conservative in nature) and/or deletions, relative to the native molecule, so long as the modifications do not destroy biological activity and which are “substantially homologous” to the reference molecule. In general, the sequences of such variants will have a high degree of sequence homology to the reference sequence, e.g., sequence homology of more than 50%, generally more than 60%-70%, even more particularly 80%-85% or more, such as at least 90%-95% or more, when the two sequences are aligned.

Alternatively, the term “variant” also refers to post-transcriptionaly modified lncRNAs of the invention, i.e methylation, phosphorylation, etc.,

A “biomarker” in the context of the present invention refers to an lncRNA which is differentially expressed in a biological sample (e.g., a biopsy taken from a subject having a cardiac pathology or a cardiac tissue undergoing regeneration or a stem cell undergoing cardiac differentiation or a cardiac tissue undergoing surgical and/or pharmacological therapies) as compared to a control sample (e.g., a comparable sample taken from a person with a negative diagnosis, a normal or healthy subject, or normal, untreated tissue or cells). The biomarker can be an lncRNA that can be detected and/or quantified.

One will appreciate that the control sample can vary depending on the situation. For example, the control sample can include a cell or sample of cells that provide a reference expression level of the same gene. Alternatively, the control sample can be healthy cells from the same source tissue as the target cell(s).

As used herein, an “isoform” of an lncRNA results from alternative splicing of the gene encoding said lncRNA.

As used herein, a “fragment” one or more lncRNAs refers to a sequence containing less amino acids in length than the respective one or more lncRNA. Preferably, this sequence contains less than 90%, preferably less than 60%, in particular less than 30% amino acids in length than the respective one or more lncRNA.

Biomarkers are one or more lncRNAs selected from the group comprising the cDNA sequences: SEQ ID No 1, SEQ ID No 2, SEQ ID No 3, SEQ ID No 4, SEQ ID No 5, SEQ ID No 6, SEQ ID No 7, SEQ ID No 8, SEQ ID No 9, SEQ ID No 10, SEQ ID No 11, SEQ ID No 12, SEQ ID No 13, SEQ ID No 14, SEQ ID No 15, SEQ ID No 16, SEQ ID No 17, SEQ ID No 18, SEQ ID No 19, SEQ ID No 20, SEQ ID No 21, SEQ ID No 22, SEQ ID No 23, SEQ ID No 24, SEQ ID No 25, SEQ ID No 26, SEQ ID No 27, SEQ ID No 28, SEQ ID No 29, SEQ ID No 30, SEQ ID No 31, SEQ ID No 32, SEQ ID No 33, SEQ ID No 34, SEQ ID No 35, SEQ ID No 36, SEQ ID No 37, SEQ ID No 38, SEQ ID No 39, SEQ ID No 40, SEQ ID No 41, SEQ ID No 42, SEQ ID No 43, SEQ ID No 44, SEQ ID No 45, SEQ ID No 46, SEQ ID No 47, SEQ ID No 48, SEQ ID No 49, SEQ ID No 50, SEQ ID No 51, SEQ ID No 52, SEQ ID No 53, SEQ ID No 54, SEQ ID No 55, SEQ ID No 56, SEQ ID No 57, SEQ ID No 58, SEQ ID No 59, SEQ ID No 60, SEQ ID No 61, SEQ ID No 62, SEQ ID No 63, SEQ ID No 64, SEQ ID No 65, SEQ ID No 66, SEQ ID No 67, SEQ ID No 68, SEQ ID No 69, SEQ ID No 70, SEQ ID No 71, SEQ ID No 72, SEQ ID No 73, SEQ ID No 74, SEQ ID No 75, SEQ ID No 76, SEQ ID No 77, SEQ ID No 78, SEQ ID No 79, SEQ ID No 80, SEQ ID No 81, SEQ ID No 82, SEQ ID No 83, SEQ ID No 84, SEQ ID No 85, SEQ ID No 86, SEQ ID No 87, SEQ ID No 88, SEQ ID No 89, SEQ ID No 90, SEQ ID No 91, SEQ ID No 92, SEQ ID No 93, SEQ ID No 94, and SEQ ID No 95, SEQ ID No 96, SEQ ID No 97, SEQ ID No 98, SEQ ID No 99, SEQ ID No 100, SEQ ID No 101, SEQ ID No 102, SEQ ID No103, and SEQ ID No 104, fragments thereof, isoforms thereof and variants sharing at least 80% nucleotide sequence identity thereto. Most preferably, the one or more lncRNAs is selected from the group comprising the cDNA sequences SEQ ID No 25, SEQ ID No 28, SEQ ID No 48, SEQ ID No 52, SEQ ID No 53, SEQ ID No 82, SEQ ID No 84, SEQ ID No 88, SEQ ID No 96, SEQ ID No 97, SEQ ID No 98, SEQ ID No 99, SEQ ID No 100, SEQ ID No 101, SEQ ID No 103, and SEQ ID No 104, fragments thereof, isoforms thereof and variants sharing at least 80% nucleotide sequence identity thereto.

TABLE 1

| | SEQ | Physiology | |
|---|---|---|---|
| XLOC_ID | ID No. | Cluster | Lnc |
| XLOC_007900 | 1 | 1 | Lnc7900 |
| XLOC_008052 | 2 | 1 | Lnc8052 |
| XLOC_006224 | 3 | 1 | Lnc6224 |
| XLOC_013471 | 4 | 1 | Lnc13471 |
| XLOC_002075 | 5 | 1 | Lnc2075 |
| XLOC_023749 | 6 | 1 | Lnc23749 |
| XLOC_008063 | 7 | 1 | Lnc8063 |
| XLOC_024203 | 8 | 1 | Lnc24203 |
| XLOC_019889 | 9 | 1 | Lnc19889 |
| XLOC_008229 | 10 | 1 | Lnc8229 |
| XLOC_014116 | 11 | 1 | Lnc14116/Novlnc11 |
| XLOC_003166 | 12 | 1 | Lnc3166 |
| XLOC_010335 | 13 | 1 | Lnc10335 |
| XLOC_021863 | 14 | 1 | Lnc21863 |
| XLOC_012367 | 15 | 1 | Lnc12367 |
| XLOC_007833 | 16 | 1 | Lnc7833 |
| XLOC_023850 | 17 | 2 | Lnc23850 |
| XLOC_029624 | 18 | 2 | Lnc-TEAD1 |
| XLOC_022865 | 19 | 2 | Lnc-SE-22865 |
| XLOC_018239 | 20 | 2 | Lnc18239 |
| XLOC_022715 | 21 | 2 | Lnc-COL16A1 |
| XLOC_013413 | 22 | 2 | Lnc13413 |
| XLOC_005390 | 23 | 2 | Lnc-MEOX1 |
| XLOC_010961 | 24 | 2 | Lnc-WISP1 |
| XLOC_000709 | 25 | 2 | Lnc-TGFB2/Novlnc11 |
| XLOC_013407 | 26 | 2 | Lnc-SLC8A1 |
| XLOC_020214 | 27 | 2 | Lnc-CYR61 |
| XLOC_012723 | 28 | 2 | Lnc-SE-12723/Novlnc174 |
| XLOC_022262 | 29 | 2 | Lnc22262 |
| XLOC_000719 | 30 | 2 | Lnc00719 |
| XLOC_004951 | 31 | 2 | Lnc4951 |
| XLOC_026589 | 32 | 2 | Lnc26589 |
| XLOC_019010 | 33 | 2 | Lnc19010 |
| XLOC_022236 | 34 | 2 | Lnc22236 |
| XLOC_011236 | 35 | 3 | Lnc-SLC38A2 |
| XLOC_012015 | 36 | 3 | Lnc-KCNJ6 |
| XLOC_012884 | 37 | 3 | Lnc-NKX2.5 |
| XLOC_004797 | 38 | 3 | Lnc4797 |
| XLOC_003851 | 39 | 3 | Lnc-SPNB2 |
| XLOC_011237 | 40 | 3 | Lnc-SLC38A2 |
| XLOC_014898 | 41 | 3 | Lnc-SE-14989 |
| XLOC_030839 | 42 | 3 | Lnc-CDH13 |
| XLOC_012194 | 43 | 3 | Lnc-ACAP2 |
| XLOC_031308 | 44 | 3 | Lnc-IRX3 |
| XLOC_026621 | 45 | 3 | Lnc-ATOH8 |
| XLOC_002721 | 46 | 3 | Lnc-TXLNB |
| XLOC_003170 | 47 | 3 | Lnc-KITLG |
| XLOC_002849 | 48 | 4 | Lnc-Novlnc6 |
| XLOC_016279 | 49 | 4 | Lnc-Novlnc25 |
| XLOC_024141 | 50 | 4 | Lnc-CARD11 |
| XLOC_021524 | 51 | 4 | Lnc-NFIB |
| XLOC_021715 | 52 | 4 | Lnc-FOXO6 |
| XLOC_020321 | 53 | 4 | Lnc-ANX5A |
| XLOC_006274 | 54 | 4 | Lnc-MAX |
| XLOC_021416 | 55 | 4 | Lnc21416 |
| XLOC_003767 | 56 | 4 | Lnc-LIF |
| XLOC_014118 | 57 | 4 | Lnc-LCLAT1 |
| XLOC_004833 | 58 | 4 | Lnc4833 |
| XLOC_009582 | 59 | 4 | Lnc-PPIF |
| XLOC_024449 | 60 | 4 | Lnc24449 |
| XLOC_006146 | 61 | 4 | Lnc6146 |
| XLOC_033521 | 62 | 4 | Lnc-Dedbt |
| | | | (Lnc033521) |
| XLOC_025643 | 63 | 4 | Lnc25643 |
| XLOC_004910 | 64 | 4 | Lnc-SPARC |
| XLOC_010967 | 65 | 4 | Lnc-miR30b |
| XLOC_002503 | 66 | 4 | Lnc-SOCS2 |
| XLOC_017764 | 67 | 4 | Lnc-ID1 |
| XLOC_020119 | 68 | 4 | Lnc20119 |
| XLOC_001065 | 69 | 4 | Lnc-GPC1 |
| XLOC_009131 | 70 | 4 | Lnc-OTX2 |
| XLOC_000264 | 71 | 4 | Lnc-FAM124B |
| XLOC_032325 | 72 | 4 | Lnc-TALIN1 |
| XLOC_002546 | 73 | 4 | Lnc-KRR1 |
| XLOC_006241 | 74 | 4 | Lnc-DACT1 |
| XLOC_029781 | 75 | 4 | Lnc29781 |
| XLOC_030722 | 76 | 4 | Lnc-SE-30722 |
| XLOC_032031 | 77 | 4 | Lnc-KCNJ |
| XLOC_020634 | 78 | 4 | Lnc-SE-20634 |
| XLOC_031524 | 79 | 4 | Lnc-IRF2BP2 |
| XLOC_020212 | 80 | 4 | Lnc-CYR61 |
| XLOC_000336 | 81 | 4 | Lnc-HDAC4 |
| XLOC_015960 | 82 | 4 | Lnc-ITPRIP |
| XLOC_004067 | 83 | 4 | Lnc-MYOCD |
| XLOC_015277 | 84 | 4 | Lnc-SMAD7/Novlnc23 |
| XLOC_020313 | 85 | 4 | Lnc20313 |
| XLOC_008190 | 86 | 4 | Lnc8190 |
| XLOC_033125 | 87 | 4 | Lnc33125 |
| XLOC_032788 | 88 | 4 | Lnc32788/Novlnc90 |
| XLOC_014917 | 89 | 4 | Lnc14917 |
| XLOC_014935 | 90 | 4 | Lnc14935 |
| XLOC_007419 | 91 | 4 | Lnc7419 |
| XLOC_006561 | 92 | 4 | Lnc6561 |
| XLOC_024370 | 93 | 4 | Lnc24370 |
| XLOC_006255 | 94 | 4 | Lnc6255 |
| XLOC_029637 | 95 | 4 | Lnc29637 |
| XLOC_010855 | 96 | 2 | Lnc10855 |
| XLOC_007852 | 97 | 4 | Lnc7852/Novlnc15 |
| XLOC_009335 | 98 | 4 | Lnc9335/Novlnc32 |
| XLOC_019782 | 99 | 2 | Lnc19782/Novlnc35 |
| XLOC_010735 | 100 | 4 | Lnc10735/Novlnc44 |
| XLOC_007917 | 101 | 4 | Lnc7917/Novlnc61 |
| XLOC_033357 | 102 | 4 | Lnc33357 |
| XLOC_023848 | 103 | 4 | Lnc23848/Novlnc49 |
| XLOC_016979 | 104 | 4 | Lnc16979 |

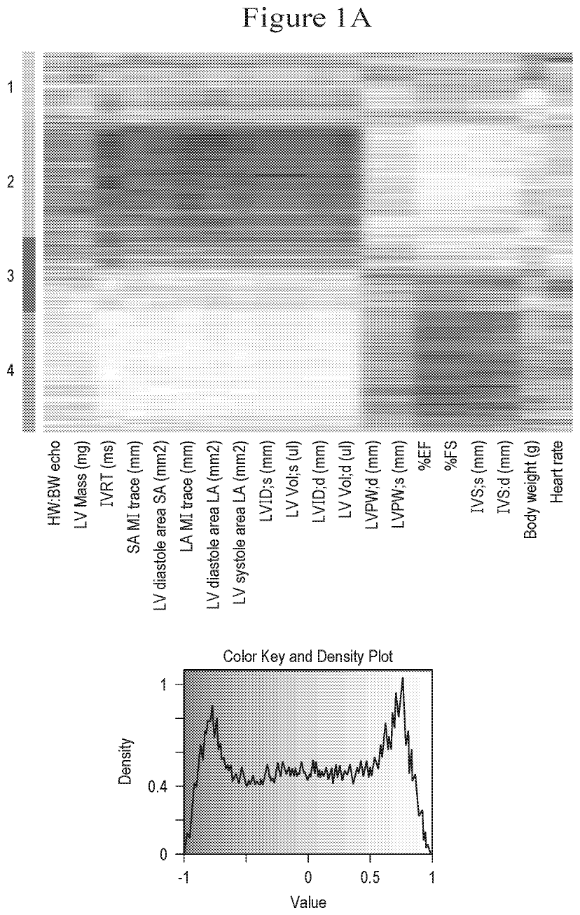

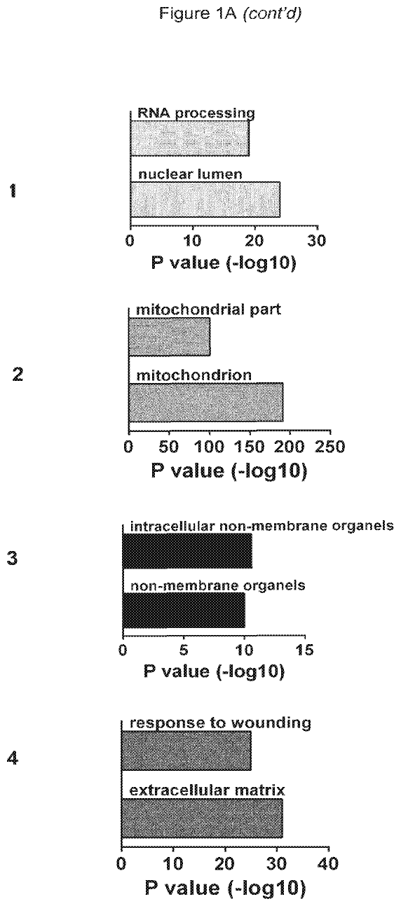

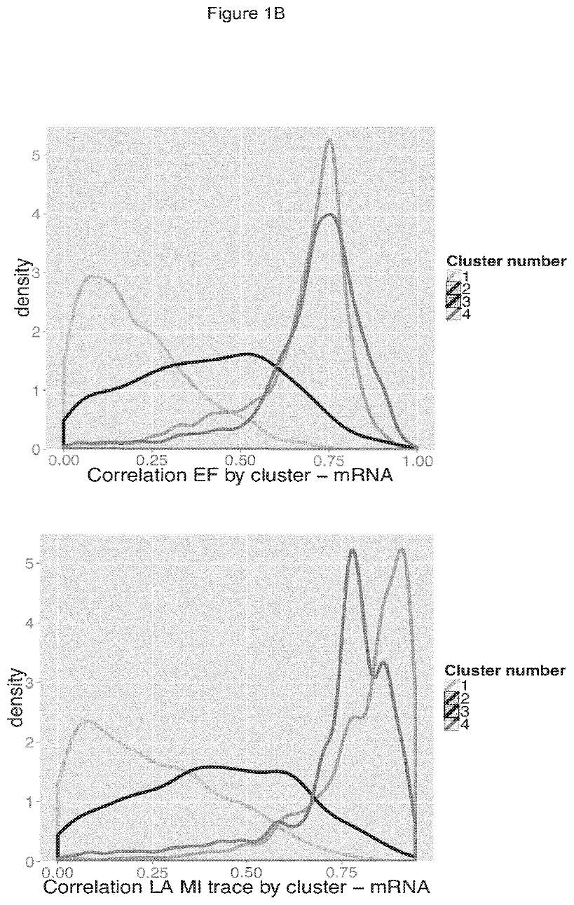

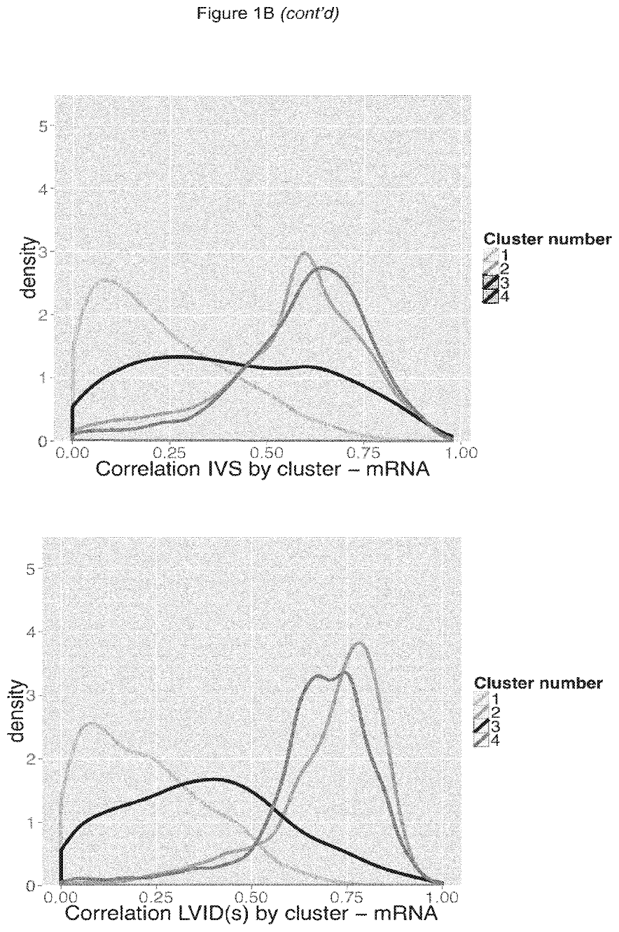

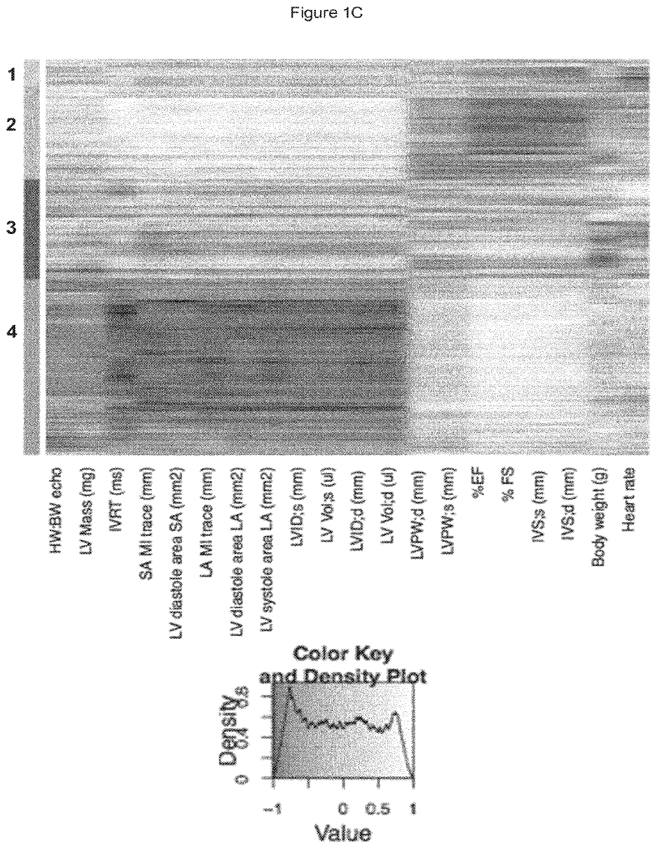

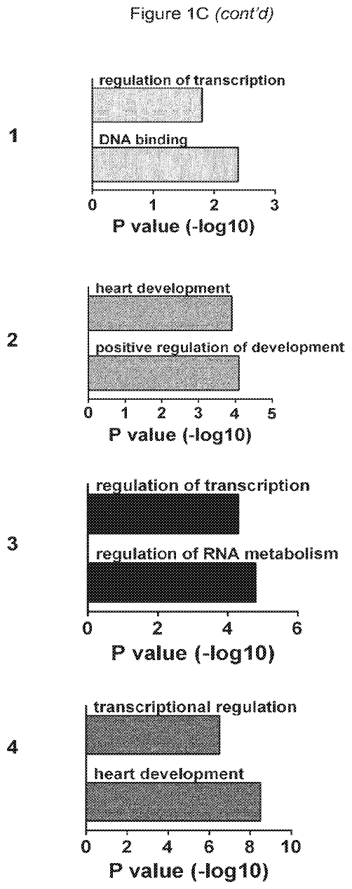

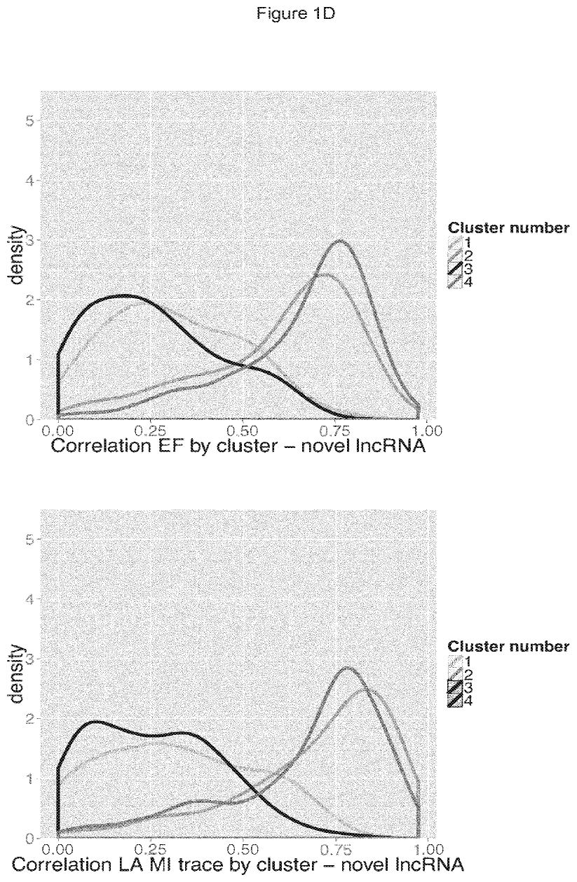

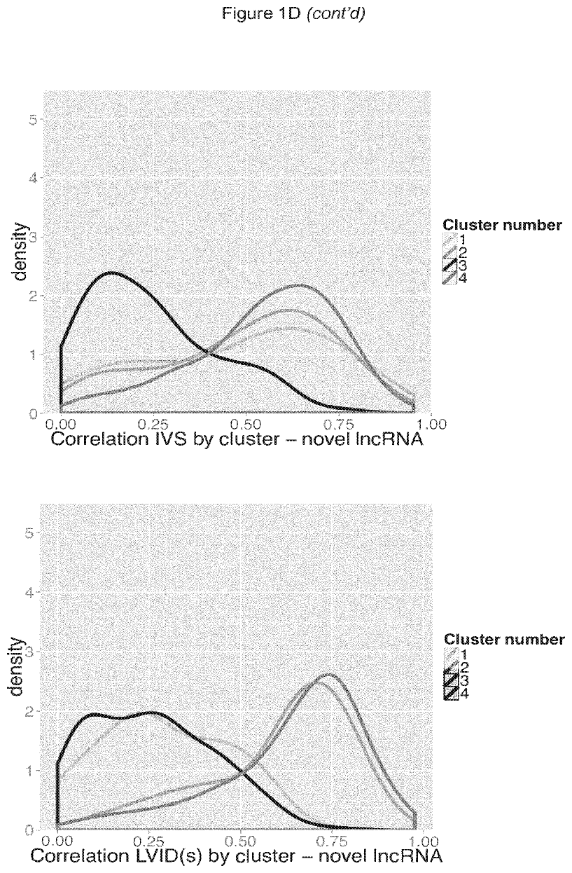

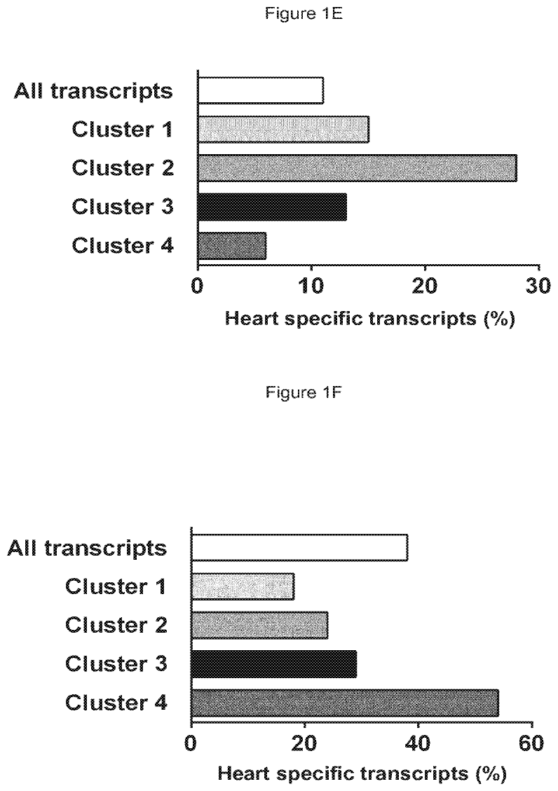

Within the identified transcripts, the Inventors identified four clusters for both coding (FIG. 1A) and novel lncRNA (FIG. 1B) transcripts. In each case, these consisted of one cluster that correlated positively with cardiac function and negatively with remodeling parameters, one cluster with the inverse of these correlations and two clusters with non-specific intermediate correlations. Gene ontology (GO) and heart specificity analysis was executed on individual clusters with GO analysis being executed on the most proximal coding genes with respect to novel lncRNAs. In the coding gene group, the most heart-specific cluster was Cluster 2 (FIG. 1E), which was positively correlated with cardiac functional traits and associated with genes involved in mitochondrial biology (FIG. 1A). The least heart-specific cluster (Cluster 4) was positively correlated with remodeling and associated with genes involved in wound healing and extracellular matrix (FIG. 1A). Within novel lncRNAs, and in particular within the 104 lncRNAs of Table 1, the most heart-specific cluster, i.e. Cluster 4 (FIG. 1F), was again positively correlated with cardiac function associated traits. Proximal coding genes to novel lncRNA in Cluster 4 were enriched with heart development associated processes (FIG. 1C). Since novel lncRNAs that cluster specifically with particular physiological traits were likely to be involved in biological processes associated with those traits, these findings indicated that novel lncRNAs within this cluster could represent a class of cardiac-specific regulators of developmental gene programs, which was reactivated in the damaged myocardium. Finally, the least heart specific clusters were one and two, which was positively correlated with remodeling traits.

These data demonstrated that unsupervised clustering of transcripts was able to distinguish physiological traits. In addition, it indicated that lncRNAs could represent specific markers of particular physiological traits. To test this, The Inventors compared correlation distributions for each UCSC coding gene and novel lncRNA cluster, with each of the following traits; ejection fraction (EF), interventricular septal thickness at systole (IVS), myocardial infarction trace (MI trace) and left ventricular internal diameter at systole (LVID) (FIG. 1B and D). UCSC coding gene Clusters 2 and 4 strongly correlated with all these traits when compared to non-specific clusters (Clusters 1 and 3) (FIG. 1B). A similar pattern of correlation was observed with novel lncRNA clusters 2 and 4 (FIG. 1D). However, novel lncRNA Cluster 1 was particularly interesting since it exhibited poor correlation with LVID, EF and MI trace but correlated well with IVS which is typically linked to EF. This unique characteristic is likely a consequence of the exquisite context and cell-type specific expression of lncRNAs, and has intriguing implications for the utilization of novel lncRNAs as biomarkers.

Preferably, in accordance with the method the method for diagnosing a cardiac pathology described herein, an up expression of one or more lncRNA having a cDNA sequence selected from the group comprising SEQ ID No 17, SEQ ID No 18, SEQ ID No 19, SEQ ID No 20, SEQ ID No 21, SEQ ID No 22, SEQ ID No 23, SEQ ID No 24, SEQ ID No 25, SEQ ID No 26, SEQ ID No 27, SEQ ID No 28, SEQ ID No 29, SEQ ID No 30, SEQ ID No 31, SEQ ID No 32, SEQ ID No 33, SEQ ID No 34, SEQ ID No 96, SEQ ID No 99, fragments thereof, isoforms thereof and variants sharing at least 80% nucleotide sequence identity thereto, in the biological sample compared to the expression levels of one or more of these lnCRNAs in a control sample from a normal subject indicates that the subject suffered from myocardial infarction or is suffering from cardiac pathology associated with maladaptive remodeling of the myocardium.

Preferably also, in accordance with the method for diagnosing a cardiac pathology described herein a down expression of one or more lncRNA having a cDNA sequence selected from the group comprising SEQ ID No 48, SEQ ID No 49, SEQ ID No 50, SEQ ID No 51, SEQ ID No 52, SEQ ID No 53, SEQ ID No 54, SEQ ID No 55, SEQ ID No 56, SEQ ID No 57, SEQ ID No 58, SEQ ID No 59, SEQ ID No 60, SEQ ID No 61, SEQ ID No 62, SEQ ID No 63, SEQ ID No 64, SEQ ID No 65, SEQ ID No 66, SEQ ID No 67, SEQ ID No 68, SEQ ID No 69, SEQ ID No 70, SEQ ID No 71, SEQ ID No 72, SEQ ID No 73, SEQ ID No 74, SEQ ID No 75, SEQ ID No 76, SEQ ID No 77, SEQ ID No 78, SEQ ID No 79, SEQ ID No 80, SEQ ID No 81, SEQ ID No 82, SEQ ID No 83, SEQ ID No 84, SEQ ID No 85, SEQ ID No 86, SEQ ID No 87, SEQ ID No 88, SEQ ID No 89, SEQ ID No 90, SEQ ID No 91, SEQ ID No 92, SEQ ID No 93, SEQ ID No 94, SEQ ID No 95, SEQ ID No 97, SEQ ID No 98, SEQ ID No 100, SEQ ID No 101, SEQ ID No 102, SEQ ID No103, and SEQ ID No 104, fragments thereof, isoforms thereof and variants sharing at least 80% nucleotide sequence identity thereto, in the biological sample compared to the expression levels of one or more of these lnCRNAs in a control sample from a normal subject indicates that the subject suffered from myocardial infarction.

Also in accordance with the method for diagnosing a cardiac pathology described herein wherein wherein a differential expression of one or more lncRNA having a cDNA sequence selected from the group comprising SEQ ID No 1, SEQ ID No 2, SEQ ID No 3, SEQ ID No 4, SEQ ID No 5, SEQ ID No 6, SEQ ID No 7, SEQ ID No 8, SEQ ID No 9, SEQ ID No 10, SEQ ID No 11, SEQ ID No 12, SEQ ID No 13, SEQ ID No 14, SEQ ID No 15, SEQ ID No 16, fragments thereof, isoforms thereof and variants sharing at least 80% nucleotide sequence identity thereto, in the biological sample compared to the expression levels of one or more of these lnCRNAs in a control sample from a normal subject indicates that the subject suffered from heart failure with preserved ejection fraction.

The phrase “differentially expressed” refers to differences in the quantity and/or the frequency of a biomarker present in a sample taken from patients having, for example, a cardiac pathology or form a cardiac tissue undergoing regeneration or from a stem cell undergoing cardiac differentiation or from a cardiac tissue undergoing surgical and/or pharmacological therapies as compared to a control subject. For example, a biomarker can be a lncRNA which is present at an elevated level or at a decreased level in samples of patients with a cardiac pathology or a cardiac tissue undergoing regeneration or a stem cell undergoing cardiac differentiation or a cardiac tissue undergoing surgical and/or pharmacological therapies compared to samples of control subjects. Alternatively, a biomarker can be a lncRNA which is detected at a higher frequency or at a lower frequency in samples of patients with a cardiac pathology or a cardiac tissue undergoing regeneration or a stem cell undergoing cardiac differentiation or a cardiac tissue undergoing surgical and/or pharmacological therapies compared to samples of control subjects or control tissues. A biomarker can be differentially present in terms of quantity, frequency or both.

A lncRNA is differentially expressed between two samples if the amount of the lncRNA in one sample is statistically significantly different from the amount of the lncRNA in the other sample. For example, an lncRNA is differentially expressed in two samples if it is present at least about 120%, at least about 130%, at least about 150%, at least about 180%, at least about 200%, at least about 300%, at least about 500%, at least about 700%, at least about 900%, or at least about 1000% greater than it is present in the other sample, or if it is detectable in one sample and not detectable in the other.

Alternatively or additionally, a lncRNA is differentially expressed in two sets of samples if the frequency of detecting the lncRNA in samples is statistically significantly higher or lower than in the control samples. For example, an lncRNA is differentially expressed in two sets of samples if it is detected at least about 120%, at least about 130%, at least about 150%, at least about 180%, at least about 200%, at least about 300%, at least about 500%, at least about 700%, at least about 900%, or at least about 1000% more frequently or less frequently observed in one set of samples than the other set of samples.

The terms “subject,” “individual,” and “patient,” are used interchangeably herein and refer to any mammalian subject for whom diagnosis, prognosis, treatment, or therapy is desired, particularly humans. Other subjects may include cattle, dogs, cats, guinea pigs, rabbits, rats, mice, horses, and so on. In some cases, the methods of the invention find use in experimental animals, in veterinary application, and in the development of animal models for disease, including, but not limited to, rodents including mice, rats, guinea pigs, and hamsters; rabbits, primates, and transgenic animals.

As used herein, a “biological sample” refers to a sample of tissue or fluid isolated from a subject, including but not limited to, for example, urine, blood, plasma, serum, fecal matter, bone marrow, bile, spinal fluid, lymph fluid, samples of the skin, external secretions of the skin, respiratory, intestinal, and genitourinary tracts, tears, saliva, milk, blood cells, organs, biopsies, and also samples containing cells or tissues derived from the subject and grown in culture, and in vitro cell culture constituents, including but not limited to, conditioned media resulting from the growth of cells and tissues in culture, recombinant cells, stem cells, and cell components.

The terms “quantity,” “amount,” and “level” are used interchangeably herein and may refer to an absolute quantification of a molecule or an analyte in a sample, or to a relative quantification of a molecule or analyte in a sample, i.e., relative to another value such as relative to a reference value as taught herein, or to a range of values for the biomarker. These values or ranges can be obtained from a single patient or from a group of patients.

“Diagnosis” as used herein generally includes determination as to whether a subject is likely affected by a given disease, disorder or dysfunction of the invention. The skilled artisan often makes a diagnosis on the basis of one or more diagnostic indicators, i.e., a biomarker, the presence, absence, or amount of which is indicative of the presence or absence of the disease, disorder or dysfunction.

“Prognosis” as used herein generally refers to a prediction of the probable course and outcome of a clinical condition or disease. A prognosis of a patient is usually made by evaluating factors or symptoms of a disease that are indicative of a favorable or unfavorable course or outcome of the disease. It is understood that the term “prognosis” does not necessarily refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition.

When analyzing the levels of biomarkers in a biological sample, the reference value ranges used for comparison can represent the level of one or more biomarkers found in one or more samples of one or more subjects without cardiac disease (i.e., normal or control samples). Alternatively, the reference values can represent the level of one or more biomarkers found in one or more samples of one or more subjects with cardiac disease. More specifically, the reference value ranges can represent the level of one or more biomarkers at particular stages of disease to facilitate a determination of the stage of disease progression in an individual.

A “control” sample as used herein refers to a biological sample, such as tissue or cells that are not diseased. That is, a control sample is obtained from a normal subject (e.g. an individual known to not have cardiac disease or any condition or symptom associated with).

It is understood that the expression level of the biomarkers in a sample can be determined by any suitable method known in the art. Measurement of the level of a biomarker can be direct or indirect. For example, the abundance levels of lncRNAs can be directly quantitated. Alternatively, the amount of a biomarker can be determined indirectly by measuring abundance levels of cDNAs, amplified RNAs or DNAs, or by measuring quantities or activities of RNAs, or other molecules that are indicative of the expression level of the biomarker. Preferably, the amount of a biomarker is determined indirectly by measuring abundance levels of cDNAs.

LncRNAs can be detected and quantitated by a variety of methods including, but not limited to, microarray analysis, polymerase chain reaction (PCR), reverse transcriptase polymerase chain reaction (RT-PCR), Northern blot, serial analysis of gene expression (SAGE), immunoassay, and mass spectrometry, any sequencing-based methods known in the art.

In one embodiment, microarrays are used to measure the levels of biomarkers. An advantage of microarray analysis is that the expression of each of the biomarkers can be measured simultaneously, and microarrays can be specifically designed to provide a diagnostic expression profile for a particular disease or condition (e.g., a cardiac pathology).

Microarrays are prepared by selecting probes which comprise a polynucleotide sequence, and then immobilizing such probes to a solid support or surface. For example, the probes may comprise DNA sequences, RNA sequences, or copolymer sequences of DNA and RNA. The polynucleotide sequences of the probes may also comprise DNA and/or RNA analogues, or combinations thereof. For example, the polynucleotide sequences of the probes may be full or partial fragments of genomic DNA. The polynucleotide sequences of the probes may also be synthesized nucleotide sequences, such as synthetic oligonucleotide sequences. The probe sequences can be synthesized either enzymatically in vivo, enzymatically in vitro (e.g., by PCR), or non-enzymatically in vitro.

Probes used in the methods of the invention are preferably immobilized to a solid support which may be either porous or non-porous. For example, the probes may be polynucleotide sequences which are attached to a nitrocellulose or nylon membrane or filter covalently at either the 3′ or the 5′ end of the polynucleotide. Such hybridization probes are well known in the art (see, e.g., Sambrook, et al., Molecular Cloning: A Laboratory Manual (3rd Edition, 2001). Alternatively, the solid support or surface may be a glass or plastic surface. In one embodiment, hybridization levels are measured to microarrays of probes consisting of a solid phase on the surface of which are immobilized a population of polynucleotides, such as a population of DNA or DNA mimics, or, alternatively, a population of RNA or RNA mimics. The solid phase may be a nonporous or, optionally, a porous material such as a gel.

In one embodiment, the microarray comprises a support or surface with an ordered array of binding (e.g., hybridization) sites or “probes” each representing one of the biomarkers described herein. Preferably the microarrays are addressable arrays, and more preferably positionally addressable arrays. More specifically, each probe of the array is preferably located at a known, predetermined position on the solid support such that the identity (i.e., the sequence) of each probe can be determined from its position in the array (i.e., on the support or surface). Each probe is preferably covalently attached to the solid support at a single site.

Microarrays can be made in a number of ways, of which several are described below. However they are produced, microarrays share certain characteristics. The arrays are reproducible, allowing multiple copies of a given array to be produced and easily compared with each other. Preferably, microarrays are made from materials that are stable under binding (e.g., nucleic acid hybridization) conditions. Microarrays are generally small, e.g., between 1 cm2 and 25 cm2; however, larger arrays may also be used, e.g., in screening arrays. Preferably, a given binding site or unique set of binding sites in the microarray will specifically bind (e.g., hybridize) to the product of a single gene in a cell (e.g., to a specific mRNA, lncRNA, or to a specific cDNA derived therefrom). However, in general, other related or similar sequences will cross hybridize to a given binding site.

As noted above, the “probe” to which a particular polynucleotide molecule specifically hybridizes contains a complementary polynucleotide sequence. The probes of the microarray typically consist of nucleotide sequences of no more than 1,000 nucleotides. In some embodiments, the probes of the array consist of nucleotide sequences of 10 to 1,000 nucleotides. In one embodiment, the nucleotide sequences of the probes are in the range of 10-200 nucleotides in length and are genomic sequences of one species of organism, such that a plurality of different probes is present, with sequences complementary and thus capable of hybridizing to the genome of such a species of organism, sequentially tiled across all or a portion of the genome. In other embodiments, the probes are in the range of 10-30 nucleotides in length, in the range of 10-40 nucleotides in length, in the range of 20-50 nucleotides in length, in the range of 40-80 nucleotides in length, in the range of 50-150 nucleotides in length, in the range of 80-120 nucleotides in length, or are 60 nucleotides in length. The probes may comprise DNA or DNA “mimics” (e.g., derivatives and analogues) corresponding to a portion of an organism's genome. In another embodiment, the probes of the microarray are complementary RNA or RNA mimics. DNA mimics are polymers composed of subunits capable of specific, Watson-Crick-like hybridization with DNA, or of specific hybridization with RNA. The nucleic acids can be modified at the base moiety, at the sugar moiety, or at the phosphate backbone (e.g., phosphorothioates).

DNA can be obtained, e.g., by polymerase chain reaction (PCR) amplification of genomic DNA or cloned sequences. PCR primers are preferably chosen based on a known sequence of the genome that will result in amplification of specific fragments of genomic DNA. Computer programs that are well known in the art are useful in the design of primers with the required specificity and optimal amplification properties, such as Oligo version 5.0 (National Biosciences). Typically each probe on the microarray will be between 10 bases and 50,000 bases, usually between 300 bases and 1,000 bases in length. PCR methods are well known in the art, and are described, for example, in Innis et al., eds., PCR Protocols: A Guide To Methods And Applications, Academic Press Inc., San Diego, Calif. (1990); herein incorporated by reference in its entirety. It will be apparent to one skilled in the art that controlled robotic systems are useful for isolating and amplifying nucleic acids.

An alternative, preferred means for generating polynucleotide probes is by synthesis of synthetic polynucleotides or oligonucleotides, e.g., using N-phosphonate or phosphoramidite chemistries (Froehler et al., Nucleic Acid Res. 14:5399-5407 (1986); McBride et al., Tetrahedron Lett. 24:246-248 (1983)). Synthetic sequences are typically between about 10 and about 500 bases in length, more typically between about 20 and about 100 bases, and most preferably between about 40 and about 70 bases in length. In some embodiments, synthetic nucleic acids include non-natural bases, such as, but by no means limited to, inosine. As noted above, nucleic acid analogues may be used as binding sites for hybridization. An example of a suitable nucleic acid analogue is peptide nucleic acid (see, e.g., U.S. Pat. No. 5,539,083).

Probes are preferably selected using an algorithm that takes into account binding energies, base composition, sequence complexity, cross-hybridization binding energies, and secondary structure. See International Patent Publication WO 01/05935.

A skilled artisan will also appreciate that positive control probes, e.g., probes known to be complementary and hybridizable to sequences in the target polynucleotide molecules, and negative control probes, e.g., probes known to not be complementary and hybridizable to sequences in the target polynucleotide molecules, should be included on the array. In one embodiment, positive controls are synthesized along the perimeter of the array. In another embodiment, positive controls are synthesized in diagonal stripes across the array. In still another embodiment, the reverse complement for each probe is synthesized next to the position of the probe to serve as a negative control. In yet another embodiment, sequences from other species of organism are used as negative controls or as “spike-in” controls.

The probes are attached to a solid support or surface, which may be made, e.g., from glass, plastic (e.g., polypropylene, nylon), polyacrylamide, nitrocellulose, gel, or other porous or nonporous material. One method for attaching nucleic acids to a surface is by printing on glass plates, as known in the art. This method is especially useful for preparing microarrays of cDNA A second method for making microarrays produces high-density oligonucleotide arrays. Techniques are known for producing arrays containing thousands of oligonucleotides complementary to defined sequences, at defined locations on a surface using photolithographic techniques for synthesis in situ (see, U.S. Pat. Nos. 5,578,832; 5,556,752; and 5,510,270; herein incorporated by reference in their entireties) or other methods for rapid synthesis and deposition of defined oligonucleotides. When these methods are used, oligonucleotides (e.g., 60-mers) of known sequence are synthesized directly on a surface such as a derivatized glass slide. Usually, the array produced is redundant, with several oligonucleotide molecules per RNA. Other methods for making microarrays, e.g., by masking, may also be used. In principle, any type of array known in the art, for example, dot blots on a nylon hybridization membrane could be used. However, as will be recognized by those skilled in the art, very small arrays will frequently be preferred because hybridization volumes will be smaller.

Microarrays can also be manufactured by means of an ink jet printing device for oligonucleotide synthesis, e.g., using the methods and systems described by Blanchard in U.S. Pat. No. 6,028,189. Specifically, the oligonucleotide probes in such microarrays are synthesized in arrays, e.g., on a glass slide, by serially depositing individual nucleotide bases in “microdroplets” of a high surface tension solvent such as propylene carbonate. The microdroplets have small volumes (e.g., 100 pL or less, more preferably 50 pL or less) and are separated from each other on the microarray (e.g., by hydrophobic domains) to form circular surface tension wells which define the locations of the array elements (i.e., the different probes). Microarrays manufactured by this ink jet method are typically of high density, preferably having a density of at least about 2,500 different probes per 1 cm². The polynucleotide probes are attached to the support covalently at either the 3′ or the 5′ end of the polynucleotide.

Biomarker polynucleotides which may be measured by microarray analysis can be expressed lncRNAs or a nucleic acid derived therefrom (e.g., cDNA or amplified RNA derived from cDNA that incorporates an RNA polymerase promoter), including naturally occurring nucleic acid molecules, as well as synthetic nucleic acid molecules. In one embodiment, the target polynucleotide molecules comprise RNA, including, but by no means limited to, total cellular RNA, lncRNA, poly (A)+ messenger RNA (mRNA) or a fraction thereof, cytoplasmic mRNA, or RNA transcribed from cDNA (i.e., cRNA; see, e.g., U.S. Pat. Nos. 5,545,522, 5,891,636, or 5,716,785). Methods for preparing total and poly(A)+ RNA are well known in the art, and are described generally, e.g., in Sambrook, et al., Molecular Cloning: A Laboratory Manual (3rd Edition, 2001). RNA can be extracted from a cell of interest using guanidinium thiocyanate lysis followed by CsCl centrifugation, a silica gel-based column (e.g., RNeasy (Qiagen, Valencia, Calif.) or StrataPrep (Stratagene, La Jolla, Calif.)), or using phenol and chloroform, as known in the art. Poly(A)+ RNA can be selected, e.g., by selection with oligo-dT cellulose or, alternatively, by oligo-dT primed reverse transcription of total cellular RNA. RNA can be fragmented by methods known in the art, e.g., by incubation with ZnCl2, to generate fragments of RNA.

In one embodiment, total RNA, lncRNAs, or nucleic acids derived therefrom (such as cDNA), are isolated from a sample taken from a patient having a cardiac pathology or a cardiac tissue undergoing regeneration or a stem cell undergoing cardiac differentiation or a cardiac tissue undergoing surgical and/or pharmaceological therapies. Biomarker lncRNAs that are poorly expressed in particular cells may be enriched using normalization techniques known in the art. As described above, the biomarker polynucleotides can be detectably labeled at one or more nucleotides. Any method known in the art may be used to label the target polynucleotides. Preferably, this labeling incorporates the label uniformly along the length of the RNA, and more preferably, the labeling is carried out at a high degree of efficiency. For example, polynucleotides can be labeled by oligo-dT primed reverse transcription. Random primers (e.g., 9-mers) can be used in reverse transcription to uniformly incorporate labeled nucleotides over the full length of the polynucleotides. Alternatively, random primers may be used in conjunction with PCR methods or T7 promoter-based in vitro transcription methods in order to amplify polynucleotides.

The detectable label may be a luminescent label. For example, fluorescent labels, bioluminescent labels, chemiluminescent labels, and colorimetric labels may be used in the practice of the invention. Fluorescent labels that can be used include, but are not limited to, fluorescein, a phosphor, a rhodamine, or a polymethine dye derivative. Additionally, commercially available fluorescent labels including, but not limited to, fluorescent phosphoramidites such as FluorePrime (Amersham Pharmacia, Piscataway, N.J.), Fluoredite (Miilipore, Bedford, Mass.), FAM (ABI, Foster City, Calif.), and Cy3 or Cy5 (Amersham Pharmacia, Piscataway, N.J.) can be used. Alternatively, the detectable label can be a radiolabeled nucleotide.

In one embodiment, biomarker polynucleotide molecules from a patient sample are labeled differentially from the corresponding polynucleotide molecules of a reference sample. The reference can comprise lncRNAs from a normal biological sample (i.e., control sample, e.g., biopsy from a subject not having a cardiac pathology or a cardiac tissue undergoing regeneration or a stem cell undergoing cardiac differentiation or a cardiac tissue undergoing surgical and/or pharmaceological therapies) or from a reference biological sample, (e.g., sample from a subject having a cardiac pathology or cell sample of a cardiac tissue undergoing regeneration or a stem cell undergoing cardiac differentiation or a cardiac tissue undergoing surgical and/or pharmaceological therapies).

Nucleic acid hybridization and wash conditions are chosen so that the target polynucleotide molecules specifically bind or specifically hybridize to the complementary polynucleotide sequences of the array, preferably to a specific array site, wherein its complementary DNA is located. Arrays containing double-stranded probe DNA situated thereon are preferably subjected to denaturing conditions to render the DNA single-stranded prior to contacting with the target polynucleotide molecules. Arrays containing single-stranded probe DNA (e.g., synthetic oligodeoxyribonucleic acids) may need to be denatured prior to contacting with the target polynucleotide molecules, e.g., to remove hairpins or dimers which form due to self-complementary sequences.

Optimal hybridization conditions will depend on the length (e.g., oligomer versus polynucleotide greater than 200 bases) and type (e.g., RNA, or DNA) of probe and target nucleic acids. One of skill in the art will appreciate that as the oligonucleotides become shorter, it may become necessary to adjust their length to achieve a relatively uniform melting temperature for satisfactory hybridization results. General parameters for specific (i.e., stringent) hybridization conditions for nucleic acids are described in Sambrook, et al., Molecular Cloning: A Laboratory Manual (3rd Edition, 2001). Typical hybridization conditions for the cDNA microarrays of Schena et al. are hybridization in 5×SSC plus 0.2% SDS at 65° C. for four hours, followed by washes at 25° C. in low stringency wash buffer (1×SSC plus 0.2% SDS), followed by 10 minutes at 25° C. in higher stringency wash buffer (0.1×SSC plus 0.2% SDS). Particularly preferred hybridization conditions include hybridization at a temperature at or near the mean melting temperature of the probes (e.g., within 51° C., more preferably within 21° C.) in 1 M NaCl, 50 mM MES buffer (pH 6.5), 0.5% sodium sarcosine and 30% formamide.

When fluorescently labeled gene products are used, the fluorescence emissions at each site of a microarray may be, preferably, detected by scanning confocal laser microscopy. In one embodiment, a separate scan, using the appropriate excitation line, is carried out for each of the two fluorophores used. Alternatively, a laser may be used that allows simultaneous specimen illumination at wavelengths specific to the two fluorophores and emissions from the two fluorophores can be analyzed simultaneously. Arrays can be scanned with a laser fluorescent scanner with a computer controlled X-Y stage and a microscope objective. Sequential excitation of the two fluorophores is achieved with a multi-line, mixed gas laser and the emitted light is split by wavelength and detected with two photomultiplier tubes. Fluorescence laser scanning devices are known in the art. Alternatively, a fiber-optic bundle, may be used to monitor mRNA abundance levels at a large number of sites simultaneously.

In one embodiment, the invention includes a microarray comprising a plurality of probes that hybridize to one or more lncRNAs selected from the group comprising SEQ ID No 1, SEQ ID No 2, SEQ ID No 3, SEQ ID No 4, SEQ ID No 5, SEQ ID No 6, SEQ ID No 7, SEQ ID No 8, SEQ ID No 9, SEQ ID No 10, SEQ ID No 11, SEQ ID No 12, SEQ ID No 13, SEQ ID No 14, SEQ ID No 15, SEQ ID No 16, SEQ ID No 17, SEQ ID No 18, SEQ ID No 19, SEQ ID No 20, SEQ ID No 21, SEQ ID No 22, SEQ ID No 23, SEQ ID No 24, SEQ ID No 25, SEQ ID No 26, SEQ ID No 27, SEQ ID No 28, SEQ ID No 29, SEQ ID No 30, SEQ ID No 31, SEQ ID No 32, SEQ ID No 33, SEQ ID No 34, SEQ ID No 35, SEQ ID No 36, SEQ ID No 37, SEQ ID No 38, SEQ ID No 39, SEQ ID No 40, SEQ ID No 41, SEQ ID No 42, SEQ ID No 43, SEQ ID No 44, SEQ ID No 45, SEQ ID No 46, SEQ ID No 47, SEQ ID No 48, SEQ ID No 49, SEQ ID No 50, SEQ ID No 51, SEQ ID No 52, SEQ ID No 53, SEQ ID No 54, SEQ ID No 55, SEQ ID No 56, SEQ ID No 57, SEQ ID No 58, SEQ ID No 59, SEQ ID No 60, SEQ ID No 61, SEQ ID No 62, SEQ ID No 63, SEQ ID No 64, SEQ ID No 65, SEQ ID No 66, SEQ ID No 67, SEQ ID No 68, SEQ ID No 69, SEQ ID No 70, SEQ ID No 71, SEQ ID No 72, SEQ ID No 73, SEQ ID No 74, SEQ ID No 75, SEQ ID No 76, SEQ ID No 77, SEQ ID No 78, SEQ ID No 79, SEQ ID No 80, SEQ ID No 81, SEQ ID No 82, SEQ ID No 83, SEQ ID No 84, SEQ ID No 85, SEQ ID No 86, SEQ ID No 87, SEQ ID No 88, SEQ ID No 89, SEQ ID No 90, SEQ ID No 91, SEQ ID No 92, SEQ ID No 93, SEQ ID No 94, and SEQ ID No 95, SEQ ID No 96, SEQ ID No 97, SEQ ID No 98, SEQ ID No 99, SEQ ID No 100, SEQ ID No 101, SEQ ID No 102, SEQ ID No103, and SEQ ID No 104, fragments thereof, isoforms thereof and variants sharing at least 80% nucleotide sequence identity thereto, most preferably from the group comprising SEQ ID No 25, SEQ ID No 28, SEQ ID No 48, SEQ ID No 52, SEQ ID No 53, SEQ ID No 82, SEQ ID No 84, SEQ ID No 88, SEQ ID No 96, SEQ ID No 97, SEQ ID No 98, SEQ ID No 99, SEQ ID No 100, SEQ ID No 101, SEQ ID No 103, and SEQ ID No 104.

Serial Analysis Gene Expression (SAGE), can also be used to determine RNA (e.g., lncRNA) abundances in a cell sample. SAGE analysis does not require a special device for detection, and is one of the preferable analytical methods for simultaneously detecting the expression of a large number of transcription products. First, RNA is extracted from cells. Next, the RNA is converted into cDNA using a biotinylated oligo (dT) primer, and treated with a four-base recognizing restriction enzyme (Anchoring Enzyme: AE) resulting in AE-treated fragments containing a biotin group at their 3′ terminus. Next, the AE-treated fragments are incubated with streptoavidin for binding. The bound cDNA is divided into two fractions, and each fraction is then linked to a different double-stranded oligonucleotide adapter (linker) A or B. These linkers are composed of: (1) a protruding single strand portion having a sequence complementary to the sequence of the protruding portion formed by the action of the anchoring enzyme, (2) a 5′ nucleotide recognizing sequence of the IIS-type restriction enzyme (cleaves at a predetermined location no more than 20 by away from the recognition site) serving as a tagging enzyme (TE), and (3) an additional sequence of sufficient length for constructing a PCR-specific primer. The linker-linked cDNA is cleaved using the tagging enzyme, and only the linker-linked cDNA sequence portion remains, which is present in the form of a short-strand sequence tag. Next, pools of short-strand sequence tags from the two different types of linkers are linked to each other, followed by PCR amplification using primers specific to linkers A and B. As a result, the amplification product is obtained as a mixture comprising myriad sequences of two adjacent sequence tags (ditags) bound to linkers A and B. The amplification product is treated with the anchoring enzyme, and the free ditag portions are linked into strands in a standard linkage reaction. The amplification product is then cloned. Determination of the clone's nucleotide sequence can be used to obtain a read-out of consecutive ditags of constant length. The presence of mRNA corresponding to each tag can then be identified from the nucleotide sequence of the clone and information on the sequence tags.

Quantitative reverse transcriptase PCR (qRT-PCR) can also be used to determine the expression profiles of biomarkers (see, e.g., U.S. Patent Application Publication No. 2005/0048542A1; herein incorporated by reference in its entirety). The first step in gene expression profiling by RT-PCR is the reverse transcription of the RNA template into cDNA, followed by its exponential amplification in a PCR reaction. The two most commonly used reverse transcriptases are avilo myeloblastosis virus reverse transcriptase (AMV-RT) and Moloney murine leukemia virus reverse transcriptase (MLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. Thus, TAQMAN PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TAQMAN RT-PCR can be performed using commercially available equipment, such as, for example, ABI PRISM 7700 sequence detection system. (Perkin-Elmer-Applied Biosystems, Foster City, Calif., USA), or Lightcycler (Roche Molecular Biochemicals, Mannheim, Germany). In a preferred embodiment, the 5′ nuclease procedure is run on a real-time quantitative PCR device such as the ABI PRISM 7700 sequence detection system. The system consists of a thermocycler, laser, charge-coupled device (CCD), camera and computer. The system includes software for running the instrument and for analyzing the data. 5′-Nuclease assay data are initially expressed as Ct, or the threshold cycle. Fluorescence values are recorded during every cycle and represent the amount of product amplified to that point in the amplification reaction. The point when the fluorescent signal is first recorded as statistically significant is the threshold cycle (Ct).

To minimize errors and the effect of sample-to-sample variation, RT-PCR is usually performed using an internal standard. The ideal internal standard is expressed at a constant level among different tissues, and is unaffected by the experimental treatment. RNAs most frequently used to normalize patterns of gene expression are mRNAs for the housekeeping genes glyceraldehyde-3-phosphate-dehydrogenase (GAPDH) and beta-actin.

A more recent variation of the RT-PCR technique is the real time quantitative PCR, which measures PCR product accumulation through a dual-labeled fluorigenic probe (i.e., TAQMAN probe). Real time PCR is compatible both with quantitative competitive PCR, where internal competitor for each target sequence is used for normalization, and with quantitative comparative PCR using a normalization gene contained within the sample, or a housekeeping gene for RT-PCR.

Mass spectrometry, and particularly SELDI mass spectrometry, is a particularly useful method for detection of the biomarkers of this invention. Laser desorption time-of-flight mass spectrometer can be used in embodiments of the invention. In laser desorption mass spectrometry, a substrate or a probe comprising biomarkers is introduced into an inlet system. The biomarkers are desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of markers of specific mass to charge ratio.

Matrix-assisted laser desorption/ionization mass spectrometry (MALDI-MS) can also be used for detecting the biomarkers of this invention. MALDI-MS is a method of mass spectrometry that involves the use of an energy absorbing molecule, frequently called a matrix, for desorbing proteins intact from a probe surface. MALDI is described, for example, in U.S. Pat. Nos. 5,118,937 and 5,045,694. In MALDI-MS, the sample is typically mixed with a matrix material and placed on the surface of an inert probe. Exemplary energy absorbing molecules include cinnamic acid derivatives, sinapinic acid (“SPA”), cyano hydroxy cinnamic acid (“CHCA”) and dihydroxybenzoic acid. Other suitable energy absorbing molecules are known to those skilled in this art. The matrix dries, forming crystals that encapsulate the analyte molecules. Then the analyte molecules are detected by laser desorption/ionization mass spectrometry.

Surface-enhanced laser desorption/ionization mass spectrometry, or SELDI-MS represents an improvement over MALDI for the fractionation and detection of biomolecules, such as lncRNAs, in complex mixtures. SELDI is a method of mass spectrometry in which biomolecules, such as lncRNAs, are captured on the surface of a biochip using capture reagents that are bound there. Typically, non-bound molecules are washed from the probe surface before interrogation. SELDI is described, for example, in: U.S. Pat. No. 5,719,060 and in U.S. Pat. No. 6,225,047.

Biomarkers on the substrate surface can be desorbed and ionized using gas phase ion spectrometry. Any suitable gas phase ion spectrometer can be used as long as it allows biomarkers on the substrate to be resolved. Preferably, gas phase ion spectrometers allow quantitation of biomarkers. In one embodiment, a gas phase ion spectrometer is a mass spectrometer. In a typical mass spectrometer, a substrate or a probe comprising biomarkers on its surface is introduced into an inlet system of the mass spectrometer. The biomarkers are then desorbed by a desorption source such as a laser, fast atom bombardment, high energy plasma, electrospray ionization, thermospray ionization, liquid secondary ion MS, field desorption, etc. The generated desorbed, volatilized species consist of preformed ions or neutrals which are ionized as a direct consequence of the desorption event. Generated ions are collected by an ion optic assembly, and then a mass analyzer disperses and analyzes the passing ions. The ions exiting the mass analyzer are detected by a detector. The detector then translates information of the detected ions into mass-to-charge ratios. Detection of the presence of biomarkers or other substances will typically involve detection of signal intensity. This, in turn, can reflect the quantity and character of biomarkers bound to the substrate. Any of the components of a mass spectrometer (e.g., a desorption source, a mass analyzer, a detector, etc.) can be combined with other suitable components described herein or others known in the art in embodiments of the invention.

Biomarkers can also be detected with assays based on the use of antibodies that specifically recognize the lncRNA biomarkers or polynucleotide or oligonucleotide fragments of the biomarkers. Such assays include, but are not limited to, immunohistochemistry (IHC), enzyme-linked immunosorbent assay (ELISA), radioimmunoassays (RIA), “sandwich” immunoassays, fluorescent immunoassays, immunoprecipitation assays, the procedures of which are well known in the art.

Biomarkers can also be detected with any sequencing based technologies know in the art.

In yet another aspect, the invention provides kits for use in diagnosing a cardiac pathology or a cardiac tissue undergoing regeneration or a stem cell undergoing cardiac differentiation or a cardiac tissue undergoing surgical and/or pharmaceological therapies, wherein the kits can be used to detect the lncRNA biomarkers of the present invention. For example, the kits can be used to detect any one or more of the biomarkers described herein, which are differentially expressed in samples of a patient with a cardiac pathology or a cardiac tissue undergoing regeneration or a stem cell undergoing cardiac differentiation or a cardiac tissue undergoing surgical and/or pharmaceological therapies. The kit may include one or more agents for detection of lncRNA biomarkers, a container for holding a biological sample isolated from a human subject; and printed instructions for reacting agents with the biological sample or a portion of the biological sample to detect the presence or amount of at least one lncRNA biomarker in the biological sample. The agents may be packaged in separate containers. The kit may further comprise one or more control reference samples and reagents for performing an immunoassay, a Northern blot, PCR, microarray analysis, or SAGE, DNA/RNA-sequencing,

In certain embodiments, the kit contains at least one probe that selectively hybridizes to a biomarker, or at least one antibody that selectively binds to a biomarker, or at least one set of PCR primers for amplifying a biomarker. In one embodiment, the kit comprises at least one agent for measuring the level of a biomarker.

The kit can comprise one or more containers for compositions contained in the kit. Compositions can be in liquid form or can be lyophilized. Suitable containers for the compositions include, for example, bottles, vials, syringes, and test tubes. Containers can be formed from a variety of materials, including glass or plastic. The kit can also comprise a package insert containing written instructions for methods of diagnosing a cardiac pathology or monitoring stem cell therapy or regenerative medical treatments.

The kits of the invention have number of applications. For example, the kits can be used for diagnosing a cardiac pathology or monitoring and/or evaluating the efficacy of a treatment for a cardiac pathology, stem cell therapy, or regenerative cardiac medicine. In a further example, the kits can be used to identify compounds that modulate expression of one or more of the biomarkers in in vitro or in vivo animal models to determine the effects of treatment.

By “therapeutically effective dose or amount” of each of the modulator of the invention is intended an amount that when administered in combination brings about a positive therapeutic response with respect to treatment of an individual for a cardiac pathology.

Thus, for example, a “positive therapeutic response” would be an improvement in the disease in association with the combination therapy, and/or an improvement in one or more symptoms of the disease in association with the combination therapy.

The actual dose to be administered will vary depending upon the age, weight, and general condition of the subject as well as the severity of the condition being treated, the judgment of the health care professional, and conjugate being administered. Therapeutically effective amounts can be determined by those skilled in the art, and will be adjusted to the particular requirements of each particular case. Generally, a therapeutically effective amount will range from about 0.50 mg to 5 grams daily, more preferably from about 5 mg to 2 grams daily, even more preferably from about 7 mg to 1.5 grams daily.

In certain embodiments, multiple therapeutically effective doses of each of at least one lncRNA and at least one additional therapeutical agent will be administered according to a daily dosing regimen, or intermittently. For example, a therapeutically effective dose can be administered, one day a week, two days a week, three days a week, four days a week, or five days a week, and so forth. By “intermittent” administration is intended the therapeutically effective dose can be administered, for example, every other day, every two days, every three days, and so forth. By “twice-weekly” or “two times per week” is intended that two therapeutically effective doses of the agent in question is administered to the subject within a 7 day period, beginning on day 1 of the first week of administration, with a minimum of 72 hours, between doses and a maximum of 96 hours between doses. By “thrice weekly” or “three times per week” is intended that three therapeutically effective doses are administered to the subject within a 7 day period, allowing for a minimum of 48 hours between doses and a maximum of 72 hours between doses. For purposes of the present invention, this type of dosing is referred to as “intermittent” therapy. In accordance with the methods of the present invention, a subject can receive intermittent therapy (i.e., twice-weekly or thrice-weekly administration of a therapeutically effective dose) for one or more weekly cycles until the desired therapeutic response is achieved. The agents can be administered by any acceptable route of administration as noted herein below.

A lncRNA modulator of the invention can be administered prior to, concurrent with, or subsequent to at least one additional therapeutic agent. If provided at the same time as the additional therapeutic agent, the lncRNA modulator can be provided in the same or in a different composition. Thus, the agents can be presented to the individual by way of concurrent therapy. By “concurrent therapy” is intended administration to a human subject such that the therapeutic effect of the combination of the substances is caused in the subject undergoing therapy. For example, concurrent therapy may be achieved by administering at least one therapeutically effective dose of a pharmaceutical composition comprising a lncRNA modulator and at least one therapeutically effective dose of a pharmaceutical composition comprising at least one addiitonal therapeutic agent according to a particular dosing regimen. Administration of the separate pharmaceutical compositions can be at the same time (i.e., simultaneously) or at different times (i.e., sequentially, in either order, on the same day, or on different days), so long as the therapeutic effect of the combination of these substances is caused in the subject undergoing therapy.

In other embodiments of the invention, the pharmaceutical composition of the invention is a sustained-release formulation, or a formulation that is administered using a sustained-release device. Such devices are well known in the art, and include, for example, transdermal patches, and miniature implantable pumps that can provide for drug delivery over time in a continuous, steady-state fashion at a variety of doses to achieve a sustained-release effect with a non-sustained-release pharmaceutical composition. The pharmaceutical compositions of the invention may be administered using the same or different routes of administration in accordance with any medically acceptable method known in the art. Suitable routes of administration include parenteral administration, such as subcutaneous (SC), intraperitoneal (IP), intramuscular (IM), intravenous (IV), or infusion, oral and pulmonary, nasal, topical, transdermal, and suppositories. Where the composition is administered via pulmonary delivery, the therapeutically effective dose is adjusted such that the soluble level of the agent, such as the lncRNA modulator in the bloodstream, is equivalent to that obtained with a therapeutically effective dose that is administered parenterally, for example SC, IP, IM, or IV. In some embodiments of the invention, the pharmaceutical composition comprising the lncRNA modulator is administered by IM or SC injection, particularly by IM or SC injection locally to the region where the therapeutic agent or agents used in the cardiac therapy protocol are administered.

Factors influencing the respective amount of the various compositions to be administered include, but are not limited to, the mode of administration, the frequency of administration (i.e., daily, or intermittent administration, such as twice- or thrice-weekly), the particular disease undergoing therapy, the severity of the disease, the history of the disease, whether the individual is undergoing concurrent therapy with another therapeutic agent, and the age, height, weight, health, and physical condition of the individual undergoing therapy. Generally, a higher dosage of this agent is preferred with increasing weight of the subject undergoing therapy. Below are examples of specific embodiments for carrying out the present invention. The examples are offered for illustrative purposes only, and are not intended to limit the scope of the present invention in any way.

The present invention also concerns a method for increasing and/or improving cardiac function comprising administrating a pharmaceutical composition comprising an effective amount of a modulator of lnc_019010 wherein the modulator is selected from the group comprising a chemical agent, an antibody, an engineered protease, and enzymatically active RNA, optionally in combination with pharmaceutically acceptable carriers, diluents and/or adjuvants. Preferably, the modulator of lnc_019010 is an enzymatically active RNA consisting in one or more antisense oligonucleotide targeting said lnc_019010. Most rpreferably, said one or more antisense oligonucleotide targeting said lnc_019010 is a modified antisense oligonucleotide (GapmeR) having a sequence as set forth in SEQ ID No 148.

Also envisioned in the present invention is a method for increasing and/or improving the conduction system in the heart comprising administrating a pharmaceutical composition comprising an effective amount of a modulator of lnc_033521 wherein the modulator is selected from the group comprising a chemical agent, an antibody, an engineered protease, and enzymatically active RNA, optionally in combination with pharmaceutically acceptable carriers, diluents and/or adjuvants.

The present invention further relates to a method of regulating the heart rate comprising administrating a pharmaceutical composition comprising an effective amount of a modulator of lnc_033521 wherein the modulator is selected from the group comprising a chemical agent, an antibody, an engineered protease, and enzymatically active RNA, optionally in combination with pharmaceutically acceptable carriers, diluents and/or adjuvants.

Preferably, the modulator of lnc_033521 is an enzymatically active RNA consisting in one or more antisense oligonucleotide targeting said lnc_033521. Most preferably, said one or more antisense oligonucleotide targeting said lnc_033521 is a modified antisense oligonucleotide (GapmeR) having a sequence as set forth in SEQ ID No 147.

The present invention further relates to a method for diagnosing dilated cardiomyopathy (DCM) in a subject, the method comprising:
a) measuring, directly or indirectly, the level of novlnc6 having a cDNA sequence as set forth in SEQ ID No. 48, fragments thereof, isoforms thereof and variants sharing at least 80% nucleotide sequence identity thereto; and
b) analyzing the levels of said novlnc6, fragments thereof, isoforms thereof and variants sharing at least 80% nucleotide sequence identity thereto, in conjunction with respective reference value ranges for said novlnc6,
wherein a decreased expression level of said novlnc6 in the biological sample compared to a control sample from a normal subject indicates that the subject has a dilated cardiomyopathy.

The present invention further relates to a method for diagnosing aortic stenosis (AOS) in a subject, the method comprising:
a) measuring, directly or indirectly, the level of Novlnc44 having a cDNA sequence as set forth in SEQ ID No. 100, fragments thereof, isoforms thereof and variants sharing at least 80% nucleotide sequence identity thereto; and
b) analyzing the levels of said Novlnc44, fragments thereof, isoforms thereof and variants sharing at least 80% nucleotide sequence identity thereto, in conjunction with respective reference value ranges for said Novlnc44,
wherein a decreased expression level of Novlnc44 in the biological sample compared to the control sample from a normal subject indicates that the subject has aortic stenosis.

The present invention further relates to a method for diagnosing dilated cardiomyopathy (DCM) in a subject, the method comprising:
a) measuring, directly or indirectly, the level of Novlnc44 having a cDNA sequence as set forth in SEQ ID No. 100, fragments thereof, isoforms thereof and variants sharing at least 80% nucleotide sequence identity thereto; and
b) analyzing the levels of said Novlnc44, fragments thereof, isoforms thereof and variants sharing at least 80% nucleotide sequence identity thereto, in conjunction with respective reference value ranges for said Novlnc44,
wherein a decreased expression level of Novlnc44 in the biological sample compared to the control sample from a normal subject indicates that the subject has a dilated cardiomyopathy.

Those skilled in the art will appreciate that the invention described herein is susceptible to variations and modifications other than those specifically described. It is to be understood that the invention includes all such variations and modifications without departing from the spirit or essential characteristics thereof. The invention also includes all of the steps, features, compositions and compounds referred to or indicated in this specification, individually or collectively, and any and all combinations or any two or more of said steps or features. The present disclosure is therefore to be considered as in all aspects illustrated and not restrictive, the scope of the invention being indicated by the appended Claims, and all changes which come within the meaning and range of equivalency are intended to be embraced therein.

Various references are cited throughout this Specification, each of which is incorporated herein by reference in its entirety.

The foregoing description will be more fully understood with reference to the following Examples. Such Examples, are, however, exemplary of methods of practicing the present invention and are not intended to limit the scope of the invention.

### EXAMPLES

### Example 1

### Extended Experimental Procedures

### Transgenic Mouse Enhancer Assay

Mouse transgenic enhancer assays were previously executed and described in Ounzain S, et al., 2014. Images can be found at http://enhancer.lbl.gov/.

### Mice

Animal experiments were approved by the Government Veterinary Office (Lausanne, Switzerland) and performed according to the University of Lausanne Medical School institutional guidelines. Animal experiments were approved by the Government Veterinary Office (Lausanne, Switzerland) and performed according to the University of Lausanne Medical School institutional guidelines.

### Cardiac Injury Models—Ligation of the Left Anterior Descending Artery

Myocardial infarction in mice was induced as previously described (Ounzain S, et al., 2014). Mouse was anesthetized by IP injection of a mixture of ketamin/xylazine/acepromazin (65/15/2 mg/kg). Mouse was placed on warming pad for maintenance of body temperature. In the supine position, endotracheal intubation was performed and the mouse was placed on artificial ventilation with a mini-rodent ventilator (tidal volume=0.2 ml, rate=120 breaths/min. The thorax of the animal was shaved and disinfected with Betadine solution. A left thoracotomy was performed. The pectoralis muscle groups were separated transversely, exposing the rib cage. The fourth intercostal space was entered using scissors and blunt dissection. The pericardium was gently opened and a pressure was applied to the right thorax to displace the heart leftward. A 7.0 silk ligature near the insertion of the left auricular appendage was placed and tied around the left descending coronary artery. Occlusion of the artery was verified by the rapid blanching of the left ventricle. For animals undergoing a sham operation, the ligature was placed in an identical location but not tied. The lungs were re-expanded using positive pressure at end expiration and the chest and skin incision were closed respectively with 6-0 and 5-0 silk sutures. The mouse was gradually weaned from the respirator. Once spontaneous respiration resumed, the endotracheal tube was removed, and the animal was replaced in his cage.

### Echocardiography

Transthoracic echocardiographies were performed using a 30-MHz probe and the Vevo 770 Ultrasound machine (VisualSonics, Toronto, ON, Canada). Mice were lightly anesthetized with 1% isoflurane, maintaining heart rate at 400-500 beats per minute, and placed in dorsal recumbency on a heated 37′C platform. Hair was removed with a topical depilatory agent. The heart was imaged in the 2D mode in the parasternal long-axis view. From this view, an M-mode curser was positioned perpendicular to the interventricular septum and the posterior wall of the left ventricle (LV) at the level of the papillary muscles. LV free wall thickness in diastole (LVWTD) and in systole (LVWTS) as well as LV diameter in diastole (LVDD) and in systole (LVDS) were measured. All measurements were done from leading edge to leading edge according to the American Society of Echocardiography guidelines. The measurements were taken in 3 separate M mode images and averaged. Ejection fraction (EF) was calculated using the formula % EF=[(LVVD-LVVS)/LVVD]×100, where LVVD and LVVS are LV volume in diastole and systole respectively.

### Mouse Tissue Collection and Preparation

Hearts and testes were dissected from sham and MI mice one and seven days post artery ligation. Tissues were rinsed in diethyl pyrocardbonate (DEPC)-treated PBS, snap frozen in liquid nitrogen and stored at −80′C until use. Note, specific care was taken to gently squeeze the hearts with forceps in DEPC-treated PBS to minimize residual blood contamination.

### Embryonic Stem Cell Culture and Differentiation

Nkx2.5-EmGFP BAC reporter ES cell line (129/OlaHsd strain, subline E14Tg2A.4) were kindly provided by Edward C Hsiao (Gladstone Institute of Cardiovascular Research, San Francisco) and maintained and cultured as previously described (Ounzain S, et al., 2014). Cells were cultured on mouse embryonic fibroblasts feeders or on gelatinized plates in standard ES cell medium supplemented with 1000U/ml of LIF. Cardiac differentiation of ES cells was induced by aggregating aliquots containing 1000 cells in hanging drops to form embryoid bodies (Ounzain S, et al., 2014).

### Primary Cell Cultures and Transfections

Neonatal C57B6 mice were sacrificed within the first 24 h after birth. Beating hearts were removed, atria and great vessels were carefully dissected away and placed on ice in ADS buffer (H2O, NaCl 116 mM, HEPES 20 mM, NaH2PO4 1 mM, KCl 5.4 mM, MgSO4 0.8 mM, glucose 5.5 mM). The hearts were minced using a sterile and sharp razorblade, and placed in a 1.5 ml tubes (5-6 hearts per tube) containing 1 ml of PIB digestion buffer (ADS buffer+0.05 mg/ml Collagenase type II (Worthington)+1 mg/ml Pancreatin (Sigma). Place the tubes at 37° C. with shaking at 1000 rpm per 15 minutes, collect the supernatants on a tubes containing a volume of complete medium (DMEM 75%, M199 25% ml, Pennicilin/streptavidin 1×, L-Glutamine 1×, Horse serum 10%, Fetal Cow Serum 5%) equal to the sum of all supernatants from digestion tubes. Add 1 ml of PIB buffer to undigested tissue fragments still in the tubes and repeat the digestions process other 2 times. After the 3 steps of digestion spin down the cells by centrifugation at 800 rpm for 10 minutes at room temperature. Discard the supernatant and resuspend the pellet in adequate volume of complete medium (2 ml each 5-6 hearts). Plate cells in 10 cm dish for 45 minutes in an incubator 37° C., 10% CO₂ (pre-plating 1); after this step the non-myocytes will adhere and the cardiomyocytes will remain in suspension. Transfer the supernatant on a new 10 cm dish to repeat this step another time (pre-plating 2) Add fresh complete medium to the pre-plating dish to culture the non-myocytes cells. After the second pre-plating collect the supernatant in a new tube, count the cells and seed 300.000 cardiomyocytes on gelatin coated 3.5 cm plates. One day after isolation, a final concentration of 100 nM of LNATM longRNA GapmeRs (Exiqon) was transfected on cardiomyocytes using FuGene 6 (Promega). After 72 hr, RNA was extracted using miRNeasy kit (Qiagen) and the knock down confirmed by qPCR. Gapmer Sequences, Scrambled; TCATACTATATGACAG (SEQ ID No 106), Anti-Novlnc6 TACAACCTGCTTACT (SEQ ID No 105).

### Nuclear and Cytoplasmic RNA Fraction Isolations

Nuclear and cytoplasmic RNA fractions were isolated using the Cytoplasmic and Nuclear RNA purification kit (Norgen Biotek Corp, CA, Cat No;37400) according to the manufacturers instructions.

### RNA Isolation, Reverse Transcription, End-Point PCR and Quantitative PCR.

Primer sequences for qRT-PCR are provided below. For TaqMan probe based qRT-PCR expression was analyzed using fluorescent-labeled TaqMan Probes (ABI). Analysis was carried out using an ABI Prism 7500 cycler and relative expression quantified using the ΔΔCt method. For end-point PCR aliquots of PCR mixtures were taken during different cycles for agarose gel analysis to determine linear range of amplification. All reactions were run on a 1.5% agarose gel stained with Ethidium Bromide. Primer sequences were as follows (Fw, Rv).

Novlnc6;
(SEQ ID No 107, 108)
GTGGGGAGGTCAGCTACAAA; CGGAAATGGTTTGAAATGCT,
Novlnc11;
(SEQ ID No 109, 110)
ACAGACCTGCAGCAGTGAGA; GCTAGGGAACGCAGAACAAG,
Novlnc15;
(SEQ ID No 111, 112)
AAGGCTTCCCAGAGAAGGAG; ACTGGGTGAGTCTCGCTGTT,
Novlnc23;
(SEQ ID No 113, 114)
TGGGACAGCAGAGCTAAGGT; AGATTCCAGCACGCACTTCT,
Novlnc32;
(SEQ ID No 115, 116)
AAAGGGAAGAGGGAAAACGA; CGTCTAGAACCAGCCCAGAG,
Novlnc35;
(SEQ ID No 117, 118)
CAGCCCTGCTTTAGTTCCTG; TTCGTTGGGGATTTTACTGC,
Novlnc44;
(SEQ ID No 119, 120)
TTTGGAGATGGAACCTGGAG; TCTGGTATGGGGGAGACTTG,
Novlnc49;
(SEQ ID No 121, 122)
AGCTCTGGGTTGGACTGAGA; TGCATACATTCTGGCAGAGC,
Novlnc61;
(SEQ ID No 123, 124)
GGTTGGGTGCCTATTAAACG; GGTTCATGAGCCTTTGGAAG,
Novlnc76;
(SEQ ID No 125, 126)
TGTTAATTCAGGGGCACACA; GGTGGAGAGCCACTGAAGAG,
Novlnc86;
(SEQ ID No 127, 128)
TCTCTGTCCCTTGTGTGTGC; CTTGGAGGTGTGGGCATAGT,
Novlnc90;
(SEQ ID No 129, 130)
GAGCCAAGTGCACACAGAAA; TGGTCTGTTCCTGGCCTTAG,
Novlnc95;
(SEQ ID No 131, 132)
GTGGACGACAAGGGAGGTTA; CGGAATGGCTCCTACAACAT,
Novlnc96;
(SEQ ID No 133, 134)
GAGGCTCCTGGATCTCTGTG; TTGGGAGGCAAAGGTAGATG,
Novlnc103;
(SEQ ID No 135, 136)
GAAATGAGTGGTGGCAGTGA; CTTAGGTCTGCGCCTAATGG,
Novlnc174;
(SEQ ID No 137, 138)
GCACAGATGCATAGCCTCAA; GCAGCCTGGACTTTTCTCAC,
Novlnc333;
(SEQ ID No 139, 140)
TCACCTCCAAGTGGGTCTTC; AGCTCGGTCTGTCGTGAGTT,
MmMyocardin;
(SEQ ID No 141, 142)
CAAGGCTTAATACCGCCACTG; AATGTGCATAGTAACCAGGCTG,
MmBMP10;
(SEQ ID No 143, 144)
ATGGGGTCTCTGGTTCTGC; CAATACCATCTTGCTCCGTGAA,
MmTbx20;
(SEQ ID No 145, 146)
AAGAGATACCGCTATGCCTACC; GCTGCTCGCCAGTAAAGGG,
Colla1;
Mm_00801666_g1,
CTGF;
Mm_01192931_g1,
NPPA;
Mm_01255747_g1,
TGFb2;
Mm_00436955_m1,
Nkx2-5;
Mm_00657783_m1,
GATA4;
Mm_00484689_m1,
Tbx5;
Mm_00803521_m1,
Myh6;
Mm_00440354_m1,
Myh7;
Mm_00600555_m1,
NPPB;
Mm_00435304_g1.

### RNA Sequencing and Analysis

Total RNA was isolated from adult mouse hearts using the RNeasy isolation kit (Qiagen). Sequencing libraries were prepared according to Illumina RNA Seq library kit instructions with Poly(A) selection. Libraries were sequenced with the Illumina HiSeq2000 (2×100 bp).

Sequence analysis of long RNA reads 100nt paired-end reads from 8 samples (4 Sham, 4 LAD) were mapped to mm9 reference genome using Tophat software version 2.0.5 (Trapnell et al., 2012) with option “Gene model”-G, using mm9 UCSC reference genes GTF (Karolchik et al., 2003). An ab initio transcript reconstruction was performed using Cufflinks, version 2.0.2 (Trapnell et al., 2012). The option “masking” (−G) was used, using mm9 UCSC reference genes GTF. The other parameters were default. The resulting GTFs were merged using Cuffmerge, version 2.0.2, using option −g with mm9 UCSC GTF as reference, allowing distinguishing known and novel transcripts.

Classification of lncRNA

Using the output of Cuffmerge, the transcripts were classified into 3 categories: known mRNAs, known lncRNAs (UCSC as reference) and novel lncRNAs. Novel transcripts were filtered for minimal length of 200 bp and at least 2 exons. Read counts were then calculated per gene from the alignment bam files using HTSeq (v0.5.4p2) with options-m union—stranded no. Genes were then filtered for minimal expression (mean counts >=5 across all conditions). lncRNA genes were classified into several categories by comparing the lncRNA exon and gene coordinates with coordinates of known protein coding genes. The categories were as follows: ‘Intergenic Same Strand’ was where all exons of the lncRNA gene were between two protein coding genes; ‘Intergenic Convergent’ was where a protein coding and lncRNA gene are transcribed on opposite strands but pointing towards each other; ‘Intergenic Divergent’ was where a protein coding and lncRNA gene are transcribed on opposite strands but pointing away from each other; ‘Exonic Sense’ was where at least one exon of an lncRNA overlapped with an exon of a protein coding gene in the same direction; ‘Exonic Antisense’ was the same as for ‘Exonic Sense’ but with lncRNA and protein coding genes on opposite strands; ‘Intronic Sense’ and ‘Intronic Antisense’ were where a lncRNA was completely contained within the intron of a protein coding gene on the same, or opposite strand respectively; ‘Overlapping Sense’ and ‘Overlapping Antisense’ was where a lncRNA gene's coordinates overlapped with those of a protein coding gene on the same or opposite strand respectively.

Differential Expression Analysis of lncRNAs

Count data was fitted to a statistical model based on the negative binomial distribution using the R package DESeq, which is useful for detecting significant RNA-Seq variation with a low number of biological replicates. To perform the normalization and differential expression analysis, raw read counts per gene were normalized to the relative size of each library. Empirical dispersion (the squared coefficient of variation for each gene) was estimated using the pooled method. Here, samples from all conditions with replicates are used to estimate a single pooled dispersion value, which is applied to all samples. The dispersion-mean relationship was then fitted using the local method and the fitted value only was used in subsequent calculations. The difference between the means of treated vs non-treated samples was then calculated using a negative binomial test and p-values were adjusted for multiple comparisons using the Benjamini-Hochberg method. Genes with an adjusted p-value of ≤0.01were considered to be differentially expressed.

### LncRNA Analysis

Coding potential—The protein-coding potential of transcripts was evaluated using the program GeneID, version v1.4.4, applied to transcript sequences in FASTA format, with parameters adapted for vertebrates as provided by the authors in file GeneID.human.070123.param, and with options −s and −G.

PhastCons score—PhastCons scores (calculated on a multiple alignments of 30 vertebrate genomes to the mm9 mouse genome) by chromosome were downloaded from the UCSC website. For each gene, scores per base for exons, introns and promoters (defined as 1000 bp upstream from TSS) were summed and divided by the fragment length. This result was used as the score per fragment. 50000 random intergenic regions were generated (size=3400 bp±20%) and the same score was calculated. Log10 of the scores was plotted by category using R package lattice. The scores of the intergenic regions were added to the 3 plots (exon, intron, promoter) as a comparison.

### Chromatin Marker Levels

For the analysis of chromatin marker levels at promoters, we used data published by Wamstad et al. (Wamstad et al., 2012), observed in cell lines representative of successive stages along cardiac differentiation (ESC, MES, CP, CM) (downloaded from data repository of the Cardiovascular Development Consortium (CvDC), part of the NHLBI Bench to Bassinet Program). Levels of five markers (H3K4me1, H3K4me3, H3K27ac, H3K27me3 and RNAP Ser5P) were evaluated within 2 kb regions centered on the TSS of each transcript, using the map command of the BEDTools program, version 2.17.0, with default parameters. These levels were normalized by computing the ratio of ChIP to input WCE DNA within the same region. Based on the resulting profiles, transcripts were distributed among the 31 ChIP clusters identified by Wamstad et al. (Wamstad et al., 2012). Each transcript t was ascribed to the nearest cluster, as measured by a distance d(t,C) based on the Spearman correlation S(t,c) of profiles between the transcript t and the n members c of cluster C:

$$d ( t , C ) = 1 - ( \frac{1}{n} {\sum }_{1}^{n} \vert S ( t , {c}_{n} ) \vert )$$

### Chromatin States

To analyze the presence of chromatin marker peaks at promoters, we used data published by Wamstad et al. (Wamstad et al., 2012), observed on cardiac differentiation cell lines, and data observed in adult tissue (heart, kidney, liver, spleen, testis) published as part of the ENCODE project (The ENCODE Project Consortium, 2011), generated and analyzed in Bing Ren's laboratory at the Ludwig Institute for Cancer Research, UCSC. Four markers were considered: H3K4me1, H3K4me3, H3K27ac and H3K27me3. Depending on presence of these markers, each transcript was ascribed to one of eight distinct chromatin states: H3K4me1, H3K4me3, or H3K27me3 alone, combination of H3K4me3 and H3K27me3, H3K4me3 and H3K27ac, H3K27me3 and H3K4me1, H3K27ac and H3K4me1, or none of these previous combinations. A marker was considered present if a non-empty intersection could be detected between the TSS region and a marker peak, in any of the replicates. The intersections were detected using the window command of the BEDTools program (Quinlan and Hall, 2010), version 2.17.0, with option −w 1000.

### Mosaic Plots

Mosaic plots were used to visualize joint frequency distributions (e.g. across ChIP clusters, gene categories or differential expression status). These plots were generated using the cdv R package. In some of these plots, a residual-based shading was applied to visualize the pattern of deviation from independence. Most data processing, statistical analysis and generation of graphics was performed using the R language (R core team, 2013).

### Gene Expression Across Tissues—Expression Heatmaps

Expression of the genes (RefSeq+novel lncRNAs) in 18 mouse tissues (Adrenal, Bladder, Colon, Duodenum, Heart, Kidney, Large Intestine, Liver, Lung, Mammary gland, Ovary, Placenta, Subcutaneous Adipose tissue, Small Intestine, Spleen, Stomach, Testis, Thymus) was measured on ENCODE public data (CSHL Long RNA-seq, PI Gingeras, Lab CSHL-m) (The ENCODE Project Consortium, 2011). Counts on plus and minus strands were summed and mean counts were taken for the two replicates per tissue. Expression for the same genes was also measured on the 8 LAD/Sham samples. Between sample normalisation was performed using DESeq (estimateSizeFactors function. Only genes with minimal expression were kept (mean counts >=5 across all conditions). Heart Specificity (HS) score (per gene) was defined as:

$$HS score = \frac{{μ}_{cardiac}}{{μ}_{non - cardiac + 2} * {σ}_{non - cardiac}}$$

Where μcardiac is the average expression per gene in our 8 samples, μnon-cardiac is the average expression per gene in non-cardiac ENCODE samples, and σ non-cardiac is the standard deviation per gene in non-cardiac ENCODE samples. (adapted from (Anders and Huber, 2010b)). A gene was considered heart specific with HS score >1. Heatmaps were generated using heatmap.2 from the package gplots in R, version 2.11.0. The clustering was performed using hclust, version 1.3.1, using Spearman correlation and euclidean distance, average linkage clustering. A scaling by row was applied. The same sample order was used for all heatmaps to enable comparison. The HS bars were generated using the HS score defined above. The filter for differentially expressed genes is adjusted p-value for differential expression <0.01. P-values for significance of difference between percentages of HS between groups were calculated using Pearson Chi-squared test.

Correlation of Expression Between Novel lncRNAs and Closest Coding Genes

The coordinates of the novel lncRNAs were compared to RefSeq coding genes reference. If the coordinates of the lncRNA overlapped with a known gene (at least 1 bp), this gene was considered as the closest overlapping gene. If there was no overlap with a known gene, the closest gene was selected and classified as upstream or downstream depending on its position. For gene expression, the same data as in Expression heatmap was used. The correlation of expression was calculated between the lncRNA and closest coding gene using Pearson correlation. This was done for novel and UCSC lncRNAs. As a comparison, the same method was applied on 2000 non-redundant random pairs of closest coding genes. To generate a set of correlations between random mRNA pairs, a pairwise Pearson correlation matrix was calculated between all genes, and 100k random pairs were selected from it (excluding redundant ones)

### Correlation Gene Expression—Physiological Traits

All heatmaps were generated using the R heatmap.2 function, from package gplots. No scaling was applied. All clustering was performed using the R function hclust. Physiological traits were correlated using the Spearman method, and clustering was using euclidean distance and complete linkage clustering. Correlation between physiological traits and gene expression was calculated using the Spearman method, comparing 2 vectors containing 8 values of gene expression and 8 values of trait measure, respectively. Horizontal and vertical clustering were performed on the expression and traits values, using the Spearman method for the correlation, and average linkage clustering. The density plots of the Heart Specificity by category used the same HS scores as described above and were performed using R package gplots, function ggplot.

### TransMap

The GTF file containing the novel lncRNAs to transmap from mouse to human was converted into a psl file using utilities gtfToGenePred and genePredToPsl. pslMap utility was then used with the new psl file and ENCODE chain alignment hg19.mm9.all.chain downloaded from the UCSC website. The orthologs discovered by pslMap were then filtered using pslCDnaFilter with option −globalNearBest=0.005 and −minCover=0.2. The 4 standalone programs used above were downloaded from UCSC utilities.

### Visualisation on UCSC

Bigwig files were generated using Bedtools suite, version 2.17.0 from the bam files generated by Tophat. The tool used was genomeCoverageBed, with options −bg, −ibam and −scale. The size factors used to scale were calculated using DESeq (as described above). The bigwig files were then uploaded on an ftp server and the link was uploaded on the UCSC genome browser.

### GO Analysis—Enrichment for Biological Themes

The genes lists were submitted online to the DAVID Functional annotation clustering tool using default parameters and databases.

### Human Methods

All human material was obtained during routine sampling used for clinical purposes, stored in a coded way and available for research purposes in accordance with the Declaration of Helsinki and the ethical committee at Maastricht University Medical Center. Right ventricular septal biopsies were obtained during routine clinical sampling from patients with idiopathic dilated cardiomyopathy (DCM) and decreased ejection fraction without cardiac inflammation. Controls (n=6) consisted of patients with unexplained ventricular tachy-arrhythmias but with a normal ejection fraction and the absence of systemic or cardiac inflammation at the time of biopsy. Left ventricular biopsies were obtained from patients with aortic stenosis (AOS) and from control patients undergoing coronary artery bypass grafting (CABG) (Suppl. Table Y). Cardiac biopsies were immediately snap-frozen for total RNA was isolation with the miR Vana miRNA isolation kit (Ambion, Austin, TX). Total RNA was reverse transcribed using iSCript (Bio-Rad, Hercules, CA) and SYBR Green quantitative PCR was performed on a Bio-Rad iCycler.

### Statistical Analysis

Data throughout the paper are expressed as mean #SEM. One way ANOVA was used to test significance of data comparisons between experimental groups, with p values <0.05 were considered significant.

### Results

Global Identification of lncRNAs Expressed in the Heart and Regulated During Myocardial Infarction

The Inventors first set out to characterize global transcriptional regulation during myocardial adaptation to stress for both the coding and non-coding transcriptome alike. The Inventors utilized a well-characterized pathophysiological model of cardiac stress in the mouse, namely myocardial infarction obtained by left anterior descending artery ligation. The Inventors identified lncRNAs expressed in the infarcted adult mouse heart by employing a RNA-Sequencing (PolyA+RNA-Seq) and ab initio transcriptome reconstruction approach. Illumina-based massively parallel sequencing was used to obtain paired-end reads (2×100 bp) of experimental libraries, and Cufflinks ( ) was utilized to perform ab initio transcript assembly on mapped paired-end reads.

This analysis reconstructed 17584 multi-exonic transcripts, of which 15075 (2204 up-regulated and 1338 down-regulated) correspond to University of California Santa Cruz (UCSC) annotated protein coding genes (See Ounzain S, et al., 2014). Our lncRNA annotation pipeline identified 2509 multi-exonic lncRNAs (>200 bp). There were 988 (67up-regulated and 66 down-regulated) UCSC annotated lncRNAs and 1521 (86 up-regulated and 225 down-regulated) novel previously un-annotated lncRNAs, encompassing all known lncRNA locus-types (data now show). To verify the non-coding nature of our novel lncRNA candidates, The Inventors used GeneID coding potential score algorithm and found that these novel transcripts encode minimal protein coding potential comparable to UCSC annotated lncRNAs (see Ounzain S, et al., 2014). Furthermore, novel lncRNAs and UCSC lncRNAs were expressed at significantly lower levels than coding genes (FIG. 1B). UCSC lncRNA exons were less conserved than coding exons although promoters were equally conserved (FIG. 1B).

Novel lncRNAs are Heart Specific and Proximal to Cardiac Developmental Genes

The majority of lncRNAs identified in our analysis represent novel lncRNAs that have previously escaped annotation. The Inventors aligned 17 mouse non-cardiac ENCODE RNA-Seq data sets (Mouse et al., 2012), and found that 16% of UCSC mRNAs and 23% of UCSC lncRNAs were classified as heart specific (see Ounzain S, et al., 2014). By contrast, 38% of the novel lncRNAs were heart-specific, a significant enrichment versus UCSC mRNAs and lncRNAs. Furthermore, differentially expressed novel lncRNAs were significantly more heart-specific than all transcript classes, with 60% of these novel lncRNAs being classified as heart specific (see Ounzain S, et al., 2014). LncRNAs have been shown to regulate coding gene expression both in cis and in trans. If cis-regulation was common, one would expect proximal coding genes to also be more heart-specific. In support of this, The Inventors found that overlapping, proximal upstream or downstream coding genes were significantly more heart-specific than the entire coding gene collection. Furthermore, gene ontology analysis of these proximal coding genes revealed significant enrichment of biological processes associated with heart development, cardiac function and transcriptional regulation. Interestingly differentially expressed novel lncRNAs were more associated with transcriptional control suggesting that modulated expressed novel lncRNAs may be implicated in transcriptional reprogramming observed in the remodeling heart.

The Cardiac Transcriptome is Highly Correlated with Cardiac Physiological Traits

The Inventors correlated the cardiac transcriptome with echocardiography derived physiological traits. Both the coding and non-coding transcriptome correlated tightly with cardiac physiology (FIG. 1). Globally, novel lncRNAs were better correlated than UCSC lncRNAs with all physiological traits assessed. To gain a deeper molecular insight and potentially identify molecular pathways associated with physiological traits, The Inventors executed unsupervised clustering and further downstream analysis of UCSC coding genes and novel lncRNAs. The Inventors identified four clusters for both coding (FIG. 1A, -B) and novel lncRNA (FIG. 1C, -D) transcripts. In each case, these consisted of one cluster that correlated positively with cardiac function and negatively with remodeling parameters, one cluster with the inverse of these correlations and two clusters with non-specific intermediate correlations. Gene ontology (GO) and heart specificity analysis was executed on individual clusters with GO analysis being executed on the most proximal coding genes with respect to novel lncRNAs. In the coding gene group, the most heart-specific cluster was Cluster 2 (FIG. 1E), which was positively correlated with cardiac functional traits and associated with genes involved in mitochondrial biology (FIG. 1A). The least heart-specific cluster (Cluster 4) was positively correlated with remodeling and associated with genes involved in wound healing and extracellular matrix (FIG. 1A). Within novel lncRNAs, the most heart-specific cluster, i.e. Cluster 4 (FIG. 1F), was again positively correlated with cardiac function associated traits. Proximal coding genes to novel lncRNA in Cluster 4 were enriched with heart development associated processes (FIG. 1C). Since novel lncRNAs that cluster specifically with particular physiological traits were likely to be involved in biological processes associated with those traits, these findings indicated that novel lncRNAs within this cluster could represent a class of cardiac-specific regulators of developmental gene programs, which was reactivated in the damaged myocardium. Finally, the least heart specific clusters were one and two, which was positively correlated with remodeling traits.

These data demonstrated that unsupervised clustering of transcripts was able to distinguish physiological traits. In addition, it indicated that lncRNAs could represent specific markers of particular physiological traits. To test this, The Inventors compared correlation distributions for each UCSC coding gene and novel lncRNA cluster, with each of the following traits; ejection fraction (EF), interventricular septal thickness at systole (IVS), myocardial infarction trace (MI trace) and left ventricular internal diameter at systole (LVID) (FIG. 1B and D). UCSC coding gene Clusters 2 and 4 strongly correlated with all these traits when compared to non-specific clusters (Clusters 1 and 3) (FIG. 1B). A similar pattern of correlation was observed with novel lncRNA clusters 2 and 4 (FIG. 1D). However, novel lncRNA Cluster 1 was particularly interesting since it exhibited poor correlation with LVID, EF and MI trace but correlated well with IVS which is typically linked to EF3. This unique characteristic is likely a consequence of the exquisite context and cell-type specific expression of lncRNAs, and has intriguing implications for the utilization of novel lncRNAs as biomarkers.

Inferring Functions for Novel lncRNAs Based on Developmental Chromatin State Patterns

Pathological cardiac remodeling is associated with the global reactivation of the fetal gene program. The Inventors reasoned that many novel lncRNAs likely represent ‘fetal’ genes with important roles during cardiogenesis. To investigate this, The Inventors utilized ChIP-Seq data generated in a directed differentiation system that recapitulated the step-wise differentiation of mouse embryonic stem cells (ESCs) to differentiated cardiomyocytes (CMs) (Wamstad et al., 2012).

A previous study demonstrated that co-expressed genes during cardiac differentiation could be functionally grouped based on different chromatin state patterns (Wamstad et al., 2012). Each sub-group of genes appeared to be involved in distinct biological processes, including signaling, metabolism and cardiac muscle contraction. The Inventors reasoned that novel lncRNAs that shared specific chromatin patterns as those described for coding genes were likely to be involved in comparable biological processes, thus providing an unbiased chromatin based proxy to functionally annotate novel lncRNAs. The Inventors mapped the predetermined chromatin patterns (ChIP clusters 1 to 34) to the novel lncRNA promoters (and UCSC annotated genes) during ES cell differentiation. The Inventors classified the novel lncRNAs based on which chromatin pattern they were associated with, and inferred a biological function based on the coding genes and biological processes previously linked to each cluster (see Ounzain S, et al., 2014). For clarity, The Inventors present nine ChIP clusters and inferred biological processes associated with each of our transcript classes. ChIP clusters 1 and 3 are associated with ubiquitous housekeeping and non-cardiac developmental processes (see Ounzain S, et al., 2014). UCSC coding genes and lncRNAs were enriched within these clusters while novel lncRNAs were depleted. On the other hand, novel InRNAs were enriched in ChIP-clusters 23, 24, 25 and 26 which are associated with cardiac developmental and functional processes including contractile fiber, z-disk and heart development terms (see Ounzain S, et al., 2014).

The Inventors also identified ChIP-clusters that were enriched or depleted in up and downregulated novel lncRNAs post myocardial infarction, providing a functional insight into the roles of novel lncRNAs in this response (see Ounzain S, et al., 2014). Novel lncRNAs in ChIP-clusters 23 and 24 were enriched in down-regulated lncRNAs post infarction. These clusters are associated with cardiomyocyte maturation and sarcomeric genes (eg. Myoz2, Myl2). The enrichment of ChIP-cluster 23 and 24 novel lncRNAs in down-regulated lncRNAs could be indicative of the re-activation of the fetal gene program in the border zone post infarction and/or a loss of mature cardiomyocytes. Furthermore, ChIP-clusters enriched in up-regulated novel lncRNAs included cluster 18, which is associated with immune and inflammatory responses (eg. IL17b), and cluster 28, which is associated with calcium homeostasis and G-protein coupled receptor signaling (eg. Gnb3), processes that are typically activated in the border zone of the infarcted heart. Finally giving further support to the notion that our novel lncRNAs may be cardiac developmental associated transcripts, The Inventors mapped them to a list of bona fide in vivo validated enhancers active specifically within the E11.5 mouse heart. The Inventors found that seven of our novel lncRNAs map to validated cardiac enhancers including novlnc6 (see below) which maps to mm77, a cardiac enhancer specifically active within the embryonic left ventricle (see Ounzain S, et al., 2014).

Validation of Novel lncRNAs

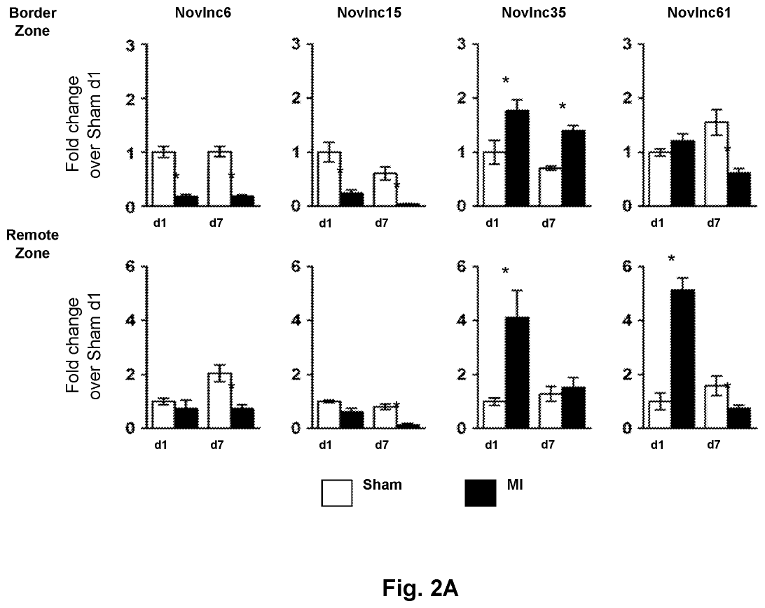

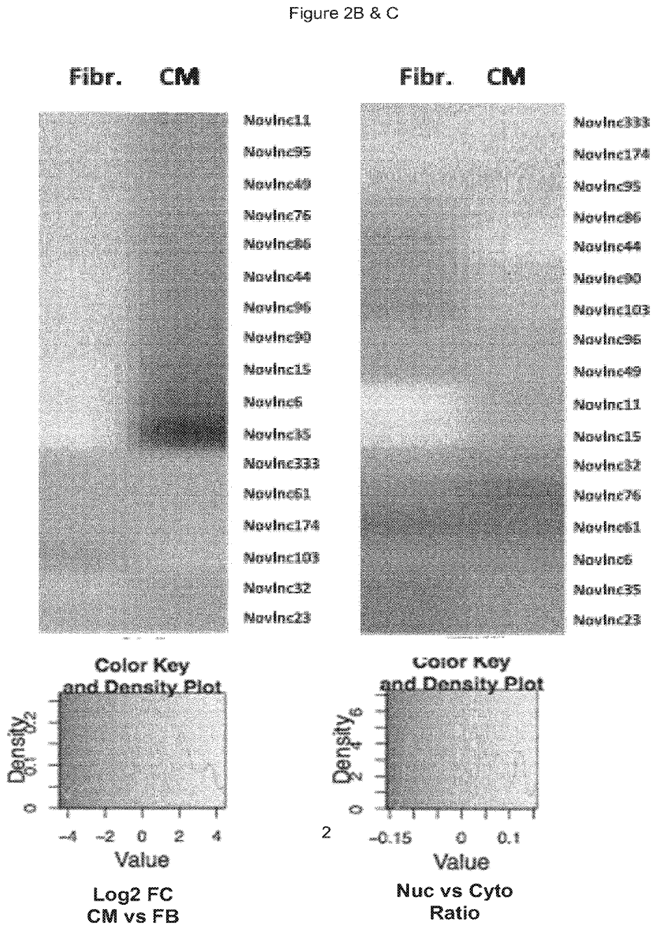

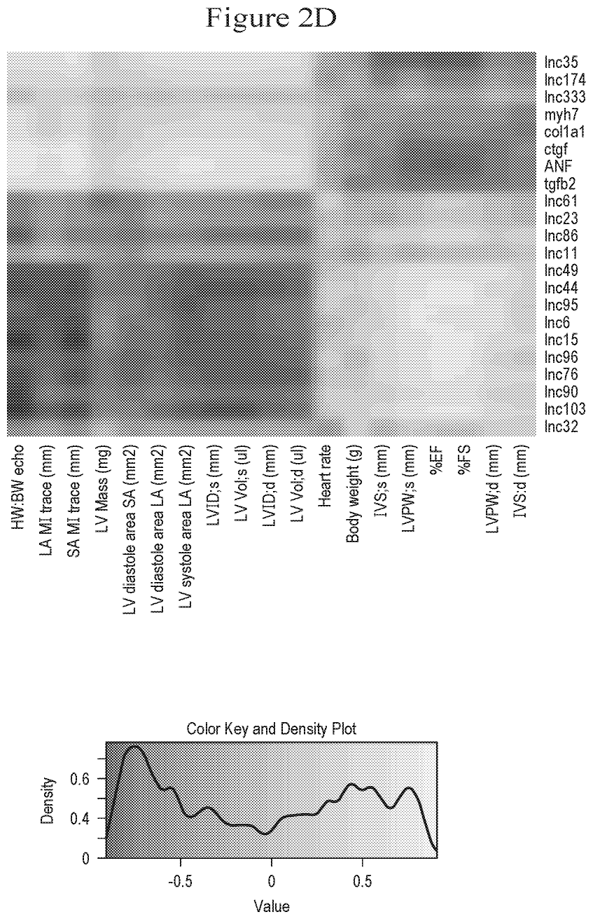

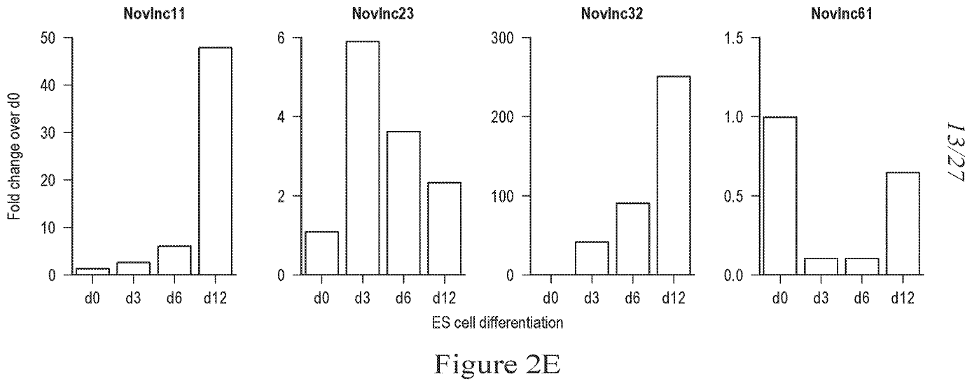

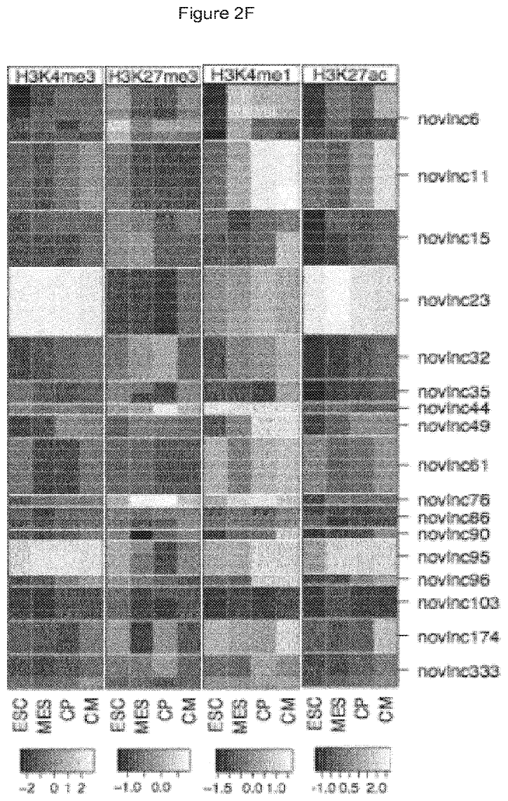

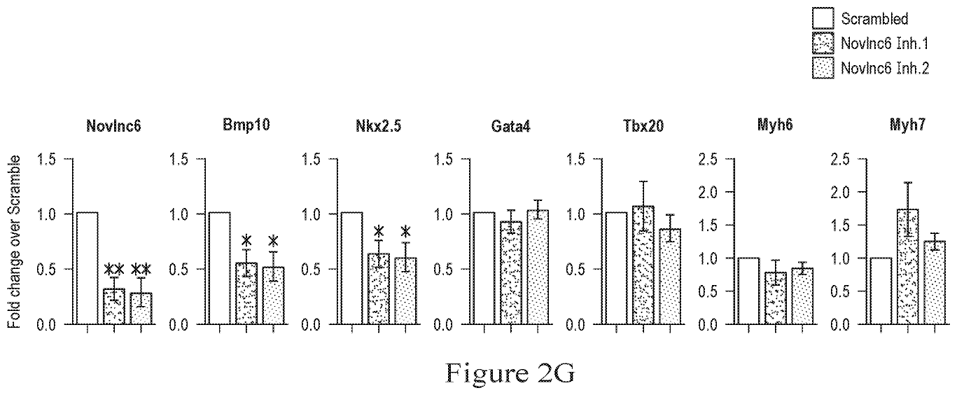

To gain confidence in our transcript nominations, The Inventors validated multiple unannotated novel transcripts by quantitative real-time PCR (qPCR). Seventeen high priority novel candidates were selected, and their expression was quantified in the border (BZ) and remote zones (RZ), one and seven days after infarction. The novel lncRNAs exhibited various kinetics of expression in both the BZ and RZ during the acute and chronic phases (FIG. 2A). Many lncRNAs were downregulated; e.g. Novlnc6 (SEQ ID No. 48) and 15 (SEQ ID No 97) some were transiently induced at day 1 in both RZ and BZ (Novlnc35) while others were gradually increased in BZ and RZ (Novlnc74). These distinct kinetic and spatial patterns of expression demonstrate that novel lncRNAs are dynamically regulated in response to myocardial infarction, and suggest that they likely play important roles in the adaptive process.

The two major cell types within the adult heart are cardiomyocytes (CMs) and cardiac fibroblasts (FBs) with both being important in maladaptive remodeling. To better characterize the novel lncRNAs, The Inventors quantified their expression in CMs and FBs isolated from the neonatal mouse heart. The selected lncRNAs were either highly CM-specific (Novlnc35; SEQ ID No 99), equally expressed in both cell types (Novlnc61, SEQ ID No 101) or primarily expressed in FBs (FIG. 2B). LncRNA function is also dependant on subcellular localization. Cis-acting lncRNAs (i.e. enhancer-associated RNAs) tend to be more enriched in the nucleus whereas lncRNAs involved in post-transcriptional and translational processes tend to be more cytoplasmic. Therefore, nuclear and cytoplasmic RNA fractions were isolated from neonatal

CMs and FBs (FIG. 2C). Validated lncRNAs were either enriched in nuclear (Novlnc174; SEQ ID No 28) or cytoplasmic (Novlnc61; SEQ ID No 101) fractions, in addition to being equally present in both (Novlnc15). Some lncRNAs interestingly displayed differential nuclear versus cytoplasmic enrichment in CMs and FBs (Novlnc90, −49, −11; SEQ ID No 88, SEQ ID No 103, SEQ ID No 25). This may be of functional relevance to roles in these different cell types. The Inventors also correlated the expression of these validated lncRNA with physiological traits in day 1 and day 7 control and MI tissue samples (FIG. 2D). The majority of the lncRNAs correlated well with physiological traits both in border and remote zones. Interestingly, some of our novel lncRNAs were better correlated than stress genes with cardiac function and remodeling.

Many of the validated novel lncRNAs were associated with dynamic changes in chromatin states during cardiogenesis in ES cells, suggesting they may have roles as ‘fetal’ developmental genes (FIG. 2F). To confirm this, mouse ES cells were differentiated through embryoid body (EB) formation using the hanging droplet model recapitulating embryonic cardiac development in vitro. Novel lncRNAs were dynamically expressed during cardiac differentiation with expression correlating with the dynamic changes in chromatin states observed at their promoters (FIG. 2E and F). Some lncRNAs were induced late during differentiation at the CM stage (Novlnc44, −11, −32; SEQ ID No 100, SEQ ID No 25, SEQ ID No 98, FIG. 2E and F), and are therefore likely involved in terminal CM differentiation and maturation. Novel lncRNAs exhibiting this profile were mapped to ChIP-clusters 24 and 25, which are predicted to be associated with heart development, z-disk and sarcomere function, cardiac maturation associated processes. Other lncRNAs were maximally expressed at MES and CPC stages (Novlnc49, SEQ ID No 103,) and are likely to be involved in more specific developmental process.

To evaluate whether novel lncRNAs could be associated to specific function involving regulation of cardiac protein coding genes, the Inventors focused on Novlnc6, which fulfills several criteria and unique features prototypical of lncRNAs identified in this study. Furthermore Novlnc6 (SEQ ID No 48) shares a chromatin pattern in differentiated ES cells with the key cardiac signaling ligand BMP10, suggesting Novlnc6 could be involved in similar regulatory pathways. As an experimental model, The Inventors used primary isolated neonatal mouse CMs expressing high levels of Novlnc6. Cells were transfected with modified anti-sense oligonucleotides (Gapmers) targeting Novlnc6 (FIG. 2G). Key cardiac TFs and downstream cardiac target genes involved in stress signaling, contractile apparatus and BMP10 signaling were examined. This screen identified Nkx2.5 mRNA as a potential target of Novlnc6-mediated regulation. Nkx2.5 encodes a core cardiac TF, high in the regulatory hierarchy of the cardiac GRN and critical for the regulation of other cardiac TFs and downstream cardiac differentiation, structural and maturation genes (Bruneau, 2002). Furthermore, Nkx2-5 has been shown to be downstream of BMP10 signaling during cardiac development (Huang et al., 2012). Novlnc6 positively regulated Nkx2.5, supporting the notion that our collection of novel lncRNAs contains functionally important regulatory transcripts.

### Dysregulation of Human Orthologs in Cardiac Pathology

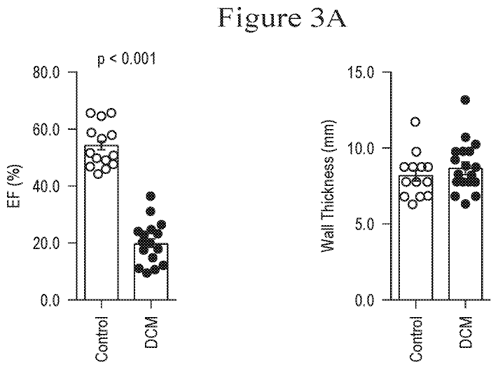

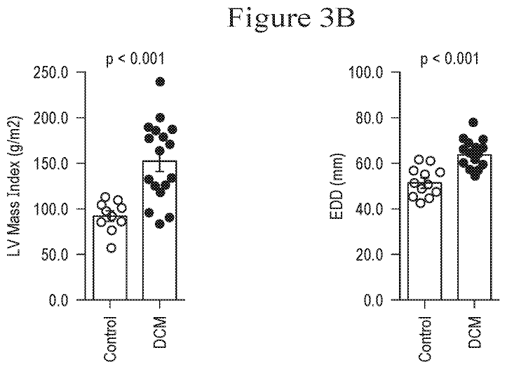

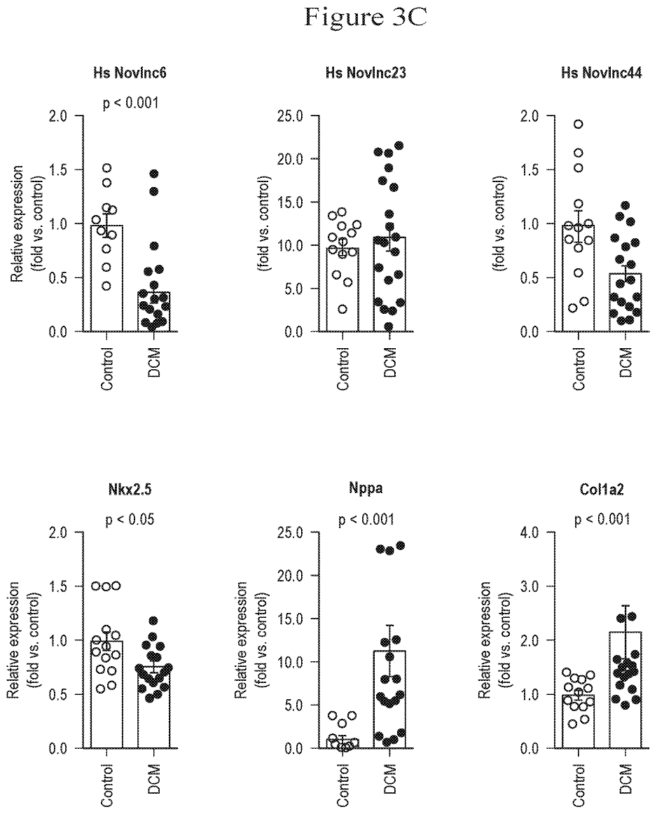

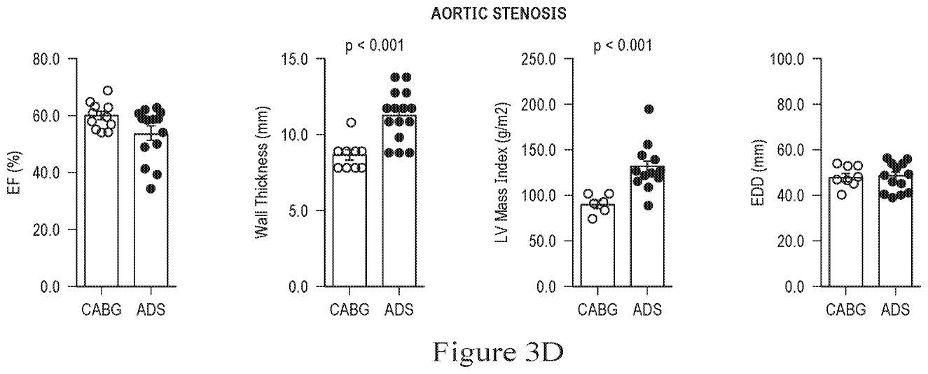

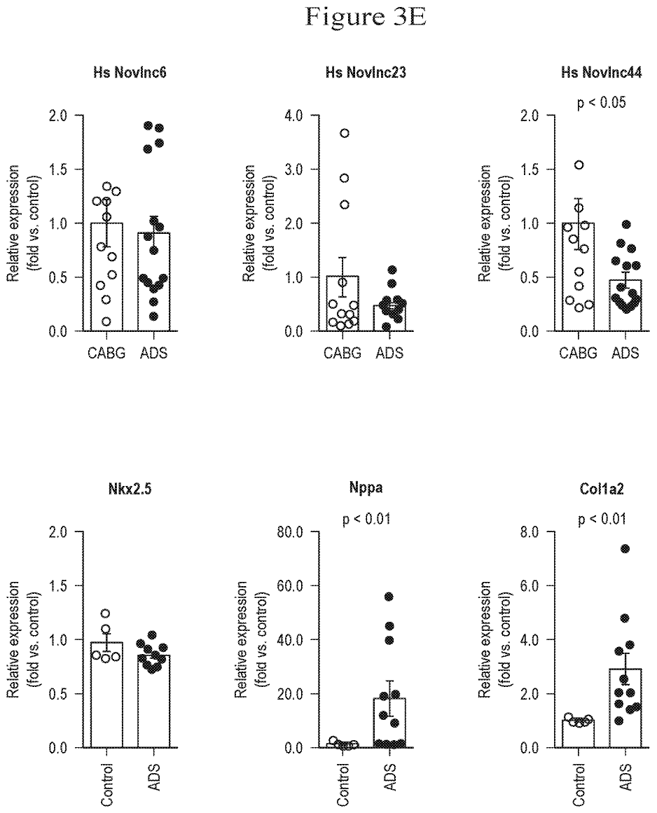

Considering the unique characteristics associated with the novel lncRNAs, The Inventors searched for human orthologs. The Inventors therefore mapped our novel lncRNA catalogue to the human genome using TransMap, a cross species mRNA aligment tool. TransMap maps our novel mouse lncRNAs sequences across the human genome using syntenic BLASTZ alignments that consider conserved gene order and synteny. Of the 311 modulated novel lncRNAs, approximately 72% were confidently mapped to the human genome. To validate and characterize predicted orthologs, The Inventors designed primers encompassed within the putative exons of three human orthologs, corresponding to mouse Novlnc6, −23 and −44 (SEQ ID No 48, 84, 100; FIG. 3A). Quantitative RT-PCR was executed on RNA isolated from the left ventricle of a healthy male. All three putative human orthologs were readily amplified and expressed at relatively high levels (FIG. 3A). To determine the potential roles of these orthologs in cardiac pathology, The Inventors examined their cardiac expression in two independent human heart pathologies. Patients with dilated cardiomyopathy (DCM), and with aortic stenosis (AOS) were assessed. These two cohorts presented with perturbed cardiac functions and associated maladaptive remodeling as expected for such pathologies. Furthermore, cardiac stress marker genes were also differentially expressed (FIG. 3C). In patients with DCM, all three human orthologs were significantly modulated with novlnc6 and −44 downregulated and −23 upregulated (FIG. 3B). Interestingly, the predicted target gene of Novlnc6, i.e the key cardiac TF Nkx2-5, was also significantly downregulated in patients with DCM. In contrast to DCM, patients with AOS were not associated with differential expression of Novlnc6 or −23, or the predicted target gene of Novlnc6, Nkx2-5. Novlnc44, however, was significantly downregulated (FIG. 3B), comparable to its modulation in DCM.

### Example 2

Cell-Specific lncRNA Expression, and Effects of lncRNA Downregulation in Cardiac Fibroblasts and Cardiomyocytes In Vitro

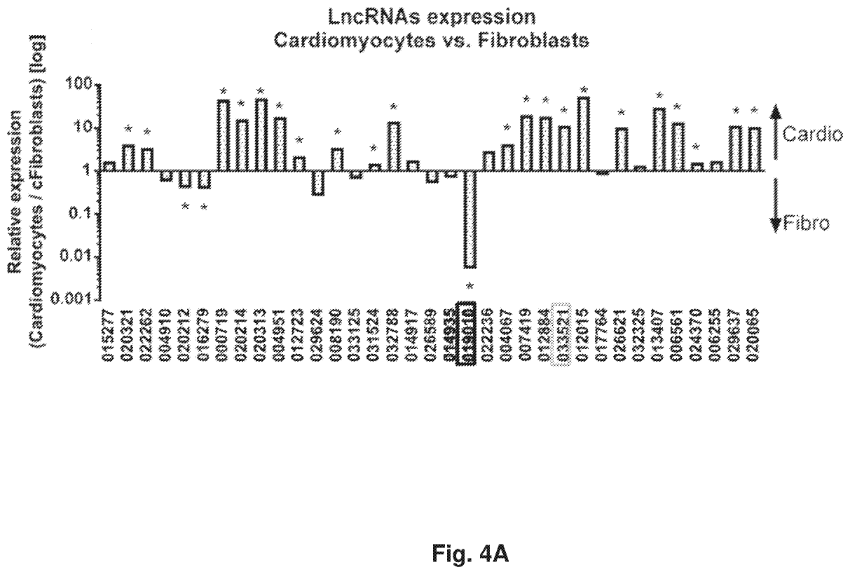

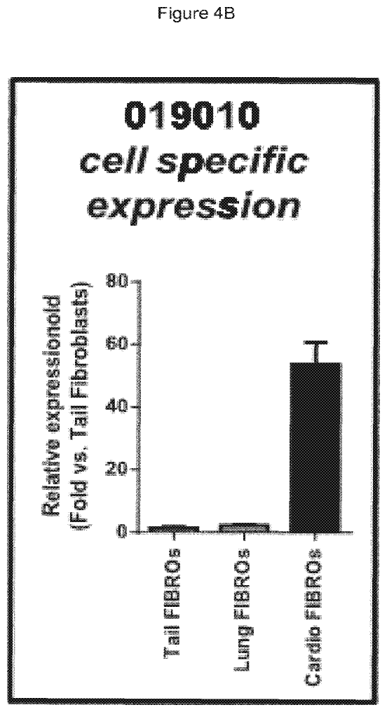

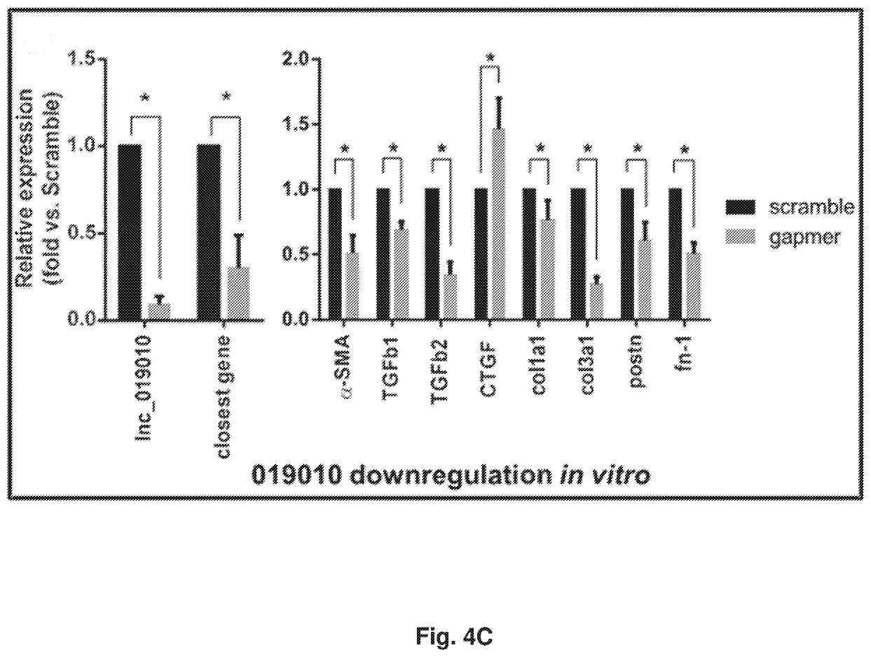

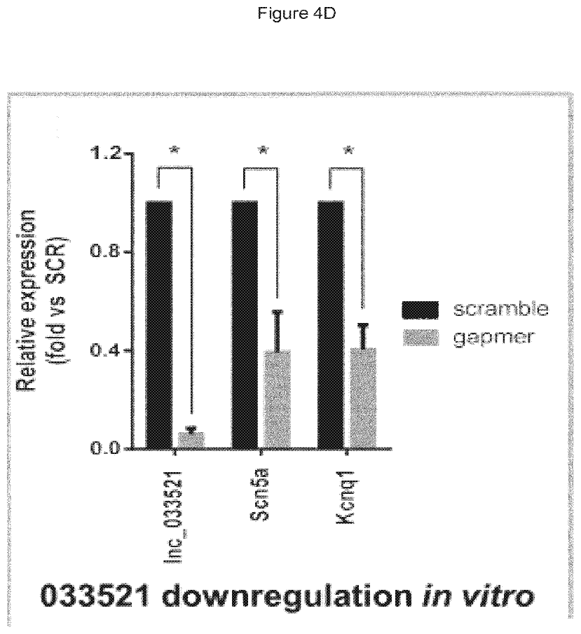

RNA was obtained from cardiomyocytes and cardiac fibroblasts isolated from neonatal mouse hearts. The expression of novel lncRNAs were quantified by RT-PCR in the two cell populations. Ratios in logarithmic scale of cardiomyocyte versus cardiac fibroblast expression of lncRNAs are presented (values below 1 correspond to fibroblast enrichment; values above 1 correspond to cardiomyocyte enrichment). Bars represent mean±SEM (n=3) *p<0.05. The two highlighted lncRNAs: lnc_019010 (fibroblast enriched) and on lnc_033521 (also named Lnc-Dedbt) (cardiomyocyte enriched) have been more extensively studied (FIG. 4A).

The expression of novel lncRNAs in fibroblasts isolated from the tail, the lung and the heart of neonatal mice has been quantified by RT-PCR. The expression of lnc_019010 in cardiac fibroblasts is approximately 60 fold enriched compare to the other sources of fibroblasts. This data show the high cell-specificity of this lncRNA (FIG. 4 B).

Cardiac fibroblasts isolated from neonatal mice were transfected with modified antisense oligonucleotides (GapmeR, 5′-AGGTGTGCGATAGAG-3′) targeting lnc_019010 for degradation. GapmeRs were transfected at a concentration of 50 nM and RNA was harvested 24 h after transfection. Compared to control (scrambled) GapmeR transfected cells (black bars), lnc_019010-specifc GapmeR cells (grey bars) show a strong reduction of the target lncRNA expression and also of the closest coding gene. Interestingly, the downregulation of this cardiac fibroblast-specific lncRNA impact the expression of important fibroblast coding genes such as a-smooth muscle actin (a-SMA), collagen I and III (Colla1, Col3al), fibronectin (Fn1) and periostin (Pstn) and transforming growth factor β1 and β2 (Tgfβ1, Tgfβ2) and connective tissue growth factor (Ctgf). These data, shown in FIG. 4C, suggest that lnc_019010 is involved in cardiac fibroblast differentiation and represents a therapeutic target for limiting fibrosis. Bars represent mean±SEM (n=4) *p<0.05.

Cardiomyocytes isolated from neonatal mice were transfected with modified antisense oligonucleotides (GapmeR) (SEQ ID No. 147: 5′-TGCTTGCTAGTGTGGT-3′) targeting lnc_033521 for degradation. GapmeRs were transfected at a concentration of 50 nM and RNA was harvested 24 h after transfection. Compared to control (scrambled) GapmeR (black bars), lnc_033521-specifc GapmeR cells (grey bars) show a strong reduction of the target lncRNA expression. Moreover, the downregulation of this lncRNA has an impact on expression of important gene encoding fundamental cardiac channel such as Scn5a (sodium channel, voltage-gated, type V, alpha subunit) and Kcnq1 (potassium voltage-gated channel). These data shows that lnc_033521 has important function in the conduction system in the heart. FIG. 4 D; Bars represent mean±SEM (n=4) *p<0.05.

### LncRNAs Downregulation In Vivo

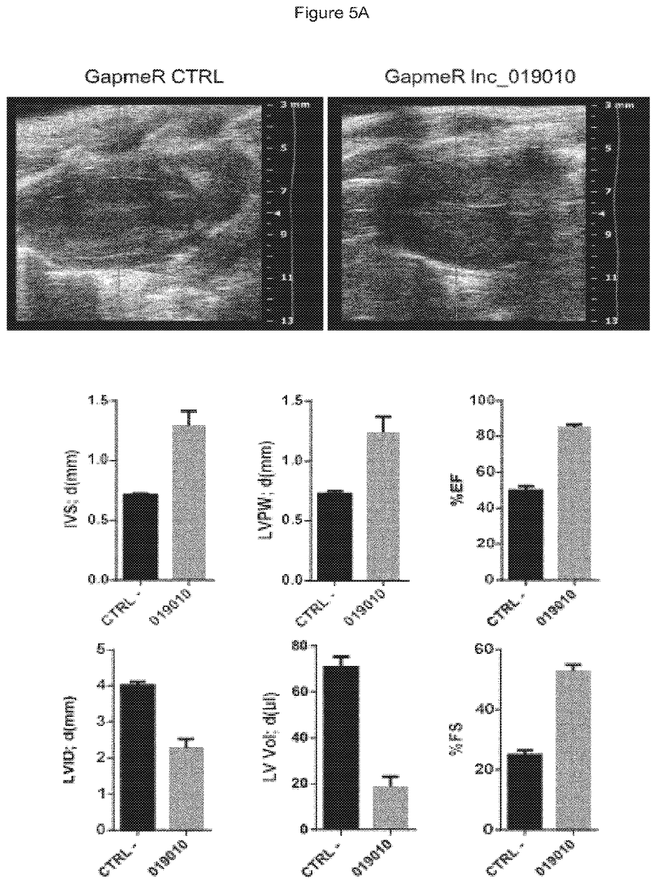

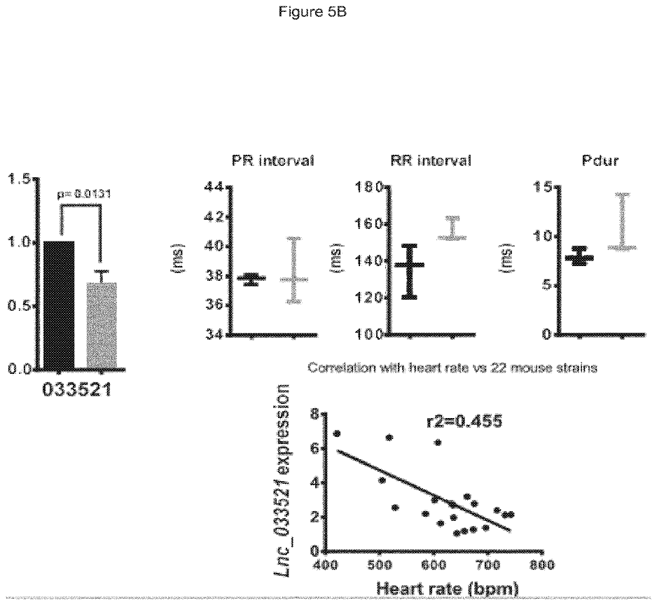

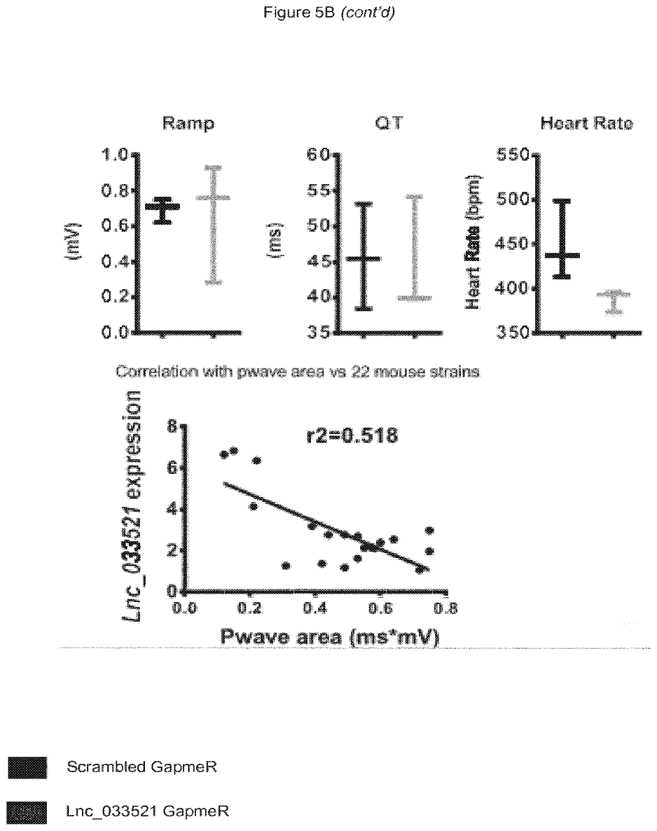

12 weeks old BL6/C7 mice received one intraperitoneal injection of GapmeR (20 mg/kg). Echocardiographic images (long view axis) of mouse hearts 4 days after GapmeR injection ar eshown in FIG. 5A. Left panel, heart of a mouse injected with control (scrambled) GapmeR. Right panel, heart of a mouse injected with GapmeR directed against lnc_019010. Bar graph shows a significant increase in IVS (intra ventricular septum) and LVPW (left ventricular posterior wall) thickness, and a significant decrease of LVID (left ventricle internal diameter) and LV vol (left ventricle volume) in mice injected with GapmeR targeting lnc_019010 (SEQ ID No. 148: 5′-AGGTGTGCGATAGAG-3′) (grey bars) compared to control (scrambled) GapmeR (black bars). These data show that lnc_019010 depletion in the heart in vivo induced a significant increase of heart mass. Ejection fraction (EF) and fraction of shortening (FS) are increased in mice receiving GapmeR directed against lnc_019010, indicating that cardiac function is increased in this case.

RNA was obtained from the heart of mice injected with GapmeR directed against lnc_033521 or control (scrambled) GapmeR. The graph depicted in FIG. 8 B, on the left, shows the downregulation of lnc_033521 expression in mice injected with GapmeR targeting lnc_033521 (grey bar) compare to mice injected with control (scrambled) GapmeR (black bar). Upper panels, electrocardiogrameters showing the effect of lnc_033521 downregulation on cardiac electrophysiology. Heart rate is reduced following downregulation of lnc_033521. Lower panels, correlation between lnc_033521 expression and electrocardiogrameasurements of heart rate (left) and P wave area (right) as measured in 22 different mouse strains. These data show that lnc_33521 plays a role in the regulation of important cardiac electrophysiological parameters.

### REFERENCES

Trapnell, C., Roberts, A., Goff, L., Pertea, G., Kim, D., Kelley, D. R., Pimentel, H., Salzberg, S. L., Rinn, J. L., and Pachter, L. (2012). Differential gene and transcript expression analysis of RNA-seq experiments with TopHat and Cufflinks. Nature protocols 7, 562-578.
Ounzain S, Crippa S, Pedrazzini T. (2013) Small and long non-coding RNAs in cardiac homeostasis and regeneration. Biochim Biophys Acta. 1833 (4): 923-33
Mouse, E. C., Stamatoyannopoulos, J. A., Snyder, M., Hardison, R., Ren, B., Gingeras, T., Gilbert, D. M., Groudine, M., Bender, M., Kaul, R., et al. (2012). An encyclopedia of mouse DNA elements (Mouse ENCODE). Genome biology 13, 418.
Wamstad, J. A., Alexander, J. M., Truty, R. M., Shrikumar, A., Li, F., Eilertson, K. E., Ding, H., Wylie, J. N., Pico, A. R., Capra, J. A., et al. (2012). Dynamic and coordinated epigenetic regulation of developmental transitions in the cardiac lineage. Cell 151, 206-220.
Bruneau, B. G. (2002). Transcriptional regulation of vertebrate cardiac morphogenesis. Circulation research 90, 509-519.
Huang, J., Elicker, J., Bowens, N., Liu, X., Cheng, L., Cappola, T. P., Zhu, X., and Parmacek, M. S. (2012). Myocardin regulates BMP10 expression and is required for heart development. The Journal of clinical investigation 122, 3678-3691.
Kurreck J, Wyszko E, Gillen C, Erdmann V A. (2002) Design of antisense oligonucleotides stabilized by locked nucleic acids. Nucleic Acids Res.; 30 (9): 1911-8.
Sambrook, et al., Molecular Cloning: A Laboratory Manual (3rd Edition, 2001)
Innis et al., eds., PCR Protocols: A Guide To Methods And Applications, Academic Press Inc., San Diego, Calif. (1990).
Froehler et al., (1986) Nucleic Acid Res. 14:5399-5407
McBride et al., (1983) Tetrahedron Lett. 24:246-248
Ounzain S, Pezzuto I, Micheletti R, Burdet F, Sheta R, Nemir M, Gonzales C, Sarre A, Alexanian M, Blow M J, May D, Johnson R, Dauvillier J, Pennacchio L A, Pedrazzini T. (2014) Functional importance of cardiac enhancer-associated noncoding RNAs in heart development and disease.
J Mol Cell Cardiol. November;76:55-70.

1. A method for diagnosing a cardiac pathology in a subject, the method comprising:
a) measuring, directly or indirectly, an expression level of one or more biomarkers in a biological sample derived from the subject, wherein the one or more biomarkers comprises one or more long non-coding RNAs (lncRNAs) have a cDNA sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO:103, SEQ ID NO: 104, a fragment thereof, an isoform thereof, and a variant having at least 80% nucleotide sequence identity thereto; and
b) analyzing the expression level of the one or more biomarkers in conjunction with a respective reference value range for the one or more biomarkers, wherein a differential expression of the one or more biomarkers in the biological sample compared to the one or more biomarkers in a control sample from a normal subject indicates that the subject has a cardiac pathology.

2. The method of claim 1, wherein the expression level of the one or more biomarkers is detected using a method selected from the group consisting of microarray analysis, polymerase chain reaction (PCR), reverse transcriptase polymerase chain reaction (RT-PCR), Northern blot, serial analysis of gene expression (SAGE), immunoassay, and mass spectrometry.

3. The method of claim 1, wherein the biological sample is selected from the group consisting of whole blood, serum, plasma, semen, saliva, tears, urine, fecal material, sweat, buccal smears, skin, cardiac tissue, liver, brain tissue, amniotic fluid, nerve tissue, and hair.

4. The method of claim 3, wherein the biological sample is cardiac tissue.

5. The method of claim 1, wherein the cardiac pathology is selected from the group consisting of interventricular septal thickness (IVS), heart failure, ejection fraction (EF), left ventricular internal diameter at systole (LVID), myocardial infarction trace (MI), heart failure with reduced ejection fraction (HFrEF), heart failure with preserved ejection fraction (HFpEF), viral myocarditis, brachycardia, arrhythmia, a congenital heart defect, diabetic cardiomyopathy, idiopathic and dilated cardiomyopathy, a pathology characterized by malformation, a pathology characterized by tissue remodeling, a pathology characterized by affected function, a pathology characterized by a disorder of the right heart, a pathology characterized by an arrhythmia, a pathology characterized by intoxication, a pathology characterized by cancer, a pathology characterized by a neurologic disorder, a pathology characterized by trauma, and a pathology characterized by a change in hemodynamic.

6. The method of claim 5, wherein the heart failure is HFpEF or HFrEF.

7. The method of claim 1, wherein an increased expression level of the one or more lncRNA having a cDNA sequence selected from the group consisting of SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 96, SEQ ID NO: 99, a fragment thereof, a isoform thereof, and a variant having at least 80% nucleotide sequence identity thereto, in the biological sample compared to an expression level of the one or more of lncRNAs in a control sample from a normal subject indicates that the subject suffered from myocardial infarction or is suffering from cardiac pathology associated with maladaptive remodeling of myocardium.

8. The method of claim 1, wherein a decreased expression level of the one or more lncRNA having a cDNA sequence selected from the group consisting of SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 97, SEQ ID NO: 98, SEQ ID NO: 100, SEQ ID NO: 101, SEQ ID NO: 102, SEQ ID NO: 103, SEQ ID NO: 104, a fragment thereof, an isoform thereof, and a variant having at least 80% nucleotide sequence identity thereto, in the biological sample compared to an expression level of the one or more of lncRNAs in a control sample from a normal subject indicates that the subject suffered from myocardial infarction or is suffering from cardiac pathology associated with maladaptive remodeling of myocardium.

9. The method of claim 1, wherein the subject is a human.

10. The method of claim 1, wherein the expression level of the one or more biomarkers is determined indirectly by i) measuring abundance levels of cDNAs, amplified RNAs or DNAs, or ii) by measuring quantities or activities of RNAs, or other molecules that are indicative of the expression level of the one or more biomarkers.

11. The method of claim 1, wherein the method does not comprise executing an echocardiography.

12. A method for monitoring the effect of a treatment on cardiac tissue, a method for monitoring efficacy of surgical and/or a pharmacological cardiac therapy, a method for monitoring efficacy of an agent and/or small molecule that can induce cardiac reprogramming, a method for measuring cardiac tissue regeneration, or a method for monitoring in vitro cardiogenic cell differentiation in a subject, the method comprising:
a) measuring, directly or indirectly, an expression level of one or more biomarkers in a biological sample derived from the subject, wherein the one or more biomarkers comprises one or more long non-coding RNAs (lncRNAs) have a cDNA sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO:103, SEQ ID NO: 104, a fragment thereof, an isoform thereof, and a variant having at least 80% nucleotide sequence identity thereto; and
b) analyzing the expression level of the one or more biomarkers, wherein a differential expression of the one or more biomarkers in the biological sample compared to the one or more biomarkers in a control sample from a normal subject gives an indication on the effect of the treatment on cardiac tissue, an indication on the efficacy of the surgical and/or the pharmacological cardiac therapy, an indication on the efficacy of the agent and/or small molecule that can induce cardiac reprogramming, an indication on cardiac tissue regeneration, or an indication on in vitro cardiogenic cell differentiation.

13. The method of claim 12, wherein the expression level of the one or more biomarkers is detected using a method selected from the group consisting of microarray analysis, polymerase chain reaction (PCR), reverse transcriptase polymerase chain reaction (RT-PCR), Northern blot, serial analysis of gene expression (SAGE), immunoassay, and mass spectrometry.

14. The method of claim 12, wherein the biological sample is selected from the group consisting of cardiac tissue, whole blood, serum, plasma, semen, saliva, tears, urine, fecal material, sweat, buccal smears, skin, cardiac tissue, liver, brain tissue, amniotic fluid, nerve tissue, and hair.

15. A microarray comprising one or more probes that hybridize to one or more long non-coding RNAs (lncRNAs) having a cDNA sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO:103, SEQ ID NO: 104, a fragment thereof, an isoform thereof, and a variant having at least 80% nucleotide sequence identity thereto.

16. The microarray of claim 15, wherein the one or more probes measure an expression level of the one or more lncRNAs that are expressed or measure the level of one or more nucleic acids derived therefrom (e.g., cDNA or amplified RNA derived from cDNA that incorporates an RNA polymerase promoter).

17. The microarray of claim 15, wherein the one or more probes comprise a DNA sequence, an RNA sequence, a copolymer sequence of DNA and RNA, a DNA and/or RNA analogue, or a combination thereof.

18. The microarray of claim 15, wherein each of the one or more probes comprises 10-1000 or 10-200 nucleotides in length.

19. The microarray of claim 15 further comprising a solid support or surface to which the one or more probes are immobilized to.

20. The microarray of claim 15 further comprising a reagent to enrich the one or more lncRNAs.


SEQUENCE LISTING (148 sequences)

SEQ ID NO: 1 - DNA, 540 bp, Homo sapiens
  source 1..540: mol_type = genomic DNA
aataaagcaaaccttgaatatattcagctaggaaataaaccttgaaattatatggttaccaataacagtaaatagagtttcaacaaataactctgctaaacattttgtttttgttaatcacccatgcgtggcaacatatattattctttcttttaggtaacgcagttgaaggaccagaacgcattcctaaacaacacctcccttcggtgtaagttgcagcatctgtgacttagagaacctacagttagaattctgttttccagatattatagttttagtggaatgcctgtaatcattattattttttttagacaaagtagggaaattgccctagtagaaaatgtaattcaatataggcaaaatagaattacaaccatttgatcatgtctatgataaatccagcattgcaataaacatttctaggattttctatttctatctgcttgcttcttgtttagacatcttaaaatgcttgttagtgttaagtgattgcagtgccaataccgtaagagaaatttatgagaaaaaaaattgagaaaa

SEQ ID NO: 2 - DNA, 614 bp, Homo sapiens
  source 1..614: mol_type = genomic DNA
cttttctataattactgttctgttttttctgacaggctatcaaatattggctgccttaagctctctgtgaactgagttattctcaataatactctgtattatgaaggcattatttgagtggctgtccactgtgatctggttatttggagctttacataaaacaggctcttgctatatggttatgccaaaaggctttgcaatagttgtatttgtttggctctgtgagatgacccaggcaccccgattccaagggccacttactttgagtggacatgaggctgatgcccagaaccaaagaggaagcagcctctgatccatgatcctatagtcacagtgctgctcaatcgtgccgtttctgctgacccaaatgcctggagttgaccctgtttgtctctccaaacctctttcttgacttagatgatgctttgaacttgatctgttgtactatgactgtaattccaagagcacaggctttcccgtcatgtagagcaatatcaatcctgcctcaatcgtttgttaccttttgattctgtgggtttcagtttcttcatctgtcaaaggagaatacactggcagagctcctttagaaagcctggttctgtatcatggacact

SEQ ID NO: 3 - DNA, 2884 bp, Homo sapiens
  source 1..2884: mol_type = genomic DNA
gaaatgctatagttgtaaaatttaaaatcaaatcagagtccagattttcaaaaactgaaataataagttaaaatgaaaaggtaatggtgatgactataaaccttgtacttgggaattttaaaatatatcaggatgggagggtctagtttcacagcctgggggtttgagttgtctgcaaaggctcagaatgagctcacagagagctgttgtgccaagagtgtggctcgccctccaactgaagtgttcatctaggatccagagcacagggcagattccctcaagctctgtatggtcagagctctgggagcaagcatcagtttatcccaggactggttaatggagcccatccagaggaagaccgagatgccatccagggatctgggagtttaagctggacaagagaagacttggaggggcctgataataactgtaatacgcgttgatcattatgtacaaactatgcctaaacctggtttcttccaacaataaccgtttgttcacagttctgcagttgggccagggagctttcctctgtatggtgtgggctcctatagatctggggccagctggacatctatggcccatcccttctcatgttctttcattcttctccacaggcctctcatactcagggaggcagcctagacatgacttctcaaggcagcaagacagtgaaagtggctgctacagggctctaaaagcccgggctcagaatttctgttgctttccactggactgagaaactcacaagagcagccaaagaagaatgtagaaactgccaacaaccacatgactgagcttgcaagcagatccttccccagtagccttcagatgacaccagccccagccagcaggttatgcaactgagaccatgaggcagaactgccaactaagccccagattcttaacccacagaactgtgagatcatatttgttgttttaagttgcttagtttaagaataatttgtagtgattcgttacatagcaacagaaaaaaacactgggtttttttcttggctttttttttttttctaggcaaacaagaaatccctgcatccacatagctagccaacaaggccttgtcctaaggggtgaaactggtgaatcagaataaagaaagcttcccaggaggcctggggcaagtggcacaagacttagtgatctttccaggcctaagggagagaattccgtgggctgctagaatacaccagcttcctttatagaagttgtggccagtgcaaatgttaaccaaacgtaatgtatgagccttataacaccaaatggaggttcctctggctgttctgtaacttaaggggagtaagttgacttcccatgactctggcttcccagttgggatggttccatatgatgttttcagaagtaatggtggaaccttcactcactatgtcctccccatctgctcacccctatattggtccagccctgtctgccccacacacacctccaaattttgaaaaaaagcatcctacaaatgcataagaacatggtcacacgtgctctttctgacacacttagtcattggtgggtccagagctgcagggtccttaatgctcatctatgtgccctaagttgagctctcttgctgcagaaaacctgtggctagaagcaaaagcttcctaaaatcagctaaactaggtaacgcttttttcttgaccttgaagtggagactcagagcctttgggaaccttctgttttaaatatccaacaggaatactgctttaccatcctttcagcggggggacgggaccagacacaccagagaggtagcacccattggacctcttaggccaggtggagacagtgagagctgctaggcattcctcagcgatgccagtatgcttactatacaaaacagtgctgagttaatttcacacagctggggaggactggaggtgcttttggggcctattgtgtgtgagccagcaaactcatctctccagctgttccttggtagaagtagaagtagaagaaaacgaagaagttggagccagaagtcagtttctgccaacgggaacaatgggtgcaattgtgcatctcacatagcagcaaggagtattaaacagaagtacacactgttactacatgagaaccttgccatgggaacttccggatgtacttttttggaatccaggggatctaggaagaccagtcagggagtggccagttcttggatgacctctccaagcaagagaaaacataaatgtgtactttaggagtttcaaaagcaatgaagatctttcttagtgcaaagccctgttttccccttaatattaaagatagtcttggtcttatttctgggaagtttccaaggacagaggctaaactgtgaccaaactcacaggaaagagaccctcagagctgggaaatgatcacttcaaatggtttcttgcctgccttgaactgcgttcaagtgagggacttctccaaaggaagacgcaggtggaatcccaaaggcaatgtggtgtgcctcagtctcttgagctgttcacctcagaagagtgtttcatcttctcggcaatggagatgacacctacctcatggatttgcagtgaaattttaacaagagtatgaataaggtttaaacactgaaaaaacatctctgagttgcatttagttgtgccggacccaccacattaacaacctaagagagctctgccccgtgccccatgccacacgagccctcatcctgggcctacctggctggctggatatggggacatctcaggtggcagagcaatttgcccagggtctgcccccagcagtagagtgatgcggagattttattccctcctcttccttccctctctcctcaagtgtggtgcgtgctccatttctcacgtggccatgtgcaagccaggaagaa

SEQ ID NO: 4 - DNA, 1851 bp, Homo sapiens
  source 1..1851: mol_type = genomic DNA
cagagggattagttgtaattgctaagttcttttgagtacatttaaccaagtagcagagcacattcttccatgaattcagaagcctgttaaaacagctgtctaatggggtaaaatacactttctgttgtttggttgaaagacaagacttcaaataaatactaaaatcagggttggtcaattttcaccaggagcccaggcacctagggaatcttgttctttttcttctgcctggagttacttcactggaattgtgagaaaaactgatatgatgcaaacactagtgatctgaagacttgggttcaaaccattctttttcgtcttgcaaagtataagcgtgtgtgtatgccgtaatatagggaaaaggtggctcatgtataaaatcataacaagaatttctatccaaaagtttcttgggagtattaaaataattatctgtgaaatccttagcccattggatatcacatattcagccctcaataaaggtagttgatgtcaagtcaactttcaggttactttctgcttattatactgtcactgaagctagaaagggacaggatcatgcactcttgttgactggaacatggctatggagagcctctcattgttctttgttccatggcaaatagagcaatgcctttctattttcccagtgaacattatatataaatatattttaaaaatcaatctaggttctctctattgcaccttcttgtcactgaagtgttgaataactgttggagtcttggctaagcaggagcaatatttgatggaatctgacattgctttaatattctgggttcatacacatatgcagcagaagtacctggattgcccatttcaaggtactctgtggatttttaaactttcgtgacagtgtaataagagaaatttaatctaaattgacttgacattcaaaaccagacatttctgtgctctaaaataattctatttgtttctgggcactgttttcaatctctaaattataaaagccctttagctcactggctcattcttaacctgattgctgtgacttcgatctgtctcctacatcgtgtccactttcttcacctctttttctgtgggtctcactctcctgcctccttactctgttcatgacgtgctttaacatgtgtgaatgagtgacataagcatatgactacaccatttatgttgagttcacagccatttatatcattaagtagatgatacagagaaaaagaaacacatttaattttaatcaggccactatacatgatgaatcaagtgcttcagcagtcataagaaactgctcactaaaactttgattatgatttaatgattaaatagagatttgctatgggggacagattgttcatttggtctttctttaaagactgatttatactcatctaagtaaataaactattcctgctctgtgctctcagacagtcttttatagccaagatgcctgcattccccacagcttagacaggtttcttcctgactttaggcttctggtctcccttttgttagagcatttgctttagaaaacttgtcactgtaaattcctctatccctttgagatgtaaatctcccaacctcttgtctgaatttctggctccaggactactgcctctgtctcccagtcgctgtgggagaataggaggagcctaactttgataagcaaatgcagctggtctaagcctattggccagcctccccgcaagccttcctccaaggacgaaagtgtcttcctttcctgtttaactctgtcgtgcagtttttatatgcggtcttagtgatgaaggttgttcctgactgtgagtctatctgtctcacttcactgtgagtcctgaggtctgggaataggga

SEQ ID NO: 5 - DNA, 2627 bp, Homo sapiens
  source 1..2627: mol_type = genomic DNA
gtttgggaagagacaggctatgagcatgacatgcaggctgtgggggctgccattcctgtctcagctcctccttcttgagttttgagcaggcaaaattgcctctgtctaataacaccccccactacttttctctttctttctttcattctcctcctgccccctgtatgacaaaataggcaaaatgagtaatcagagggtgagatggagaaaaagaactatagttggagattttggacggtctcttttaggtgggagaaggtaggatagtggaaacatgattgcaaagaagaagccatataattttttccctttgactttaagtcctaagatgggcaggctggaggcctgggtgaggagaggctgggaggggacaacgcaagtggtagagctgtctggagaggttttctttgtgaaaaaggcagctgaagtctcgaaagtgatctggactgagaatcgggagaagtggattcagttcatactcagccactttttatgtgaccttgggactagactattgatgcccagtttctttatttgtaaagaaaacactgtactttttcttctgctttaaaatgactgttagaagttatgaaaattgaaaacagttcacaaaagcccagtaacttgctttcttcatcattctaagagagaaaagttaagcgaactctccagttcattgttaaaaaacaaaagaaaagaaaagaaaaaggcatcttttctctttgagaaacaccaaaattactctgcccctaatagtcgcctgggcagaattttccatcttagcacaaagcagctcgaccttgtttcaactaatcccttctctgtatttaacaagaggaactggaagagtcagacagtgtgtgggaagactgaagaaaataaggaaggtatagaaactactagagatggagataagttaaacattctggcaaattgtcatggtgtattgctcatgctgaaggttaggcagttgggaaggtagttaagagaatggctttaaatgcttcatctaattgcaagcagctgttgattagtggagcttgaccaactgtttttaattcagtggtgatgaagagaatgagacttttaagggaaactggcacggttgtgtatacgcatggagttgcttacactttttctggaggtcatagagtgcagtaactactaagagatgttctctgaaaccattgcaggctttcatgataatgactttcctaaattttaccagcatttgttgggtttggcaggaggcaaacttctccgttatccaaattctggaatgattttagggactgattcatcaatcgcgaggtcttgtgccaattcagaagcaactgttcagtggaagcagtaaatctagaatgagagctgataatgccattttagagtcttcttggaacatttaattttttgaaacataccacttacctgaaacaactgtcaggttgggtagagaacgtaagtgtcttatgagagttctaggtagagtgctctgggacttcaaagaaaaaaagatgcctacctttaagacaaaagtagtaaaattcaaatgctaaataaaatgctcagctccactaatggaatttttgcgcatgaaaaaaagcaagctataaaggttaagataaccagactggctgtatttctgtccctgctgctgtgggtggcagagaaagacaaatcctaaaaaggtttttcttccagttagtgttgggccaggataagacctgtttatgtcaatgaaataactttttaagcagaagatttcagagccaagagaagaacttgcagtcaagacaatctgccacagtgtgaacgatccatctgaggacaattgggctgattgtgcaagctgatgaaggaacatggcatcctctggaaacagggaacaactgtcaatgttgaaaatgtgtggaacataaggaaagcatgccaacttttggtttcaagatattgctcaatcagaaatccggaatttcaacacatctgaaagtaaggcggagggactctagcagtctgatctttacgaactaaagactcagcaaaattcccactcaatggaagtgcaaaagtctctgcagacttttaaaaaatgccaatcccatcagatgggcaaacacaaaacaacaaaaaccctaacacctaacaataccagagatgatacggagcaattataactcaaatacattctggcgggaggctaaatatgtacaacaccttggaactgtttggcaccatctccccaagatgagtctgtggcccagcaattccactcctactttatacccaagagaaacaaatgcatatgtgcatcaaagacatacaaaaatgttcccagcagtactattcacactagccccaaactggaaacaactcaaatggccatcaacagaggaatgaataaaaattgcagtgtattttacagtgaacaaactatgacaaactacaacatggatgaatctgaatcccacaaacagaatgttgagtgaaataagccagacaccgaataatacatagattaatgtgccatttctataaaacttaaaaacaggcaaaactaatacatgattttagaagctggggtaatgtttcctttgcagcgaggtagtgagtgggaagggccatgagag

SEQ ID NO: 6 - DNA, 915 bp, Homo sapiens
  source 1..915: mol_type = genomic DNA
ctcttcctcctcctcctcctccccggcagagctgtaggcctcacagtggtgacgcggggtcatccgggggcccgttaccacctcattctcataggcgcgcggaggcggtgcgcgcgggacgactcggccaactgaggagggagaaaggggaagcggatcggcgggcgctggcgctcgagcgggacgcacggctgcggccgctgggtcggtcagcgaattagttccatgatgacccccggcctgaggccgccgccgctcgagcccgggttgggagggggctccctctcgccatagggcggcgggggccggggagaggcggggggtgagaccggctctgcccctgcccggggaaagcgcctccgaggggaaatggtgaaagggggggaggggagaaaagaaaagaaaagaaaggggaaaggggggaaaaataagaaaaagcgagacagaggcgctgccgcgtccgctcgcggggaaggctggggagggaggggaggaggaagaagtgccggcttcctggctccgccctcgcggaccgatttcgcccactccttgtaaactacgcggacgtctttcacccttcctgttcctccccgccgccttcccggtccaacgaaagtacagagaagtgcggccgaaaggagacgggctggggaagtcgttggcactaggatggaaggagggaggacgttttctgtggctaagaaaagccgaagtcttgagactgtagcatttctgcataatctattgtaacataaccattgttagaccatgaaattgcttctgattttcactccaataagaaaattgaagtgtcttctgaacacttgaggataaggtaaggtaaagacaaaaacataatggtggagaaatatggtgagaggaaccaatctttatggggcagaaacttccctcctc

SEQ ID NO: 7 - DNA, 1001 bp, Homo sapiens
  source 1..1001: mol_type = genomic DNA
gcaggagtctgtagtgccctgaagaatccaagccgtcacaccagtgattcgaacatttcagagaaacacaacggagggaaggagcagtggcttccccggtgggctagggcgtgtcacaggtgtgaaaaataaattcaaactcaaattaactggctatgtatctttgttacaagggctcagtcaggaagaacaaaggttcctgttggctaaagtgtgtcctgaaaattatcttcaatcctgcatgtactcctcgtgttcaaagatgagctcacaggaggcaactacctgcagggggcaggacatccagggacctccagtgtgggtactgggctctcatatctctggcagtgtgtgcctctcctcaatttctcctttccttctggaccacctacagacacagcccactgccagagacgcccagatataaacacaggtgcaaaacacatctgcattactggatggcagcacggccatatggttggagtaacagtaataccatatccctgctgtagaaccagctttatttacaattttcttattaaaatatgcaaatagcaagtaaatagaaaaccacagagctctgttttgctgtaaaattgattggagacgtttgactattttaaggacttttgatatacagatctgaagccaacataaaagaagatggttaagtccacttaaaggcatttgctaggagcacaccatagtacgattaaggttaaaacaaaaggatgtaaggggcttccttaactaataggtccaatccaggaattgctaaaacagagttgaaacttgcagtcctgcagaatttcaaattgtcctgtaatcctaacagcaaagcatggcaatgctggtggtatttggtgaaacatctcaatcgtccatttattgtcaagatgtatcaaattttgctgattttgtgtctttaagtggccactcagcatattttgaactagacaactggacacaggcatctcctccaactctgatgtacattctta

SEQ ID NO: 8 - DNA, 1203 bp, Homo sapiens
  source 1..1203: mol_type = genomic DNA
ctgctgcaaaatggctgtggtctgcaaggctgggaacagttttgtgggtttgcagtgatatttgaagtttgatgaatgaatacaaaatgcatcactggtataatctaaatgatgtgacataatttatgctccaactgttaaactccaataaatcccagccaatgacaggaaaatcccagatgttggccattctggaaactatcaagcctataacttgatattaatgcctgatgaatagcataaagcagcagcattttccatatgtgtacttagagtaaagttaatgtaataaacataattgcactcatatagcttagtataagcaattatcactaattactggtagagtttctctctcttttctaatgaagagtacagtggaaggatagtttatctctagatactaatttaatttaaacgttaataggaatagttgtttctgcctaaaagatttgatgaattttcattgcatctaactcaattccagcaatgtctttatccagcctttcttagtctgtcacttggaggatgttctatcctgtcctactgtccataagaccaagcagctcggaggactcccataatcagtgaaaagcaccttgcaccattctgggaagcagtgggactcagcttctgtggattttaaatcagtcagtatttcggagcatcggtgatcagaggtgtctcctatgtttgtattcctataagcatccaacacaatggccagtaggtagttaatgctgattaaatgtttactgagagaagagacaatgcaggtatttatgtatatttgggtttgggtcattgttttcttctctttttttcctttctacacataggataaagagtctgtaatacacgactagaataatttgatacttaaaaaatggttgaaaacaatgacacattaaactatctactgtgcaaaccttcaccaccaaagccacctctggccacagtgcaggactgttagaagttgaggggacctttaatttctgccagaagccccaagtggttgacaattatatcatttctctatctcttaatcttaactgatttcctgtttgtggacaaaagaaagccatgttacagtttgttcagaaaccaagatggaagccttttaattctgtcttcattggtgaagctatttattgctgtgttttgtgctttgaaaaagttgaataaagatacaaatacagagca

SEQ ID NO: 9 - DNA, 1029 bp, Homo sapiens
  source 1..1029: mol_type = genomic DNA
cacaacaatcccatgaggtaattgctactatcttcactttatagatgggaaaacaggttcagagatgtgcggagggctcacaggtaatacacagtgcactgcacctacaggatcctgcttgaatagccagtcagaaagggagaagcaaggaaacggctggctgtgtagacagaggagggatgggcaaagaagaaaagccagggagaaaatcaagacttcagagggaaaggaggcatgtgttgggtcagaggtcaaaagactcagcatcactctccagtgtgggaaggtggggcacagaaacacaggaggccaagtagatcacgcctctgctcaggtctgagtgtcagagctggagccaatccatacccctgagtagtctgtgaaggctgccagagacatgtacagttacccgcctggaagggcatgcaagtgcctcgatatgggccaggttctgggactgaacacagggagcagcctgacctctaacccccagactcctgggggagctggaagagggcctagtacaggatgttggcgttagaagtgactgagaggaggtgtatgcagggatcccttctgagccatccaccaagtcatgtcagtgagttagcaacactatggagttcctgggccccagaccaggtgctggagagtggaggaacccagtgatactacatgcagacagtagccccttggagatgagggccgagtggggggaaggggagaggagagatggagctggatggccagaaattggtgggtgagagggtgttcaaggaaggatggatgagcacaatttccgaacagcaaacatttcactgctgggtgctgagaatacaggagtagacatccaatcctcaaagagcttcccgtgtactggggaaacagaagcagttacaatagagcatgatgaattgtaagggatatccagaagtgacaggggagatttctaggaaatgacatgtagtataaattgagtcctgcaagatgaacaaacattagccaggtgaagggtcagggaatagcaga

SEQ ID NO: 10 - DNA, 1948 bp, Homo sapiens
  source 1..1948: mol_type = genomic DNA
ggaaattatttcagcgcatgtttttcttcaactcaaggcttttatgaagaaaacatttcttaaagctgttattaaggtattcaagctggaactcagacgattctaaaacactcctctatattcttcctcatctaatgtctttggaagaattgttagggtcctggaaaaagaaaaccaacagatcaggtggttgaatatggcttcctggtggggcaggaggcatctggcagtgcacaaaagggcaaagcttccagagagtagcaagtcactgaagaaattctgttatagagaaaaactgtgtgtgtgtcacatacatacacatatatcttcaaattaggtcatttatgaaaaaatggagaaaaatgcttctataagatattcacactgttaactagttttcaaaagttcatcaagcttactctccttttctttgaatacttttggaagagacacaattttccaagccaaaacacagttcaggcaatagaatagatgaaatttttacaaacaggatgaaagcctggcacataagacactcaatatgtattgaaatatctttggcgaaataaaactataatgctctttgcgatatggttttctctcttaacaatatattttgggcctcacctattagttactagaaatcggtagcatcattgttaatcgctgctttgtattaccttgtaggggtgcactttaggtgcatccaatttattttctgtcacgaccaaagccacaaagaacatcattttgtgttacttttgacatgagtgtaagtcctgtaggataaacaactacgagggaaaagactgggtcaaagggaatttcaggataatccacttcttcaaaaactttaagtgggaagtgagaataagatcaggaactcttctgccttgtccttttattccaactctctactcaacagggcttcaccaactgtgttagtttgagcaaatctaagagcagacccaggcacaagtgccagtagcggcaggagatctgcaggcctacaagtaggggtgtgcagatgagacaggtaagaaagggtgttatcatgccacttctcactgtggacagctgggactaatcttgctgggaaacaatgtagtacacacccagagttaccccagctgtgggacacggaagctatggtattcattcacatggccatatcgaattgttggatgagaactgaatcaggggcatcaggtatctgctcttccgcttgtcccggggagaggtcaagtgcttctgcagctggcagagagctgcagtgttggaaaatcacctctgctggtaggggtgagtgcagagaggatggcagggctacttagtgatagaaatcagaagtctactctagataacacatgccaatttttagctgcccagaatatcccccttctttgggggagacttctttattctgcatgtctttgagagagagagcccccttgccaaagcttatactctgcctgctgcatgaggccaggcagtggcacaaagattcaggtacagtcagtgactatttatggtggcagtagccaagttcaaagtccagcagcagcacggtgagtgttgggaacagcagaacaggcaacatgtcataataaaatggaatttgtgttctggaaaatcaaataaaggaaatctctcacccacacattacaaaaggcaaaaaatatccacaacaaccaaaagcaaatactaaatataatgagtacaagataagataataagtaggtctgggaggtctgataaataaataacaggagatccttaaggcaaaagataactttaaatgtagaaaaatttacattttaagaactaaatggctgcaaatcaaattccaaactgcacaaaaagccccaaactcgtcatctcctggtgaaatttcttaaatctgagattaaagagaaagttcaacacttttccagggagaaagtttact

SEQ ID NO: 11 - DNA, 1075 bp, Homo sapiens
  source 1..1075: mol_type = genomic DNA
tttattaataactgttttctcacttatatgcttatatgtacaacctgagttaaaataggtgcttcctatgaaaacacagcttcatgacccctccaaccagagaaattcccccacctcccaagctaaacgcattctcttcctctgggggtgttgcctgatggaggggctaaagggagatgctgcagggggggttattgttgatgtcctgcacagagggtggggaagcttctcagtgtcactagccaggtgtggcacattgtcatctaggcagccaaggagacgccaatgcttggagagctagtttccaggctgagacttccaggcactcagaggaaaatgtctgggctagaccccgggtgggcaggatgaagccttctgccacttcttcccaggcaatctctggtgaggctgggatcaaagactgttgcgccacttttacacataacagacacactttatgtgtttgtgattattgcctttcaaactccctccccattttcccaaatatctaaagtcaaaatggccttttggagaagagggtggggagtaaggcctccaccagctgttatctggcctctggaaaaatcctctacattggagtgcattgagggaatacctggtttctgtccctactgcctttgaataaatgaagaaacagaaacacaagcgaatgaagttacttagagccccagaaccttagagctgaacgagaccttccaaataccttgtcccagtctcctcgttttacaggtgagaagacacctaagagattaaatggcatgtccaaggtcaccaggctattagtggtatagatggaacctaactcttcagactgctggtcagatgttctttccactttaacactgcagtgcaaaggtcaaacacaacctggactctgtaaaccaccacagtccatcaccaacatttattgaacatctactagtcagtgaactaaagcctctgcactccctccttctcccgtctttgtctttgggactaaagagttgaattgcaattctgcttcaatagaatccaggtgtaatcagaattgccttgctttctgacattacataca

SEQ ID NO: 12 - DNA, 1113 bp, Homo sapiens
  source 1..1113: mol_type = genomic DNA
taaagttacctacagtgacaatgaagttgatcagcactctcaatgacccactgagattttggtggataaagttgcttcttctcagtatgcttagctggaggaaaattattagtgaccgctattatctgattggcctttgattttctttgccctcaggatgaattcttttcctgtagactttcacctttcaaataaataatagcctagaattcctgtcctacaagttcttcaagtatgtccagacataaatatttgccaaatatattaagaaatatctagcaaacttgggaggactacttatattcagcacagaacaattaactgactctgtaataccaataaatgaatacagtggggaacatatttttgaagattaagagatgcacaattgttagtaccaaaaattacagagccctataaaatgaaaggaaaccttggaaagggcaggtgggtttgaggctgggccacagaccatttggaaccaggaactcaaattatgccatcatgaatgcattgctgctgctatttctctgtgtgttaaaaacgtgtttgccaacagcacttgaattttgtgtatgaccactttagccatccggactctctcagttctatttctaaaaatccccagaaaggactcttattatccagcttgtgaagccttagatctgttacgggttgaattacgtccccctaaaagatgctaatgttctaaccgcacctgtgaatgtgatctttctttttgtagattatcaagttgatggttcattagagtggttcctaatccaataaaactatgagtttgtaaaaaaggacaatttggatacagatatagacaggtatacagggagagcaccatgtgaacgtgaaggtggagattggatgaaacttctacaaggcaaagagtgccgaagtttcagcaaaccactaaaaactaggagagcatgaaagattctccctcaaagcactcagagggaactaccttgctgacaccttcctttagccgctagaaactgtgagacccttctatttaaaccattcagcttttgagactttgttgcaataccctagcaaatgaatacagaaatagtggcttgggggtgggattatcagtac

SEQ ID NO: 13 - DNA, 614 bp, Homo sapiens
  source 1..614: mol_type = genomic DNA
gttcaaatcacctgttcttgtgaatatgctaatttacatgataaaagagactttgcagatatggtcaagtgggatcttcagacaaaaaatggaaagtggacttagtgaataataataaatgtttatgtaaagcttatgccaagcactgcaaccacataaatgtatcaaatcatgcaaaacgcaccaactcaatgactcctctctgtgacgcagatgctgtcattgtgcctattttacagatggagggactgaggcattttccaggctggcacacagtccaacacagctaggaagtggtgggctggcttcagattctgtgcccctcatgctatgctgcctctctgaaggaagtaggggctctttgggatacttttgacattaagaaaggattaaatagtacaatataatgaaaacgtttgaatactcgttttaaaagtacaaaaagagagagccacacacacttttatgtgggtcaggagtgactgttgttgcactctttggcatctgccaagcatgatttctcaggatgtggtggttggccgactgcccttgtggccactggattccattctctctctggtttttctccctttccccctgccctctgaaacctt

SEQ ID NO: 14 - DNA, 233 bp, Homo sapiens
  source 1..233: mol_type = genomic DNA
gtttacccacactttttccacctactgcaagctcccaggggcaagctgatccctagaacactttggctgaatgagagaagcctcaagactgcaaatccaccccatctccccaagggcctgccaagactaaagaagaagaagatctacattaaagaggctctcctaaggtcttcaggcttgggatttgtgtgagaaaattctaatttaagactttcttgtgattcataggcaaa

SEQ ID NO: 15 - DNA, 1618 bp, Homo sapiens
  source 1..1618: mol_type = genomic DNA
attctggagagtccagaggtctctctgcttggcttgcagatggccattcttgctgtgtcctcacatagccttttctctgtgcatgctcaggtgtttcttctttgtgtaacagcagtcctattcccacccttatgacctcatttatttattacctttttaaagacctgtgtccaaatatagtcagcttcaacatatgacttttaggaggaccttagagcaatgaccttcatccatcaagaaactggagggatctcactgaccatgagatgctccattggttgtactctagaggagaccgacgaggatctgcctcttcctcttgttccctatccaggtaggctgaacatgaggagaagggtctttttttttcttagtgggcaagttattggaatctttaaaaaaatgatcatttatgattaaaacttggaaaaggaattatgcccatttttttcagtttactctcatggctgtactacaaagcactagtcagtaacctccccttgaaaagtctttggataagtgcatcattattctagctaccagcacttggtgaatccccatgttaaactcaacgtgttttgaaattaaacctcagagatgttcccaaccttcttttatgtccctagagataagtttaccaaatgttaacaaagaaataattgttctcagaacttatggcagaaagtgatgagatgtagggttgctttgaccgaggcacaaatggtgctctgtggcaaacgtaaagaaactgttcaataattactgttttcagcaaagtaaaactctaaaccaggggccagcaaacttcctgtacagatccagatagtaaatattgtcagctttatgggctacacggtctctgtccaagctactcagccattttaccctgaaagcagccatagacaatacatatacaaacaggcatggctgtgttccaacaatactttatttacaaaaacaggtggcagatttgatagtttgccgtcacctcctcaaacccaggcttgctttccctctttctgtagtaggtttgaatgcctaatacacacattttctctctcctgccttttaaaacttcatgcttatgctttccatatgtttcaagcatgatcttgtgtcatcttttttggaggtaaaatgttgggttgagtgggttaggaaactgacttgtgagtgtgacattggagatgggtctaatcatgatgttggcctggacatatctcatttcttcccatgggacccatctgatttgatttgactttaggaataagagttcagatttgggattgtgcttggtttggagctggcttggtgagcagaaaatgctcttcaggacgtaagctctgtgtttcggagttcagggagtggggcagtcataaagagcccccagggagaaggcccagggtcctggcttctccgtgggactgtttcccgggtcacagaaagggagacccaagtgtcagaggcagattgaactgtccctgccgttatggggagcctcttttaaatctccccattttatatatggaagggaggtgggaactattcagtcttggaaaaataaagtttaaacccctgtgaatctcaaaatatcaataaatgaaaggacacgtaactg

SEQ ID NO: 16 - DNA, 1611 bp, Homo sapiens
  source 1..1611: mol_type = genomic DNA
tcctcctcctgtccagcggctggctgtaggccctacgagagagctggcttggggcgcagcccgtacctctgggagaggccacaaaggggtcggagagcgcggtcctgtgcacagttagggttaagtcagtctgccctgtgtgccctccgaagggtgcccagctttgctcagtgggttggagaattgcagaattctcggcccgaatgaaaagctgtcaaaaatagtgaagttagtttggccttccaaggttaggtggtgtggtagattgcgggaccgaggttcagaatttctgggggctgtatccccgagccccactcctcagaatgcaactctgtacgacgaaggaactgggtaatcagatgactaaccacctaaacggtccacacaattatcattgactcctaggtaccgaattggcatggatttgacaattagcgattcacggttaagcagactttatctttaatccatcaaaggagaatttctgcctgcgtgtttggagagaaacgcgtggatgtgagttaaactttgttaaacccaacaagctttaagataaaacgactgatatttaaaatggcaatttaaataaaaatgttctctgatcagcctttttgtggtcttaaccatgaaatgtctttgcatattcaaaagtgagtccgaaatcttagaatgcaatgtgtcaagaaaacttgttcaagtgcagactccagttaagatcaatgtttaaactttttgaatactttttacatataagtttgttgcaactgatgtctcaagaacataactggtgaggacagctgtattgccttttagaaaattgttacagttatatttacaaaaaacaatgttgcttcagttaaaatgagtgaatttagtacaggtaagggtgtttgtagttaggcttatgtggtaccttcctaatgacaaaggccaaacaacattatgctgtgctctggagatattacaaaatatatagaaaactcccccttttggaagaaatgtgtatctttggaacagttttttctccatgttatattggaggcatttgaaacttcctagtcatgatgttttctgagcttttatacaagattgtatgtatgtaactgccaccttttttttcttttacacctgttttcaggaaatatgacttattttcttttctcctgttctttcatgaatttaaaagtttgctgcgtgttgaaaattacttaatagtaaaaaagaaaattttgattttgactccagctgcttcggaaattctgattcttctaataattatatactagacttttcaaaaatatgggatttttttgtttcaaaaaaagttattacttgcaatcttagctcaaggatgccatttccagtatgctcccaatgtctatggcaaatgtgggtaattggctcccacgctttcctgctggcttcaaggagcctaagaatcatttactcagcactgtcaatagagaaggcaagaaaggtgaaccctgtttgccctccttggtttctagtttatgcctaatcaaagtccatgattgttttcatctctcagctggatttgagtcagcttcagcaaaagccaaatatccactttatataatgcttatttctttc

SEQ ID NO: 17 - DNA, 8709 bp, Homo sapiens
  source 1..8709: mol_type = genomic DNA
gttttttgaaatggttaccacattacaagttctttgtacatgctatttcccaattctttcaatcttctttttttcagattttagatctgaggaaaagacattgaattctcacagcaaccttctgagtgcctaccattatcacccccattgtccagatgagaagaggctttgagaggcacagattctttcaaggtcatacagaggtgggatttgagtctgttaaccccagactgtatgcttctccatgcctcctttctgctgtcggcagaagccggcacttgggcatgaaaggccagcatgcattatccatcttagacagactttttgtttctaagactggaaaatgctagaaagaagttggcagcccaaagccattccttttgttttgagtgtttgccctgcatgtctgcatgaagctggggtttttctatcagtgagcatctctggaggctggggaagcaattgtgttttcgacattcctcttcattcgcccaacacctctctctcatcaagagcctcgtggaatcttgagcgggcagacgctattcatcagaaagagttgccagccaacccgctatttcttgtttccaacagttaaggggaaaaatttcctagatatttttacttcgcaattctatttttgtctcaacttatctgatccttaattctgactttatttccttgtgatgttaaagcccctgaagaccctattcagggctaagccagatacccatcagcccttgtggagtcactcaagtcactcacttgcttacaaatcctccgaggattttaaggagttgggtgacagatagcaactgaagagaaccatgctccaggggaacaagctttgcagactgagtcccagcttgggggttcactgccaatcactatctgacctcagaaatatcattggccccaccatggaccagtgacatttgtcattcacccggctatgactttttctgacacatagacgaattctcagtcccatcttctggttacccacccatgctcaagccttgactatctgggtaaattacatcatcccccaccccatatctggcattttgagggtatggaggagggattgtaaagctatttgctaaactttctctggacgttccctccctcctgaccaccccaccccacttcctggcaagccttctggctgtgatttcatctgaattactggaatcacactgtctcctgtcagcctcccccggccctcctgctgatgaggggtcccggcaccacccagcgcatgcactcacgcacgccactggctcacctcctccatcccatgcctcccctgtctccaggtctctgtctcttactttatttttctctccacctccttgtgtctgcctgcctaaagatttctgatgcttaatcctcctctgtctcccttgctgtctttctctgtgtcttctgtttctctctctttctccttttcccttcagaagcattttaccccaatgtctctgtatgttccttggcattccctctgcagcactgtgaggctggcctgcttgaatgcacacctgagctccggattcacaggtaggtgtgtgacctttcttaacttctctgggcctcagcatactcctttttacagtgggaataacaatagcacctctcacacgaagttctggaggtgtgccactacattttgggaaatttattatgcagccatagattacacactcactatttcttttttctgatgccatactatatttttgtttgctgatttttgtctctcccactaggcattaattccataagggctgactgtgtctgtccccacagcctggcatatggatcactaataactcaggctgggtaaatggagatcactcattttaggctcagcctaataaatctccctagagagcagtaaaatttgcccattctttttctgtctttctgtattgatttctcactttctgtggctttctgctgttttattcctggctgcctgacagagggtggtgcagataaagaggcacctgtgggaggaagggctctgtgtgctttctcctttaataagctgtgtctaaaaaaaaaaaaaaaaaaaaaatagctcccctttgcaaaagggacgaaaatagccaggattttccagttttaatggctgggaggctgaagactgaggcagccctcttctctctgagcagcccccaaccccggctgatcacttcacacaccccacttgaattattattcatttatccatctttccaccaagtcattaattaattccctttatttgcttatctatttatacattcattatctatctttttacttatgaattaatccatttatccatactcagctattcatccatccatccactctccatctatccacccatctatacatttatccatccattcacccaccatccacccatctttccattcatccacccattctttcatgcatccatctatccatccaccaacccatccatctacccatctatccacatatccatcatccatccattcacccattcatccaccatccatttgtcatccatctatctatccatccatccatgcatccatccatccatccactcatccatttatccactcatctatccacttatccatcatccattcattcacccatttatccacctatccttccatccacccatctatccacttatccatcatccattcacccatcctgcattcatccattatccattgatatacttttcatttaatcgcctttatgttaattaatgaatttattcacttaaccatgtgtctatttatgaataatacaaacctgccttttaattttcactgagctctgctttgtgatcccagggagattgttacttctctgagcctccatttcctcactataagtgcagcttagaaatacttccaagcacatgggcagcgtgggtgcctagtaagtgttggctgttgtcattcaagaaggatggtgtgagcctgcacatgagtcagcaggggagagaaggcaagattctgcccagaactcaaggaaaatcagatgcagttttcaattattgagtcacagtctctcgtggcttggaaggagatagaggatcctcctcccaaccaatacctttacgatgttctttccacacagctctgtggtttacaaggcaggcttaatcttcaacctgcacgtatgagtggggtccctccacccctttttcctcagagcattagaacaagcattcttagagctggtccagtccagagatcttggactgggcctgcaggctggcgactctcatctggtcccaaccagatcaggaaagcaaaactcagtgaagccacatggctagtgttgggtcctcgggtggagcccaagaagcttggtgggcactcttccttcctcacctcaaatcatcttgaactctgtgcaagtcccagaatgaccctggctgtcttggcctttgtaaaagttgtcttattcctaaaattcccttcggtagaggaacaggagggaagtcaggagcagcaagcaggaaatggaagctaggctggggctgggaagagcagcccgagtttcatttccaggtggcccagttcccatccttgaccttggtgttcacctcaagagttttcagccagtccaggatttgcgtagaaaagtctccccatctcgttttcccagccaggtctcagatcggttcacatgaggcgaaatatacatcagcgtggatttttttccttttccatttgcatttgtttgtttcctgcaggctgacactagccagccttgaatttggtcctgttctctgcacagctggactcagggagcccaacccagatgtgctagaacaggtggcctgcctgggggccttgggtacatacagcgggtgctgcatccctgggatctgtaagaaaatgggagcaggtcccccaaaacaccagcagactgcactcccatcatgacctaatgagatttgaccaaaatagcccaatcctactagcaacagccaacacgggaaattgaatgaacccagggcgcagaccaagagacctggctagctggctctttagccttgggagcctttggtttcttcatgtttcatgagttgttgtaaaggttgccagatttagcaaatagaaacacaggatgctcagttagatgtcaatgccagataaacagtgtaagtatagcttgtgcaatatttgggacacagttatcccagaaatttttctttgttcctctgaattcaaatttaatggggcatcctatatttgatctggcaaccctcgatgttgtaacaataaagtgaaatggtgtgtacacatccttcacggcgtgcttcaccccaggtgatcagagcccagtcaccatttctgttagaccaggaatactagatatctaatactaattttatcaattactatattattaatgataattaatattaatcagcacaaatattaaaactcatttactgagtagctactatgctccaggcatgtccttcacatgactttgtcatcacatcactcaatgatagaggaacactgttctgagtgccttgcaagggctgcctccttaaatgtcatcaactctgtgagtagatactattgctaaactattactagatctctgttttgcaaatggggaaactgaagactaagcacgggctcatcaatggctatgaggcttcaaacccaggcagtaggctgcctggctccatccctcctctaggctgctttatcagccacatgaatagatagaacgagggcaactgtggatgtccctagagggagagcacagggctaccaggctgtggcacattaccagctgtgtcccatttagccctctccccctgtaaacaaagacatcactattcctatttctgatggtattgaggcttcccctgtatgcagtgagggtgtcaggacaaatgttttccagagctgagcactggagcaactcggaaggtcaagatagggttcccaacagagaagctctgacacttcctggctgtgtgaccctgggcaaggtctccacctctctgatctcgcatctcctgcctgtaaaaagggactgatgtcaatccatgagagtagttacggtcatttttagggcagagcttgagataagacttcagggcaggtggttcctttgggaggtgaactcaggatgccccagtagaggagtgggagacaggaaaagaagaaccaatacatgtgtgaatgagcaggtgactgctgccggcacttggagcacagtcctgctgggaactctggtaggctgcacagagcacacctgggagttgtctctcccaggggtgagggatgggatatttatcctccaactcccctacatctctggcctaatcccttgcacatgcccagtgtactcagtgggccagagaaagccctgggcaaaggcagtagaagtgagtgggcatgggaaggcaagcaggcatggtcagggcacccactacatcttgacaaatctctggaagatttcagttaaaaagcaaccttctgctaaaataataacaatgacaacaacaatggcgttgacaacgataataataattcaggttgaaaaccaagggatctgtccccacccacagacccccagaccagtgttctcctggggtaaccagctgtccaaagaagcaacccagatttccttcggctccaggaagggcaggctctctgagttctaggagaggcctggcatcctgcagaggccgtgcccataaagcattttcagagctcctgtcagggaccagcactcagctttgatttagcgtcagagtagataaaagaaaaaggaggaggaggaagccattggtgagcgaaaatacttgctaagtggttgggccatgagtgtatctgtcaacctggaacaagaagagaactactatttagataactatttgctgcacctcccaaatgtctggggctttacctggcttgcctcttgtgtccccaacccagccacttaagaatatttttatttccatttcagagatgaaggagtgaaggctcagagggggaaggtgacttgcccagggctacacagtgaggaatggtcagagctgggatgaggaagtaataaagaatgaaatcaagtgactttccctggataccaggccacgttagctcatggcccggtttctgggtcagggacaggggtgtgtgatactcttatgcaaggcatttggcggcagagaaactctagttcctaaagattattattttaactaacccagtgttgacgtgtccaaaggcagcgggaggctctgaagggttttccaagaaagcgtgtgtttcttggggttattttttcagtcaagttcagggagggggctgttttgctcaactcatgtttctggaaaaacaggcacttggaaagagtcaggatgtggatggagagctgtgggcctgcggggaggggacggaggaggcagccagggccggcaccctttgctcagtctcttcttgagctggggagcctggaaggaagaggctgaagatgaaagctgaagtctctgtgcctcactcttgagaaatatgatcacttggcctctggagcaggatgacgacaggaggtggggttgagggttgcttctcatggagtgctgtctagatatcgctgactcattgtgtgcctcggtttcccctctgggctcaagagctttgaatgctttgtgtaacccagtccctgcttgtccctgcaggttcattccacgtcactctcttcttcacgggcccccacagaagcccacggtcctgcagccttgaggccttcccttctgcctggaacccttctgctcctaacctacctctgtcgtcttcctggcaccttgaatatcaccttccctgatctcccaaatgagccagggcctcccttgtgcattcttttccttggagcacatttctctataattccttttccagtgtctacctctctcactgactctgaggcccccttctcatttactgtggtgcctgacagagcgaatggaaacacctcccaagcagggctacagccgtgggtgtcccaaccaaaggcagagcccaggacaccaggcaggcagacaggtgatggacagatggcttctacaccttcttaagattatgcagaaagccacactctaggccacccaccttcattatatggtgatcaccagtcaaaagagggaaattccatcccaacctcttccctggccctttcagaatcctggggacagagacttcgacagactccccacagggtctcatgaggagactctgttctcacttctcccctgctcctggaggacccacaactctttcaagctcagaggtgagtcatccatgcatcacgtgtcttggtgaatgtcaagtgtctcctgctttagagccttgagccctggactttgttttatggggtttggctgtctggggccgccgacgatttcatccatcaactcaggatcgctccaacactaatgctgaatttatagaagtggttaaacaaagtgttgacaatagggaccgcgtgtttctgattaacacgcaattccaagggaagctgccaagtgtggggaagtcagtaaaacacagcagcgatttcccaggctcagggggatttgacatcacgggattaacagatgttgcgcaaggaacttgcaaagcggaggctgctgcacaggcttatatacaaaccggagttgttccttcctgtctttcttttttttttcctgagagattaaatctgaatcctctttcagccttaagagtttccatcaggaattgctgtcagcttcagcaatgctcccagaatcctgtccttactcctttctgagagtgaacagtaacactggctaacatttatcaacttactgggtaccagatacttctacaaatcatctcctgtcatcggggaagcaggtgtgatgatctacactttaaaaatgttttagtctgaaacggtcgaagactcacaagaagttgcaaaatcagacaaagggttctcaggttcccttcccccagcatcctccatgataatatcctatatgaccatagtacattgtcaaagccaggaaatcagctttcacacatcttatgcagctttatccagtgtgtactgcatttagaaaaatagcctatgatttttagcacatgtttaggttagcaaccaccaccgcatgagacgataaaattcccttgggttaccctagaagtcattgagctgttccccatcactataagttcatttcttcttgctgctggtcagtattcctttgtatagatgtaccgtgctttatctatccacctgtcgaaggacatctggtggttttcagtttgtgactattgtggataaagtgctacgaacatgcatgtaaaggtttctgtgatgatctgcacccagcagatgaggagatggaggcaagagaggggatgcccaagctctcatagccacacttgtaatgagatttgaacccaggtagtcactccagaggcttcatgcttaaccacctccaagactgaaaatcttgtaggtaagaataacccaccctctgctctgtctttcttacagaggggctatgaacaggcatggagttaattcatatgcaaacatactttctccctgggctctggcctccttcactcgcctgggacaaccagacccaagatcccagagtcagccagtgccattgcagctgcttctttgtctgcaggatgcgag

SEQ ID NO: 18 - DNA, 5221 bp, Homo sapiens
  source 1..5221: mol_type = genomic DNA
gtttggctccagttaattactttctcctgagggcagaccttgttaagaagaataaaatgcactgggtacttcaaaatggttcctctcctctctctgctagcagcacgaggagatttttccccaaaatcactgtgagaccctgtggattctagcagttaaaacccactaaagtgtagggtccctgatgactgggaccccatggagtttttatctccagcaatttatcagctcagttcagatttctgtaccccagcattggttctgaggaagtttccgcatgtggtttctgctgtgatttgatcttttgtaatttattctagtatatagtgtgaagaaagaacagggcagtgccgagtcctgctctatcattgatttgagcaagggcagggtctttgccggtgttctgggccaaagaggctccatgcattttttcgggactttggattgtttgcagtttgccccataactcccacacatttgcttcttcatgtcccaaagctctggctgctgcagcattcccataggaatgtggatgcctgccaagcgacctcatgtgcattccattcatctccttggctgggaggcagcagggaagcaaattggctggttgcttaactgtttgggtcccaaccctgtgttggacttttccaataattgaagaaaaaacagaagagagatatttatttattcatatgaccatctggaaaacagctattttcaagtgtagcttaatgaaaatcacatcctttggtttgtttgaaatcatcttccaccagaaaggcattttgaagcaatgaggctttgtgaggtgtgattaaaacaatgtgagtagttttttttaagcaatcacaccaaactgtagctactcaaattcaggttgttgccttccaataatctcagtgggctgtgacccattccaagatgctccaacctgtgtaaaattcctaaggaaattactacactctttgttttgctttttgtttatcattcagtcagcacatatgggccattcttgtgcttagctttgactacttctagtcaaagaatggcaaactaaagaaatacaacggcttcttaaggcagccctggcaatgtcactggaatggtgactgcttgaagcagaaaatgctgatctgattggataattctgaatgttttctcaaaacatctcattattctattcctggaatattgaaccaccctcaaatgctacttatttgaaaaatggtgacattttagtccagacagaactgccatgaaacccctccaggacgtataatacatagacaaatctttctttcgctggcagaaagagtcaaggatgcagagatttcagacagccactctagaagcagctgttaagattcccactctaatttgctgccacagcttgaaacagtgaaatgtattaaaatgtgccacatcagttaacatgggttcagttagatggaggttgccagtttggaaggagggcaaatgttccttggtggccatctttgtgatgcccaggctgtgtgtggagctcaatcagagagaacttatacatgaggttctggcacaatccaggtacaaataagacccagggattagctttcctggatgaataattatttctgaatcatgggaaaggcacacgtggaaaggaagatggaagtctttctaagaagtctccgctgtcagaggcaccagtcctcaaatgagctccatgcaaatgaaaaccggtcccaacagcatcttgagccaaagaaaattcttggttggggttgaaaaaaatgccagataaatagtttcttcttgcttctcagcagggcccagctatcactgcaatccagatctggcctctaagcagttctggcaggtttagcttggaccagctgctgtgatgaaattgcaataattccaggtttccctcaaatctggactgagccaaaggcagtctaggcttctagcaaccgctgacttcattgccttctttcctgtccctcttcaactttcaagcttctggtttttattttaacttagatagggtgatagaggaaacctgttcagtgcaacccagaagtctggaaaatgtttatttcaggtcaattaagaaacctatattgagtacttagtgagtctatttaggtgagctgcgtggtataactactgcatcagtgatacgccaggctttgttgcttcctacacagaagatctcatttaatcaaccaacagcctatgaggtaggaatgattatccctgtatacaaaaagtctcttaccttaaaacaaacagctggcaaatggaagtatctggatctgaacccagtgtagcctgaatctcaggcctctacttgtgctcgctatgacactggccataagcaaatgagtccttaattagaggtgagtaaaagagctcggacaagaaaaactaaagtgaacaaaggcagaaaaccgtgggagaaagtagaagtaggcagataaagagtgtggaaaaaacagtagaggctagattcatgctcattctgagcccaacctctgaagaccttgtgggtcctggagcctagtggggaggtttccccagattataaagaacttccagccattaacacaaagactgaatataagttgtccaaaactctttgctcccaagagaaacgttcaacatggaagttttaatgtactctaagaggttcaagatttcagataagtcccagggcactgagacttgcagtgtgacgcatttaaaacaggctttctagaccagatgggtggctcactgcccctactcctttatgggcacccaggagaatgaagtagatctgtttctagagaataaagcatctttaacccagaaagggaaaagaactctgggcagagtacacctcagcaacccagcagtcttggaggatccagaaccacctggccaacaatgtcttccatttttgcctgtggcatctgagactcttcggctcatcccgaggcagaggcccagagataccagaggctggtggtaacaaaggtgatattcaaaatggtggaaatgattccaggaatgattcaccaatggttctgacctgaggaagacttatcaaaaagtaaagagagaagcagcatcccaccctcttccctgactgcaacattttaatctgtgaattatttattgggaattaatcgtgaagccaagaaccagcagaaaatgagcagctgcccaactttgcatctcaggtaggggagggggagcccccactgaatagagtaaaaagtacaaagagacagacaaatattgtcttttcacagtaacttttgagacctacatatccacatactggcttcatgatatgttttttattatactctggaacatttagtaggataagggacatgtagaatgtgattcacaggaaagtggtgggccctgagcaaggacctgagtgtgtttttggtctgaaactgagtgatttggggcaagtaacctcagtttcttgtctgccaaatggaataattatcctgaatcttgatagaatgttatatatttccaaaatcccaagcacattatgtgtgctaattaaatgctaggtaataaggagaaaaatgtagaccaaaacatataaaaaaacccacataactaaacaaggagcagaaaaggagagatgaaatggatggatgtttttggaaaaccatttggggaaagtggtgatcttctctagaaaggatttcatgccctctccctaccagctttgcaaataacacaaccctttcccagtggttctaaagggctgtagagcagaggtgaaagccatggagtccctcggacgatgacctacagctctcgtctattaatagcttttaagtccttagatgcagcccaagcccctcccacctcagggacatgagagggtcacaaggctgcctgatacagctgcagaaaactgtggtccccagaattctcccagttaggaatcctgttcaaggtcacacaaaggacctttggttttaggtctcaatccgcctgtctcgtgctgtaacatttgtgttccatcttgaactctgggagacttctgggccttcaacaagaccattaaatccctcaatcatgttggaaagaaaaggagactgtggggggagcaaagacacagctggcctcacacttggactcggggtcagaaggcatcgatgtgtgcattgatacaactctggtggcagggtgttgctgagcatgtcagtgtgggctctgggcgcaggcagggcccttggccccttactgcatggtcacatctgattgtatcacctccaagccttagagaccttctgtgatttcctctgtgcaggacaaagtcaggtcccatagctcgatgtcgacccaactcctgtctttcaaaactcattatattcatagctccctcattgtagtcatcctgagaggttcctcctgcctcctcagtgagccacatcttggatcacaacattgctgagtcttgtgtacagagtgtgaatgtgacacatttctaccacctttggggacatccctggggaggcaggatatttggtttaaatcctggccctaaagctgcggcatccctgggctggaatgtagagtaacagggctgccctgaggaatgagcgggaacttgatctgccaaaggaggggctcgctgagggtgtgtgcctcctccacagagggcctccctgggaactacggccctggagtagcagggcataggacccacgcaggtctgggaagcctcataacaatccaaagaccccctctttcccaaaaagccagaggaaatgtgccatcgaagaatgtgggcgtgggacaggggttggatcccacctctgccacgtgcaagcagtgcaaccttgagcaactcacttacaacttcttggagggttttatcatctgtaaaaacacacctacctcagagggtgctgtgatgattagagaaaatgcttacatacaaataacattcaaacatgtcagcagttttaatatcagccccaaaatggagggaaggggggtcagaggtccctggagccagctttgccatcaagggcaaactctctcccctctgcagtggtggggctagatgcgattatttctaagacgctgagcgtcccacctccaggattcttctttctcttttcttttagcagtctggtctcagggaggcaggttctgagatggagatatgagcaggggcttactggggagtgagcccaggaacaacaccagtgaggga

SEQ ID NO: 19 - DNA, 3815 bp, Homo sapiens
  source 1..3815: mol_type = genomic DNA
ttgaatgactctgagaggagccactggtggtaaacccaactcccctgtcaggaattgtcacgctgccaccataagtaccacagcctgcagcttaggggtagacccctttttacacatgaagctcagagacgtaaatgtgcccagtgtcctgcagagcagggacgccaacccagagctgcctggctccgaagtctatgcggctgctcccaaggccctgagctgtgggctggaatttcccacagggtcgtaagtgacagcccccagtgctcgcagaggcaaagggccagagcctgatttgcaacgtgcgcctcgacagccatggtgctccaggggggcagcctttcccatgccttcctggggccacagagtgcagttgagctcagcagcttggaggaggtgcagggggccggcctcagcccctgggtctccaggtgtggggacctacatcaggaacagacagtctttgcagtcccagctgctctgtcccctggaaccttgtctggaagctcagtcctcccaggaggggaagaaaccagcctccgcttgcagatttcacctccctgtgaactcccaaagggcgggaggccaagccgcagctgaggtcagagaaagtagcagggtgaccgtggggctccacgtggccctccctcactctctcacgggccctccaaaacaggcttctgcccctcagcaacagtttccccaccaccagacaaaagatgagctgagctgcccagggtggatcagggctctgaggctctgctgtgaactccgcttgggcagggacattgtatcatttttacgattagaaaacatggctagtctgcgcagcggagccccactcacgagcacaactcaagggattgtgttcttgacatgattggccccaaactgggtccatgggaaagatggctctgggtccagcagaggcagcctatgggtccccatcccagaatatagacctcgggggtctatatctgggatggtccaatcctcccttcctacagagacctcaggcctgtcagcaccaaacgcctccccagggagagaagaaagttacagatggcagggacaaagcattcgggttttcatttcctccaggggtgttggccacattggaacgtctgtgactgcgagaacggggccaaatgctcccacgtgcttgccaagctgcgagtacaatcctcctcctccatctctccaggggatgctcgcttcccaagcagctgcattttttttttctcctcctcctggagaaggttgatactgagttcccattttacagaaacccaaactgagtgcagggcctgaagccaggatgggaatgacagagccagaaggactggctcccacctcccagaggtgggtcctccatgtggtggacaggatcagaggcctcgttagggagatggggtttcaacaacaggaggacaatctaccaagaagcccaagggcgaatctcagctcatgaccttggcgtggcccctgctatttcctggcccgtaacactgcctgcccagctctcgactcagctcacaccactctccttacacagtcctgtcattcggacctcagccaaaggccacctcctctgactgtgctgaccccaccaaagctgccctcaaggattccatccacctggcatttgccctgtgggtggctgtgcccacatctgtctgccccctcctgccccctctctgttctcccctctgtggacgcgcaccagcccagtgctcagcatgccacagcctggagtgaagggacatcaggccacttcttgtgcccgcctgggtcacccttcaccccggccgagcccacgtggatttgggccctgcatcttcttggggaaccatcgaggatctgggctgggcaggcagcagcgaggagattggcagagaaggcggcaggaggccggggcctgtgaccagggtctcgtcctgctctcctgctgacccacctagaggtttctgtccatgatgtctggtccagtgggctccagtgagtgagggcaaacctttggggcaggagcctggggcatggccattgcacttttccagagggaggggctcttcccttctcctgaaggtggagctgtcccgtaaggggtggctcaggttgaagagaagaagggttgggggtcacgcctgcagggccacagatggaatgaggcccccgacaagggtcccctgtccctgtcaagggcagtcgccagggttgtgtgaccggccaagggcattggagagggagggaaagccctgagcttctgaagtacgagagctcctctgtcccgagccctccatcctcagcctcacctgggctggcagtgggggaagaggcaggtgacaacccccccgagaggtgacagccctggggcgggagccgccgccacctgagagtgggtgaggagcaggttagctgggtgaaaagttcacagtgaggggagctgtctgttccctcgcttaatttatccactatttggctaaccttgctctgaacccaggcccgagacccctctcccttctcccccgcctcccactgggcttctgagccgccaccagacctcccgcccaagcccaggcagggacgtgctggccttgaagaggcctggggcccgggcgccgggagaggatggctacatggctgtcagttatttatgaccgcagtcttcccatcttcagtaaccaaaataaagtccggttgccgcgaggtgcaggcccccgaacgccagacatccgccaagcctcggaaggcgccccgcccgccgccttcgccaaacacacaactgctcgggagacagtgacctttccttgggggccatctgtcatcctggtttgggcgggagccagacacatggacccggcctcggaagggaggccggcctccttccctggccctggggtggcctccgggccttcggtctgcctctgatggctcagggtctgctctgaagacccttcctcacctcggtctccctttcccttttttcattctttcattgaaatctttttggcaactataaaagccatgtgttggcagcagccagtggaacaaagatagacaaagagtgaatcatcccagcccccccatatctcagtctccacggtctgcactcacaaatgcacacaagcgtgtggacacctgggcgtgtgtgagcagggaatcgcttgtaataataggatccccaccacacattcctccgcagctgtttactaacctgcttgcttttgtaatggggtaggccccacacacattctgaagtgtacagctcagtcagtgtcacgtgtgtggccacccctcagatcatgacacccaacattctgcccccagaggttttccccccaaccatagcagccctaaaggtggccactcgggttacctcgatctctctttgaacctcatcatacagaagacagtcttttgtatctggcttcctttgctcaatgttttgtttgtgtcacccgtgttactgcatgtggccgggaggtgttcattttcattgctgtgtagtgttccattgtatgagtcctaactgatgcaaatttgcatcgttttcttgtttctttttcaacacaaatgatgctgccatagcattggtcctcattcccctttgtgtgctagcacttttatacatagagactccatcttcccatgtgttaatgagaatgaaattttttctttaatacttgtccagaaagataatgtttcttccactctggaaaatcacccacaaaacttatggaccctcattggctggaccaggtcagaaagagcctatcctgtgggcaatggggagccattgaaggttctacacagaa

SEQ ID NO: 20 - DNA, 4291 bp, Homo sapiens
  source 1..4291: mol_type = genomic DNA
cagctttgcagacacaaagtctgaaaaagcacttttggtttttgccatcatcacaacacaagatattattcacatcctgtaagaaatggccctgaaaaagtgatgactgttctcttatctggagcacagaattcccagtcccaaacattccgtttctcaggtacttcaaggccaggggcttttgaacaaatggcgtctgaggatgaatacattttagatcaatgattctgaactcaagtgaggtgagaaaggggacaaaggaatgggaatacgggctaggcgtccattgattcagagaaaataacaagtggaatttattgctctgattttgaactgaatcttgtggctgtttttattgtaattcaaggcactgtttaatcagaagccattggagtgaaaatgttcatgttgcagatttactttgtgacaatgatgtatgggaagattattctgcaagaaccagaaaatatgatacttgttggaagaaattgaaataaagaacatccaatcccatgtgctaacaaagtggggattcctttccaaagctttgtactatgcaaaggatacttttctggtgtgccgagaagttatctctgggactgtcacacttggaattccagcagattaccctatcatcaagtcatcagtatgctgctatttgaataaacacagctgttgttttccaagaaatatggctaggagcagcaaggaaaaaagggcaattagtgttattgaaatgaaggggtactcaggatggcatatatatatttatgagcattttcgtttccgaatagctatttaccatgctgtgacttaacagtggtgtaggatgcatctttgtaaatcataaacatacgtagtgtttttggagatggcattcactcccccatattgatctaccctacgctgactctaatcctgtcaatctgcatgtctggcataggagccccatctccttttcagactgattttataatttatactgtagcaacttactctccctatacccaccagaaacctcctttcctgttggctttgtattataactactacatctttggcattcccacaagccctcaggaacaaccttcagtctgctgtaggtcacatacatcagtacctgggagactctgtctacaaatatatcttgaggttccccttgtgaatgtgaggatcttggagaactatgagattgtgtgtgaaaattatcatcaccatcatcatcatcatcatcataacctcaaccatttaattatctattaaaggagaatgaggttgtcaaagcaggaccagctgggattcgttggctgactcccagatgcgactgtgaagaagcggatattgggtgctctcccacctcgtcaggagcagcgttctcctgcctgctcttggcaccccctagcatcctgtgccctgctaaggtgttaactgacaactgacagattttactaagagcctactcttcccagtagtattttcacttcaaaagttgacttaaaaacttaaaaaaaaaaacctctgagagtcaaggcataaagtttttccactacagctcataaatagatcacagctctaccatcttcaaggcaaataaacaaatgctccctcgaccctggagattccattagctaccatcccatgtttatcaattttcaagcaaaattttcaaaggaaatttcattgtctcttctactaactgtccaatcattacaaatgaaatcaagcttcatcttctcccctcaattgcgctctttaagatcaccattgttaagatctaattgccaatctgagggacttttcttcaatgtaattcagtactctttgggagagttgtgttgttaaatttgtctgtctgtctctctccctatcttggaggtgtagttctttatgtctgagtctctgcctcaaccgtcttcataatactgttgtcttacataatgcttagccccttacaataatctgatatctgttgaaaactaaatgatttgggagccagtgctggtcacatagttgaattgaataaagaaatgttagactgagggagcaggttaattcatctatcagcccttcaatacccacccactcttaagattggctctgattgcacaaattaggaaaagctgggcaggaactcattgaggtttgattgaaagaaaagagtggcatctacagtataacactgagtaaagcatggaggtcagagaggaggagagagttattctgacgcagagcaagaaagtgattctagttttaatcagacattataaaagacacagaaatatgcatagcagtggaagtgaagatggcctttaatcacttttaacggcatctctaagcaagtgagatagtgctgaagagataggtacaggatttcaactttacacatcagttttcaaaacactgtagtaaaaaatcctgcttctgaacattcccccacctctcaatagttcaaatcctgttttgggggtgctgggaagggatagtgcataggggagaagggggcacaccacccaagggataaatttgctaagctgaactacaagtaactttccttccattgaacacctccaaaattagacagcataattttacatagagcctgccctgagtgtaacagaaatagagtcattgtaatagaagtcatcacttctaaacaaacatcttggctgtgcctctgtgtttgttttggcttcagaagctgagtcctttatcaaacagtaggagactattatctggtgataatgtattttgattctattgttatcttaaaactggtttcttctgggcccatccttttgtttaatgacatgtttaaaaatctttgtaagagctgtattaaaatgacagatattttgtgttgccaacataaaccacaatctccaaggctggtgataaactccactcctggcctcagctactgtaggctttgaagctcttagctgcttttcaaatgttggcatcgttgtcaatcattctgtatcaaccttatagtttgtgtgagaagccatttgttaaattgcagcctcctagactcaaagaacatttaccaaatgctttcagataaaatgccatcttattaatactgggaagctcgcaaaggaagaaatacttgagggaaatcaaattgcaacgcattactctattctccattgcacttttgtaagaactgagaacagcacttattcccctctcttctgtgtgtgtttgtgttattcatttgtaaagctctttggggcagggtacacataccatactggtggtctaaattataaaggggccaccccaccatggtaactcactcctgagccaaaggagcatctcttggattttcctcatattctataatcctttcctctgcttttgagtgaccctactcttctctgaaggtcttctaaattatgggaaaatgtatggcagagatggtgaaaggtcagtgaatattcctttttttcccaacactatcaccagtctcctgctccaaggttgcctagtagctggtcccatagtctattttcataaatcctttttgtatttacccctctcatcctgttattttagtcaaatatgcaaaagatgcagactctgaagtgccaactcaaatgtctaccattgtcttttaacttacaggatctaagctagtggttctcaacctacatctgtacagaaatggaaattgcaaatcatcttgcagcagagttctctcattataggaagatttaagtaattcttcttaggcaagaaagtgccattgtgctgggggaatcgcctcttggttttcagagttagtgtttggggagaaggatgaagaattaagattctctcttatctctgagattaggagtaagggaaacctacctgggtggacaaagctagttggtgctaaaggatgccaaagtcctagagaggctctgactataagttaaatggctggcaacctcatctcagtatagattctggtgcttttataggagattagagctagtgtggcatgtgtctgagccaagatgtacctgcaggtcctgccaccctttggactgaaggtactaggagtcttgtttgaggaactatggaggtgctgacaggaaacaatgacaaaggcgtcactgcacactacacatgttccttgtgccacagggtattattgttcagtagcaatacgccaatatggcagtggtcatgaaccggtgggatttagttctttctccacttggtgtttagaaggacatggtatattttttccaagtaagcccaaatattatctaataggattcatggaaattttgtaatagaa

SEQ ID NO: 21 - DNA, 7731 bp, Homo sapiens
  source 1..7731: mol_type = genomic DNA
ctctccgtgacggtcacgggtccccagcttcctggcgctcgagctctcccggctgtctgctgcccggcgcacgcctgccggggactgccggccactccgggtgggggaagagggcggttccagggggctgggggcctgccaggacactaatggaacacagctggaccggcccgccagggaggaggaggaggagggcctagagggtccagcggcaccagaggcgctagactgcggggtgcacagccccgccccgggtctggccgagctcccaggcccgcccctcctgcctcaccgccaggggaatgggccataaacctggggtcatcctcgggtggagcacctacagaacaggccctaggaaggagagagctgccaggttcggggatcccgaccccaccccgccgacagccacgcctccagcccactctggccacgccactggtctgcctttggaccacactcatggccgggtgccttccctgactccgccctagccctgcctcctatctggctccgcccagccctggccaaccttagaacctgtcccaaggtccagagggcctagatgaggggtagcctgcctctgagaggcatggagccctcctcccgcgtggggtcagggaagaagggcgtcgagggtggcgggtgagggaacggcgaacgggaagcctccaggctgcaggacgtagctgcgggagtctgcgaggctggagtggccgaggccgccccgagcatagaaacagccctgtatacgtgggccccagggaggcgcctgggcagaagtgtctaggacgatgcctgccacggagccctggagtgggggcagctgatggctcaagacacatgagctctggtgggaccggggtttggggaggcccaagtcaaggtccatccttctgggatgaggggcggaggatgggaggcggagcccggtctccaggctccacctagcgggaagcttgggaatcgcggagtgcagaggtggggtgcggaaagcgcctgggctcctttttctccttgtcccactggcgcctacccaccaccatcctgcccagtatctcggggattcgccccagatagaaaggggagtcattctaacatctatcttccatctccaggtcctatccttcccttcttccttgcagttatccctgagctgcctctgccggttccagtccctggaacacctgacaactgctcttcctgcctaccctctcccccatccatttttcatagccatgcggtgtttctaaacaggaatctggttgtgtcactttcttgattagaatcattcaatagcatcttatggtccaaatagactccaaacttgggacttgagctcttcaccatcagctccacctccctcagccccagtccctcttggggctttctgtgtctcctcatcccttttacgccctccctggcctcatccttgctgttgggaggtagttagctcccaccacccccagtatgtgcctgcctctctcagagcagtcactgcctgggtctctaatgacttattcggcaggacaattgattgagatattaattgctttgttctcctgttttcccctcccacttgcaagccaagccctttgatttttctagcctcagggaactgtgcagggccaaacgcaaaagctgcttgaagtctgatggcttgtcctgagtttggaaagagtaaacttctggaagcaggcgtgccatgtgttcccaagggatggggagaagaggacagagaattgagaggcagaagctagctgcaggggtgcagaagctgggggtcctggtggtagggactgtccttgacaggtgacaggtggggatgtgggtaacgcacctggggtggctggagtgcaaacacttgggaaccagcagtgctccaaagtagcaggggaacagcttagtcactgagcctcaaggaggacaatggacatctctgtgtgaccaatgtagactaagacaggaggcacctcttctgtggtgcagggagggccagcagctccttgcatgggaaaggtgggagggagcccagtgatggttgagattaaattttttgccaactcataggaaagcgtttagagtgagtcagactggagttggtttaaagtgaatacagaaacagggtatttcttgtgccaagcttcctccctactacacctgtctgtgtgtctcttagctgcagagctgagagtatgtgcctcattcagaattgtatcctcagcacccagcgtggtacctggcatatattgactgctcaaaatgtatcggttgaattcgcttttcctccgtagagtctaaaacccaagtctgcttccgtggttcccctctggccctcagaataaaatccaaaccccgatgatatcccacaaagccactgtgatctcaccgccccgtatccgcagcccatcctctccagcgaactgcactatttgcaattccccaaacttgccaagttgtttcatgcctcgtgcctttgcttatgaggcacctctgcctggagtgccctttcccctttctctgcctgctgagctttcaattagtctgagctcagtgcaagtgtttccttgtcttccctgacaccctcttctgaaggcagaggaaacttctcctgctctgcagcctaatgctcaggcccatgtctgtctcctcccaagcctggtttccgggcttctctagtagagtgagttccccatgcccaacatggtgaataaatggggctttagccttgatcctccaaggactcatggtgtgacctcagcaacctctccagcctcagatttcctttctgcacaagggaggagtggaactgaatgttctctagggccctttcttccctcattgagcctggaaatgagagcatgtaaaacacaactcacctgtgtgcacgcaggccagctaagggcagaacatgctcagacacagagctgcccatagcaccatgcatggccaccgggagcaagcccttgcccagcagctgcacgtgactcacacaaaagtgcatgtggccctctggacgtggatgtgctgtcctcccaacccccatcccccatcaaccactggccctgacagtgaaaaattcagcagcagtcagggatgccagtctggcagtgagttaatattcaaggaaggagccttctgcaatatgcatgccctgaggctggtgtcctatggggctctccccttcccaggcctgcacaggcaaagccctccccaccccaccacaggcacagacacagtctgcctgcggcagggcactgagaactagaccttcagctctaattgcttcctggacagccccctcccaatgtctcctcattcattcattcagcaatatctatgcagttcctatcctgtcccagcaacagtatgaatgtatgaccctgttctgccctcaggggctcatagtccagtgggggagacagactaaatatataaactcagatcaggagaaatgatactgtggaaaggattaggtgatgtgatttttaaaaaatgactgaagagactttcaaattgagggcacagcaaacacaaaggccctgggaagggagaatccatgaaatgtgtttgaggagcagagaggaggccatagctgctcagacagtgacagcaagtggcaggacattggacagagcggcaaccagagcatgccgagcctcctgtgccacagtgagagcctttgttttagtcaaagagcaatgggaagcccctggaaacagtatccatcccttcccccagctcctccatcttgggagccttagagactatgccttcctcatctttacgttgtcagtgcccatcccaaggcccagcatactgataaataaatgaccaatcaagttctcctggctcaaggcttgattcaatggctcagagtaggtcaacagggccagattaagacatttacatgccatccagaagagtctggcactcccatacatattcacaatagaaataatactaatccatgacatacttctttatgcacatctggaatgaaaaccacttccttttggattttcaattgcaaaattctggattagttcatggaagcagaactttgtgtgcactgttgttactgtcttgctggcatcctaaactgaactcagtccgcccattgatcacccagtgctagatggcagggcttgcccaaccatcccctcaatgacatggtcttggcttttggacccagccaagcctttaaatggggtctgcccttaccccagcccctctttcgctacccagctgagccctaaacctctaagctcaatgctccctcagcctcctctctgggtcctagagaggaccacatctcaggtcccccatgtgacttccacagcatcttgtgctctatgtatggccccgaagggaattagactcaatgtggggctgggagaaaagttgacctcccctccccacttcactgacctccttaggaaactcagacaaagctgattctcaggggattggaagctgggtggttgaggggagcacctccacacaggccagtttcccaggccaagtccaggctgagctctgcctctactaggctgcaccccaccccacccccagtgagtgggaagtgacaaatgagcctgaggaaggaggagggagctggcgatcgatggcattaacatggtgcccaaattaaatattgactttcttggttgactctagggacacttggattgatggctgaggctttgccatgccaccacaggggataggcacataacagcctggagcaggtccatagggaagctgaggcctggagaggaggggctcacctttgcttccacctgctcaggaggagtagaagtgagggcttcagggagcccggatgcctgcttctccaccttccccatggagtccagagtccccgtcccgaaggtcttggagtcaggccttgttgagtgtccccccaaccccattcccacctccaggaactgtggctaacctgaagtgcaacaaatgagcagaatcaatctctgtgatgataaccattactggtctcaggacccggcctcaaggcgagtctagggctgcaggggtaagggtaagggtgagggtaagggtgaccccttcccccaggtctcctcaggaccttcccctacttccccacacctcctccctacccaccagtatccccaggatatgtggagcccaggaaggctttggacctcccttgtgcgggcactgtgctggcaactttccctccatggccccatgaggcaggctgtgcggaagtacctccttttcagagggcactgagtgctcaaggtcactgcctttgttctgcctaagctgtgctgtgacatggtagcctcacgggagtactgagcagtgtgagactgacttgagatgtgtagcacaatatattgaatgagatatattattaaaatgaatttcaccttttcaaactaagactgtaggagttgtcattatcccaattttgcagatgaggaaatcaatgttcagggaggccaacacagatgacagcagttctgtgtgatcatgcagtagtgacagtgcaccaaggctgtgggggcccagcgagtgctgactgctgggccaggggttcagggacagcatcacacagggggctacttaggctgaaggagagtttcccctctgtgaggaggagggattcaggcagaaggagaggcatggaggtgagagatgggccagaggagggaggcagccacatcaccaagggccttgaactctaggctaattccaggatccacagaaggcatctgagcagtggcctggcagatctgcattttggaaagatcacttgggccatgtggatgaagggctggagactggaggcagagaaaatgagggcaatgatgagaaatttgagtccaggaaagaaatgcaggttgttagatattagctcaaatggaagggagatcgagccaactcattttacagatagggaaacccatgtcagacagggtccagtggcctgtccaaggtcacgcagcaagtcagtgcctgggggagcagggactgagggcaagggagggacaaagccccaccccaggaaagctaagtggctggtctcaaagccagcagaggcctctgcaggtagctgtggtttgtgtttgagcagaatcaacaaggtaaatacaacttccatgcaaagcaagaatggacagggcagggacgtgcacatcacacctcagccccagagggccccagataagaccaggaaggagggagagcacaggacaaggcattaagagggcctttaggtgatggtcagaggaaggaatgtggagtaggatctgctggtacatggtcgggctttgtttgtgcaaacccccttttaggaacaggcccccctttctcagagacctgctccctcccccatggttcctgatgggactgccaatcacagtgcctggggccagccagagctcttccttccttgggggttttctaattggaattcaagagagaaagtttcttcccctctaggggcaatgctgagtgatggaggaggaagcctggcaggcccagggggaagaggccttaaagaatgaagcatgccaggagaggatgaaacacggtggcactcgtcccatcacgaaggccagccgcacttgcccactgctgtccttgcggcagcgcgctcctgggtgaacaaagctcccaggtgcctgctggctggtatcggattctgccactcccagccacaagtcccagtcacaagctgagctgtgggagcttgggaaagtcatgtcccctctccaagcctcgatttactcatctccaaaatggggcagtcaaatacctacctagaatcaaatgaggtagtgcttgagtgaactctgtgtacttaagtgctagggaggcggaggagtgcgcaggctagaagcacagtcctgagtggactctgccctcctctgaccaggtctcacagcaggcaggattggaatgagaccagaaagcctgtgccaaggcctgcaaaatgaagaggcctccgcccctcctccagcacccctgggcaggccattatctgtgataccccaccccagcagccagtcacttctgtcactcccatgctgcagcacctctctccagggacctgtctgtgtctccgagtccctgggggcccagaggtgcccatgttgctaaatgaccaggcaagaaggacctgtgaccctgctactgctccagctgctgcctgactccagaatatgtttccagatgtaatcctgtgccccctctttctgggggcctgaaactcaggcatgtaatgaatgcaactcagtacaagcctccagccccccagcctggctcccacacccctggcccggtccaacacctgcatctccaccacaacccagcatctgcggctcccctgcagccatggtcaagttccaggaagctgagctgaagccccacctccatcaagcgtgggtttgaagtcccagctctgcctctgactgtggccttgagccgggcactgcccatctctgtatccacaaaatgggcaaccttccaacagggttgttgtgaggggccaggggagtgtgtgaagctcctagcgctaacccgagatcctgtcccctgataacaaccctgtgtaagcggctctagcccatgccccagtgaatggcagccacgaccatcacagccacccagccataagaggtcccctcccacactcccaactgcttcccacagcccagatcggggaaaggatgaatacccaga

SEQ ID NO: 22 - DNA, 5273 bp, Homo sapiens
  source 1..5273: mol_type = genomic DNA
ctttctgccaccgctgtcagttgtgctggcagctttggattttgataggagagccataacttctgagatgaatatgcccacatgtgttactgaaacatgctcatgcaaagaccaggtgtgagataaaactggaggctgcagacgcctctcttagtctgtgtaagggctgggtagggaggatccaggaaagctgatcttgtagttagagggctgggaattggtttaggagagggagaagagtggctggctggcaaacacatcctggttgggtacagggagaaacccactgaggcagaacccaggattcattccattcaattaattagatacaattttttgagtacttgctacatgccgagcactcttctaggggctggggacacggtgaagaaaacaacaaaagttactgtcctcatgggggcgctatgttgtggctccatttgtttgactatgaaacctcatttaggtttcaagatcctagcctcatcattgtgcttggttgcaaatggctcaggtaggattcccttcactcccttctgttttcctttacacaaccctggagttattatcatgtttctcagtggtcagatccaagactcttcttaagaaagatcccaagttctggtatagtgccatctccagggcttcttgataaaggagagacctttggatctgaggtcatagacgatacctacctcccatctcccatattttttctcactgcttccagggagactgactacatgctgaaaagcaaacacgaaggttgaatcttaaaaggcagagagaaagacacaacagactgacaactgactctcaagtgaaactatggactccagaagacagttaaatgaatcttcaaagttagaataactgccaactagaattctgtggaaagaaaaataaaaggaaaatacagacacttctagaaaaaaaatgtgataaatctcacaccaactgacctgtagcaaaggaaatcttcggcagaaggaaaatgaactcaaatgtaatctgaaagacacagaaagaaatgaagagcctgtcctggttgcctggttaggggtagatcaggggacagccaccaggtgccagctccctcctcctcccggcgccgctttgtgcagtggctgctgggccgcctctctaggacctagggctctccctgtgagcggagggagggaactttggccgcaggaaggactggaagccagctcgaaaaagcacattccttggcaggataaaaaacttttttttttttttaagtgtaaggacttaacctttgctaggaaatggttgcagtattttcctttaaatgaaaagaccattgtgattgaaacttcggaaaacacacaacatcctgagttccgtgtaggggacagaggacctggtccctgctcaggagaaggcctgagacagaaagggcttgttgttgcttacagtgacctccactcaggtcctgctccataatttgtggggcccagtcaaaatgaaaatgtgagcccatggttcaaaaattaagagtttcaagacagggcatcaaaagtgcaggatccttctgagctgactgcccaggtcgcatgccaggaagctggcccagcctccagtggaccactcttattctgctggccaggcctgagtttctgcagcttctctctcatgggtagacacagaccccagagattcagggcaggactcaggacttcccccatccttggctcccatataccacaactggcaacaatttcccaacctggttcttcacttgatttctcctcatggtttagagacagtgcttcccggttgtgaaaacattctggtttcatctgcgcttgcttgtaacccgggggtctggggaacacagaacagctgcattgagtgctggtctacttggcgctcttcaagctctttacaagcctcctcgtaggcctactgtaaataggaaagtacaaacacatgccgattttgccgcagattaaaaccatctcctgcgctccctggatcgtaacacataacagtttgtgtttatgcagcatttattagcttgcatgctacttagggactttcatagttctctgctaggcgaggccttagtatttgggctctccagcgtctgggaggcaccatatggagagttgtaaagcctcatttcgggtcagcctgaccgcacaaggccagaccagagctgtgctgtgcaacagtgtggccacttgccacttatgtctcttgagcacttgaaatgtgctagtccgaatgtagagtacatactgaactttgaaggcttagtacaaaaaatgtaaaggatcttgtttctttttccttttttaaatgcagctactagaaaattatatgctggcatcttatttctgttggatagcgcttaaagcctctctttgttgtatacatacacacacacacacacacaccatacaaactcctgaggatcttggaaagaaaaccaggccagtgagatcatgtgacttaaccccaatctctaggaacattcctcagaagatcccgtggccacctaatactagaggcttttatttgccaatggtaatctgtctgccttgccttttaaaattaaaaaatttattgaacacctatatttgcaagggagtatgacttagtgatttcagagtgcaggctccagacttggattccttggtcttctcatctgcaacgtgggattatgacatcaaggacagaatcaggatggtgacccagtttctacttcatgggcttattgtgagtgaatgagatgaggcacagaacacccttggcctggagtcattctgctgtacagccaggcctgatctggcccctggactttcacagggctcagcagactctgaggaagggagggaaccatgatcccagagagctccaagacagattggaaagaccctagaaatgtaatgtcaaaagttaggatggaaaggttccagtcacaatgtcaagcaggcccagggtggagggtctgggtggggctaggattgattcgggaagccagaaaatgagtgagaaatagatgctcttagtacctgaactcagagcatcccctcactggggccacaatggccaaggctgaggaagtgggcccaatcacattccaagtcctacattgggcctaagtaggcagatctggagtgttggacccatttccttgcagtcttgtccctgtaaggcaactgcattagggtaatgcgggttgatgaaacaaatgaatcccaaaatttcagtggctgaaccactatgggtgctcctggtcggtaggagggcttccacgtggtgagtcagggatccaagctcttaatgactctaccctcccttaggcctgagtcctctgcatctagagagcagacaggaaagagtgggggagaagacacaacctcttctaaccaccttggcctgaatgtaacatgtatcattaactttatgttcaatctgtgacaactagtctcatgcccacctagatccaggctgggaattgccggctctacacatagaatagagcttccctaaggcatttgccagtcaccatgcccaggccaggctttgaaaggaaaaatcatgaacacttctaagggaataatgacacaacaagcacactcaaagagacagcacacacaccatagtctcagcacttcaggcatcctgagtggttccaaaggcctctctcagagccctcaaaccatacccaggcactggcctttgagctcagtgagccgggaccagcaattgcactggcaaaatcccaagtccttactggccaggactggatcaaagggagaggtgtgtgtggggtcgaggcaggtctcacttattatccaacacaggcgcggtgcttttaaccagtgcttcttccacacctgctggcagggggaggggcgcaaagaaaaaccagaccactgaacctcaggttgctcatgattttgtgacaagatggccagacatctgatgcagagaatgtcatgcctctcaaagaggggaagtggtgaatgccagaggggcactcataagtgaggtctgcagagccacggagcccagcaccagtggagctagaaagaggctagcctgctccttgtgactcccatcctgttggtggggaataaattttctccaattccgcagccccatctctccctacaaagaggaatcctcccatgttcaggcctgttggagacctggttctgtgtttaggaattgctgttggtgtgtgtgtgtgtggtgtgtgtgtgtgtatgtatgtgtgtgtgtgtgtctgtgtatgttgggaggcgatgacaactaactctgaagactgggctatggactttgaagtccttcctagtgagggctccaggcacaagctgcccaagtttattaggtacaggtttatctggggctaatttaccttgttaacataattgtgccagaggatgcagccctgaggccctgcctcagtaattacccttctcttgatctgtttcatctctaagggctgtgccttcatgcagagcttgctggcccccacttctccttccaaggccccgggaggggccctggcaatgctttcacatttttgtaaaatttgcaaaagtattgtatcaaccacaatcattcaaggctgctgtttcttttcaccctgacttcccctctgtcacatttcccttctgtcaagggatatcggaactgtggcattttggggatccagcttagggaagcttgattgagggatgcatttaatgtgacaaatgggatgtatttatgtgggatgcaatatatgtatataatatttcatgagtgtgctcagtcacttccatgttcatgacaattattggggaggattggtttccaggaatactcctaccacccatggtgttgtggggtgatgcagcatgagaagcatgtaggaagtggaaatgaaacaagattgaaagcacacagccagaagctggtctgtggaaagtccctgtagtcttacagctcatgttagagtttactcctaattgacaactgtgtgattaattctgtgtgtcagcccagatattttgggatgtgtcgtgagagtgtttccagaagagatttatatttgaattagtagattaagaagactgccctcatgtgggtggggattcaatctgttgagggcccaaataaaacaaaaaggcagaggaacctagtgctccgggctttggacttggactgaattacaccaccagctttcctggttctctagcttgcagatggcagattgagggagctctgagcctctgtaatcacataagccaact

SEQ ID NO: 23 - DNA, 2775 bp, Homo sapiens
  source 1..2775: mol_type = genomic DNA
gaggtgggaaagtaggtatctttggggacagatgaaccaggatgccaggaactcttgggttaggtccaaggctcttcaggccttggagctgctgagcatctcccttcccacctctctggaagacttagaaacttgttatttattatctgagaacagagaatcctagagccttggacatgagcatggccaaaaacctgcgggggaaaaatccaagcacaatgaaaaatgagtgctggacaaggagattaggggagacacagtgacgcccacctggacagccccaggcagctcaaagttgtcctgtcccatgtccccacgctgggtgagactgcacacccaggccggaagcaagtacagagagccacagggagagggagcactggagtctgggccaagaataaagaacaggtgcccaagcctagccaaagaggtagcgagcattctggggaagaaaaatgggctaatcccagctacccctgaggtcaggaaacctttccctctctgggcctcaatatccttaactgtaaaatggctgcaaggctattaagcgttcttttccgagttgtttgtgggcaggagaagccttgaaattgcaatgaacagaggcacctggctccccagggacaatttccgagtccatctccccaccttgtttatttgtaaaatgggaacacagacacaactggtgtatgttttcattaaaccaagtaggagcttgtggcatttctgtctgcacttgagacatgtttatactgctgagtccattatgaatctgtttaatatccttcgaattctccgagtccttccatccctgcagccaggaagtgggtaatgtgcacacagggaagaaatttccaggcactcagacctttaggtaaatgaggactctttttttttctttaaagaggggaaagcctgttgcaaaaaaattaaattcttcctgttccagtgatcctttccttgtaggaaattggttggtcctagagagatggtgaaggagcaccagctaggggagcctgggagtgagaatagcatccctttgccctgggatggaggcatggaggtgagtgactatccatcaggggatctgtagccaggagctcaaggtcacctgagggctgccttggctggctcctgggaagccagggaacaggcttcccactggagagctccctgcttgctggcagaacagagagatattattttaaggagaaaaatgagattcctgttattgctttattcctcctgcagaaacgttggtgcgtcccagccagggccaagatgagaggcaggtggtgccaaggaaggatgagctcatcgcatgtggggacgtaggtgattccagcaagcacagtgtggaaaaaaagctccattcttgggatcccaaatccctgtcaaagcagaaccctgcttccctcccccacctcctccccatccatctaagcccactcccatttaatctctctctcttcttgcatcttcaaccaaacactcaccacttaccatatgatcttgcagctagatgagaaagaaaataggaaaatatctagcacatactagatgttcccttaatatttccttcctcccttccttcctgtggaagcaaaggctatagtataattaagaaattgtagcaattgtgagactttgaatctacaaatgtcaaattcctgcaggactctggaaaatatgggtcacaggttatttcttggactcatcatcagattcctggcaatggtctagggatgacacaagtcactgtgaatggaacctggcatgatgaagaagggttccagagagtgggggaactttcctggtcttggccaagaaagaaatgtcatggccaggagccaggtcagagagggcaggagagtgggttggagggaagtagcctgaagtttgcggatcaagaagctccaaaaagtggccatgaaatcggaatagttggaaattctgaaaggtgagagtgatccatggggtgagtcacctttggtaggacacagagcactggcattcggctgcctcaggcaagggtctgaaacaccaaaccaactttggttttccatctaggaatcataggaggtgttattaaccacaggccttctggaagcttcctatggaagggccaaatccctttgtttctgtgtcatgcaggggtgcatgtgctgtcaaaatgttaaaaatgtcacaacagtttcactttcatcactttccgcaattattgttttaaattccatatcctctaaatttccagccccctgggcaaagcccaatcacactgccctgggtaatggccatgatggggcgaggtatctctgtgatggaaacttgattgctttctgcttcatttcttgggttccgttgactactctttgtagtttacgatttttaaaaatccatcctgtctccattttgctaaggaaaatcaagaaaagggatttgggtccacacgttggtgaatggggataaaaggtaggcaaggggtgttaggtgccagagcaggggcgcaaagggatgacaagtgagggactgagttgagataaggctaagatgctggaagaaacaaaaatataacacagaagagaattttatttttctctcactttgaccagggttgggcaggcaaccaggcaaacaccaggagcttgtgtgaccagattccttctgctgtgttactcctcatcctggagtgttgtccttgtcttcatggctgaagctacctcaccatgtgagcccacattcagcccaaggcaaggacagaggaagggagacatg

SEQ ID NO: 24 - DNA, 2679 bp, Homo sapiens
  source 1..2679: mol_type = genomic DNA
ggggggttttattaaagcaagatctgacgtcagggtgaaggacaaaccctgcattttcctctgaaaaaacttttcctgcctctctctctttttttttttttcttccctctgtccccacctccagcctagcttggccagcctccccgctctgacagttcttaaagggaaagttacagacttcacacagagcaatccttgaactagcgtcatgtatagatcatcttctggatacaactgtccaaagcctgccctctctgtttctcagatccctgtggcaaggcactgctggctgcttctagccaccaagcctgggttttgattctttaatgcaagctgagctcaaggttccaaagtgttcctaggaggaggagagtgtgggttggaggtttggcagacttggtggaagatctgggtgcacgtggggcttggaggctgactggtttgggttggcagcgagcaggaagtctcccttttgcacgtgtctgtgttctaatctctttaatgacaccaattataccacccactgaccttgctttattgcatctggtgtcagcagacccaacagtccatgctggtacagaagggatgcaaacatgaacaaagcgacaagccactgcagggatcacaggagaaaacctagaaaggtcatagaagggctaaaaggcatataaaggatgaataggagtccggtatctgggtaaggggatatgaggggactcaaattttaggtggaggaaaaaatgtgcacattcacaatacaatgtagcatttaccaagctgtttgtatgtaccaggtgttatttaaagcacctcacacgtagtcattcactaatcttcaccatgaaacaaaacgctttccttctttcagctccatttgtatgtagagaacagagatgcagagaaatcaaatcacctgccccaagttccatggctggaaaatggcagagtccactcctcaactctattctggtctagctttctataatgctgatggtgaatctcccattgttcactgaacacctcctatgtgccagacactggaaggagctgggattacagtgctcaaataattacagtcttggcccccaaggacattaatacataagcaaatagtcaagttcaatcctatgccataggtttgcagttgaggcaagcccaagaattatgagaatacagggagacccttcactcagccagagccatggggtatgaagccagaggtgggtgagagttgaagcatagaattaagttaggtggagaaagcaattgtggctttccaggcagaaggaagagtagtggtccagggcgagaaaaattgccacttgattgggggccaaaggagttcagcctggctgggcactctgagataagagagcccccaaaacccctatgaaaaattgtggcatccctctgggatggcactgaaaggtgtcaacaggaggctaatgtcagatcttctagaatattccctagggctaccttagggaaggggcatggtgcggagagggttggcatctgggagtccaggtggtttggaagtggggtgcagcaaggggctggggctcagggacatgagagaattagtgaaatagaggctccactttgaaagagacagtgtcaggacagaggtgacttcatcctctgggtggtggagagcgatggatgaatgaagtcatatgcaggattggatgaatggatgtgtggactagtcgtgttggaaagaaagaacagaagtggggagacagcctctccgtatccatatttcccaggataacagcctggagaggtgacagccctctaagctgagttgattgacttaataatctgaattctgcccatgtgggcaacgctgcatctgtgagaaaagttgagtggagagtgagaggaaagatggctctaactgtccaacagtgtgcttggtgctgcagcaatggcccatgtcctcatggaatgcacgcctggaggaatgatgggagcagagattccgtattgaaacccaaggcagagttggggatagctgctgctttgaatgcttttaggtcttctcatgtccagtgtgtctcctccatataggtttaagcctctagccagaggtttctctccttttgattctttggttttctctgtacagttcaaatgacagttacacataataaaatatacatgtgtctcatgtattataatatttggatgtactgtcaattctatgcgatctgaggactcattcacctgtgtccctgtgctgtatgtctgacctgtgttcagttaagtgtttacaggaaggaagaggggaatggaaagaggaagagagtgggcagagaggaaaggagaaaaggaacagagagcagcaaaaataagtataaaaaaaagaataaattgggaaaataaatatatggataaatgaatgaatggatgatggatggatggtggatggatgggtggatgggtggatggatgggtgggtggatataaggatagatgaatagtgattacatagatgagtaaatgaaggaacacatatctgggaaactgaaaaagatcaaattctgaggtttgtaaaccaggggatggcagggcctgccctacgtctatgctctgggggtctcagagaacttggcgctagtttgaggacccaggcttagagctcctgactgaatttatgaatatttttatgagttt

SEQ ID NO: 25 - DNA, 10820 bp, Homo sapiens
  source 1..10820: mol_type = genomic DNA
atccagaaattctgacaaaatgtcagtattccaaagctccgcaaagactgcctatataattattatccctattgtttcataggcatttggcttgatagaaagctaatgatgggcacttaaaagtcaaatgacatgagtaattaagaatagccatgaaggatgatgtaaaggcatctccatttcagactggggaaagcacagaaatgttctacctagtggatcatagataaaaatagctgccatctgctggtgccaggtacttttcatgatctctcattgaatactcacagccaatttatgagatggtgctatattattcctatgtaagacatgaggaatccaaggcaaggagaggttttgccacttgtcaaaggttacagtcactggggctggaactcagtcccacattgatctgatttcaaagcctcatctcttaaccacttaaccagtgctgagatgagactgtctcagagtgtggattacccactcaaatttatcatcagtaaaataataacgaagaggatgacttaaataaggacaggcatttgcttcaaagtggcggtatggaacccgatttcacagtggctgaggtcagcctcacatggatgactcacacacatggctgatctacataaatagcatgtgctaagattaccatctggtataacaatctacatcacaagactccacatttcctgttacccaggctaaataggtaaatcttatctctggcagaaagagctatatattagtcagtaacctggaggatattacttttgtcttccaagttatttacatccagttggcacggtatccgagtcttcaaaaggccatcccactgttctaccatgccgactgctttatagtgattgggaacatagaaagaccactaaatccatgagtatgagccccacgttgcacttaatttgctgtaaactgaattgctttgtaatgtgtagtgatagcagcattctgtgagttcatagatggtagtattggcactacaggaagttaaggcaaatctaaatctagagtaaatgtctgttctagtaacaacaaagtgctggtgctgtctgtaacctgatggaattgatccaaaatcagcctccaaccaggtgccttcttaaacccaccaggtagtggtgcccaagtgttgttcttgctgttggtaggacagacccttagagtggctaagccaagtcagccttgcccatgcataacttctaccctttcttttatgcctactttcttcatagagccccttgggcaaggatgggggtagctggagagagaaactgactgatgtccataagacaggttatctcatacacctgatcattagatcctcctctgcttagactgctctttggtgcaaattcaaagaggtccatcgacaaatcttttccccagacctctttttcactaattttctaatagggctttccctaaccttccagatatactcatagatacagtcaatgagtcactgtacatccatactttttccctttctcctttcaagcaaaatgagcaactaggtgcacttgttgaagttctgcctgctggtaggttttttccttagagtggggctatagggatatagctatctacttttgggcattgaccacacatcatcaagagcgatctataaaccaggctgaaatcgtttcttctttagtcaacatctgtgcctttgcatgtgcaatttacctttgccttcaatacctgcttcaagctgatctcatttataccattttcctttaataatggagcacttctatcgcacacactttatgagaaaaatatgtgtgtcttaacccataatcttaactattgtgctcacttctgtcataacaaacaggaactagcatatttaacatagaagaaaaggtcaggtgaccatcattcttacatcactgtagcatttatcgtgttagcacttctaattacgagtataaagcggaattaaaggaagagctatgacttaagcagcttctgagaaaataacaatagaggaagtctgatttacactcttggagataatgacctgtgactggagaaaagatctcccagattatatataatttaaattctaagtatcttaagtaaataagtgtttcacaattttttttctgttaatgacacattcccatagtaaaagaataaaggatttggtctcttcttgaccttttatttcttcaggctttctaaaataaaactttatacagatgatggtattgctcaaaaataacatttattcatcaccaacctcaaaacattctgcatcatattatgacacttataaacataagcttggttcaggcaggtttgacccctgaacttataaataaaaggagagagaagaaattccttaacaaaacaaaacaaaacaaaacatgttgcaggaaacagagtgcaaagacctggaaaaagcacagctgtaatgtccatgttgctacttcacccatcatggaccaaaggaaggctccctataagcagctatcccttgaattttccttctagtcttctgggtgacttttacactctgggttcatgatggtggctgtgacctgcaaatcttggctctattccctaagcttctagtatcttgggatttctctagtgacataaatgagaaatgaagataaaaatgcagtcactgtcaagggtataaaaaaaaggaagtgataacaagcataatccaagaattgcagttactgtcaaatgcaagagaatctaagaattgcagttaagcgagggcatgcttagctttggaatggatcatcaaatttagatagccttctaatataaataagtctgagccctggggatacccaccattatagttctgagaggccagcctgcatttcacatttttcttcagttgaggtacaaagtggacaatccacataaacaattgtatatgttgagtgttatgccagtggggaaaagggaagaagagatgaagcaaggaggcacatttcacatgtagccaggcgtacagggcctccaatcagatccgtcattctatctcccacactttagatgatgcttacaatagtcacgggtcacaacacgaaatcctttattcccatggcaaatagcatttgaaaatgaagctccgtgcaattaaggtttgttgggaattttttcccttttcacatatggcaatatttatcttcaaagtcttctcttgggctcacagtctcctttataaagcagctggtcttgtgtggcctaattgtgagatgatttgtgtgattcagtagcttatttgtacagcggttcggtggaatttataaactgcataatattgttctagactgttttccttttagtaagaagccacattttctccagcattcatgactgggattataattagtggagcactcaagatattttcatcccaacttcctctgttaattaatgacattaaacaagaaactcttgcaattaaatgaaaagatgtgagcagaacatggaaagagaactagagtgatgaatcaagttgaactcaatctttgttttgcatatgagagcttgtaacaaatgctcacatgtaactaatacaagctgtaagttatttctaattagtcctagctgttctcaggaaaaccgaatcaaatatggagtgttttagaaaagatctcttttgctatttctgtccccgagaaaccctcagcaaatggtcccagttacttgtcacacgttcttcaatgatgaaatgtctccagtgctgccaggcctataaaaagttctaaaagcaatttgacctttcgtttcctttattaaaaagaagctgcatgtgggcattttggtttttttccaacagaatgggggttaagtaatagcagagaacgaaaaaggagaaaattctacctgctaggctaggctaaggaaatgcctgtggtaactataaaatttagctctgaggcagccaagataatctatgcattttattcttataatctcatttaagaaagtttcctgtgacatcaggaaagaattttattaacagttgactataagagaaattattatgtggtattttatgtgtggaaccataaggaacatatagagatcatattttaaattttacccaaaacagagatgtttctctggcaaccatttctcatcttctctcaatgctgtccaagtgcataatgatttgcttttaaaattgaatatatttgtaaaaacaaacttaagtcacccccacatacgttttgcaagttcagctcaggtggtccatgttcatgttgtaccacgaagatcaaccacatttagccacactgcacagcatccaacacaccaccacacctagacttgtggataggcagaagacatagtaagacagtagagaagttgaggaatttgaaggcttcatcacaactccaattggttcccttggtaaagttttcctttgttgttttctttcagggcttgaccaataactcatggaatgtgtcagctctaattgccggtctggattggcacaagtcatgcctcaaaattggaatgttgtcacatataacatagtcctgcagggggataaatgtggaaaatgttttatccaaacattccttaaaaacaactgtgttctgccatgcaagagacagacaacctttaagaaccagctgagtgaactcccaggatgccaaagtttcaacaggtgtcaggacaccactcttccttcctccaaggacatcagacaatggcaattcccctctggggtgctcttagccagggcagctttccaaaagaaggtaattggtgagttgtgagtatagaagagggatctatgaacctgggctggctttgtgagtgcctcctaattgatgagcaacttacacaagtgttgcctgatggccaggtaatagctgtcaagcaagcatcattggaagggtttgaagaaagcatagaagctatctgtagggggagatttgccactaagaaagatgtcatgaggtaagcatataagaagaagaatggacacggacatctgtggagcacctattgtgtgctaggcactatgaattttatctcaagtacttacattctcattcaacgtctgttttcttcagaaataaactgtggcaatctggtcctggcaaccaattagtagtaaatactgggctacgcagacaaatcagttctttgcttattcattctcctttcctctgcaccaggcagacaggtgctggggaggaaaacagatgcttgaaccaaacactacaacatagtagggaaaacatcatttcaaatgtagtaagctcagataatgttcctcttacctcagaaacattcctcattaaaggtgcatatgcagataccaaaacgttgttgaacatgtaaaaataataattattattttggctacctaaaactaatcaactcattaatttaatttttggagcagaattgaattgctcttgagactgagttggctgtagcaaagccaaaaccattagttggggaagtaaaatcaaaagctgccaccagggaagggctctctacaagagaaacattcaaaaaagttcccatagaaagctcaagcccacaggaaatgtctagttgagatgatggatcacagactgccccaatcgccagacttccatggcaaccttttgcaggcaccatcatgtgaaggaatcctttttgtcctaggatacgtcttttttgggaaatgacaaaccgtgatcttctaaacatttcatgtttgatatttgacattttcccaaggcctccaaccctgaccttgccatgcaatagaatggggagaaagatgcttctttagagatgggcaacattgcttttagagatctgggcagatatacaaccatctcttaacagatggagttgatatgattgagattcctggagaagtgagtatgagtctgttaccagaccacatcatagatcctcatcttctgatagtagttcaaatatttgcagttacatgaaaatatctttcattgaaatggcctcatttttgcgacacttggttacaaatgctagcattgtactgcatcagatttacctggaatatatttctcctttactataaaattccctttctattcccccaaatggtctcagtaggtttgggatagactgaaaatctgtaattttagaagctgtcaaggtgactgttgtgtgccctggtttgggatcagtgagtacagggggaagtgcccatccaagaaatgtgcgaggtttgctctaagtctacaactctagactttttcagaagaccgggttacagagtgatctaattgactcaaatttttccccattactggcagaaatatctggcctttagcaggttggtaaagcccatttttcattcagtttattattccttgaatttccccagaaataactattcactctcttctgcagttcagagttaaattgttataggtaaattgtgtcctctggagtcctgacccctagtacctcagaatatgaccttatttggaaatagggtcagaagcacagactgggaacactcactgatttcctgggaatggggtctgacaatcccaaaaacccagcacaacagggtggatacagaacaaggagcatttctacaagcacttcttttaaagactcctgttgaaaacaatggcacagtgagactcctcactgaatcttttgcttgaagttctcagttgaccaatttgttttgtttttctctctacacttgctgtaatggtatatacaggacatcaaactcgactaaatgtgtggcacacatagtcagtgctcagggtcccaactccctttctggctcaactccatttgtccctcctgcctttctacttctatcttgccctgaggtatttcacttttccaggtcctggatagagattaaataaatcaaaatccattctagcattcggttgaaattgtttcctgtgctgccccattgatgattttaaaactatatatatatatatatatatatatatatatatatataatatgaatgtatgtatatgcttgtggaaaaaatcctacaagaataattcctcatggataactctcagagctgtaacaaaggggtaaattaagggggttgctggacttccttggggctccttggtgcctctcccaccagtttctgcctaaatttccctttcagaaatttgaatggagtcagtgatttcttggagggccaggttatgggcaaggggaagatgtgcacacgtggtgatgtgaaatttgatacacaactgccacttgatttaacattctcccagttttcggtagagtttaattttggttcaaatgaagaaattagtgttacgcccatgcttgaggacaagccaaactgcatgacttcacatcgcttttagagcagtattgaattacataacttcttagcttgtttgctcccaacagcctgagcgggattttcagaattgagatttgggcctgaggagagggtttccaactccagggctggccagacctcggtgatgtcacctgaggaacacatgtgtgttctttcagttgggttggcatctataggagagaaaatcccatgttggagggaaagcctaagagattatattgggcctcttaccactaatttttttttctgtttaattatgtctgttctcttaggctgaatgattttcctagttctgatcacacagcagtctctctcagtacattctgttccctttacaaggtctctgcccaattacaggaacacactcatctcttctccaaatgaccattacgtgctgaaataaagaaactagctacaaagtatttcagggaaataaagactcaatcagggaaaaacctgaagcaactctttcaaaaaaaggcaattaaaaagcattacctttatatattgatacaattacctcatatttacattaattatttctcgaatctcatcagatctagtcaggaagacaaagcatgtccaaccatagaaggcttattagcaaaaggagcacagctcagcttattccctagaaaagtaatgccatctcaaggctagtgactcaaggaacagtgagtttttggcttttttattctcccctaaaaatggtagaaaatgttgtcacaggtacattaatgaagattttaagggcagaaatcatttcatacaatagcccaacttgcttaggggaggttgtgctgttccacttctttgcttgtacgtaaccatcagtttcacaagggctttgccagtggggctgtattttcattaccacatttttcagtcagcatcaccttttatgttgaactctacagaatgtgaatattcaacaaattctgacctacaaaaactgcactttcataagatatgacctaatattgttcaatcatatgtaaattacaaaaacaaaggctggtaatatagcttatgggaataccatggtttgtaaacagagctgctttcaaattatttgccagattttctggtaactgtattaaataagtgaaaaaaaacaggtaaaattaataatatactttatttaatcccatatattcaaaatagtatatgtaatcaatataaaaatcattaataagacattttacattttacatttttttgtactaagtcttcaaaatctggtgtatattttacacatctggcatatctcaattcacattagccatgttttgattgctcaatggccacattggacaatgaagtcctggacccttcacttacaagctgagtggcaagtaattagcattctgagtctgctcacttccctttaaaatggagttatattcttttaaaaacctacatcctaggatatgagattaaataaatcaaaagagaatactgtagttagcattatcatgttagggaggtcattcaactagccaattttcattgcgtaaccaaaaggttaacatatagagttatagaatattctcaggaattactgatgtgagataagggtctggaggttttatacatttagtatcaagatggagaaatatgagcccaggttatagtctggagagttgctgacaaatttgccaaagttggaaggctgttaggggctcttttcatctctctgattctataataccttctattcttactcatcaattaaatataatgtgggttgggtgatagctctgggactagccaggctgcacagaagcatgaactaatgttagagtgacaaacttcctctctctaagatcaaacaagatgagactgcagaaaagggaaccattttctcttctatctcaatactcgaattgatggtttgtttctatggtgtttagacaacacaacaggcacacactagttctttcactagacaaagcaagacaaccagattgagtcacaattactttttttaatcctatttattaagagattggcaactcgtttgaggagtctggtcttgtaggtagcagccaaaaaaattggaggaaatagggtgggtttttttgtttgtggtgcagtggtaccagtgaaactgacaggagtagctgatcccaaacaacaaccccagaaagcacgtggctcttgttatatctggctggctcagcaactatccaagttatggggctacaggggatactgtctagaccagttcttttggccctggcgttggcagaacatagaaccagaaagccaaagcatggtgcaaacatctctctgcattttttctggtcggcagtcaagctggatggaaactaaatctcaaacaattaccaaatagcaataaataagggaataaaaagacggcacagggtttcttctatccttgatggatatggcctgactcttgtgctttaaagcatgacttctgaagtgactgtccttagggagaaagcacagagggcaatcggaagtatgacttttccagcttgagatagtcttattgcatcacaggtgttctagactcgatgatggtactgataggaaaaagacatccacatatcatcgaaataatttatttaccactagagcaccacaaaaacagacatacatcgtgttaaaatacagcgtaattggtcatcaaaatacaaaacagctaatcttatattccattttttaaccatgccaacgatcaaattgtactgctgattaacacaaaaaaattgctgcccacttgcatactagcacttcaccccttccttgccacctccactcccatggcaatatttacttatgggaaaaaagacctacagaacccccaaattaaaaaaaaaaaagattcaagagatcttaaaatagagatcagtgcattcatgaggaaagacaaataatacaaaacaaaatgtcatcctatctgagaggaaaatgtctgcagaaataaaagtgatttacacataatagaaaagtggaagacaaaaaaataatcaacacacactcaaatctgggattgggttacatccaacacaagggctgtttactagcatcccatctcttgctgttttcaaacttgcaaatccaattctttaattcaggaaattccaaaaaagaagtagaccatatcaaacaacttcactttggattcccgcctccttatgctgctctttaggtgaaactgcaaatattccattgctttgcctattaatttttattttaaagctccagcatgaggatacctcttctgcaatgatggcaaattatttaaagcaaggtaaactttagcctcagatatagataactctcactcagaggaaagaaagaattttttgacataggaaaaattggcttgtgccttttccccttcaaagaacatttataaaaaccttataacttcagtgaaatacacaaaatgacttatgctgacctggattaagaaactgaaggg

SEQ ID NO: 26 - DNA, 1764 bp, Homo sapiens
  source 1..1764: mol_type = genomic DNA
aaagccaacctaatgcagctcagtgctagacatggtagatgggtgcttacccaggagccattcaataccctctcttcccttactttttctactttagaagttcaaaagcaaaaccttttttcctattgtccctcacacctggagatgacaaggcaacataatattgtccaaacagatgtcaataggcctgggtgggacttcaggaaagctcatatttgttaaaaaaaggctcagcttttgcccctctcttccacctttttccttagagagtaaacttgaagcctgaaattgcagcagagtgggcaaacagaaggacaaaggctacaaggtaaggtaaatggagcagatggaaagaactgcagttcttgccagtattattgagtcactacagcgagaaaaataaaacgataattaatacaaaaagtagattctacattatgaacccagaaaaaagctctgctctggatctgactctgttacttactacctgagtgacattgatcatgtcatagagttctattccctcctctgtcaagagaacacttgacttctttagcctagattatcctatgaaataatgcagttatatatcttcatacattggaagatgttgcttatttaacatgccaggtggttgatacataggacacgcagtgactccacagttaaatataacagtgcccaggagtctgctttccgttcaaagaatttgcagacacttactttgactggcttaatttcagacttcttgggaattctgttctgaagttaggaacttccaagcttttaaaagattacaaatattcaatccttggcaatttaatatttgtcttcctttgtctatacgaggggaatctttaaagacttgttgaggcatatggcaagttttgaaagaatttgaaggtgaataagtaatgaaatatattccctcttttaacattaaaaacaattgtatttaaccaataaggaaaaattcaggtaaaaaaattccatgcatatttttcttttttaatttaatttctactaatattatgactcagaggctgaatccaggataaaggtactatcatcagggctctctttcagggcagaaagagaacatataaagactagaaatgaaaaaaagagattaaacaaggcttccacttcatctctggattctatccttagcatagtgttttcaaagttcattcatttgtatcatgcatcagtacttcattcctctttgtggtggaaatgttgagttatatggaaactctatgtttaatcatttgagaaactgtcagattattttccaaagcagctgcaccattttatactccatcagcagtgtaggagggttcaatttctccacatactcaccaacactttgttatctttttgatacttgctatcctagtgcctttgaagtggcatctattgtggttttgatttgcatttctttaatgactaataatgttaagtagctttttatggctcattggcctgtacgtctttcttaggaagtggctattaagatccttagctgatttttaagttatttgtcttttatgattgagtcatgtctttatatagtcttgatacaagacccttatcagatatgtgatttacaaatacttttttccactctgtggtttttcactttcctgatgctggtctttgaagaacaaaagtttttcaattttggtgaaatccaatttattatttatttcttttgctcatgcttatataattatatccaacagattatttgccaaagctaag

SEQ ID NO: 27 - DNA, 12761 bp, Homo sapiens
  source 1..12761: mol_type = genomic DNA
gtcccggcggagaaggcgcggagggcgcgggcgctgctcgggggcgctctcgctcgcggtctgcccaggcgcccggagcccgccttttatagggccggcggaggcgccgcgtgttgcagtgacgtggctctgtctgcgcgttccagaattctcaaacatctcaggaatgctggttggcgcgggctgctgccaagcgcctcccctggctccattgcaccttttgtgtctgtctcccagacccgtcaagaaaagaaaacagttcgttttttccaccttaaattacgacttatgttgggaagggttctttggggcgtgatggtggtgatggtagttggagggtcgtgaggggcgaggggggccagttcagaactttgcctggacgtgggaggacctgagagggaggaacaaaagtcgttttgtttggtgatgctcacccgggaaggcagtgagctgtttgcttttttacagcgaaggtgagaggcaagttatctgttgagagtagaacggggaagaggagtgtgtctctgagtcaggaatatttgtggacttcaaagaggcgtgttcggaacaagtcccaaagctttggcatgatgtttattagcaaagttcagtgaattgcaaattaatacccacagtggcatttatgaatggggggggggggggaaacttgcccatcgtctggcaaattcccaggcagaggtctcccccactcccccttccctcttttgggtatgttgtacttgtttgtttttggaggttcccactttagttccagcccactgcctgggttggaagtccgagaggaaacgaacgcggagttctccgggaggctgacttatacttcctcacggatgcaggagacggggtaagcccttcccgcctccctccgcgggcaggaggctatgctaaccaagcagccagccagaagtgcaggagaccaccgtggagtggcctgaaatcatcacctgcaaagaccgcaaggccaaaccagattccaggaccattgtgccctatgaatctctgggaaaagcagactccttcaagctggacattttgtggggtttttaactcctttcctagggaccactggaagcctcccattttaaatgaattgcaatgcctctctccaagtacagaaaagagatttaggatgtaatattgtaaaaatgtcagcagattttaagatcccgttctacttgcaacagtagaagaccccaccacaagacaatttacttgaaaccattagtgtgattgataaactaggataactacttatcctttttaatgtttaaggctattcagacttcataatttagcagtcttgcttgaaaaggcccttagtgctaatgctgactttcctgataaggagaatctgtcaggcacactccagtgaagagaattcttgcaggaaggagttcagttcattcattccaaagtgccctttcataaatggaagcagcatttgctagagggagtgggatctctaaagctctttaggggtggggacttttcaagacaatggattgtcaggtgttatgcaaatacagtgaggaaagtgtgggcccaacacgtacacaggttaatttattaaaacactgtttttcctgccttcatttcattgtttccttcttaatgaagtgacggggggcgggaggggggaggaggggtgcagggacagggttgaactagagagagaatggagaggagggttcctggcttctgttgtggcgtcttttctatttgacttcatggttgtaagtatttccagatggtgaatcagacaccagacgtaacaatatttccatatttgggtatagcagtagcctgcgtttttaacatttttctgcctctgttatccaaccacaaagcattcttgacagcttcaaatgttgttaaatatagatttaacttctcttcccagagcaggaaattctttggaattccttgtttttcacgcaatctgtccatcatgatttaaaataaaagcacagtggatcatccaactggccgtatataccttaattggaggttgggggggggacggcagagatccagtctgccgcactgcgttcaaacacacgccattccagagattcctttaaaatcacattaaagttttttaacaagggtgtgtgggtttgtttctggacttcaactggggaatcttgaggatgagtttgccccagaagagaaatttagagaaccttaccgtcagctgcccatttaaagcagggggtgtgttgtgggagggggtgggaagctggagcaacagggccaggaggtgtgggagcgggagacactagagtaacctatgtgcacagcctctccatatagcctgctagtaatcaagagaaacagcggctcctcaagtcctgcccaaagaccgtccagaaaccccagcctcccgtcgccttctcgccgcctccgctgggagccgcagatcagtcgttgacggacaggaggcgaaatgtgcaaatgttatgggtaagtgttgcttcgggtgctaattgtatggctcttctccttcaccctccccatacccaggaggcttgtttcctttggattgcaatccacggagaaggggctacttgtcttctgcagctcctggcttgggctgagagatggggtctcaaccgctgctgccagggaattttaggttacctaatgaagggctcactgtctggcggcaaccgagaataataacggtaaatctggcgactccagtttcctagcccgaaggctctcagactagcgtgcatccgagtcgcctggagggctagttaatgaatggttctctgggtcccaccccagagtttctgattagttacattttaacaagtccccaggtgatgttgatgctgctggtccggagatcacacactgccctggacgaccggagtgctaaggtggcgcccccgtaccatcccatttctctcctctccatttccccgcccccatggcttgacagccaggagagccccaggaagccaggatcctggtggctgcggcaggaactgggtggccgacgcgcgggtcgggagggagcggaggcaccgggtgcgggacgcgaggcccggacctgctaccgccgccccctgccggccccccgcccgcgcgccacagtcccgcggttgccaagaatcgaggtttccggcggcgcgcggcgcaggacgtttccagccccggcccatatttggtgaaagtctttcaagactccgtcatctagcttcgggggggcggcggcctgcgacccggcctggcaggcttgcggccgccgcgttcctgaaaagtgcgccgcggcggcccgcgcccaggcctccctccgcgcgggaccccggagccggcggggtcccgagtttggccccagtcctcacccctctgagggccgaagtccgccctgcgctgcgtgggaggctggcgcggagggcgccgcggcagttgtgtcacttttcccgtccgcccccctgttgactagtcctcccagcggggcagcgagttgccgtgctgtgccagacgtagagggtgcgtccggcgcaagggtgtggggctgtgtgtgggaggatggcgtttgcgtggacaccaagcgcacccgagagcgggacgtgtagccagctctggggaaaagccccgaagggcctggattccattatccccagaagggacacatggggctctcggtttctcctggtaacaccattttctatctaaatgaagaaatggaggcatggaaggtaatgactggtcactcacctagcctggatttgggcccagatcggtctcacacttaactactgtctatactgcctactctctatctcttacctcagacattcaggcccatcctcttccgtaatgtactggctcttttgtatttaggtcttaatgcagatacattagagtggtctttaagcaccttggcaggctggtggggaagatgtttaggacctgacacctcataagccacattgttcaggcaatccctacttctggaataaggcttggcctggtaggagttgaatttgtcattgagataaaggtttcagcatcacttacttgaacccatatatttgaggactgtatttacccttccctgaatcccagatttattcagagcttgtgatgtcatggcatagtgctaggtactggtgacatgagaaagaggattttaatattgaacgtcccccgcaggactgtaaggcatattgcagaagtgggtacatgagttagggggaacgactaatctggggctgaaaggatgaacaggagtttgatagcagggcaggacatttgagaggaagggaacaaggccttgactgggcataacaatcagtctgactggaccataggattccttctgaaaactggcaagagatcagttgggacatggccttgctgtcatggcattggaactttgcagagtggccatgtgagctttttgcctaggtttccttgtctataagataacaggattctcctagctcagaggacacacactcaagtgcctgcaggattcagccaggtaacaccatgccatgactcaggattagtgctggctgatatttggccttggccttgggaaccatggggacatggaaatgagagtgaatggttactaggtgaaaatgtaggttcactgtttgcatattttctcattttaaaaagagatactagagatctgatttttatgtgaagtcttcaaaattttaaaagttggcaattaattacaatgttaccatctagactaatccttggaaaacccaagaagacaatctgtgggtgccatttgtgatctctggactgaatatctctaagaatctgtcttgagcattctataactcccagccacctgtgaaattgtagggaacagcaccggctccatttaattgtcaagttgactagactttatttaagagtcagggctgctacttacctcttggttgtaggaatttataacctattaagaaaattggaaacaaattttctacttcatccactctccagccactactctgtctccctccttccccctgaattgatgatgtaagcagctggcattgccttggcaatatgataggaaacacttttttagggcatccatatcccgtgggaaccttatctgtttattttctccacttggcatctatccacaggaagccagcattcctaactcccttaccttacagaacttcttgttttgtgataacacagaaacattttattggaaaggactggaaggcacctctcccaccacaccctgcaatccatcttctagagatgattgagcaatatgaaatataataattgtaagtcattatcaaatggcttggcacctctgcctttcgtgttttatttctggagatacctgtctcttaaccaaccactaacatcacacgtagcacatccagaatttagaaaggatttgctctgggcacagtagatgagcacacagattctacagagagattgaagtgatttatttgtgacaaatttttgcaagtcttggggattgagtgtaagggagtattcaagcattggacttactatgtgaaagaagacataagacatatttactgctcagcctgatgcctggcacatagtaagtgcttaattacaattattcctactgccaagcaccatcattatctttactttcactgcaataacatagaatgagctagtcttattctagagaatactttctattctttagaactctagttataaaatcactttggtatatggtatatagatgatatgattaaaaggatccatccagccaggtattctggctgcactggacaccagtgactatctaaaaaaccactcaaatgggcttacctctcatggcactccgcagtggtgagccatgtgagttttgctttggtttcctcatctatgagatgataggattgcaccagctggagtcactcaagtgccccaggaatcagagaggtaaaaaatgccatgcctgagcgttagtgccggctgacacttggccttagcaataggaatagttggaagcatctatggggcctgggctttgcctcccattggctaaatggttagtatacacaatctcatcagactttataaacaactctgtaggatattttattaaaagatgagaaaatgaagctttggatgtcagtaattggtgcaagggggcagggttgttggtagagcagaattttcaaagaccattcctttcggggagagcatggtatttgtccttcatgaaatctgccttaaagttcaggaagttgcctgcaacttcttgactccccttaacaactgaatctgtcacttaaatattgttctaaatgtgtctttagcttgtgttttcagtaactgtggcccacagtttcttaatgagagtctgataataaacaagttaaaaaagggttctctccttagggcccttgcctgagcttgtttctatttcctctgggaggaaaaaagaaagagatgtgtcagaacagaggtggaacttctcagagattgagagatccgtctactgtaacttagtcatcaccatcaattttgttacctgaggcaggcagagctgctacctcccacagcctgtggttatcctcacaacagttactaagcacggagccctccaggcccctgccaagcgcaccctgcatgtgccttaggtgtccctgagcattgaatgggtgggggtgcccaaggttatagcaataaagaaaaaacatagtgccaggtgcttctccatttaccccttcttttgtagttaggttttggctttcaaaagacaaaaaaaaaaaaaaaaaaaaaaaaagaaaaggacaaaggattgtatgcttaaaatttccaaggctctggaaaattgggagaattagaaagtgtagacatatgctaaagcattgcagacccagttcagttcattctaataaacataacatgtatggcatgctgcatgctaggcagcaatgcaaagaggaataatattggactttgcttcatggctctagctgcctaggggagatgcagatgcttaaacagataatacaatactgtggattaaagctctagaagccgtgcagctttgggagtacagacaagggatgtccaactctgagtctaccctttaaagatgtgaggtcttgttagtgtgtacatttttatggatgagagggattgggcacagttattcctaacatacacattccctttgaagctgggaactgcaaggaatgtgcacattcctgagagattagaaaacatctaaaagtgaaactttcaggtgagcctcttaaaatgcattgattacagaggaccgaccctttggattgaaaattcttatcattatggatccttcttcagaaggggaaagagaagtgacatttactgggccaaacgccttacaggttcatgttcaggagagccctctgagatgagctttttgacatctttctaagcttagggaatgaaggctcatgatgctatcttaaaatagcttcttacctaagaataaaaatggtagtcaattctggcttcattatttggagggattagaagcaactctgtcaaacagggtgacccttgatgccatttgcaaattcttcttagaaagaaactgcaaaaggaatcattcacctactaatccaacatccaataaagtttagttccgtgagctagtatcatcaatggcagcaaaattagaacagtagccatagaagtctaagttgaccttcctttgggtgacaagagtttagtaatgcccatggattcattgtggcaggcagttctctggtctccatcattacacctaacattttaattgtgtctttaattttaattttaaacgtgactttaattttaaatgtgactttctcagtaagaaccgtgagagagtacttaacacaatataatacactagattcttaatatatgtaagagatcacagtaggatatgagggagaagagaacacatgagaaatgaggcccagagacatttagcatcatgtctgtcagctagttagtgatagggtagaactaaagtagacctaatagttctcaagtagtttttttacaacatctgtattttttatctctttggatctagtcctctagccatagtatagtaatttttataaattatagttaacactcttatagtacttgctattttcctggtagtttttaaatgctttcaatatattaattcatttgatcctgaggtaggcactattttccctacattacaggaaactacaggtatgaagtaacatgctcaagtcatgtaaagctaaggttggaacccagatagtcgtctatatgtaaaccactaggatacactccctgaaatggaatgacattatctttttgttaaagtcgtatgaggaaaagtaaacaaatatttaatgattagaataacataatatagattttattacacaaagaacatatttttaaaaatttcagttaattttgtgttaaagatgctcattaatttgtttacaaaaaatgtagaatcaataggaaaaaaaaccaatgtttatacattaatattaattagagacacattaaagattctcattagataacatacactttttcaaaatcagtttcttataagcaataatcagtcagtcagaattgatacttagtttctccaaaggatttaagccttattctttttttattttctatatacttccaatgttaaaatcattctaatgctacttatttttttaattgattgctctggggtttgtaaacttactagtctctcttcaaatagtattagactacttcatatataatgtaagaaccttacaataaccccactttgtgctattgcatcatatatcgtacttctatatatgttacaaacgccatacttcattgtaaatatttttgcttcaattatcttttaaagaatatttcatatttactcacatgtttatcatacattcattattcctttgtatagatccaaatttccatatgatttttcttttactagaggaacttctttgaacatttcttatagttcaggtctgccaacagtgttctgtttgcttttatttttattttgccttcatttaggcagaatattttttctgggttcggaattgtagattgacaccttcccctgagcatggatgttgttccattgttttttgcttacatatttctgacaaaagctgtttatttcatctcttgtaggtaattatttctctctggctgcttttggtattttctccttggttttcaatgatttgtcttggtgtcattcattttttatcctgctgggatttaattagattcttggatctgtgggtttattctttttatcaaattgggaaatttttcaccataatttatactcgttcgacaagtttgcatgtgctgcttgcttattccacaagacaaaaagctctgttcatttcagtctttttctgtgtgcttcattttgggtatctaatttgctatgccctcatgttcactggtcttttttctgcagagtcatatctgcttctacactcatccagtgaaatctttattttagatgttatactcttcatccctagaagttctatttgattctttaaaaaaagattttcacttctcatcacattatgttttactttaaatactagtacatagctataatagttgttttagtattaccttctaatttcattatctccatcatttctaagtttgcttctattgatcgatttttttcctcttttggatcacattttcttgcttcttggtatgtccagtatttttaactgaaagctgagtattatggctattatgttgttgagttggggatttgttgtcattctttaaatagtattgagctttgtcttggggagtaaattacatgtagatcagtttgacccattcgaggcctattttaatatatttctagggcaggtctagaataactttcactcaagagatagttcagtcctactactaagatgtgactctggatgacactgaggtctctactagtcaaagaaaagtcatgcaatttcaaaagaatggtcagagatacttcactgtgaaaatggtcttgcacattaatactttcttatacctttcattactgagtcaggggtatgaattcttttaataatgatatataatgtcaaactgctttccagtttatacttccactaccagtttagaaaagtgcctgcttcataataaccttgttagtattgaatttaataaattatagtatatcctttgggaggaattatgcagcattaagagcatgatttttgagacttgagaaaaggctctcaaaccaattaattaaacaaaagcagagcaaaaaattctatgtacatcaagatcccattttgacataatgtattctttggtaatagcatttatattatgaatgattttcatattcttattttattttattttagtataacttcaaaagtgtctgttgttgaattttggctcctgtccaactttgaacaagatatctggcctttctacacttcagtattctcatcgaggatggcacaagcacatatcttataacattgttgtaagcattcaattatttaattgatgcagaactcacagaatagtgcatgtcacatagccatacactaagtgctcaataaagattagctgttactgttatatcaaattatctcatctgtgtagctatttaacactgtacttaacacggagatatagtggtaaatgcaaaagacagttcctgtcttcactgaagagagattttcccctgaatctagatgttggtcttatttctggtacttttattctactttaaaaaattctgtatagaaatacttttctaagggatactgaacagtcaaagcatgtttgatctctctgactttccctcattgaaagcattgattcttgttatacgtggttggtagatttagggagatgtaaactctgaaagaaacttgaaataataaatcctatatctcctttatgattactcctttttttctaccccttaaacaacacacacacacacacctttagctgtgcggacacattatctaattccacgtgcgcaaattctgcatctagataatgttgtcttgtggtatctacctcattaagcagtccttctagaatgctttgatgactaaaaatacagatgacgtgtccttcgtacaccgctttctcacccgcatgtaggaacatgtgctttgggtacattttcaagcaaggacttgtggtgatctacctcatacattaatcctagctgtagtaatactttgggaaaggaatatgatcccaaaataaccaccaggagaagagtgtcatgaaaggtcatggcaatggggcagaggatgttggacaggagtgttctaaaaatagagagtaagagatcaaagaaccagcagtaatgaggaacttttttggctttggttcccagggaaggaggaggaggaggcggcggcggtggcactccccagcctgttcgtccgccactcatatctgtgttgcagaagtagggaggggttagcacattttcctgtcctgggtgctgactgcctttaaattatttatttatataatgcttaaaaacatgtcaaaataagaagcatggagattttaccttactgccatattgctactattgcaaggaagcctaaacattttggatcaagaatcctcaatttcagctttctggaccagacctttttttaccagttcaaattgactttttgtcatcttcctaaactatacctgatgattaagagtgtttgcagagccaagcactgagttttgtgcattcatgagaagttgggtcgatcagccactctgaggtgtgggcatatctggccgagtgaagaaatatatcttagatatgctgttcaatccctcccctgaaatatagctttgacaataatgaggcgagtagatgaggacatgttaaaatcacaggcctcttgaaggaatttttggcagatgattgtgtgtatgtgtttgtgttcatgtatgcacacaatatagtaattccatacagtagggaattaaaaaatctgggatgaatcccgtaaccttcctaaggacaatttactcaatagtgaactaaacttattgacacttggctgacagacgataaatgtatggaagtgcttccaactatacaatgctgtacaaatgttccttaaatcctgtggttgtcagagaaccagtctatatgttcaagtcgaatccactatctcctattcaaggaggtttctttctttttgttcattccaaaacttttactgtgccactccagtgtgcaaggcactatgccaggcaccaagaagcatttaaagatgaatcatacccagatcctgcctcaaagaacttaggatataaggaggcagataaactactgaaaaatgctctgcttggtctctaatttagcttcaaatgatgtcataactgagcataatatgacatttaaaaagggactgcacttcttaaatatttaaataacaatgtacatgttaattagtatgcatttgcctgttaatagacttattaacatgtatgctagtagtcaagagtactgagaaatgcctgtgcaggcaaaggtgttcaggacaggcaatgtgaggtactgtagtgattagcaatgactgacagtgcactaatcacagcccttgttgagcaatcagtcttcattaggctccccaaaccaaaactgacccaaaaacaattgattcttctggctttaaagccaatttctccatttgttttgatttgaggctttt

SEQ ID NO: 28 - DNA, 10907 bp, Homo sapiens
  source 1..10907: mol_type = genomic DNA
gaccaaagggcaggtgcattttgagtgcgccaagctgctggtttgacggcagcgtacaggcaggaagagagagggagcccttgaaatggaggaagggccatggggcagggtgagaaggggcagaggggactaacccatctctgtttttatagctgtctagagtttgttagtgcctatgtctacacagaaatatatgttatttttataatacagtgagatcatatatattaccgcgtacttttctcctgactgcgtcattaggattttccatagggataaatttaaaagtagtaaacttagaaattttcacattgataactttaaaatttaatgcatgctaataaaaatatggatataaattaattttgactgtttttgattttatgaaattattcagtcatttttattatggaacatgcattcttttgctgggcttctctcaccaaattttaggattttgagatttctatgttattgcatgtgcctataatatatgtttattgcaatatagattctattgacagataaatcttttcacatatcaatgattatttgctcagcacaaatttgaagaagtagaattgataggccaaatagagaaacattgatagatatatgtagttactctcccaaaaggatacacttactcttccactaatgtgcaggagtgtgtctaatactcacccacggcctgcagtgagtatgaactttatttttgcacctgtgccaatctgagagacaataaaacatattgttttataactcctcttagaacattttgttagtaatttgcacaaatgcataaaacatttataaatcttttactattaacatttttctttgtctctaaatccacaactgtgtcaccattttaaattaatgcatcataatctataatatggatatgccaaaatttactttctgagaggtcactgtataaaagacattgcttttttttagtgaactcactgtcataatttcccttaatcctcaagaaaatcccatgaactgtggtctctgaatatatgcattttaacaacaaggaaattgaggcacagaaagattggagaataactcgcctaagattaaacagctagtgagatttggaaacaaaatttgaacctagtcaggtctggttttaacagcgataattttaataccctcaaaaactctcttcactaattatcatacttttctctccttcaaaacaaaaggtggtttattcccactgcattttcatagcctcttgtagattgtaattgcccgataaatgcagcttcctttatacttcatttttttatgtcttaagttttatactatgaatttttattggtccaaagaacaattctaatatacttctagattttagtttctacataggacaaacttcttaaactcagcccggtaaagtggaagagtttatgcattgtccagataacagaataattcttgacagtttctccagaattagcagataactggcttactcctcattacatttgtagctctggaaattgacataggataaaaggtaatagaaggattattgtattagggttattaaaaattccagatacacgaaaaagtgtacgtaaaaaccacccacacaattcaactgttaactgtgggttaaaaacagtgtgttcagccaacctctttcaacagtagtcacctaaattgctgtttttaaaagctagtgccttccctgcgaagcccaatagccgtcacatgattaatgtgctagtgtcttattaactgctctgttccagaagctgaccaattcacagccaccctttctgcagagacctttttgtaaaaacctagttgaaatcatggtgttctgactgcaggctactgcatcccccaccaaaacatcataaagagcaaagaattactgtttccagtaatccaggccataaatcaacctgctgtgagcacaggaaaaaaaaaaaaatcacgagcacttcctgcattcatattccaaagcctagcggtaccagcaggttctggaacacgccagccccgtgtgtgtggtgtttgtatgtgtgtgcttgtaagcagacgtgggggagggcaaaagaaatcccagtaacataattccaaatgctttgatcttatcacgaccttccttgctgtaacaatattttaagattgttaaaaattgccaccttatctgtctgggggtatcacccactgcttaacttatctgggcccagttgtttgagagaaatgcagtatggcatgaccttggtttcttagcacctgactttaatatctaatcagttgtgtgtctcggtgaagccatccagacatgtcacacaggcgcctctggcacccagcaattctaatgatgtttacttcattcaccccttttagagagcttccagcagcagactcaaccaccccacccttccaatctgggctccaggtccttccaggcctagagcgtatttttaaatcttggatgccctttccattgaaaataatttggatgccttgaggtcagtcgcaaaaattttctgagatcatatagagacttctgaagagaagcctcaatttggatcaagaaaaacattaaacctcatggctgttgtagttttcaaaacacaaacccttcccctccagcaacgtcaagtcttaaggtcatacaatacaatgtggagaacacacatgcacagcttcgttaattttaaaaaggtttcttagaaaggtcccaaatattttggccccagatcgaagtaaagtcttaaaatctagaaatgagagaaaagatgtcagttattagagtaccatataataaaagtgacataaaaggagttgattttctctctctcagtgacttaggaatcaaatttccttcgttgagaaagcaatagagttcctgccagcctggcgggctcaacctggaacctagctgttttctttctggttctgacgctgtcaccaggaataaatgttctaccatcgcgatgctgctggggcaggagggcaccctggttactctcttttctttattatagcattcattgacatgacctgctacactgtagtgaactcttttggactaattctggagaatgattttctgcaaaataagcagtgatattcttctacacttaagagaccaaccaagaccctgtgttttcttgcctcctccctaataaagctggacactggggccccagatgctctcccggttgagggggctgtgaggacatccctgaaacttattttaattgacctgcttgtccctccccagatcacacagcctgtccagatgtccgaggtagggtatccatgagacgaggcaggtctgtgctgggggcacctgcagtgcttcattggggaaggcgtgggcttcctgggagagagtctcctaacccctgccctggtgagctcgaggtcagactgtggcacccaggggatcagtgcccggcgcccaaggtggcttagtccatccctgtctgtgctccatctgctgtgcaggcagctctgaaacccttcagcaacttctcattggctcccagctgggtgcccctgcgtggttcacaaccaggctccaccgaactggccttcaaagcacactaaacctgcacagctttttattattttaattgacaaaaataattacgtattcacagggttcaatgtgatgttttggtctagtatacaatgtcgagtgattaaatcaaacttattaacatatccaccactcttattgtccatggtgggacacgtgaaatgtactcccttagcaatttagaaacatgcaacgcattattaacgatggtcaggaggtcagccacggatctcaagagctctgagcctgagaatccgcctctgggggaggggggcctgacgagagaggcttacactgaagagcacttggctttgcggcccttgccatacccttgcacttggaatccaggtttgtgtctctctccaacaagaggcctgtgacgctcgccctgcttgttctgttgaatgttactttgcgagcgcgtttacacaactgtcacttgaggtttgtttttagcggaattgtccttctcctgaattcctctcacctcccccacttatctcccggtaaactaaagccaccgagctgggtgggaagacggttcacatgctgacatttttttgtgtttagaggctggtcttcaaaaatcctcacatgtacaacaaggccccagatttgaggaggaaaataactctgcagcccagtcagccgttccagccacctgtcccactcgcttctggtaggtgtgtggctgtgagggcccggttggagctccaggctaaataaagctgcaggaacagagataaacaagtgctcccaactcaaatgcctgcacacaatatcctttatctgggcaaggagagtggccgccccccatagacggacccacaggcgccccccgaattaattgggacatattgttaaagggaaggcccctcagaatagatgccaaactccaaggacacaatgtcaaattccagaaggacagcggcatgggccaatgggaaaaaccccagccgttccgcgctgaatcggatcccatagagcccgcctgcttcacatgctaacgtgatgatgaacgtatccaagtatcagcatcaaatccttgaggactatatttgcaatgtcatgtaaaccgtttcagggagcaaactgtcctgctgtgactttatattaatctctggcttctggctggtcacagggtgtcacatgtactataattagattttggaagtgtgtacagggaggaggtggtgtaattaaggtaacctatcagatgcccaactgcatccttcagaaaggagctgaaacgcccgccctcccagctttcacactgcccttcaagggcagaggcaccagaacctattctccacaccagaatcaatccggtgaggttgttagctcgtggtgcatatccggggaaagccggagggcacttagactttgactttgcatttgtacaaagtcccagatgggggactataagggagttggatgcggggcacttaaatagttccaacaagtgcacgggggctctagaagctgatttggccaaggtgagctacagcgagcaacaccctgcacctttctgcatcttccactcaaggattggcgcttcacaccaggcagctagctcctaacacccacctctaagagcaagaagagaaggaactgacagaaagtctaacccagctgtccaagtccaacccccgggtattgagctctgcccacctaggacttagtgatcaggcctcagtggtcaaaggaagcatgtgggttcgaatcccagttcctcctcacctgggctgggttacttcagtcaagttactcaacctcccaggccacccgcgcctcttcttcacctccctgactacctccccttcccactgcctctggtgttggctagaggatcactttggccactgggatgatgaggagtgacatggctgagatctgggagtacctgctcaccgccatgtgctcccctggtaaccctctcaccatatttggaggagccaaggccaccaggctggaggaagcaggaaaaggccagtggccctgtcatccaccgagctctacacccccgcaggagccattctggccgaccctgaagcctgaatcagcccagcctcacccgggcaaaatcaacagtggttgtttgaagccactgaattttgggaaggtggttaacagcaattgaagactgacatagttcctaagcctccatttcctcatttccagaatgaggacaatgaaagtcattccctgaagggcagtcgccaggacaagggaggggcaccccagccctgagcctgggcaaacccagaagcttttccaaatttctaagttttaaaaacaacagttgggcatgtaagaaatacaagctctaccttttttttttttttgtcagagcaaataaaagaatggcagtgagcttgcatcatttctcccaccttttcatccccagtgactgaaatgatagagcagcactcacggcacagggcaggttttgtagcccccaaagctgaaaacggagggtgagagagggctggtgccaggtgccccagaagcttatgtatcctcctcaacagcagctcccagccctgcacacgttggagacaccaaaagtttttctccctcctggtcttcagacaaagcgtgggaaaggcagggctaattaatggggcaccagaaggcatttgccatagtctccaaaaatcaaggaaaaggagaaagttgctttctgaaaaaagccctctactttattctgtcaataagagctaaattacacacaagtgtgtaaggagtggaacgtagaagagagggaggagaggggcccgccgtgcacccccccaggtgcagcccaggagcggagaggagccgggttggagccttcctgactccagcgtcaggattcttcctgctcctccatgccagccagcagagtgcctgattcagggctgggagaaacgcagcgcacccaggatgaagctggggctgcagggattacctctgtggaattaagggaagctgataagattcattattacaagagcgattggtaaggtctgttatttgggcgtgtgactggagcttctgttggaatgacatttttattcattcaggaagcatttattaaacactgaaagaatagctcgcggaaacaggagagcgagcttcccgatgttgggtagatgacacgcgagggagggggcatgagtgcccacatgttcgtgaaggaggcaggcctggagctcgctggaacagcccactcagggtcaggagataggaaggagcttagcggaaccgtcgctgggatttaaaggggacacattggggcccctgaaggctgctgtggactttcccagagaaagtcccaggtatctgtgaaggtgatgttggtcaatctcagtgttaaagtaaaaagaatcaggtttcttgacgataaaaataattgccaatcctgcgcattttgtcaggtccaagtgaattagaattagggtggccaaatttagtaaataacaacagagtgcccagttaaattgaatactttagcagcatgtcccaaacatggccagggtgtcgtattttgtctggaaactctacttgcaagcaggaggcaggaaagaattctggtgggacttgcattgcattgaacaagagagcagcatgaccacagtgccgggggcctaacaggaattccccattctttgtcatattcaaagtctttgaaggaattgaatacgtttttggagtagagagtatttctgtaatggactcggggaaagaaagcggtttgaacagtaagactgcaaagtcctgaaagaatcctgaaagcacctatctggaagctttgctgagaggccccgttcctctccggctctgtgggaaactgaagggtgtgttttgacccagaatccgcagggagagttgaattttacaaatcacttcaggatgcagcgttgagacggggattggagaattaattttttcctcctgagtaaagcagctcttcctaaagctgagaatcttagcagaatgaataaacaatggcagcagtccggtaagcactgtatgaccctaatcattctgaccattaccacgttttacaggattaaataggagagaatccacgtgacatggtgctcctgagaacaaagctaaagtatgccaatcaaaccatgtgccctggaggaaaaacttgggcactctcttctagcagctaataaaaaataggacaacacttgaattctacacggagaactgtttcttacatttagttgatacagtaagaagtaaccagctacagagaaatcacaagactggattagaccagaacatttgtttattaaaatgtaatttaagtaatcagaaccttgtgaatgtttcaaacggaaaccctagaaaatctaagttttctcatgagatattttctagtaaacacttccattgccgtcacacctataaagtgagtatgcacctgtgtatgatcccataagagcacttttaaagtaaaaaaccactataatatgaatatgataatttccttttgattttatgagaagcgcttgatcccaaaactgtatgacagctcactcttctacatagaatcaatactttattgcctataaatctaagatgcatataattcttaagtcttcttttcaaacactgctaactgttaaagtatgtccattaaaactaaggaaaaatcatttttataaaatatggatgaacctatgtctaaacgaagatattcataaagatagcacaaaaaaaatgaataaaaaagaatattaagaaatagctttgaaacagaagccttagaaaaaataaatgcccatggtcaaacaagtttaggaaaaactctttgtacaaaatgttttcatattaaaggcactgagaagttctgcaacaaagaatcatgtttaattgtatttaatccaccatttccaaaatttgaaatgagaaaaccccactgctcataccttttattaaccaaaagaatcatttcttaggaaattctaatttgagaaacaatagatcgtttaaagagatctgtatttgcctttcttaccaaactttcaaaaggaaaggaagtgtattgaaaaaataatttgaggttttattgactcatgctgtcagtcgaagctttctgtccattttttacgaattgggtatattccagcctcctggtctccagccttggggagtgaccttcgtttccaggggaaatacaggtgcccagaacttccagcagggggctctgtccgctagcactgactgtgaaggactcaaagctggttcaaggttaagatggctcaacaagtgacgagccagaaatgtcatctggccactaggacgccgcattatgaaagtgtgatcatggttcaagaacgttgccatgaggaaaatctgtgacggaattctattattaggttttatcgtgtgtgcattttcaaacatagtaagacagcccaaaagggaaaaagtagaacaagccaaaaagtattttaaaaaaataattatccaattatagggctcctgttgaaaatattaaaagacttaaaagtttccctacatggttgacaactcaagtatgcccactttctatcaaaaataatgcagaaaatacaaaatgcgagagaaaagttgaaaataaatggaaataaactacttccattgaaacgacaaatataactcttcagaaataatgtctgcaaatgagagaagcacattgttttgaggaagctgtattttgatacccctataatctggattattttttcttaaactctttcttccatatccccaacatccaccttctccagccaaagcaaaaacgtgctatttacaaattaaattgtaaagaatggaaaatacttgatttaaggtgaaagccgcaccactttagccagaagagttggaaagaaataaagtggccaagcaagatcaaaggtgcaaaatagacaatggacacacacagctttctgcccttcctttattgccagggatggtggcttattgaaggcattagtattcccactgggagagaaattgcagcatgacaggtgcaagtggctagtaaattacggcatttgcaggagagaggaacactttattccctcccaagccttaggagagtgtatacataacacacccctcatgggtaagacaatcaaacgtggccattctcttgttgaaatgccacaactatctctataaataaagcaaatagaccacacactgattttcatgtactcatgtgttgcttctgagaacatgatttgttttgatcttactaaggatcaatgttatataaaggtgttatttccacatagccagtacttttatgtacttttcattttcccggaatgggtacatgatgtacactgtaaagttttggcaggaggcagaattgcaggggatacaatgtaatgtacgtaaatgataataccaagcatttgcgcttagattgggtctcgtcttttcattggaaatgtgacttcagggaagagaagctgaagaatgtccattcctctgggcatacctcatgcaagcaaggccatgtggtgactatttctagcagctaatgatgaggatctggaataacagctctgactaaaggggagaggcagccccaagacgcattcacagagccacttgccactcacccatccctagctgaccaagccccgcctggagggctttgagacaaaatgtaattctctggagtttatgtcactgtagtggaaactaagagctaaaagcaaacaattgctccatagaatcagaccagcatggaattttcagagtatcctaaatatcagcccaacagaagtgttggcaaactttgagaacctctacagtaaaggacagggaggcactgctgaggactagatggggtgggggagggcagaagaacagcaggagcctccagagctgtccttaacccctggcctgtgttctcaaaaccgatgcttggaagccacctccatctggggcttctatcctcttgtctcttagtgccatgtaaaaatgaaatttcttcttatgcccataagttataaagctctatgtggccatatgaacttcaaagcatttaaatttcttatggatcttatgtgattaaatgtgtggcatcctgaataatgaatatgagggtaacaaaagtggtactgttgagaaagacatttataatggtctaagatacagtatcaaacacttagtctggacaataagatcttgataatatgtttctttttttaagttttaaccaagggtcaccaatgacagaaaaatttataataaaccacttccaaacaatgaatatcttagatgaacaaagctgaaaaatttcagattccaattcatctttttacgattc

SEQ ID NO: 29 - DNA, 3962 bp, Homo sapiens
  source 1..3962: mol_type = genomic DNA
atctcagaagcaaggaaggctctttggagcaccagagcatagtctgggaacaaatgagtgacaggctgaaaggtgttccaaagaatattttatttcaaggctgctttgcacaaatgaggaagatgagacctcacattcccaacgctaggccactgcagaacttcattccaacctaacactcctgaccctcatcctccgcctgcacatgaggtcatagacaaaaagaacttaggtaacctgccttgagtcacaaaacaagcaggtgaggaatttaaacctgattctactcagacccatattcttgagccacctgctcattatatgcagaagaatgttcagaagttaacggttggggggtatatatgcactcagaaaattctagaaggaatgccatccttgaagcagccttttctgactatactaaaagagccctcccaccttgttgttatctctttaacctgcttggttgtctcatgacacttatgtaacatatactggatctatttattctctagcttccctctagaatgaggaggaggacaatgcctagacattgttgatttgaaaagaacttaaaatggtctttaaagtgcaaaaaggtaatctcccccttcctggacagagcttctctagagcagaaagttcttttgattgcaaaagagaagaccattggaaatctgcttagataaacttgctttgagtcaaaagtgaatcattaatgtttcaactgctaatccacttagcgtcctattcacccagatggcttttgagcactcacatcatgctttttgaaagataaaatttccagtcaccagaattcaattgagaagacaattgatggtagatgaagagattgaagtttctgagatgagtgaacacacttatctggtcaatgtgaaatgaatgaaacatttgaaatgaactgatatgagttggttgggttttatccctataataaactctttccctccaaaaaggattaggtcttctgcgaatagagttataaccaactcatctctgtcctcaagccatatggccttgacctttcttagtgacgcctagtcactttaaacctgttggagatttttcttgtttgcgtccttctgaagggtgactttcccagcttcaggagccatctaaaagccaggagcaatgcatccctgcattccctgaggctgctctggctggcttcctccctcttccccgaacagaaccaagctcctatagaaaattcataaattcttggcaggaaagaaatcctcccattggaaggaacagatgttgtacgagtaactttgccattatgctattcctaatatcatagttttaaagtgtggggggatgttaacttttttacaaatgtcaacatgttctgggcagtttttccatctccatctgtggcatgcacataccaatccttctgctgattttccagggagtagttggaaattggaaatatattaaaaagtgaaggcggaaattcaacaatatgctaatgtatagtaatatgctaaatgtcagaaatattctttctcgtcgcctagaacttggatggtgtacagacttctaacaaagtaattaactgtgggaaaatcagtggagctagctggggtcccctccctggggacagcgatgcccgctgccagagcgcccgctggctgtggccgggcccagaacacgcctgatcgcttttgggttcaaagtcagaatcgggggtctgggtgggatggggtggagtgggaatcgctgcgcagaagcagaggaaggtgaccgggggcggggcgaggaagcagcggagccagatttaagtgcgccgccggatcgtcccgcagtttccgctcaggccaggtttccctccgccaggtcgtacaaaccagtccccagatccgtgtggggcccggaagcaccccagaacgttcttgtccgtgctcaagtggaggtgaaatgggagcgcgtggggcgcggctcgggggagagccgatcacccagtcccagtgggcccctgtgcggcccccgcatccgggaggcccgagggccggagctagaggcgggcgcagcgcggtctgcagtgccccccaccggacggcccggcacaggacggctgctggagggagtcactgcctggcttccagctcccggggactgacttctaggcttccctttggctaacgaatagacggaggtcatgtaaccattttctacctttccgtagaaaaggaacctgagcacccagctggggtccagtcatctatggctgtggcctcagctgtctcctttttgaacctaatattctcattgcaaaatgggagtaatcatatccacccacagagtgagaaatgaaggacccttattcggggcatcagctggcgggtcccagaccacctgtactgtagcacgtagcaggtgcagtgcataacttgaccctactcacaggcggcgggtccttttttgaaacacatagtaggtactcaatgtgtgggaggttttaactgttgggattcttcttccttctctccccttgacaccccttccacaaatgcctgggatcctttctgaattggtgatgttagaaactttaagtgagaaaaaaaaaatacagaaaaccagtgttcaatatttacatggcctcaccagtcacagtgcacttctgtgtacattatatccatttaaccattttccagatgaacctcaaagaggtagagcgactttggacacagtgctcatctctacaacacagagtcttctctcaattcagccttatctctatgctgttagacaaagatgctgctttcctaggtatcaaagtgggacttttcaccggccatctgagcaagcgttctttgagactctttggaaatgataggacacaaactttattaatacagaacattgaacaaattagagagtgtttacatattatgacaaggcatgcagcagctgttactttaccaaaactattcattatgggccgtttgtttaaaggttgccctggtggcttggagacagtgccttttgctctctgactcaaggaagcaaaaataagttactatcaggtgatgagcatacttagaagccaaactaaaaatgagagggtttatcatcctctggcgtaaagcagtcggctccactttcaaactggtaaatgccaggcagttgaaaaccatgttattgaatgtggctttaattcatacttctgtgtagtattccactgtgtgaatagaaatcttatttatttgttggtcattatgcttatttgtagttttttttttttgaccattatttacaattctgctatgaacattcttatatctccagggctttatctaggtagatctcaagaagtttattgctggatggtaagataatgagatgtatcttggcctggcattcagaggtatccgtgacctggcctaggataccccccacctggctcccacatccctagccaagcactcccctgtgtgccatttccggattcaccctgcacttccatgtctctaggactttaaccatgctctagtctcctgcctgtgcccctcttccctctcctcctcttttaaagtctggctcaaagcccctcctccctgcctgatagtctcttctgtgcctttgcactttgtgtttggggaggaaagatccagttaagccccggctttattatctcctggcattgtgactctagagtgtttaatctctctgacctcaggtccacatgccacatgtagaagggaaatgctgcaacttattgtactgaggctgggcttactttttttttagcatccctcaattaataaaatgaggtgtcacactgtggttaagatcatatgctttgggattagatcatcctgggcttgaggattaaatgagacaacgtatataaatgatttagcactgcctgcccacagaaaatttccagtaatttctggctgttattatactcatacccttctacaggagtcact

SEQ ID NO: 30 - DNA, 1533 bp, Homo sapiens
  source 1..1533: mol_type = genomic DNA
tgaaggttaagtgtaaaaattattgaattgtgaaaattacgctgcatccctctgggagaagcatttgcagcctaggaagggggtggtgctcataataaaaactcgagaagccaaaatcctaggcctgattttcctttggtttgaaatgcatactgttgcaagggcaagtattgacgaaatcttgaacatttctttcacagtctgttgctgcatgcctgaccgagcaaatcctgcttaattcctctgctccctagaggaactcaggaccacctggaaaaagtgaatgatcgattaatgatgaattggccaacttgccaataacgataaattgctttgttcatccccaaggatgccacctgtgcctagagacaaaggatgggaactcttcaattaacatagatgagctttcagacctatactgcacaatgacaacatctcgtttttgaccaacttaaaatgtttcacaagccttatgatcagatatttatatttgcagaagaaatacaatggagcagtcaaaattgttacagcaggcttctttttttatttgggtggatgtgagtgtcgtggatgaacatgcaattttaatgtgtataaaactctctgaagaaaaccactagaagatccactaggaatctgcagccccttgagaaaataaaatctgcacatgaaacaacaaaataaatacaaacgacagtcaacaaggacaaattaccaaacagtcactttttatttagtatttttgaaatcatggaatgaatttatgtttcaagatatacaagaactttaaaatgctccttaaatataaaatcatttctaagccattagtaacaagtagtaagttagccagattatagaatttgaagtatcctgggagttgacagattgagactctgagttcatagtagtcccctttagtcagtgactcatataaactgatatttatccagtaactacactggctgcagacaagctgaggctatcaggtttagcatgttatgaaaaactcttaattatacaaacttctcagtggaaagtgatctctagatttagttttgtttttgtattttgtttgaatggattagagcaatcttagctttctctgtgccttgggtgcattttgtttaatacttcccagtttacctactaggatttttgtatgttaatatatatggaaacactttgaaataaaacaaaagatattattagctaaggacttttattatacaacctcataaaataatttggaaaatgaattaagattccatgcagagtcaagtagctgactggcgtttggaagtatgtgggaaatattttttgtagatatacgtttgtataaatgaaaaactgagcatctttaaagagatacatcactaaaaacatattattaaaaagaagacagttattctgtatacagcctaatgttgtctttttatgatcgtatttgaatttgcattcaggcatctgatgctgcttggggcaagaactatgattagttataacccatctttggtgcctgggacagcgtctgac

SEQ ID NO: 31 - DNA, 77 bp, Homo sapiens
  source 1..77: mol_type = genomic DNA
gtggctgctgtgtcacatttgtgttattaggtggcagagagaagagaggctatgtctatgctcagtgttctgcccca

SEQ ID NO: 32 - DNA, 298 bp, Homo sapiens
  source 1..298: mol_type = genomic DNA
aaagaagagataagacaagcaagtacatagtttatttggaggtgatattcaaaaactggagtagggaaaagtaacacaaaaaaggaggaaatgccaggacgcggtgtgtttcctaagcagcagggcctcagctcctgggaacatgacagaatgcccccgtctgaagaactaaggctgggtcatttgcccctggccccaatccctactggttgaggggtgctcctgggatgttaggctacacctgcacaaaggtgagcttctttcagctttggagaaagcccaggagcagaaagtgaag

SEQ ID NO: 33 - DNA, 598 bp, Homo sapiens
  source 1..598: mol_type = genomic DNA
agagactttggtccattgaatgtggtggaagtgaccttgtgctagttctgagtgagcccaaaaagaatgcagtccttgcagtctctctctctctctctctcccaccctctgctccccaccttcccccacccccctggacccacccccgcccctcctccccatgatcactttaagtcacaaacatggtctcaactcaaatttaaaatgtccagtcaagctattaatccaaactcccaattggatctatgtcctgtccttctctggacctcctcccctcccccacaagtgggggctgtggagatagtaaggggaggggaagggtctcacttgagctgccgccatcctgctggcttccagctgacagtgtcacagatgtcccacagatggccatctgcagggctccctcactcttgaccctgggggggatgcacttctccttctggaggtcccttgcaagtccttccttcaggaatctccaactccagcttctttccaccctccaggaggccctagggaacaagacacccctgcccccaccccagcccacaacgaatccccaaacccttccatgtcccctgcctggcatctggaggggcag

SEQ ID NO: 34 - DNA, 5448 bp, Homo sapiens
  source 1..5448: mol_type = genomic DNA
cgtggagaccgagagcagcatcagcagacaccagcactgcccgttcctcctgcaagatgtggagggagaaaagacagccgctgagctccacagcccaggaaggctggaatcagtgatttccttatatgatggacaaggagactgaggctcagagaggtggagcgatgtcccaaagtcccaagctgcactgagctcctagatgctgtgacccaccctgcacgcctgacacggaccgacttgaatgcccaggagtggacctgcggggccataagggatacatacaacgtaactgatgatacccaattcactggaatctaaaaatgaagtttcccaaagcacacttctcatctaatgactggaaatagactcgtgttaaggttgttgatttgcacctatttctcctgggaaattcaagaagggctctcagtttgcagcttgtccctgaactcaggcagatgaagaacagggagggtctcaggcctgggagctgaagtgggggccaaatggtcccctgtgggtgtgggagggatggggcaggaagccaagcaagacattttacctcctgccagacagtgtttttagggcttaagtgatttccttccattcaatgttaagttgtagaaatagccaagaggaagcttcgtgtcatacccaagctgatctaaattgtttaccaccgttcagtgtaatctgtgagattgggatgtgtgaagactggggatcttatgtcccaactcttacagaactggactaaattaataggctgtccattttcccataatctacccaatgtccctgataactcaatatccaaaagcattctccatactgtctcaagcagctggaagggcacaaaaatctgtggaaatcatctaaaacttggtttctgaagaagttaccaagaattagtgcatgtcattctagacctcagtttattcactacatgggtacaatgcctcccttgtgtctacaggaggtagttagagcttgcatatcatctgattaatgtgcagaaaaagtagatttaactagctaactagaatagaatttactagaataactaaagagatagctgcctttgggataaccaagagaaaataatgactgtttagcccacaactaccttccaaggaggacttttgttctgctgagcatcttgatgtgtttatgcaaagaaggctgagacaacattggttcaacataagagaaaatgatggatttgaaaatctgacaatccaagaggaaaagaaaaaggagggagaatgtatcagacaccttgtgccctgcaacatctgtccagatcccctccatcttgtcatttatgtgcagggtggctgtcttccactgccagcacctgcatcccttcacctgagcgctttctctggccctggagcctgctctgcccacaagcacagtagatgtatttttctcccttcctcttccagatgcactgtgcgtaatttataaggcatctcagaagatggtcacacaatgcttgcagctattgcctcttatgggctcttcctcctaccctccctttccttttcacttctgctctctaagatcacactccctacatagaggcacacaagacttgcctcagatgctactatctgaataacccaggatgagacaggggagcttatctctcagcttctgacctattcatgcatctaacagagtatctaaggcaattgtcccttcctgctcactatcttcagtcagcatgatgtcaccagtataagggaccagcaggatagtggcatgttcttatggactatattataacaaagagctgttatgatactcctggggcaaaacagtgaaatatactactgtatctgccatgtaaaagtgaactggttttcttccttgtgattccacttgccaggtcactctgcatcccaccaccaaaggctatgttggtatattccagtaaagataccgtatttgcatggcagctgcaatggatgctaccatctggccaagtttatggaaatccactatcatctgccactatcaatttggctttcgcaggggccttaccataccctcatcatttaagtcatgataggggttctaacccctgcaatactttctgagaggtgatttgcttctgatctactgccttgaagtggaggaaaagtttcaaggacttccacttgacttttcctacacaagtcaagttgctaatgtaagggttctgccagaagctaagactatctatgtatttgagatcaggccgggtggtttcctacaaccagtaatcagttaaggaaagaacccagttgcttcacacatgagcctgtgtgatatgtcagtgctaatgagctgttgctgcattgcagccttctcaagggtggccgcaaaggactcctcccagggggtagaactatgagtggggcactggtcaaactctttgaaagagagagaagtagtattatgtagatgcctgagcagtgggccttcgatgttggccagaggcctgaaagacaagattggatgatctgggacaggagatctagggaagcataatgtagattgacctacagaaatgagcagtctttgtatctcaatgctcacgagaaaccatccaccacagaggagattcaacagccaggatcctgtggatgtcagccagtctctctccttgcccagaagcatgggccccatccctctaaagcagatatagctgctgacattgatgaatgcccagcctgccagctgaaaagactgacacggagccctctatggcacccctccgagaggccagccaaccctttggtgataagttgattgagtatgttttcctcttcagcatcagtacaatggaaatgggtttgccctccctgaccaaatacttgggtctggaaaccaaggagtagtggtggaattcagtccttttcctctcccagtgatccactgaaactaagctctggtggggtagggtgctagtgttcagcagaactcctccaaaagaacaataaggccactaacttgaagttgtgaccactaggtcaccttgagctcctcatgccagtgggcaagcaggcaaaaaaagagtgactgcataagtggggctattgagccctgatggccaccgggagccacagtagcagctactttatggggcagagaagatatatttacaaagggaatgtcctggtgtatttctggtacatgcatatcctgtaatgatagcaaatgggcacatgagcaatcaaggcagctgagaggtcagacccttaaaggataaaggtctaggtcacctcactaggctagcaagccagatcatccaaagtatggactgagaataaaggaaatcagactgggtgggggaggagggatatggcactagctacagctgagggtttggcttgtttcactctcctgcttctcagagatctaagttggttatgatcttgaaagggattctgactactggactcgtacccctcttcttagaaaataaggagaacatcatattctttcaaaattgaaggtcctatatgatagcatacacatctctaaccatttcaaattctctgaatttcctgcctccaagtggtcattcattccagccacagccattgctgcagagatcaactcaccatccaatcaattacgggtaaaactggcagtgccttcatttttgataactctcacctcatacttagggacttcattaatcctgagacatgagatgctgtagggttcatttagtgcccacacataagcaacttggaaatgcgaaggaaataatatgtggccagctttgaacaatgagagatggaaaccagtggatgactatttctccctccctatcacacatcagccctgagcagcagcttctcaaaaaaacacaaggaggacagacctgttatttgctcttacttcttccctgcttcttttccccctccaagtttgcccgacacacgaatgctataaaaatggacttttgtggtatcaactgagacattaatacaacgtcttggtcaatagaacagaacaagcacacattgatgagttataaccagaacttagaaacaagagctcagcatcagaggtgaacataagaagataagcttctgggaatgtgcttaaatattgaaaatggctgtgggcttccaaaaaccacagaatagggggagatagaggccattcccaagggacaaagaagtccccatccaacatctgacaaatgagcaatataaagtatccaaaatcagacaacaggggtttaaatccagagactgttctttaccagctggaatatccagagatcttcaagcctagctccactcatgcccagagagaagaaggggattgcccagggccactgtcaatccaatgtcctgactcctatccaatgctcttttctctgctcagtttccccacattcggggtcaagaccactacaaataagaactggaaatgcatatccatgtttatcatgtgtctcaagtctctgcatgctctgctaagcactgccctagagggtgtcactccaccaaacaacattggtcagccacggttgactggtttttttacctgtggcccgcctgcatcagaatcattttgtggaaagcatgttaaaagtacagatttctagtctctacacaatcaaatcagaatgtctatagcgggctcgagaattgcaattaagaagctccctggtcttcttatgcacggccattgacctaaagtcacacttcggcacctgttcctgaattctatcttggaatttcttgcatgggggtagccccaggtatttaatactgtctccacgatgatgcatttgaactttttagctcagacaaactgccaagacctctcttagatggattttcccatttgcccacccttggaaaagcaagaaaaaaatgtattctgttgagttaaggcctttaggatccaagaaagcagccaagagctagaagggaccttgtacctgatcccaagcccaatagaggctctgctgtacaaatttatttaataaagaaataagacaactaaggaatcttctaggtaccatttgaccacagcctgtcaaagagtttgtgggcaaaacgccacacgtttgggctgcttgccttccattctacccagctccctcatcagtgcacagacaatgactgccgcttccacagtactgtaaagaagatggggcaaatgtaaaaacactacatttaactgctatttttgtctcaaggatgagggttgataaatgtgtccttgagataagccagagacggggacaaaaatctgtccaaagagatagatgttcccaaatggcagggccacaccttactcttgagagtggccagtgacagactttctctgttcccctttatctgcctcactgcctcctgcccataactgttagaaacagctctgctggacatgactgtgaagtggtcactcggagctgccacaggttgtggcttc

SEQ ID NO: 35 - DNA, 6087 bp, Homo sapiens
  source 1..6087: mol_type = genomic DNA
cctctctagttacctcaacattcctcataaataatatttgacagaaaaaaaataactcatagtacagctataaaaaaaatcttcaaatatgcacatctaatgtttgctacatgatttcatcatccaataaagggagaaaaatgccttaacatatcaattaccagtatctgccttgatcaaaatagccagatagttaccaaaatcttaatctcacatagagttacgtggataagtaaagtaatgacagtatttaacaccaccacaaatgaaatttccatattatcaacaaacatcaattgaggaatcactctattagactagaatattgtgctatagtgacacaggtaccaaccacaaaagcctagccagccacttttttactccactctggaaagtctaaagactgaagagtagaatcgtcctttagaatgaaggagactgagaattaaagtcagcagagcaggatttgaattgtggtacaagccgtctggtcagacaggccttgaaatcatttaatatcctgagcccttaattttctcacttaaaacagaaacaaaatatctcttctttatttcaagaggttgggatgcttgaatggaaccataggaaagaattttatgaaatattatgtgaatatgaagctgaaacgaactgaagtcctgtgtcacattcaaacgttcttcactctgactctatttaatggtatgattcaataaattgtaggttcatcttaatctcctcaaaatactcttggcctttgcaaagtgggggaaggaattagtaactaaaatagcaagagaagcttgctgtcagttagtctcctggaaaagtactatactgaagattcaataagcaatactgtaacttactaagatatatgtgcagataaaatgattgcttagctggagaactcaagagaatcagctgaaaaactattacaaatagtgaaagaatccagcatggggcccaggtaagaaattatggaaaaatcaatactattcttacatttccacaaccagaagacataatagaaggaatcattctattcacaaagagggcaaaaagataaaaaattccaagagacaaacttaacagtaatttttttaaagactgaagcaaattgaattatgtttttcattttaaagacttggcatcttaagatgttgattctttcagagctattttataaatactctattgtgaatcacatccccaaaattattagtaggattttcaaattaaacaagttcctacgtaaatctaaaaaagcaataagagccaggaaaaagcatctagggaaaaataagatgaatgaggaaggactgataataccaattaaaaattggagaggagaaggaacaaagagagggagggaaggagggaaaggagaaaaaaagaaaccctaccttttatataaatgagcaattataaatggaataaatgaacaactaacaagtagcttttaatcttaaattaagcacaattttaataaaataggaaatataaaatctactaaaatccttaatataatccctggcacatagaggcatatttgttgaataaatagtaagtagtagagatatttggtaaattgtcttttggagtcctaatgccatgagatttagacattatgctaatatgtgttttttaaaggttaattagtattatttttaactcaattttgaaaagctgaaattataggaatcaggaccaaaagtttcaattcaaaagtaacgaaaaaaggaaataaattttatatttttaagtaaatatagcaaaaatttctaattgttctcctgtgcttggtcatacaaaatccccaatcattgtagcattataattatagcagcaaaactaaatatccatcaatggaggacagattcgaacaaatcctggtacatttatgcaatggaataccatttagttattttaaaaaataaagtagatcctagcacttgatttgtttcttccaaaatattatgaagcataaacaatgtctcaacttggactgtctctttttttgtgtctctcttggttaagctacgagcttagagaggtcaggaactacactaaccccagtgtcagaacacaataagaagagtcaataaactgaggtgaactgaaactttggaccagttcactgaactttagtacctattgtgctattctttagcaaattgggtaacagataatatggaaggatttaaaatgctgaaactaaaccactaaaataaaatactagattacttctcttgagtatggagaaaatgagccttgcttttaagaagcttaccacattagcaaaagtctggaaatacaaagcacactcaaaattaataaagttaagattttcacgctgccatttgtgcaggctttatctgacttctacctgttatttttttattctctaattctatattgatttctgtgatgatggaggtgaagagcctgtcactggcaacatagcaccatcctcgtttagagtaaataaaactatttaaagcaaattggaaactgaatgacagaaagtgccacagatggaccacactgggaacaggagtggaatgccgcgttgttcatcatccccaccatccaagaatgaagaaatgtttccccttaacttgagtcattattgctcatttcctctttcttttcatgtcttcttttctctttggagatgtctctagaatggaaatacatggaactcatccaattgacacttaactgtaacctctatgatacaaagttacttcgtttaccataaagcttaagcgccccaatgcagctctttattaaagtcatgataattcagaaacttaaataaaagtattgctaaaagcatcatgaatggcaaaagtctgaggacttctttcagggtgaaaggaccaaaggttaacactttgctgcctaccttagaaaattgcttattcttttaaagaccaagtattcctttgtcctgttcaatccaggaagacatcagaggagaatgaaagaagaaaggtttatgagaaccgagcagtgctcagacaagaactctacccactgaagtcaactgggtaatttacattacctatgcagtagtaggtctacccagccttgctctaaatgtgtacaatctcccaaactgatgtatgacctataattgatcttacctcctgctttccctataaccactattcattgctccgagcctggaagatcagatattcaaatttaccagctcaggctccatgttcctaccggaactcttgacctggttacaggaaggaaaaatagtgacacacttttcactgcctaaatgtttcagtctttctgatttgagagcccactgaaatccattatcttggttaagcttaaaaaaaaaaaaaaaaaaaaaaaagaagaagatacctgtggctgggaatagcggggtatactggctcaagatgggagaaataattggtaaaatatatatcttcagtttctttctgtatttttctgctcttttaccctttttttccctttccacccctcacaccctatgcttctgtcaatgactgcaaagataccacaattggtaaagttatttctgttcccttctccttttcattcgtcaggtagtggcaaaggtttaatccaagttagtgtagactaaccaacttatctaagtgctctgatatttactgagtagtgctctgtactagatgcagtgaaagcgaagaggaggaccttcatccttgcctgcaagaagtctacactccaaaggggggaaacaagcatgccaactaaaacaagagataggaaacagagggaaaagcaactaaatgccaatgaggagatagtttcactaatccctaccaggaaattatactacagaaagaacacttaggaagaccaaagtgcaaattaagtttcactggatgtcatgcaaggaatcctcaagctcaaagagcatctctcctctcaagtttttgcacagggtggggaattgcacagtacaaatgcttacaattccaatcacgtctactctgtgttatgtttccacctgtggtgactctattttgagaacagaacttattttgccaaaaatggtacaaacgaagctgcaaattgactagggttgcacaaacaagctaagcatccaggtctataaacattaaaggagtagatatctgactggtaagttacccctctttccttttttttgttatccagggaattaagcaagttcagagaaggtaattacccaattaaacattctccctgatgcatggaaaggacaacacgcaggaatgcagaaggagacaagtgccaagaggcccttgctataaataattgcttttcccacgtccttgtcacctcttcctaaaattagaattagcttcctggcctgaaggctaaacctcactcccacaccctcctttctcacaaagcaatattccctaatgcaataaggtggtgggatttcaacaacagcaagagtttaggagtcaggtaactctttgctcaaccctgcttttcatttcaagctttattaatctgtgcaccaagcttatctgaaaaatgggatgaggatgatgatggtaacagctagtaatcgtaaaacaagcactgtgcaagtgctttacatgcattatttatgtaatgggcagtattcatgattattactagcaattgagtcctcctttacagatgaagaaactgaaattctagaggagttatgccaactcttgaccctttctactactataataataactgctaaacaaggattgaaagggttaacattaacatatatggacacaccaattaatgctattgtcatcgcttttgatgttgctgagcatgatactgtttttgttgcttttctctctccccagatactagcagttctaagatcaaataagcacagaccatccacccatatactggcaagtgtaaaccaatgtctgggaacatttttgagcaccaaataacaaagctgatattagatctttcattattaaaatatttctgatgaatgagagaggaagagaaaaagaaatgggattcagtactaactaaaatgatcccccatgcctaagtttttaaaaattcagaataacaaatgaaaggtacactgttcttacatgtgtcacttagatttcttaaaataaaattatgtatcatatgctaatttttgactattcaaactgcactttgcttcattcttatcttttaattcacttttcattgtaatgaaataactgtaatacatgtttaaacctctaacacgctaaaatgccaatcagttaaacacaatagcaagcatttggcatacacctggagactttcctaaataaaataaaattattattacatttcatcacttcctttcttattgccaactaatgtccccgtgtctgaaagggtcattttcaactacacatctatttataatagtgtgttcattgtcttccttcctctggtgcattatctatgaccttgaccatttcaggatggataaaatggtgccataaacatatgtttatgaagctccagacatcttgtaagagatttcgtggttattaaaagcagcaaagagtgacaaggacagttctatactataatatgcctgtcaagagaaaaatgtcacccttaccataaagtacccagaggcataattaagaagtggcaaaggaatgatttcctgggcacagtaataacagggctgagatgaaatccagcagcaatggaagctgcctctccaatgtgcctgagtggagcccgtccacaccaagggtccagggcatagtcatgctcccacgtccaaaatgtaacatcaaggaatgcccaggacagccacactcagaagaggtggcaacaagaatttctgggaggatgtttctgactttcctaattgcatcattatgaacctcctgggcaccaactaagaacattaaagaaattgaaacttcacagttcctaatgctccatacccataaacacagatccatacagtccccacagaactataaataaatcctgcctgaaggtctccttcctgttcacgtttctccataaaacttggtagtatctctttccttttatctaacttttatccttgtg

SEQ ID NO: 36 - DNA, 2007 bp, Homo sapiens
  source 1..2007: mol_type = genomic DNA
ggaaggatttgcggacgcacgctcttgaacgtgcgtgttctcagaactctgatatgtgtgtctcggcagctgacaccacgtgccacatctatctttgcagactgccccatccagcttagtgaacagctgagccagaacttttgcaaacatctgccgtggttgagttggggatggaaaactgcggagcctgatggttctgtggagatggaccaacaatggcaccacaggtaagaagggttatcttcttcctggcagaaggtggcaggctgatgctgtcgtcgttcctgtgtgtctgacaaatcacaaatatgtcagatcgtcacagctgagtcaaggatttccatgagccctgctggggtcagcttgcagcgccaggcaccagtgtgactcgcctgtggaagccggggctgctcagtggggctgagaggagggggagagagaggcccatcaacaaacagtgcaaacgccgcagctgccaccaccgagttccagtccctatgagacgttttatctttcactcaagaggactgaggctcaggaggtttgaatccaggtcattctgacccaggagttcagactttttcccagcccagcctttccactcgaccaattagcacagtcaggtgccaaagactttgcttgcgggatccatttgaaatggttttttaggcaaaggagactagatgaacgtggaggagagggaaagtataggcgcgaactacagaacaggcttattccagtgcactccaaacccaggcgtggtgttcttccttctcgcttactggtatcacctgttgtcctctgggcacctgaagctcagcatttcccaggttgcttttccctgcatagccctggcaaggctactctgtcctcactttaccccagccacatttcccatgtctttgggatttccttgagactgatctgcccaagcctccttgggggatgggatgatttcaggattttgagagaaaataaacatggagctgtgatttaggaaaactgtaatcaagcagctgttgggaagacccaaagtggggaagaaagatcatattatattattcgtgctggctaaggacagctgccataaaatatacacatatattttatgtgggagacacgtgctgcatgctttcgtcttgtccgtgcacagacatacttggattgagtaaagggagttacaaaaggaccaaaaattgctgaatcatttcaaagtgcttgacagttgtgtaaagaccttttggttttgtccagtgaaaattattttttaattcaatctaaagacttcagagcctttaagatgcaatgatgggtttatcaaaatactgttcatgctattcgtttcttagcttagaagtctctataaattagcaccagttttccaaatgcatttttatcaaatcctatgatcctaggctggtctaggtaccatcccccaatcttaccttgtgtgtttctgttttaagaccctctcccatgctggcatgcctttgcttttggctctctctttttaaagcctgcccaaacctaagcaatgtcagggcatgtccctttcttcctcatgaggccatttcttactgcttacgcagaaactgatctactttctttacattccagcaaatttgttgtgcctgtccctcttcgtttaggtatttctaaaactagactcagttacttcacgtgtgaagtgggaagcagtattttctcacagggttttgtgtggtcacttagtaagggctggcagccacaggcttgttgaggacagtgggggatgttactttttctcagagtcttcatagtaacttgtcaaggacatcactcactgtactgcaagtgttcatcatggccagcactcaggaactttcagaatattttgagtggagttttgtcctgttttagttgcctgttgggatccctgagtataaaaataagtgtgtgcctgctcccagcattgacgagcttcatgaaccttgaaaatggaaactttatttctctgtaggatgtgag

SEQ ID NO: 37 - DNA, 16213 bp, Homo sapiens
  source 1..16213: mol_type = genomic DNA
ctctggatgtgtccgggcagcaggtagcgctgagcacaaggggcaggagaggcgggaccaggccagaggagggggactcagcctgccattgggccaggggcctcgatgacaggagcgatgagcagtttcgtgtcacctaatatagtcatacggaaaaaaaaaaaaagccctgcccagcttttttaaaggggccacgacgcatttggaagggtctcctgctcctgcagtgcgtttgctccctgtgagcctgaaaagcctaccagaggcatgcagagtcaggcctctgggcagggctggatgggtgtgggtttgaagaaatattcctcgcacccaaaccagcatgggagagagaaggatggggtgggaagcgggggaggagagtgatttgaggaaggctttattactgaagtgggatgagggagagtctgaataggaagagaagcctggaggacagggctgctttggggatagcagcagggcgagattctggacaattcgggggtttggtttggcttggcctacagggtcagtcatgccgcaggctctgggtgcttctgggccgaaagtaagggcaaggtgagtgatggtggggtaggagttgctgcctcctacagacaactccagggggcccagtaccctcccagggaaaaggaatcccagctggtaagctcagcttcccaggagacacctgtcttgtggagtgcccaccttgtttcccctggatcccacctccctccttcccgtgccctcccaggccctcatcagggtcctagcccaggtttagctcgctcctccaggatttctctgggcctctacttcgtggggcagcggctcggtcaggcgcgtgggggaacatttttagcccagggtcagtagagtaggaaatctgccagagtttcaggagcccagatcctgcccgggtgcatttctctgggtctgatgaaagcttggggtctcccagacactgcagagaggagttttattttgcttacaattcaggaggggcaggcagagcgggcccaggttaagccttctagagctggggatgacaggatttgtcagtggaaaagcgttggcttctcacgcggagtgcgggttggcaagaggcgcttggagtagaataaaggcggcgcagggccaaaaactacctaaagacacagctcccgaggcgcgcccaaggtgcggaggaaacgctccatgtatggggacaaaatagtgtctgtccagaacgtacatcttgggtgctcaaacccgacagccgtctgagacgcagtgaagtcagcattggcctgatttgggacttttcctgtccctcatcaaagccacactcccaggacctggggcctccatgctgggggagaggtgagccccgccccaggaggcgcgcaacgcccgcttctaggcgccagccgtcagtaacaggcctgggcgccacgccgaaatcacgccccagggactactgggggtctggaattgttagtctgccttgcggcccttcctcgcctggacttttggccacgttaccgttatggctgtaaattaacctagcttgggggcccgccttgcttgaagattgttttgtgtcgttggagaggatgcctatcgccgaggcggtgacgtctggcaaacgagccacatccgattcaattaaatgtctcgctgaaaagagctcggaacgagggtcctgggagcgctgttaagtgaatctgctggccaggcggctcgcgatgacagtttgaaagattagtgtgagcggacgcctgaaatattaccgtttaatgggggacatcgaggctacatccgggatccctgtttttagtttttttttgagagctctggagaaagaacattgcccttgcaaatcaaggtggcaaggatttatttcctttttcgggtaagggggcttccagaactgtcagtcctgtgggtcaggtgttacaaaaagggaagaaaactctccctatttgaacaagatcagtgactgttacacctctatgctctttgtgaatcggctcttggcttcctcttgggagaggagatggggcaaaaacggacaaatatgcaaaatagaattggggcgacccatgctggtctccttagaggaaagtccagagccatttgcagcttttcttcagtcctcactgtttccgagtgagttaaaaaaaaaatagaaattgcaatagttgggggtgggtgggaggaaacttgattctctgccccaacagggacacatcttttttcccaagggccgctgacccaacctggccgcctctctgaaacaaacagccttcagccgaaaggcagagagagacaaaaggagagagaagagatcgaaaattctgtgtatttccttgacttttaaagagttggtgctttaagataatccaaattccagggtggctcgagcgcattctccgtgaataaattattattaattgaaaagcgatcggcgcagccggagcggcccccctttcgtgcaatcagccgcaaaaggtatatatgattaattgaaagataagctggaactatcaccgggaatgtcattaatgcgctggggagacgtccattggagacaggcggcgttatccgcggctttatcttcaacaacgcctcgctctcggcccgcgcgggggaaacagatgggagtttctgtctgggacgctcgccccatgtttatattttgggaagaatcgcaactcgtgggagtccccggctggccccctgataaatgaacattagcactttttcggactactgtatcaacaaaggggctgcggcgctgcaataattggcgagaaagcaaacagagggccgagagcgcgactctgctcttcgtccggctgatggatgagcagcgcgccggactcccggcctggcccctgcttccttcctgcggggggcgaagggccgccgagctccggaacaggcggcctccgcggcttcaggtgctcagctgttcagtgcccacggcgctctcacggatctcgtcacggatcctcttatccgactgcaggcctaggagaatgaggttcagagaggagtaagttgctcagggtcactggaggaaagcaagacccccaccatcaccccccaccccgcagccaggggagaggaccaggctgttggatttgagaatagacaagggcataaagtgagaaagagagggggagagaggaatttaacatgcgttcacctcgctctcccacttttcctttcttttgataacactttatcgagatataattcgcataccatactccacagacaattttagaacattttcaccccaaaaacctttagccatcaactccaatccccccttcccagccctaggcactgctaatctattttctgcccctatagatttgcctgttctggacatttcatacaaatggaatcatacaatatgtggctgtttgtgtctcgcagcgttcacttaacataatgtttgttcgtccatgctgtagcactatcagcatggctgaacaatattccattgtatgtctatgccaccttcgtccattcatcacttggtaaacatttgggtatttccacattttgctattatgaatactctaccttacagtctgagaaagcttccccctcctcccctattggcatctgagtctcacagggtggtttaatgttccgctgttctgtcttaaaactcttcataagtttttaagcagagtctccacatttctattttgcgctgagcccacaaactatgtagtcaatcctgctttctgggctgtctggcagagcactggaatactaactctctattgcctagctgggttttggctcccctccccagtgctagagctaggacagacagaaagtctccaagctgctttaagagcgaaacttgctcagccataggaagggctagagcccacagaaggaaagagcagatccacatggctaggtgtgtgtgtgtgtgtgtgtggctggagaggccgtagctttcacccagctgccaaggactccacagcttggtcactgttggattcttggcctgattttctccatcccttcacctacattgagtggatccactttattgaggtctaatgtacctagaacaaaagtgtacagttcgatgagtcttgacacttggatgtgcacccccataactaatcaagatacaaaaacatttaacctcaaaaatgcttccttctccccttctcagcccaagctgccactggttttaaaaatatatcctcttagagttttttttgcaaatttaaaacatattataggatgtatatatatttttctttacgtttcacaaactcctttgcattttgtccttgtcagttttgttttgttgtcagttttcttaggctctggggagtatgtttcccaacccgaatttgtcctggattagtggatatgatcgattccgaggatccgagggagtcgctttcagacttccggtgtcctttcgcttaatttcgtcctcttttcttctctgggctgggtggtggctgccacatcccaccccttctgccccgacggctgcaaaccgcttcagccaaaacgggcggagaggcggagagatcgagacgctgtcagttcctcggagtcccgtcgctggaggtcacagctgggaaaatcaggtgacattcgagttggacaacgactcagggttgtcctggggctctgcccggcgggttgaagcccggggaagcgactttggaagaaccttcggcttggcggttcaggtttgggaaaagagagctggcggccgcctgtttttgtgcccgcggaccagccgggccgacggcagcgctgcggggggtcgtcagaggtgaagagtcgcacctaccaccgggtggccgaaacccggccgcagctccgggagcccccagccctgtgctgctgcggccggctcagtgctgaattgatgctggaaacggctggccagcgggcctagggtcgccgcctttccctcctcgcctcttccttccttccgggtcgtgccctccaacctgctgtgcgttaccgcagccaagtttccaccgcccggcggagcgcattgtgaacagcagctgacaaattgtcacaccaaaaaaacccaagtctcgtataattcggaagcggtgtctgtggagagcctcccggggtggcacgggggacccggaggccaacccgcctttcgcggagggcgttctgcgcgcaaattggcgaaatgcgggacgagcgcgctagagggtcgtgttgacacatcccatactttaagtgtacacatgacagttgcagacattcaagcccttaaaaggagcagtgggtgcccctcgccgcgtccctgccgcactctagctgctctgggaacacacacgtttgtgcactctaagcggccgaattgtgcgtcgcctgtgcagaccctccgttgtgtggccttgtgtctttaggcagggaacatcttttgtctctccgcaagaagctttcctccaggaaagataaagtaatcgatagggtcttttaaatagctccgcgtttcctgtcgggagaggagtatcagcgcgcgcaccaaatctgctctggtatgtcaccttatctctcgtccccgctgttgtccccaaacgccgcctgtcaaaggagacgccacccgcattaggacctagactggggcccttccgctcggggtcaggcgcagcgccctcggctccgccgcccttaggggtccgggagggtggagagaaggggcgggagccaggatgagggtcctagaacccgaggctggtaggagagcaaactctcactgaaaccgggcgcatgtgggcttccttttaaccacgaaaatagggaacaggctagtagcgaatcctcctttgtccgcgtggaggaaaatcaggtttattcttcagagagggcattggactttaggaagtgcctgccgggtccaagtttaccttccggactgcgacgaggtgaggtttcgccaccgtcctgcctctgtcctctccccaacctgcgtcagctctgactccggcggggccggagtgcagggcagaggggaaagaaggtgccgccaaggctggtccttcagtccgctaggccctgggccccttcagacgtccaagcgctgttatgcaaatctcctcattttttttctgtattcagtcggctgtatccagttttttaaaaataaaaataaaccctctcagctgccttccatgcacaaaccctcaatgacctctataaacatagtcagcctctttccagcctcacaattacccacggggtcttgagaagctgtagggaaatacctcttggcttcaacacagacttgctcccttccgattgcccgggatcactctggattactccaactgctgacggaggaccacccaccaaaacacagcctggccttgggaaagcgtgggttctcatttatcagggtttatttatccaaaataaaaattactgtagaaaagtaaaggagggaaaaacatcttcctcccttaggaggcttttttgactctttaggccttcctattccctgtggtcctttgacattatctcagccttaagtgaatttgattattatcagtaaccaaaggttgctggtggaactagctgtgttttcttatgatggttcatttgcaccgtgtgctatgctggaaccagaagaacatgaactggacagcccaatgcttgaatgcccttgtctctggcctctttggttactctggtgactagttactcagtcctgggtagccctggagatggctgcctctcccactcaggcggggccctcaagcagggctttgttagaaggagccctgggagagtttcttttcttctaacaagaggtgggagatgaggaagtgacaggcatcgccctctccctagatgtgacagtcatcgcctagggacccaggctcttttcctaacagaagctgtactgcccgttcattagaagtgggagaggggaggtgggaccaggtggcactgctctggtctagtgacatacttctgaccttgaggttgtagcactgctagtctacatctagggtatctgtggctgggatattttcttgtctggactaccaggcctgccaacatggtaaaatcttgaagacagaatccagggctggttctgcttgtttttctcatagttactaaagctttgtctattttaatgacccattaaatgggaccctttgctgaattcattatcatgcatccctttttaatgggtcagatcttacagagcaatgcaaccgagtgtaaattctttgagtttggaaacatgcacagtgtaaagaaggagagctgaggccttgattgctgctgttcaggaagagaacatgagtgaaatgtgggtataattttgaacaatccttctgcaatttgggaattttcaaaaaatctttacaaaatacaaggtgaaaatagcataaaattagttttagttattttaatcagtgttttaaacccgtttgtatttttctttttatatcttaacaattggggtattatctgttttcatctttgatctttgagatcttaggaaccctttgtcattttattttgtgctgcaaatatttttgccattctcccgtttgtcttttcaaaacgttcattttagttgaagacacttgataatgtacgaaggggccactcaaacacggtggggttttgattcccagttgggactggtcaagagcaatgtcagggccagctggcccggtggggattccagaggacagaggcctatcccactctaccttgggcttcagagactggggaaacctggttccttgaagcttccatcctgggccccaggctggagggagtgccactggctactcccagggaagaaggaaattgagagcaaatccaacagaaagaagtgaggaagacttgagtttgggctggtgggtggaggggacttggggaggatccagaagaaaggagaacatcagccagggaaatctgatgggcagactcaggctctggagtagggctgcaacctactgcatggaattgtttaatctccctgaacctcagtgttcttatctgtcaaatgggataatacttgtactttgtcaaaagattgtgagaactaaagaagataacattgtaaattcacaagatgaggatctgaagtcagtccaagactgtctcaaacaaatgctggaatccagcctccagcttcaccctttccttctgtccgactggtgggcattggggtagccccctgaggctgggggaagggcagtgaatgaggagtctcatttgtgggaggccctaccccataggagctctttgtgtcagcagagggtttgcagaggttgatttcattcttgaccactcgtggctaactcagggacaatcgaactgctgtgacctgaggggaaagaccccagatactgttaccggctccttcgggaaacctccactctcttcccccctcctttctgtgactaatttctgaaatctctgtaactgagaaggcattggatgaggtttcccactctgggagggccctgggtgccagactggggcaagggggggctgaggctcctgtgcagtcacatgccatctctcaagagcaggagcgcaggcctccagattgggtggcagcagctgtggggtattgggataacctcactgatgactgggtagatagggctctcttgtgttagcagagtccagaaaacagtgtgctccctgatgaaaaggccacggtcactgtcttctgggctctgcctggctctgacagttctcagctgctccataaatcttccatcagttccatctggccaaaaagatgtcatcaccgttttctcctagcgacgaaaagtaaaaggtcattaactcctctctgcctgttcaaaccaaagaagatgatcttcagtttttcagtggaaggggttgggttgtttatgtaagtaatagtttatatttatatgttgacatgaggataaggaggatgattgttttgggtattattaacactctgggcacccacatacttaaaacacagaacattacaatctggaagttaaaaggaacattagtgcttatccagttgttttcgagtttttttttaaagcaatggaacactcatagcaaatgatatgtttatgcaaaatcccaatacatcaaacagaaggactgagcgtagcagaggccccggctccccctgacctctggtggctcctgtttaacttctctgtttttagtacctgtgctggagacggtggatatttccaactgctgagccactttgcttggactgtgtggtctgccggggatctggttcatcttgtttgcaccgtgtggatgctgtcaggggctgtcacccctcctccctcgaaggctgggagcctgttcttaccctctggtctgatgcttttccggcacacagggatagccttgggttttcatgttcttttctcatttcttttcccccataattgcatttcaggaagcgtcgggttagttctttttctttagtttttgtgtctagagcagaccccagtgtgcagcaggaggatccctggacttggttaaaagaaaacctccaaaccttgacctccctgcagctggggccccttcacacttgttttggcctttgtgaactgcgcagaaccagggctgaccctgccgaactgtggatgtgtggccaagacaggtcccagtgtcctgcccgggctgtgcatgtgagggaggcggtgatgcaacgtgatggcattgtgacagggtgagggaaagattgctggctggggcttcaaggggtgattagattacggcggagttgcctgtttggcggagatggaagacttggttctgttcatttcataggcagcggatcccgcagtttcagactggccagagaaatccatttagtagtaggcagaaatgtggagctgagaggctcattccacaatgctcctgagcttttgctttttacttcttcactttttttccttctctttgctttttattgtttttctgtatatatattttacatagttgagatcatatatgcaattttgttctgattttctacctaatttaaaattatacgcatttcctcatatcagcaaatattctcttcaaatttgattttgggagctgaggttcctgtggtggcccagggtggcatacaacttcatggaccctctttggacaaggtctctattgctggtgagctgatgggactctgaggtgggcaagctgtccacccatttgcagcttacaacttggggctcagagatttgtgcggcttatgttttgctttcactggatctttatgatcttattagaagttgagagcacagtgactttcatctgtggttgactaggaaacatgtttaataaagtgacgtgaaactccaaacaaaaataacattattcataataagcgttctctaatcagaatcagagaacagcctctcagaaaccagtcaagagtcctgttcatccccactcctagattgcatgtaggtgacacatgtttctgtttccccttccttctggtttgttctgggcggttcctctctccttttcccacgtatagtaacgccggttctgtaaggaatcacatatcttgggtgctcacaggagaattccttctgaggcccagctcccacccttcctctgggcaccttgcggctctgtcttagggtcctcacgtcgtagctcagcccaagcggctccagtgccggccactctgtcggctctctgcgtctgagagagggtgcctcagaagggatctgagtgcccacagccttcagaagtgcaactaggaagccaggtcactggtggctggggatggcttgtccatcactagcagccagacaatcgaatagcgccctaggacaggaggagcgcctgagccttcacgccatcccgccagccctcatttcttgttggagcataatgactgaggacagcaaagcccagtggcttctcccagatggctgcgagctgccttctggatcccaggggtaactactctgagtggctagtgcctcagaactgaagtcagaaacctggggttatggccacagatcgaccaccctggtgctcagggccagagtaagacctgaccctcccaaactagaggacaataatatactcatgatgataatacccagaggttaaaaaaacaaccaaaccccgaacttagatgatcccatgtatatcatcacgtgtctcaacaaagagcagggagaccaggttcttgctggaagctcagaattttcatggtgcctctagaaaaatcatcccatcccaggccaagctgtgattagaggtgcttttcatcaggtctctttcttccagttgctctccgctctccagctttgttctggcttagactctcagcccaggccacccaggggcagtggccgctgaagggggcagccccgcagctgcttcctccaaatgctctgggattcaaggccgggtgctgggtgattacaccgctgttatctgggattagctattttcactccctgtatttatatcatggaagctgcagcgtgatgctgccagatgccaatgtgggcaaacttcttccttttggacccacctgctgttctgctgacacccggcaaggtggcagttggtggggaccagacctgccaacccctcctcccacttcccacatgtccagagcagtgaactgcgtcattgtgagagactcaggtcagcagcaacaggcgaaggctggaacagacttgggggcgggaggggtggagggctggcagcagtccctgcagaagatgcctgtcacctccctaatcccctgaagtcttcaagtctaggaaccctctcgcctgccccactcctgctccaccccgacccacgtgtatctccctgttgcagtgaggccagaaaaggccaagtgcctgagagtcccaaatgtaagtattaaaaggccctgctgcatttacttgctccatcttcgtttccatctcgccttccatccctgcttgtcagcttgtctttcaggaaatggacaggtttttttgaaggtatgttctatctcactggggctgcataacttacctcagcaaacttcttagagagcagagactgtaggtgggatttgcccccagccccggtgcattgagggaggcttctcccacttgcctaggaaggttgcctgcaggctcactccctttccggcaaaaataggcaatgccaggtgttatcacatcaggaggaggcttcagatgagccccagggattctgcccagaaaaaaacaatatgtttggtgtggccactggcttctcatacccctgtggagctctgtccgtctgggcttctctgccctcatctctcagggcagaaatctcaagggtgacaagttagccctcccaggtatgtttaaaaatcacccagccccgtctatatgggagtgaaagtcacctggggcaagctgagcagagaggccagccagtgaagtggctacccgtggcggtgagagtacttaactcagacttggaataaggcttcactttccagaaacttccacacattcagatcactaagttcagatgagaaagcaaccctttatctctcataatctttgacatggagcccgctccctgcagcccctgggagacagatactcaatctgtgggaagttggcctggagcctggctctggctgctgcgtagacaccattccgtgatgaaggctggcacaggaggcccctgggcagggttcaggcttccagacaggcccacgtgggagtgttttgaatccaacttgtttgccgggttattctcagtgcctgaatgtgctagaagcgaacagaagccaggatgaggcccagaagagcttgagttaataaacctggggaaaaaaatcaggaagtgttgtgaatacatgacatcagagagtgctctgaatgcaactttcaatatatacttttatatcttttctccttttcccacgttctgttaacgtaagatgatagggaatcaaccaggttagggaggcggcagggcccagctaaatgcctgcgtcctctctgggctctagagaggtgctgagtggtgagggggcctgggtggacgctgggcttctcctcagcagggaaggaggcccagggcagaaatagacctgggggaggatgcatgctgtatgagccagatttggccacatatattaggaagacatttctaatcacaaatgtcttaatgaaagaacaaaatgcctcatggagcaaggatgccctttgcagtttaagtggagattggaagttctaggggtgttggtattttcagccagtgtcactttgagggttagtaaatttcctacctttgctgtcagctgtgtgaagtggcatgcctttgctttatctctagaattgttctttaagtgtcgcgagtagtttggttctgttagtctaggctgggagaagagaggagaggggagccccctgggtaggttgaaatccaaagctcaatgttgccttcaagtgagaacatgtaggaactgcagggtcgggagatgggtctctgccttggatggggcatgagtttgggtgtaggattgagatctttactggttcagagatggacctggggcaattagatgacgatcaccagatagttgactttctcgattcattttcgattcagtttttattccattcagtttatttccttacgaagaagaggaagaagagggagagggagtaaaaactgctattcgccagtcttttgctaggtggtttacatacatccatgctaaattttatgataatactcttataactactgttttacaaatgagaaaactgagactcgcgaggtcaagttatttgcccaaagtcacccagctgtcagatgcaggaggtccaaataaagtctgttgggttctaatcctgaccattttgctatagcaggctgatctccgatgaaactgtcaggacgatgcagatcccagcccaggttgattggcaaatcttcggtgactccatggccctgctattctcaccggagctctgcctctcggccccactggaagccctctatgtcagcgccagggcgctttacaagcacgcagtacctcatgcatcctggttcctcaaggcagccctggacagttaaatggcgccccctgtcctgaggctcagtcaagtctcctgccccagcactaaactgtctcccttttaaaaacattaggttaaaaaaccaagaacgtgaagaaaggaaaagcaaacaaaaaatcttcccaatcccaatttccagatagagagaatacattcccacggcaaaacagtcattcaaacagcacagaaagcagaaaaaaaaagtaaactgctgggtttccttcaagaattttcttaagtaatgcatctagttacacacatttagaatccacaaaggggtggcactccaggcgcgtccagcacgttgcttttctcccgggatctttgcaaaaccccattctcgcagagttgtctggtttgaaggtgccccattggttgctccagtttcctagagcactctcagccacgcccccgccaccccagcggcgctgcaggaaccagctgcttacccggcggcctggctgtgcgcggatgctgcagactgctgcccccgtgtggcctggtgcgggggctcctcgcgcgtcccggtaaaccagaacctcagcccatcaactcactcgtggggtctgaggaggagaatctttggttctcaccgtcatggctcacccagtggctgaatcctgccgcatccaggcagctgtggaaactggttgaaaccaggatggtcagctgctaccctgtgtttgggggactatgtgacagtgcctgctgtgtccctattctggtgctaaaaagcaggagctctcttatgctcacatggaactgttggagtcagataaacttggctccaccacttatatgctgagtctgttcatccatcatccaccatccatctattcgttcaccctcccatccatccattcatccgtccgtccactcatccaaccacctgtccatctattcatccaccacccttcaccttgagtgctgagctctatgactttggataagtttgagcctcagtttccacatctgcaaattgaacataatgacacctaaattagttgttggaagacttaacaaaataatgtaggtgaaatacatggtacataggatgctctgtaaatggcatttataatcttgtagaagcttatgtattagttagggaggcctgacgtaggtacatgcaattggagtataaaatattgcaagactgttgtgcacatgtgtgtgcttgcattgtgtgtagcctgtgggctgaagcagtgcaaagtgaggggctagagtagtctttaagaaccaggagtgctagagagaagagggtgtattcggacagggactgctcaagcaaaaatgcctatcagccatggaaccaaacaaaacaggtgggccctgtccctgtctgagattaaaaaacaacgagagactttagccacagattgctaaattcacttagctcaggagtccactgtgaacctagacagaatttgctagattgcttgcaggggcgggttcaaagattctgtacttgctacttgccaatgattattaagtgctggattcctcacatacctataaggtggaattattacactcattctcgaggaagagactgagggtcagagaggataaatgacctgcccagggccacacagacttttctcctatacctcctctacctttagcttcaggtaagaactacctgaagctaattgggtcttccatgttggcccgagcatctgctcttcccagggaggacataagtggccctcttcatccagaatgcttgtcaggactctctctctttaaacggtaaccctgaccacaagcactgacttttcagtctcagagtttattaagaaaggtgcacctgactccagtttcataaaaacatactgcaaattcctctttgtgcatggaaataattacttagttgattgtgcatcaaattatttatgggtacataggggttgatatttctctgattttaaaagtttgtgtcatgtaccgtaagtggctggcctgcgatgtctactggtcaaagcgggcagtctccctctggaggaacaccagggatgcggggtgcacccaggctgtggggtgcaaaggcgggaggggggaagtagctgtgaggtagaccctggctgcgtggggtgggggccacttcctttctacctctc

SEQ ID NO: 38 - DNA, 2693 bp, Homo sapiens
  source 1..2693: mol_type = genomic DNA
ctctttctcatcaggtctcacctcaaatgtcactcctccgagaagccctctgatctctcacatcactcagtctcattttcatcacatcccttatcattacccaatatctttctgttcattcatgtacttccccttgtgagaaggctcagagcagcagcatggtctggggtcccataatcctccaagtccggatccatctccagcggcttccagtccagagttctaggcacagcaccagccagcttcttgaacgagtgttggcatttctttctcagtggctttgaaatcagacgtcagcccccaacctcgtgactcagccggcagcgtgagaaggctggcttggggacacaaagtccttcgatctttagagagatagcagccctcctgggaacggggccacagatgaaggctgagcatgaagcctgggcactggcctgccactggaagctgaccctctgctggaccatgccaactttgacatttcccaaactaggcaatggcccagcttttgaaagccagtatcgcctagctatggaatcgccatgcagatagtgctggaatgaaattaaatttcccttttcagaacaaagacaaacacactcctctcacccatcctacgagagctgggccgggagccctgcctccgtgccaggattcaaagcccacgggctctcctgcttgcttcagcagttcctggcaagtgcaacggagttctcccgctttaaaatctaattgcctgccacgcccgtgaatcccatctccaaaggcaagaaaaaaaaaaattttaaaaaaaagttggtgggggtgggagggggggttctcacacattcagcccagatgagcccatttaagcttttgacattatgccttttggggaggccgattcaaaataaaccagcccgagaagctcctgttttaaacagaaagatccataaatgcagctctgtcagtaatgagaaaatggaaatcacaagcaaaaccagaaaactcattcccccagaatcttaatgatctccagaataattgtgctggtctctcaaatcaaggagtggacggtgacagccctgtgagggtcagatgggttcatgtttttctcatttctaagcaatgtgcttgtcttaatcccttccctgtgcccagcaaaaatgctttctttgtctccctcaaacaaatgcagcttctcaacagggaaatgagcttccctgtcttgcaaacagatttcttatcatagactgatccactgtgggtgaacttgcatttctcttgcataagaaagcaatttcctgaagagagaaaggggggaaaatcaaaccgcttacctctccttttttccatattagcatccttagctgaagctcctgaagatatgggaaagtttatttcagaggatgaatatcaatctttcagatagcagtgggtgtttaggctctaagaggcagccaggttttcctgcatttgtttacaagaaggaacaaattctttgacaatttcaaagtttctccagacaaactgctaccttctgataaaatctaaagttcaacaatgacaaacaaattagaaaattctgccattatagcggaatataaattggcaggttcatttaggaggcttaaaacaaagaatgagatcttaaaataataaaaaataataatgagctttgggattaagaagcaccagtgatttggcagacagtaaccatagcacacagtctgggggggcctcagacctgtcttggccatttcacttctcttgtaggttctggctttcagcttcgttgcaccagagagggtggtgagcccagcacaccagcatgaggatcagcccttgggctgggggtggggtcagaaaaagaggaaagaaggagggaggagaagggaaggaagaaactactgttttaccgttggttattcaatttgaatgcttgttgtaccccactatgtgccagctgaggccagaggccaggagaaaatggtgagcaaagcaggaacccagctcagcccttttggagctcctgggatgactttaaccacgcctgcaagaagactttgtgagtgtgtggatggatgtgacgcccttggaggtatgggggagatggggcctgggaaggggaagagacagattcctggggtctccatggcatgggaagaaaggggggacatgagggatagcattggcagtctccctgggactggtatggtttaaactcactgcaaggccccctctctccagcaggtttgagttgctggtgaaataagtgggtcaagggaagttacgatgtttgggagatgctggcatctgctaccaagagcctaccacctacagccaggcagtacctctgtgctgcccatgtgctggctgcccaaggatgctcaaggctcagcgagccctggacactggcaaggaggccagcactttcctgggtcttggaaggcccagggagacagctagggaagatgcctcagagggtggcatccaggcgtaggatagaacaacacccacagcatttctgcagccaccacagaagtgcaatattggtttctttctggcttctcttgaaaaactagaagattccatggcccttccctcttaccatggcagtgcctttaagaacttctgtccccaaggcccacggcagtggcctctgagggcaaccgtgacttaatttaaatctggagttttaaacaacgttaagt

SEQ ID NO: 39 - DNA, 1861 bp, Homo sapiens
  source 1..1861: mol_type = genomic DNA
ttttatttgtagctggccttatacttggttgagatagggagaaaattctaagtaacatctcaatgccaggtttctgaggtcatctaaactttaattgtccatagacatattgagaagcaaagacttaggaaagttgccaggattgctcctcgactagagtcagtcacttaagtcagctgacagtgttaggacaatgtttccaaacaatgagagaaataagtcaatatagggagtttcctaaagcacagtttattcaatttaaaaacgaagtttatatcttttactttttggaagttaaatgtcctttattttatgacatgatgtgacagtgatgataaaattcttccttggcaaaataaaaataaaatgattcctcctcatctttttctggacatttagggattattgtttgcattcctctctttgtttacaacacccttcccctgacatctcttgctctctttggcatcccaatggcatttactattctccaaacaatttaaaattcaaggtcatgctggaattagtacccagttaccatgcatgctttctgtgataattaccctcaggttttgtaggaaaacagacccccagatgcattcacatatgcaaatatgcactcagtaagcaatgactaagacagatgagtcggcagggattatggagaaatttccagtttatgtgcataattaccagaataactggtggtggccgtgggaaaggaatgggccacttcagatgggggccgggacagctaaggaggaagtgatgtttgaactgagatctgaatgaccaaggggagctgtccatgagactggtggaagagtgttccaggcccagagactgtgcaaaggccctaaggtaggcctttgcagggataagagtgtggagggagggaacaaaatcacaagtgtttactggccttccgtctgttgcactctggggagggctagtgtccctatgcggggagggcattagagaggccctgcttactcaggctagggattagggcagagaaggggaattctctagagacaagaggtaggttgtagttgtcaagggctggaaggaatggggaatgactggtaatggatttctttttgggttgatgaaaatattctggaattagcacaacttcatgaatatattaaaaccactgacttgtgtgtttcaatgtgtgaattttatggtgtgttaattacatttaaataaaagaaatgtgaacaaagaaggaatggtgaattttggagcaatgctaagtgctgtcaggaccagctgctctccccaggtatcctgcctgcattcataaacattttgtgtcatatattttcataattttcattttgtagtggattatggtccactgtgtaccacaattcacaaagagatatctccaagttggtcatttaggaatgtttctttttttgttataggtgtcaacaagggcaatagagctctttcactctgccgcaaatcttggtttttcacaaaccttctctggaatttatattcccacactactgtgaatatatagttaaattgtttgctatttattcttggtgcctttttccttttacatggtctccttcataatcttttctcaactctgccagctctctgcttacctacctcacccacctcatcatgaaaatcaagctcttctgacctcatctgtttccactgctggacttacagcactgttttgtaatgtatttacccacctggctcctttgccagactgtgagccccctgaggtcacacacagtgtcttattaatttctgactccctgttgcctttgcttgcagaaggaaggtgtaaaatatatgttaattggatttaattgaattgctgaggctgaag

SEQ ID NO: 40 - DNA, 2864 bp, Homo sapiens
  source 1..2864: mol_type = genomic DNA
attagacagatacaggcataaatacaaacagatgaagaagagagtctatcctaagccttacaggatcagtacaaatcaaaagcgtcaagaatctggagtgggaagagaatagtgaataacgaagcccttatgaaaaaaaaaaaaacacagacaagaagtgcaatttggaagagaagggtataacacgacaggagtgtgagagggcaaaggcacagagacaaaacggtatgtcctcactgcagggactttgcacttgctgttcccttgactgagatgcttccccctagatatgcatatagctcacccctcactttctccaggtctttactcaacaccaccctctcagtggctaccttatccagtcaatcctaaatttcatattacctcttccactttagattttctcttcagtatttgtcactacctaatctactttatattttaattatttatctttgttattttttctttcttccactagaatgtaaactccctaaagcaggaaattttgtttgtgtttttactggattcccaaaacctagaacggtgcctggcacagagtaagtacccagtaaatatttgtgaaataaataaatcaagaggacaaagggattgaagaagagctattattaaaatatcagttgacagaaatagaaattttacatgggagaacagattcacaaatttggacagtatggagacaatttggagagaagctagaatgtcctgccactcataaataaaaggctcctaggagaatctggggtcactttgtctacctctctcatttctaggcaattccacacctaagccatctcagacaaaacaagatattcagctttccattgcatgtttcttagagttgaggcaaacatcacaaagctcaaatttctagccttgccactccattttcaaaatttacatctttgtggagctactgaactccctcagtgtttcaggtgattgggccaggatactattggaaagcaagggtggtttagtgttgctctgaactcagggagaaacagctccctgaaacctacaagaggccctggtgcattggcattcaccctatggttgtgctcacaagcagaggaaattctaagcagttggagccttaaatgacaagtctacctgttcgtgtgccctatcctaaactctacctgcattcatttctgcatctctcacctgtctatatagatgggaaggagaggtatatatatttcaaatattctatatgaaaaggaatcagattctagccagctgtcactccataccgaaagccacagagcattcacaggtgcagtatccattgcaatgtaagtgcactcctaccagcaggaagcatgcgatttagaaaagaaaatgtgtaaaaggaaatgagaaaaagaagtcagatggggcagaaacgggagacatcaagtcaatcacaaactgcacaaaatattcacttccttaattacttgttgaaagctgaatttgttaagaaacatttactcctacttagtttaagtgtgctagaaccaacaataagctgttgcaccaactagcaaaaacaagcttctgactgcatgctctgattgatgagtgaaaaacattcctaattagcatttaaattcatgagtaaaatagtcaacagactgcaataaaaacaaaataatttaaacaaagcaaattaccctcttttcttgactctctctttaaataaggcaggaagataactttctgtttttaaattatttttattgcattttcacattacatataatacttgcttttccagaacatcacacagttcatatttactttatagcaggaaaggcatagataataaggagccaaagttcagccacaagctagtacggaaactccattaaacaacaacagctctccaaatctcagtttctccctgcagtgtttgcattgcaattatggaaggaaaactgcaggtcctctgataatagctcaggagagaacgctgcacacttcctcctccccaccctcagaagagcgtaggagagtacagccaattcctgattatgtgtggtaaggaggaaggagaaggaaggacaacatggacagcccccaaaccacattttatggctacaagcattgtctcctttaccacagaacttttgccagaactgaacattagaaaccaaactggggcctgcctttcccccctggctgccagtcctttttggtagaggaggttataagcaagtaaagggcccaaaagcccaagggagatggcagggagaatatagcaggaataatccacagaaaccagaaaggcagctactcatttctgagggaatcaagaattagctacattcttgctctctaagcaacaactgtcatgtgatcttgttaaatactgaggtttgatcaatttctaaaaattattttgatttcccaaaacacatatgtataatctgctccatctcaatggttaaaaatgtgtactcaccacccttagtcatctgattctaatttagaacacatttcctggctttcacaaccaaaactattcattgcactgatcataacaattctcctaaaacaggcaataaaaggagcagaacttcctcctgtggccagaaactttcccaaagcttaggccttccagcaattacagccttttcactgcagtaccttaacctattgctaataaagcctctttggccagtcagagttgccatctttagagacagagggcaaaaacaacaaaaaaagtctcagagcggtcatttattcaaactgcccatccgagaagcctctattcttggtaaggaggagactacataaaagtacacatcaagagaattctgagaattttatatcctgaatgattaagaatggtg

SEQ ID NO: 41 - DNA, 4579 bp, Homo sapiens
  source 1..4579: mol_type = genomic DNA
ctgcctttgagtcacccatcccggtgaatgtttcagtggggctgacccatcatagggcacttttggtagaggatgcctgtgtgaagttgggaactggcaaggcgcagaggatccgctgggagatgacaagtccctgttcacactctccacagaaatttggttacttgtttaaactcttaagtaactgaactaaatggattctattacacaatcataattgcttcaaattaaacaataaacgttttactgtttttctacaccctaggtattataaagaaagtgtaaagcatgccagtttaagcgcctggagctaaggttccttttggggagttgtcaaatatgcagatgcgagtggtggttgtttttccctattgtggaagtgtgacaacccaggactgtggccacaaagagatggaggagaacgaggagcgttaagaattggcccaaagtctggtctttattttgccttcataattttttccaaaatgaacaaatgccctgctatagaaatgccaagcattttccaaatgacatttcaggtggagtccattctgcatttctgaaaaagtacttccgaacttcagagcagatgtcagcattttggaccctcctgacatgattaaaggtgcaggtggcagctggcgctggggagggacagggcgggagctggcacaggcaggaggcaacacggttcatcattctcccgttaaggaagttggggaaagaagcataaacttgaaaacacacatttttacttaaaattaggtgtgagcattataacacaaatgatcccaggctgccactgctgatgaccctgagcccatgcccaaatctggattatgtccagggatctcagatcattctctctggattctgggtctcagacagagtgtgcagtagcagtctgtcagggttgggaagaccaagggtcccagcccctggtgtctctgctcacattttcaccctggctgcctccatgggctgtcctatcccctgacagtgctaccggcagggtcctgcctggaacaagggggtagatcccatttcaggagaaacgtggcctctcccctaattgtaggggggaagcagaaaggcaaccacaggccttttgttggccaacagaaaggctgaagtttggttctccctggttgcatttcaaagaagtctaaaaaaaaaggtggccccagtctcatctcaggcctgggctcaaagtgagttgactcactcaccctcccctgtctactcagccatgccatcggagcctcccctcttgaccttgactcgagctgtcatttagaacttatcatcagcacatagctagacttttgattcctccagtcacatattatatatattttttatttgaaaagtgctttttcaggtatgacttggaagagaaatttattgcaggaaagttaggaaaccttccaactttaaacacacgtgcacacacacacacacacatcatgaactttttcaataatagatgattaaagaagtctgtggagagttcttaatgagctccttctatgttgtctaggagcaatgatcgttaaaatgatcggggggattgaattataaatttccagggtgtcccacacggggctggggctgatggccttcatccggcttgttaaaaccagtagttgtgtaagactctgactagttaatttgaaagctcccctttgggatcgatatatttaatttcaagaccttaattcatgagaatttattcgaaacaggcacggggcatgagcagagcaaaaagtgtccaccaggcccgctgcaccaacgctgctcttggctgggcgcgtcatggctacgagaaagcgcatgaccacttccctgtttggtttggtttgtgttgtgtgtcagggcgcaggggtttctgctttcactcaagttaatttatttccttttccttggtaattgtgaaaaaacaaaataaaacctcctgtgagcctttgaacttctggaaaagccctttgctgtgaaccgctgactctgagaaagctttgagcgggctggaaaccatttttctgcaaccttttctttcctggggtatgtctgggtgcacacggctccccacaaggcaaaggctgtccctggatggttggcaaaatgcgccacaccagagtgggtttgtgttggcaggaggcatgagaaaacctgctgatggcaggggaggacggcgacacctgggaacaaatcctccttacctctaattacaaagaggaaaaagtcactgaaaaaaaaagtaaatgtcttaatatgtcatatataatccaaagctaccaccccacttcagggggatttaaagtggtgatttggttccaggtatgcgtcctgccaacctgggtgggtgttcccttacaaaaaaacaaatgatggagagtttttactaggattggtctgatcagggtagacacactgccagtgttcttggctctgactccatcagtggcccccatggatacccagtctccttctggggctcaaatcctaatgcctgtcacgttggatgctgccctagtgggatgggcagactcattctctcctctctgggtctgcttgggccatggaaggcatgcaaagtcctgtgatgtgaggccgattatggctcagaatcttccagacactaactctgcatggctccccgagtgcagaataccatatctttagaatcctgtcttttgtttttctacattagtacagttgcacagccctcaggagaaggtgggaggtgagaaagctcttccatggaagctgtgtgtactacaggaggaccttgaggttaataaatgtcactccacatcagcatggacctctaggtaggagagatctaagtggtcccttggccctctcatttcaatatctacgacatcaccttgaccaccccagaactctcatcttctgaagtctgccttgtctgttgccatagctagctgttgccataggctggggctacacacacacacacacaatttatttctcgtgattctggagcctgggaagtcccaatgaggccccagcagattctgtgtctttgctctctagtgacagccaccaccttcttgttgggtccttacatggtgtaaaggatgaggctcatgactcaatcccctcccaaaggctccccttcctaataccatccccttgggggttgaatgtcagcatttgaagtttggggagcattcagaccatggtgccccatctgttttctcatgggactggcactgtctggggactgtcatatctttctatggaccatgtcctgcccatttttttgttgttccctcaccttctcctctcaacctcataagacctggtccagtcttctgcatacccacttctagaaagcatttaaggtgattccaagataaagaaaatagagtgagatgagagaaataattttcagattaaataaattctagttctttcagatctgagaacctaaaagttgatgcctacaagatgccttccacatagaaatacacacatgagaaggtccagggtctggcactgcacacttggttatatggagatgcttgtttttctccagtttcatacatgcatcttttttcactcgctggattatttgcacttagaggaggtgtcccgagctacatttgcatccatcataaggcataccaaaacgggcctacaggaggtgtgcaatccaacctttggcctgatggaagccctgccttgaaactgaaagttgcccagaaaggttaggaaatgctataatgttttcattactcattcccttgctgcatgcccttaagtcttttagaatgtttgtattattttatggaatatccaatttctgcaccagttagggatcagattacaagctaactctggtgagtttataaagaaagaatttaatgggaagatgtgaggtgcctgacagcatctgtgggaggtgttgagagccacactctggaaataagcagtaggggagcaagttcactctgcagggacggtcccgttgtgggtgtttatttccataatcgggtccttgtcgtagccatcagcactctctcaaccgtggtggggctcaccccttcaagagttaaacctggggtttcagtcaatgccttaagaagcaaacttagtttttcctctcacataaactgtccaattctattataggcagccacctcatcctagaatccctgaatcctgcttctgtggcagactcagcccagtcccccacctgccctcaataagcacttcaatcctcaggtcataatttagctagggctttagattcctcctcagccagataacagtccagctttcccctggagttggttatgggcagcccactcacagttagcaatgtctcttcgctagattgtaaactgtggaggagcttcgtcttattcattagtgtggggccattggctagaatattgtgagcatgcaataggtatttgatcaatgtgtgaataaatgatttgaatagcattctgcacagaactatacttaggattatttactggaattctaaccccaggaactggctgatggtgagaatattaaatgaaaccaaacttcagcaaggtggaag

SEQ ID NO: 42 - DNA, 2397 bp, Homo sapiens
  source 1..2397: mol_type = genomic DNA
ctgggatgtaggtctgagcttttggtgcttccatctctctgtcaccctcagacatattctccttccagttcacagctccattttctcgcctgcatttctagctaagctctgagtcatcgatctcaacctttaattcaagtttgtgcctggatctgaaccaaaccatagatagatactcataaggttgtgcagatgagaatgtgtggttgttctgcttttgatcagcagcaggctgagattgcttccaggataacgggagccaagtctattttcttaggttacaatgaaggactcaagtcctgcctggagctggctgagtgatgaactaaattggagaaatgtgccatgtggctcgccgtgttgattgagttcacttgagtcccctttttgcagccaagtgcattcgtaacttccatagcatggtactataaatctgctgactatttctgtggtggttactattccagtttgaagagaggggtgtggagaaacagtagtgaaagcaggaaaaatttagttgtatgtgcaacacacataattgcaagatgtttattattccattaggcagtgctggtctacagttaggagtggtagaagaggagaaaaggtcatgacaaggagaaccctactatttgccagacgctaggccctgccctgtgagcctcttggcagcccaagttgggaactacttatttattttactgttttaggtgaagcttctggagaagaaactgtttgctcagagcccaacattgaatgtgtcaaatttggtgtttggactcagcagttcaatgtcttaactattaattattgcctttgccaagatgctcaataatgtatgccttgccaagtgattttgaggattaaatgcaatgttgtaggtgaacacttagcttaataagagcctcgtaagtaaggatggctgctattaatgtaaaaataataataatgaggaggagaaagtggaggagatggagaagaagaagaagtagggaggaaaagaaggggaggagtagaagtggggaggaggagaaagaggagacaagttattgccatcaccacaatacgtgccagggctatcttgggcactttacgtacattatgtaatttaatgctcacagcaagcctagccaatactcacccgccctttctgcctctaattttttctggtatgacgtagcaaagtccgcctgacacttgaatatttacttccacttcacgactcttcatcgtggtctgggtgcgtaggaagcttcagatggcaagggaggtgctggcagggactggcagcaaatgagcaaacgagatgagagggtgggacagagcctgcgtttttggctgccctaattttagcttctgattcacccaaggaaattttgagcagtgtccccatccagaaactgcgaatactaaggaaggtctgaccagcggctgaagatatcagtgagggtgggaaagaaaaaagaagaacgacaatgatacctttaataaggatctaattttaaactaattaaagccaattggaatggaaggtatttttctttaaggaattctagaaactgatttcatgcaattggccttgccgcatacgttaattagactaaagaatgaagcgggtactttaaatcaaaaagccttgagagatgtgaggaaggtggtgtgtgtctaccagggtcctggaaacttccaataatagaatttatatccgatcctggtcagacacacaagggcacaaagttagtaagtaaaccatccctactactctatgctttattaacccaaagcacagagcttctcactcaaagaccaagacagatccccatcctccaaaggtggtggatagcaagggacaacgttttccgacttcttgccaggcccaggtctagctggatttgaatcttcatgctaaccctctgaagtattttttaccctcaatgtataggtgagattgaattattctgccaggtcatttaattacaagttggcagagtgcgaatcctaaattacgttgatctgactcccacatccttgctgatctaccacgtcatgtggccttctctttcaattccacatcatgactagagctttctccacctacaccggtctggctgctgggacctcccagggtacagagctgattcacttcaaccggcatccaggggcgtgccttggaaagccatggaggggtagtaggaaattgctcttttgttaaatgcaacttgggtttatttggaaagaaaaagaaaaaaagaaaaaaaaacgttctgaaaaagaatcaagttgctccatgtaatctgtaaattgaaattagatggttgtcaataattcttactatatatataaaattccatatatatgcatatatgtatttgcttatatagatatatagtttgggtt

SEQ ID NO: 43 - DNA, 5727 bp, Homo sapiens
  source 1..5727: mol_type = genomic DNA
ctcattctgattttggggtctgggtgggcttgagcatgggtgtctctggctctggactctccaatagctggcttgggctcagcttgtcaccacctctccctgtggctcccgttctgggcacagtgaaaggcaggggtttgcagactggcaagcacacgatcaactgtcaacctaggagctgcaggcagtgggaggagggacatcgggctctagctttggcaccgcgtgaccttgggcaagtcacttcccctctctgggtttcaggctcctcttccgtcgaaaggggcaatgtctgcctcggtccttgaagatggcgtgaacgtggcctttgaaaccagtgcagagccgctggtaggaagcttcctgaggaaggacagcaaaggggtttggaatgagagctcaaagtatcagcctgcgtacaagatgccctcggggggagccttgtgggcggtcacactgggccaggcccctgctgaggcccagcactgcacaggcacccggagccctgcccttctccctcaggcaccccagggcagtaccgccctctggcccagctgagggccaggctcccctggcttcccaagtggaggaggaggaaatggtgccagcaggccaccaccctcgaggaggtggggtcttctttggggctgtgggctaggagtggacggaagggctcagaggccaagctcagatgttccctatggctgaggtccctgacaaggatcctcaaacagggagaatgctggtacccttccagtttgggcctgggtgtcacttacctgggacacttgtcacatcccctttaaatctctgtccttgcctggctctgggattctgaagagcagtagggtttccagggttctctcctgaagctcgcagtggcttcagggtaaaacagctggtaaaaagccatgggtctctcccttaaacccaacccagcagatgtctactctggaggcccgaaaacaccttaaactcatcgcacagaaaaccgccaggcaataattcaatgttccagcgatgtcctttgtgacccattcatttcagttgatttggttctattgcgatccctttttttcatttgttcattcattcattcattcattcattcattcattccagacacattacagccacagaggtgaggaggattcaggtcctgcccgcagagacagttgcacatacagccagccacacaaggggcaagaagtagtctgtgctgggccacaggggcagctctcaggaggctctcgcccacgctgtgctacattcatcacaaatctgacagtatgcaacaaaaagcaggccacaaaataatggatggtattaacagcagagtcaagatgtgggaaaatggaataaaatgagctctccaggtaccatctatttttagaaaatggagaagcttgatggataaatgaagtaggccagacagggagaatacaaatagcagcagctcctgtgggaagcctgcccacatcatccgaagttggtacgattcccattttacagaagaaaccatgacaggaatgatgtggctggcttacagtcacagagctgaactcagtctgtctcactccagtttctgcttggaaaagattcctcatttccaccagcttccttttaatcaaggcagattagacattcatgcattggctaatctaggctttgagtgtagatgtagtgcctacaaagcaagattagccattacacgttttgtcgttctggaaactgactcttggcaattcccagcggctttatctcaacatacttcctgaaatctcaccatgtgtattttcctacagagtggctctgaaaataaagtaggctggaagtcaggtgactgatgtcatcctggactcgtaggcgattaacagtatgaccttggagaagtcaccttcctcgcaagaattaggactagaagagctgggcattcagaagcatgcccaagtgctcatggcagtgccgcctgtgagcatgagaaacgggaggccacctgtgagaatggagaataacccggtgaccatatgtgtctccaagtcaatccctgtgatctccacctcctggcatccccccagttcctcttccacactgaaccagggaagatctgcttgaccaacagcatatggcaggaatggcatggcactcccgggcttagtaaaacctaagccgcttccatcttgttttctctctccctcttgaatcccttgccctggggaagctgcctgtgagcagccctgtagagaggcccacgtggaaggaattgagtccttgccaacagccatgggcgttagccatcttatctgtggatcctttaaccccaatcaagctttcagatgaccacagccctgccagctcagctgcaaccccacgacaggccctgaccagaaccaccatgaggggctgtttcagggctcctgatctcagaatctgtgtgaaataataaattgtttttgttttaaagctgctaaatttggggggtaatttgttatccagcaatggataacaaatacaccccatactatagattatggagcaaagagtggaacaggtctatgtgtactgatacagaaagatttctgagatatgtgttaagacacccttagaatgtgttgttagaaaataataattacaaaatatcttttatgtaaaaaccacaactcactatatttatgcatttacatatgcaagtagatatgtagcaaattacccatcatgtagcaatgatgaacatcaatttaccctcagtaagagaaggaaggtgcaaagtggaattttcattttctattcaatagattatatatataatatatatataattttaccatgataatgttgtattacttatactcaatttaaaaattctttaaacgaaggcatttgagtacttgatcactgaggtctctttcaactctggtgttttatagttctattaaaatgtcattggaatgtaaattaaaagaatgggataacatttatcacgggagggcagggctggcctttgaatccagttatattgactccaatctagggcccttcctgttattttacctttgtgcctatagtttacatgcaggtgtctgtgttagaggagcttgtctctggcctctgttttgctgaggacaatttctcatttcccattacttataagagacaggctctgtgagagcctccaatttgccagctgcttccacttcaccacctctgagctccgggtccagcccaactaacttgactgccgagggctttgctccgtcttgggatggctggggcctccctcacctgacgggtgttcagctgtgatgattgagcttcgctgtcatgaaacttccccagagttggtgccacgcctccccctccctccagcttgccacaccactcgggctgaaggatctgcctgcccaaccctgggttggtctgcataaagacggccgggaaagatagcatttatacacagtaaaatgcctctgcattatctcgcttggagaaagaaataactgatactggcctgggagctaagaagaggggttctctggctttgcggagaaacctgtgctcccctctccattctgacggaatggtctgggggcttgggcatgggtattgtctgaaagtgcctcagatggctttaataaacagccagggttgagaacatctgtattggagggaatgtgaaaaggacattgaggtaaatggatgggattaattatgtttgctaaatcaggaaactctctagagagggaagtgacacgcccaccgttttgcctactgttggaaaattaaagtttcatcttaacaagggcggaagagctttggggtggtcccaagaggaagaccatggggtagtgaagcggaggagtctgtcgcaagggctcactatgggtttttgaaaagctgtgatcttccgtaggaatctccacctctttgtggggggacagtctcattatactctcttgggcagaagcaagctcccatccctgcagctattgaagcagagcatgtataacatactgacagggacgtggtgaaggagatccctgaattggatagacaaaacgccctttaacggcccttccaaccttgaggttttatgattctataattctatgtattgtaaagaacttaacaaaaaaattacttccatcctattcaaattatagtgacctgccttttagcttgcccaagggaatggacttaaaactgctgtgcactcagaatagatgatgtgattcccacactctcattccaggtcagttctcctaagtatgggagtaattatgatggaggagatgcaccgcgtgctgactggagagttagctgcattcgtctctctagagcaggcatgatggaccagctaaaaacagagtgtcagtttgacacatactctttttgtttgctgatgactttttgaattttttgttactaagctcacattttccttgttgactctgggttttgttctctttagagaattttatttagttcttatgtcttagaagtagctcactaaaggacatagcaagaggaagaagagatgtcttatatgattctggctccaaacgtaccacagatctctgactgatggcaggacacccaattgaatcgtggttactccatgtgtcctggaataaaacacaggaaataatttcttgacctcatgtggcatctgaacatactgagattaaaatgagaaactattgataccatccttggctgtttctaaatagatgttaaactctgatacaggatccataatctataggattctttagccagaatagaatgagaaccttcagcaagatggaaagaatcctagcctgagtacaggtactacaacagcaactgacatttagccctagtttagacctccacttagactcaccatgaagttaaaatatcatgattcttataagaaatagttacttgtgtttaccaaacaggtgggtagatgcagggtaagttgatctactgtaaagtcccgggagaaagtagcctctaggaaatgtgccacaaacacctggataaaatgtaaaaaagtaaacaatagccaagtgcaagcaagaaaagaaaattaccagacaccagaaacaaacaggggataaacagctacagtggagagtgaatgagttggcctactcttggggtaattgtggggagagattgatggtggcgatggggtggtggtcatgatggcatcagtttcagcaaccagggcttgggccttcatgctcacataaggtcaggaaatgagacctggagaaggaacggagacagaaggagactctgcacaccctcagggaaagtagagattcggggaaaacccgtccactggcacagcaagatacaaaaagcatgtctgtttctgcttgggcacagagtgaatcagggctttcatatagattacatgtgaatttatactatccagaagtccggaaactaccaaaccaaagcccttaaatacaaggggtcctaggctgctgatacttttgatttgcctagagaaatacagcaacactttagagaaactattctataatctaggatgaaagggtttcactgaatacacaagctacactaaagatgagaaaaatatccactaagtacaagagtctgatgatataattaacaggactaaagttccaggaaattgggatatagccagataaagaaaaagaacttgcacataaacaacctatgtacaagatttctgcagtattgcatgttaaa

SEQ ID NO: 44 - DNA, 7301 bp, Homo sapiens
  source 1..7301: mol_type = genomic DNA
ttgattcaaactctgcttttaagctaaattgaacactaaaagatggctgggaaccaatttggacagccacagcagctttgggagataagaacctttctttgcgatttggataaagcaaacatgatctctctgcctcctttgtttttagaaattaatagagcttttcaacagaccgccccttgccactaatagtgaacagtcaaatcatggcttttattcccagctcactacgtcaagtaaatagaagcttacaggatagaccagggaacgataagcaattctcactgcagcgaattcttaatatataaattcatccagcagcagtttagtatctactggcaaaaccagaaacaatggggaacaggcaggcctggctctgacgaggtcaaccatcaaggttatctatattcaattactggtatctacaaacttgcatagctgtcagctggtcagggcccaaatgaaaactcaccatgcactcagaaagcaaatatagtttccgaggagagcctcaaattagctctgtgaatgacttgacttcaaacgcacaccaagccatcagcctttggggacgtttctgccagcagtgcctcaatgtctaaatttgggcatcatcttataaatctctgttcctgaagtaggaaaggactggaattgctccttaacccttggcgatgataaattatatcacttaacaatagataaacttaagacacttttcctgtactgcggacctattgtgaggtaactgctaatctctggcatctcttctctgtggtcggtaccactaaagacctgtcggtggtgaagagacacaataatttgttttctaactcctcctccggaaccatatatcatgtgtggaaacaaagcttcccaacaacagatatgtatctgtttcctttagagttgcccttcccatcagactcagagaggacatgttttcagtgtgacacttccctgccccagaaagggggacatttccttcccaggccccactggaagtggggtaaaggttaatcacacccccattcttcctggcaggtgcctttcaaccaggaccttttgggggcccaatggagatttactctgcataagtcagggttaatcaatgccaaggtaatttctagattagcttggggaggccataaattgtggacctgttaagagctagatgtctttgattttctccttgggtcagctgaattgttactctctcctcatccattccctgtcaccttcttgataatccctttccaggaccccccttccaattctgcttgaggctagtatgccagccatgggctcactctatttgagccacgaaatgaaagactgtacattcatcaatgcaatctgtttagattgcttcaggacagaggtaaaaccaagagtatcttcacaagaaaccgtttgacatgagttcatatgcggagggaaaggcacatggagcaaggtcagtcctgactttcaaccaggccagtctgggcccctgggtccacccagacagagccgtcatatgtccagctgtgcaggttgtgcactgcacaaggccatatttaaggggtgccattcacatcatagcttttgtagatttctatatttattatgacaactttctggacaatgacaatatcttgagactgggaacattttctaatcaacacaaagccaccatggaggctagggcagtcccgcaaaggaacccacttcttatccctaaagtcaactggtcactttctagatcccctcctacatgtcggggagggttattgtaatgagatgatctattcgtgagtctgtctcctcctttgggatctccttgtccttgtttgtatcccagcatagagcctggctcgtgaaagtgcctgatccatggttggctggtcgtttccatcctgcagggcatgtcacaggtctccaggaggggtgcttgccaagggcataagcagccgttaaagaaaggcagctgttccctcttttcattttccgtttttgtccgccctcgtccaaaacaccaccctctattctgtttttcttcatgaatcccacgtggacaagagaatagattactgaaggcacacaacaatccttcctttcttctgtcttaaagcaaaatttcaaaaacatgtcttggtttgtttagaagcagcaagatgactgctactttatttttgatgctgcacacaccgaggcagaaaaacaaaacagtggactcatcagccagagccggtatttatgtgtaagtgatatgagtaactcaataggtttcagggctgctgccattaagaaaataacactaatacaagacacatgtgggtgctggccaggacatgtccccagagctcatgggcagcccaagctggggatcaaggctgagagctcaccctagctcacaacctttgctgtgatcccaggtcagaacctggtaatgtaatttggggaatccatgtgttcccagtgacccatcaggaggttttgttttgatctattagacgtggaatccttgatctttaaagtttgagatctttaggttttgcctgccctttttcagaggagtagcccagagaagttaagtgatttacacagtgagcagcgtcaaaaccagcactttgatattaatagctatgttgcagtcagtgttgccaaagaaataaggtgaaccacacaccacttctacttagcaccatgcttggtttgggtaccattctgagaaattgttatgagaatgtattccaatgatattatttgggattcctcaactttgatggctgcttcctttgtgagccatttctcttttattttgctcatgttttgagtctcagcttgaaggtcaccagtcaggagacccagtccaaagttaccttctccccaccacatcctagcctgtcacctttcttgtttccttcccagtaatgatggtggttattcatctgtttacttatttattgtctttttgttgaattcccagccagtcatacagtaggcactcagtgttgaatatttgcatgtatgaatgaattccactgtggattcaatttcttccctaaaacctccatgctttgtcttttacacacttatggcactagcattctgcctgccccttctaccagattaaaggcctttgaaggcatctgtgtttgcacctgcagcagtgcttagaatcacatcctgcccacgttggttctaaaattgatacatagatcaagatttcggagcttttccataaaaaaaccctgggctaaaggaaggcacatttaaacgcagagttcccatcacaactaattaatgttttggtctctccctttctcctggacttgaagctgggcacagccaaaatatggtgttaaacaaagatatcttggaccaaattcttgtttgtgtctcctcaacagctacttccagctggctttgtttaagtgatcatgggataaaatttcatcacaatcaccagtgaacatgacgaaagactgcttcatctatcaactccagggctggtgttcacaatcccttagcccaaaatgggacataagacagggagggagccatcacccaccctcaggcagccaggttacaacgtgggcaagactgccatgtagaggagtgagcagtgtcgttggaaccagatagatctgcgttctaatcacaggtcttttaacgggctggttggatatcagttttctcatttggaaaatgggtataatacatatcttacatatttgcacaaaagcatggtatagtttagacaaaggaagctttccaagcagctgagagaacttaagagtgtaaccaaaagagagcgtatgttctgggctaatctattgaaaggacaggaccttttcccactctggatgttgttagcatgtttcaaaagtcaagtggtgaaacttcccagccacgccctcccacgccgtccatggcaaccggaactgagcaaagccaagcaaacacatccttaacctcctcccaacccaaaaccatgagatctgcaattgcaagggcctcgccttggctagaaaccagcccggcatcatgatccaggcctcggaggctaaacccctggggggaaaaaatcctgccctttacaagttcgttctctcaattttattaaccagaaggctgagagctgactgtgggtttgcaataccacttaccagctggtggaatcagagagcaaagccctttctcctggaaccagcctgaaggccacgccaagccaccagatgggcagtaaaatcttttaaacagctgctgtctatggcatgccactacttgtgtgtaagttgattttatagggattgtagacaaacagagcccttcagactaaaaatttacaaaagctggttcatcttcctagcatcttaggattgtagctgctccctggatttagcttggggaaccctaggctcttatactctggggccagaatgaggaagtgttggtagtaagatatgacaaccgtgtaagtaggcaatatttgatccattcaatagatgggtatttaggcttagaggtgttaaggatcagaattacacacctggaaattggtagagaaatttgaatcaaggttacctttgggtatatttccctacttgagtaactccaataaagaaaagggtctatggctcagtgctcagtaacacatggctacacagcttcttctgactccacattccatgtgctgcctcaatttccccagctataaaatgaggatgctaagattatcccttcattgggttgtgttgagaagtgataagataataaatgcaaaatgcttagcgtgaagcttgacctatagttaagaatccaataaacgtggctcttcttattgtttgtcttattaccattatctataaaacgatatagggcatccctttgttctgttcccttaagaccatttcagtcatgtctgtactgggaagaaaacctcgctttgtttttagaagccaaactagtcccctctttctttcaccatattggtagaaagttcataacattcaaaccagatcctgtttaacctttgcccaacatagctttacctacttcaaacccctttggctctctgaggcctggcttccttcaggctcaggctgtaaatgtttgactttgatttgagatacatgacaataaattaggggacatttatcaaagctgctttttgcataaattactgcattatacaaatacacttagcaggatcctgcttctttgatgttgggaccaaagatttatgtgcaatggagtttttatttatctgccctgttcacctctacaagagctggacatattcctcaattagttccatacttctcgaaatgacatgtatctcttgcaacttgagtagagggaacaaagacacttaaatgccttccgagtggctgttaggagaggagaagctttatctgacgagagtgtctctgagagcggattgaaaggcattcaaacctgttgctctggaatcctcccccaccaaaaagaaaaggaggaaggatttattctttaacattatttatatcattaccacattattaagagagccacagaaacataagtgtctattttcaacagcaagtaggttagcaatacagagatctccttgtcatggcttctggtttctgaatttcatggagtaaggtttgtgtgacctggggaatgggcaagatttttaggttcattggccattaaatttaccttttccagaatgtcatatagaagatatatgtagctttttcagattggcttctttcattttgccttgtctttttgaagcttgctaattcatttatttttatactgaaaacaataaaactgtggttgaacgtaccacaattcacctattgaaggacattggttgctttcagttttcggtaattatgaataaagctacttaaacattcgcatgcaggtttttgtgtatgtgtcttcaagtcagttgggaaaatccctaggggttgaattgctggatcatatagtcagactaggtttaacctaccaaaatgtcttccaaagggattgcacctttgcattcccaccagcaatgaataagagttcctgttgctgcataaccagccgttggtatcatcagtttttggatttactcctcctcatagatgtgtagtggtgtctcattgttattttaatttgcaattccataatgacacatgatgccaagtattctttcttacccctatttgccatctgcatctctttttttagtatatttgttcaaaccttttgctcatttttaaattgagtcattatgctttctacggttcaattttaagagtgctttgtgtatttttaataaattattttttctgggccagctccactcatgttgtccctacctccctgcatctgaagagtttagattcatgttcccactcagagaattaaaaaggggagctccccagctgtgaccacaacttagctgcctaagagtcctggatgagccgaaaaagctcagtcaggagcccacccttttggacatccccaggaccccagtaaacttcagaaattgcccatgtacccatcccagagctgccccagcaacaatttcaaagcaatggctatatgttttaagctgcaaggtccttgaagagctttgttcattttacaaatgaagttcagctcatagatgttactaggtgttgcaccagaaaaaggcaaacagattttgatatatttaggattaatacagttaaactcttcaactccttgctattcaaaatgcaaaatcagcatcacattggagcatacaaactcttagcccccagttcatacctgttgaatcagaacctgcatttgaataagtcccccctccacagtttatttgaatgttcataggaactttgtaagtcaatggtttctcaaatcattatcgagcatctactacatggcagacattgtgccgaggattcatcacaagacatcatacctgatctcatggagttagtcttgagtggacaccaacatccacaaattacttctcaagtaactggttccttagctacatttgtgatttataccataatgaaagagtatttgagaatcagagacctggagggtactattaccaagagttaaataaaggaaggtatgccttcatgctgaagacttcatctagcaatgtgacaataacacgtgtcactgtcagcccaggccctgctggcaacaccttgaaaactatgcagaggtttagaaaataaacagcagtctctctgattccagtgataaaataccagtgtaaggagagcctcctgagatccataatctggaattgtcaggggataagaattgtc

SEQ ID NO: 45 - DNA, 2299 bp, Homo sapiens
  source 1..2299: mol_type = genomic DNA
ctggctttcagtttgtcctttgaaaaaagccgcctttgaatttgttcttccaaagcctgccctatgctcggcctccagaaggatggacacttgatcccatccatggctggccaagggcgtggccttcggagcagctgcaaggatagtgttgactccagatggaaagtgtctggagaattctgacctgacatcacaccatcacgaactgcacaggcagtcagtgatctgggggtgccctgaagctgttcccagcctggcttcttgtcaaaagctggctttgagttggagtccagaacaaaagcgtggcccagggagaggccagcttctctgagagcagcctggaggcaaacgggcgtccttcatatattccctgtgatgtgagcccagccctgcactcacggtactggctgcaagcggggcccggtatggcctctcttgactctcctgcccaccctggggccttgccatctctcccctccccgctgggcctccactcccaaaggttctaagtccccacatgtggcctcatgcagctcagcccctgccctgctgagccctacttagccaggtccagaataaaaatctcaatgcaaaaggagacaatagaattggggcgaagagaagctcttggaaaggaagggaagtggggtagccttgccagacctgacccccagcatgcctggcccccactcctgggtgactgggtctgaggggtctatcataaacaagttccttgagggccagacccttccctttccccagagatcaatgaacctgccactgggtgccagacctgggctctggattcctttgtgaaggtctctggaaaaagatctgcatctaacaaaaggaaagaagacaaagtgcagggaaattgctttcaaaggaagtattctcaccactgtcatttaatctggaaggaaaaccttttccagaacctctccaccagaattcccctatcttggccaaactgtgtcacaactggtaagagcaaatgagattaccccttcccatgggagggattgtttaggacaatcctgattcatgatgggggagccaccttctctgagcacactgcagctggatcatgaacaaagacaaattctgttatcaaaatcataggaagcaatgattgttggtttggccaagagcagacattaacaatcaatcttaactctctcacagtcattacaaatcaaaaagttggcacatgagataaagcatatgtattatctcagtcttagataatgggaagtaaggaagccgtaattcaaggcatctttagtgacccagttatcgtgtcattgtctcatggagtgcttttaagtccacaaattctttgtcctccctacaaaaggtgagctaattcccctggctctagtgattcatttctaaggaatagaaagtagcagaatgatactgtatgagttttgagagtcctaaaaggcaggatagctttcaccctgctctttcttggaccacttgctgtgagggagccagccaccatgccatgaggaagctccagcagtcctatggaggaggaactgagcagttcgccattttggaagtggatttccagccccaggcaagccttctgatgactgtagcactggctgacatcttgactgcaccccatgagacaccccgcgccagaacccccagctaaaccacttgcaaattcccttcccacagaaactcccagatgataaatgtttgttgtttgagccaccaaattttgggtaatttgtgcagccagagctcacctaggggaggcaaatggcacaacataaggagatgctcccttcagggctatgacagttcagggtcacacatgaccctgagacggaatgtcttcttaaattctgttccctgctcctcgcttgcctcagccccgcccctgtgtctagcagcagatatacctcttctgaacattctccagcttatgttgggaatctgaagaaattggcttcccaaggggagcatgtgactacgccagtctccattaccctttggtcagagtgattggatcagtgtggcatgtggcccagtcctgtccaatcagagtaggtggtgaggctttagcaaggacaaatatgaacccacttttctgatggaaactgacttctggcagcctttttgggacctccaaggggagccagttcagggataaaactgatatcaggaaacagggaaactgggtccttggtgacattgttcagctgttagattaacctgaagccagcctcatctctggacttttcacccagcaaggtaaaaatttcccttttaaaatttaaac

SEQ ID NO: 46 - DNA, 2685 bp, Homo sapiens
  source 1..2685: mol_type = genomic DNA
ctggctgtgttcttacaaggagagagaaacccaatacacacacacacacatacacacgcacacacacacacacacacacacgcccacaggaggtgggggtagggaaaggaacatctatgacacagcaaggggaaaatacacccaagcttccctagctgttgccaggggaaataccttagagagagaagcagctggaagatcattagactctccatgttttgtcctttcggaaatttaaactctgatatattacccccgaatgcagtctttcaccattgttgtccttttggtagagatcagtttttcttacttcttgggttatctccgtgtggtggaagattcagcactagcttaattagtgtcaggtttcttacaggaatcataaaagaaaaacaatggcataagaacagggaggtggtggaaccattaattctgatgtcattgtttggtttgtttgcaaagtttgaaataaacatgtagatggtttttggagctgaactgagtataatctttgcacgtgtggtgtgaccggagctcccatctcatctgctttttcttccttgcctgtctattaatttacatccatttgccttttactttctggagcttgtgttcccatacttccaaagcagattcaggctaggcatagcatcccttagaataatgccacccaaaagatctccaacctgtgaatatgtttctttacatgcaaaagagacttgcagatgtgaggaaggatcctgaggtagggagttatcttgaactactgggtgggttcaataatcatagggtctttataaatgagggaggaaggcagaggattggaggagatttgatgacctcagcataggtcaggaagctttagaagctgggaaggcaaggaaacattatactctagagcctccagagggatgcaatcctgcgacattgatcttagatcaaagaaattcgttttggatttcgacctccagaaatataaaatatccaaccttgtgatgctttctagaggtaataacataaaattgtagatattttttgggggatttactttatcatttttaaataaaatacttgcaaagagccccacctcttaagtagccttattactcttacatttttatattaacaatttcatcatgaacttcttataaataatgcagacagattctaggataggctcctttataccaattctctgttgaattcaaattaatatgacattgctgcatgcattctacaggggacatttggctcatgtcaatatttgcttgaagatcagaataaggtgtgttattttgcaggaatattggacctccccatctttgaagagctcttccttatcccattgctggatgtagtagagattcagaagcatagaactgcatgttgacttgttcatgcagagtgtcccctgagggtttggattttagtggctactatgggctgcagcaatgacattccctgttgcaacccctgcatgcaagaggatgtgaaggtcaagtgattggtcctgattaaagtatgtatcattggcttgttccggctggcccctggctgctcctggggggagggattgccttgttgctgcacatggtaagaaagcccagctggggtcctgtctagagccacctgatctgtcctaggtagatgacatttgatgaaaaaaaaatggctcttttaaataagacagtacacttgccaatagttaagtctccaaagatgatagatccatgatgttttaccatacaagcactgtgactcctttcagaggatacaaatagcctttttgaggttttctaactaactaaaaggtttctctgtctagtatgacagttcttttgattcccctccctaagggtcagaagttcttctctcccttacgtgagtgtcatgaagcccctcctccccgctgatcacaaagcaatctcaggaagggtctgttgaacttgtgttgaaaggaaagcctgtcattttgatgtgttagaaaactccttcttgcaatgcacttatcaaagagcatggactaatttttaaattctatgtgtggttttttctctgtaacaattttgtggctttgataggatgtagaacaacgcaacctgggaaagagatggtcaacaggattgggactgagtgagcaaatgcttagaccctgctaccttgcctctttattatgtctcttttttccctccctttcatggtgaggggatgcctaggctaattgtccattgataggcttaaagggagcccattacctgaacctgtctgcctttcggctttcagagcctttctgctcctaatgtatcttatatgactcctttgtccttagaagagaataacttcccttgtgtgttccttattcaagctcaaggactacaaaggacacatctcttctattcttctttaaactgtttcagggcccccattctgtataaatttggaattgtgccagttaggagcccaaactgtagatggatcctggctgagatgcttgttacctaactccaggggtcactgttcaaacaaaatagatgtgggaccccctgagtttgtggaacatagagttcctagactcaacttgggaccttgataccagaaactatggctaggcgaaatcacaaactcctagttgctttctcatatatacccaagagaggttgcaaaattaaaaaatatgaaggaaaa

SEQ ID NO: 47 - DNA, 4757 bp, Homo sapiens
  source 1..4757: mol_type = genomic DNA
gcatcgtcttatagctatagtcaagtacaatttaaacttaatagtacaattataatattcaacaactctagaatttttaatttgaaattcaaagatacaataatatgcttgtggcaattaattttgtttttaaaaatatttataagccagtaattcagtactgtatatataccaactgcaatgtgtacatgtgttcacatacctgtgcaaaagtattcataacagcaccaagttccaaacaacccataaataaattataagttatacaatggaatgctatacagcacaagactaaacaaaatctcacaaacacagtgttaagagtaggaagccagacacaagtgagtatgcattgtgtgatttatttatgcgttcagaaagagacaaaattcatctatggcattagaagtcatgatagtggctatccgctggttgggagtggagggacttaagggccacaggataatctgagattctgctaattttctactgggagctggttaaatgatatgttcagtttgtggaacttttacatggtgtatcattagatctatgcatttttcaatatataccttgtactttggtaaaatttcaagatatatatacataattattttacatatgtaaatattcctcttaggaaaactggcattactcaccatatggagaccaggctattaatagaccaatagcgggaacagaaattttaaaagattggaaacactgatagcaatagtcaatgaattcttgaagggagccctttcaaaataaaataatacaaaactaaaaatacaaaattaggcacacaatgaatatttagaacaagaaaattacaaaatataacaattttattaattacctgtaacagctataaaacttattttctacatgttataattataaattctttgtttcttcacatgatatgcttaatatttggtagagagattttgtttccctgtatagtatggtcgatgcaattttttagagattttataaacagacatattgtttgtattgttagtacatgcttgggctttaaaatcacaggaattctgataaagtctacttcttgcaattctcgtaaaatgtatgatgcttttttttattccttacatttttaaatataatccccaaaccacagaacttccattttgattggaaattttaaagaagcgaatcccatttaaaattatgcatatctaacaaacaggaagaattttcaagagaactagtctcctttccctccactacctacattcttccaatctctgtggaaggaacatgtttataatggaacacctttatgttaagactctgggtaagtctacccaacagcaagtaggagtattcctggttttcctgaaccaggacagataggagaacttaattatagacagaagtgacaaatatatctcgctaaagccaataaatgtatctgtaactcagcttccccacttggatctcagaaatgcccatgactactctcacactctccacacaaggcgaaatggaaggctaaggaaagagactgtgatcccaaccaattgctgttaaaatattataattttgaaaatgttacaaaaacaaatgaccatatgaacccagtgctagccccttgctggtgccttgaaagatgccagcgcaagcttcatttgcttctctataaattcactctgctgtttttcttgcctcactggttgcatacagcttctcctggaagtgagctcttcatctctaattactgtcaaaacactacacgcacctatggttggctcttatgttgtgattctccccacagtcaacactattgaagtttctccttcagtgactttttataatttcatctgactgcttcatagacagccctttttagcttagtggacagtagctggtgaggtatctgggttttgttatcgaccattgttaggagatgaccagctgagcacagtggggcggccattaagctttgttgcagcagcactggctgtgcttcaagttcttaatgtttttctcttgccatctgatgtagatttgataccagcacatagggtggagcctctatggatacacacaacagcagtattatcagtgttcactttgattacacggttcttgattcaattcatcacttgcccagacaggaactgcatttacttacttgtgctgacacctgactctgttttacaccttgaaaaagtactcagagcttttatttcctttcacccgtaatgtagatttgctcagcaatatgatatacatttgattttgcaagcacttagatcagaagaatacttctataaggaaaatgtttacctaacaaaggaatttttgcagtctaatgttattctgtgttgagcatcttctctttcacctaaaaaagtagttcattattctaagttaatctaaagcttttgtatgcagcacagaaatttatttaaactggcaaattggattaaataacatgaatttatactagcactcagacaataaccattgaaagaagcttttaaatttataattaaattaattatttcttctttagttgcactaacagtatttcacaggtattatttcatttaattctcaaagcaaaactgtaaggaaagcaatgttagccttgttttgaggaaactaagatctaatagagcaggaagataggagcctgggtccctcacaatgtgaagcttctatccttccctgaaaagcctaccactggatttgttttacatgaatgagaaataaaccactatttttcagatttttattttctactatcccatatatccacagcaatctgaccataattatttacaaaccaggactttttcactgtatcaattgatggtgacatccttcataaatctgggcatttttcttcacttgccaacaaaaaggtagttgtgggtactacggctccaatgaagatgagataatacaggcatcatacattacatgccttccggaagtgagatgtgactctcatacatgtaagccagtcttctaaaatgatattctgcattttctggatcactctgcctgctcatctactatcagtgcagcatcggtcagcaccctaccaggatggatttctggcctcactcagaaacagctggacaaagagttttacgaactcatcaaagatccgtaagcaatttatataggaacttcaaattagctgataagcacttttatttttggtcctcgtttatttattgccacttcacactgatcaacttcaactaaaatgttgctgttttgtggcaatcttctaatcatcattgtattctgaagttggagagttcaatacttgaattttacatttgaggaatgtaaatggctaatcaagagccatccgctaaattcaagttagaactgagagccccggctctcagttcatcgcttttctctcagctaggcttccctagaaggccattcagattcctttcctctacttgaaatttaatcttaactcgtagcatatactcactcagcatttagactgcagatagtgctcagaatgggttaggtacttactgtgttcacaagaactctatgtggtaggcacttttttaccttaacagagaacagagagaaatagcttttgcatttacacagctagtgagtaggagagtgaggacttgaacttatgcattctgactctagagtctatactctaatcctcttatcacccggtctcacaagtagagtgggtgttccaaagaaagcatactcttcctacaaggaccatttccctacatcttcctttccagcttccatggtaagtgactaaatttttttattattctgataatctttacagaagggacttaaagaactgaaaaataaaccctacaaaggtatcatgcaaaataaatgatataacattttagcataaaatttttatcagataaacattattggtccttgactcttaaggaaattggacatgagacagacagtaagtcaagttttgaaatttggacaaattaaaaagtttgaatctgctaccatcaaagtactctatacttacttctgtcttgccttgcctaattttgaatatattcattttattttagattatatttctttctttagctcaagctgcatatcttaagaagtattattaataagcatatagacatatttctctcacctgaagaaataatctccaattttatagtataagtctagtgggaaagctgtgtctaatatagaattttgctttagcaattattttatttacatcttgtttcaagacacagccattgagcttgacagccaacctctaatttttatttattgcctgccaactttgtgctttttaagcttctaataaagaacagtttaagcaatttttagtcatccctcatgtatgggcctcatgtaacatgttttgtaatgacatacatcattttataataaaatttcatcttatgagtctttcaagtattttgatttctcgcaggataaagttctacatgtatattatacatgaatcatctgttttctttagcaattcactgatctcataaagcatgcatctgggtaaagaactactgaaatttaaacctctttatttggaaatgaaaaattgttaagctaacagtagcaaattccatgtttcatcaagcttttctctaatccattggattttaaaacaattcatagttacttaagagtttacttaaatagcctcagacagcagaactttctctggaagcctccctcaggaaatagtcttgtgtgttctcttc

SEQ ID NO: 48 - DNA, 5297 bp, Homo sapiens
  source 1..5297: mol_type = genomic DNA
ttttattttgaaagaagttaattaagacattattagtttgatcattatactttttcctagaaagtaaaaatggcatgtctttgtaaacatgggcagaacagaagagctcggtgagatttgtattttggtgtcatctacaggcactgtacatagtttcttcaactgatttttttttttctgttggtgcggttcatttatctactttatggaactattacaaaatacctacctttgctggtgtaatttaatagtcacattggtactgctgttttatcactttatactcaatttttaaagtaattcaaaagtatgtaatttggaaggaggctgtgttttaagacttggctggcattcacattctgcccagctaggcattaaatgttgaaaaatagacttgagcttgcaacagagaccatacgacccaaaagcctaaaatatttcctgtctggccctttgcatatgcttctccatgctatttctaaagcaggttattgtaaaacagtgctagcccatgaattctcaagagaaaaatgtcatgagaaagatacctaggaaatgtatctatttccttattatattcctatttcttcaccgcttttgcctgtttatccaatacctatattttgcctggaaacttcgcttaatattggggatcctggcaataagataggcaagatagtgttttctctcataaatattggggcttaagagcagtttctggcctcaaatctgattttgctacttccacgatgtaaacccggatacattattgtgctttagtttccttaactgacttttgtttttaaatctgtcaggtgtttgatgagatgattcctatgcttttaagcccagacctctaatgaatactctaaattttcagatatgaatttacaacctgtcattcattcaactgtttagcaggaggagttggagatttggcaatgaatctcaaatagatgcctccttcctcagagagattaatctgtgatgaagacagatagtaaagaaaagatagtaaaacatatagcatatcagatggtatgaattatctgaagaaaactgaaagcaagaagttggaagggatcttttcgattttcattgtggttcactatgaagatgagatttgaaaatttacctagctgagatgagggaggaggccaagcactatctaaaggaagagcgtgtcaggcagacagccatgacctccaggaggccaacgagagtgaagtaagggggaaagttggagaagaccaagctggaaagggaaggaggtagaggtctagacggtggggccttctaggccatgaaaggaaggaatttagggttttatgtagaggagtgtcacgttctcacttgttttctaaaggatcactctggttatgttaagaataggctgtcaagcctcaaaatggaagcagggaaaagcgcaacaattcaggctgacagggttgtgctgtgtcagttaattccagcaggcctggaattggcctgagctgttttgtcgtaggagatgtatctggatgacactgatccctagtcaaggcatgaaatatacaggataatagtgttactttgtaaccatgaggccaaggcaagtttcagggtggttattaccaggttttcagcattattttgtttactggtaacagtgagattgatgatttgtacctggtttcagtgagacttcccagggctctgatgctgaggataaataataagagggtctagaatgactcccaatttctttatcctgggcaattggcatgatacagttaggaaaatgactgagatgaagcctaattagcattcacttagttaacaaaagaaaagaaagatgagtttagacaactctcttggggagatctgatttaaacagaagcagagaaaggaaaatttgttggagaatcacagaactatgaagatttaaagagctggtaatctaggcataaaagggaattatttcaacttgaaaactcatttgaataaattatttcctttttccacactggctattaaattttagctaacacaacaaatagaatgcagtaaacatcagtgccatatttaattatattatgggttgtcctatatactatatcattctatctctcaggtattttcattccaaagtgatattccatttaacaacaaaataaaaggtgattgcaatctatagcacaataaaatcagaagctggagcctagggaaatgtaaaaattgactcagtttctatgtggaatattatatttaagcaaaatattattccttgtgattctggacccaagattaatcctagaattatgtggatattatatgagcccaagaaaagaatcccattaaaatgaattaaacctgccattaatgttgctatggcctttaatatttattttattctgtatttacatttcatctggaagtcttttgtaggaaatctgggatgatttttacttgtttgaaggtaaaaactttgaaaaccttaactgctgtgttgtgcatgcagaataaattttgctaaatggatgaatgcatacattaatgaatatatgaaaaagtgagagttgcatgaatggttgacaaatgaatatatgaatttagtgtggcatatataatgcaaaaacattgcacataaacagagaggaagtcatgaatcctttatgaatccctttatgcgtgaataggaaaaatattgagagatattagcagaattcaggcagaaaagagcattatcatttatcatcttagaataaaccaggtggatggaatatgggcagaatgaggggaaagcgcaagtctaatgcagattacctacagtagcacaatttgatagaaattaatcaaagaaatcagctcttctagccaagctgccacactgcccactaaaaccacaaatggggattccattcaaatagttgcaaaatattctttgatacttcagactctattaatggtaggattgtttcttcagcgaaacagaacaatcctgccctctgtaatgaaaagaaaattaaattaaatgttaaaagaaggcagtttgttgtttgtctttagacaatagacaccagacaccaggaggtaatgaaataaatgcaaagagaaaagatgtagaatttccaaagtttttcccacggaataatagtccagatgctccgagaaaagcgtgctccatgctgaaagagttctgcagcgctccatatgctgccctctctaggggacgcatagtgctcattaaaatagtaaagtccccaaaatcctgaactaaaaagcccattgagtggtgtttccaaaatttatgagtccacacagcttttaaaatataacacacatcaagatcctgcagagttagtgatatggaatatgcctggggctttcctatacagacaatgcaaacacatccaaatgtacacagagctaggccctagggaaatggaatcacttcgtggtaatcaaagcctttggtagaaaaacatacctcttgatggtcagagcatggaaaaaaaattattcaggttgggaacaatgtactataccacttagggatatatggaattagaaaaaatttgaaagaagccaagaaactggaaacaaagcttaataattaaatccactagactgtattttaaattaatctttctggtggtactggacacaacttttctaggataaaggatattaatattatgatattgttcactacaccagtgccttctaactctgagatacatttatctttggaaaaccccacttcccaggggttccaatggggagaataaaaagactcacaaggcatcttggacatcaaattcctcttataaaacaataatttgttgaattaagtgctaatgaaacagatggattacatttccatcacaatgtgcacaaatttaaacattaaagagtaaaagcacgtggtaaaaccagctttgtgttttgctgattctgaaacagttaggaaaatggtttcagatgccaccaggcctgtctggctccagacaccagcctcattgactgaaatattctattaagcaaacagatgggggagtggtttgttactgatttctccataaaagtgaaacatgaaatgtaatattcccggaagaataaaaaccttgtgcttcatactttacaaagctgcattgagattcatctctagggcactctggaaaaatagaagctgggaatttattttttacagagattaattgtatcagtatgagataattgagaaaaggagaaataagtacctaaagaaaacaaagtaaaaaaatgtggtagcatattggtagcataaattgtgaatagcctgaatttctgcagctatgaatacaattattgaaatacttcttagaataacatttatagataaaaataagattttgtgtgtgctttataaaaattaataaactttattttaagagcagttttaggttcacagcaaaactgagtggaaaatagagtccctacccacccccacacaggttgtcctatcaacatcgctcatcagagttgtgcatacattacttcagctaaagtccatactttacattaggattcactctatgtgtgtttttaacaaggcagtaacttggttatggattatttatcttgtcaataattgacaatgctatgtaaagcatattattcgtggatatttgaggataaagaagactggaattaatgctttcaaaagaaataatttaattacatggtttatttgatttcaaaacgaaataaattacaggaaaatgtaagttttacacacttaactttgatgttcatcaagcagataagcctaataaattatgaaataccattgctaatgtaaaatgtagaaatattatttccctgatgtgacccattctttctttggatagctatctttggaaagggcacatacaggaaattagctttatagcaatgctttacatagattatcgtaagaacaaaacaaagcccatattcatttctcttgcaaatgcttccatttcaatatcatgctgagatcttcagtgcatcaattatgtatctcctctttacagggacactaagatatattcatggaatgattttgatgctgagactctaaaggttgaactttggttttctgagctctggatcttaatctattccctgacattaaaattcctgagaaagattagaaactccacaatcaatttagagtgtgaatattcctgaggccttttccacacatttctggcattaactctcctcagatatattcctgaagaggatgactgaagcatgcctttggcaacccaaagaatcctttttgctcagca

SEQ ID NO: 49 - DNA, 17936 bp, Homo sapiens
  source 1..17936: mol_type = genomic DNA
atgaatgagtgttgggtcatcctcagaaagaaaagtgtatttacctttaggattctcctcctcaactttctgatcaaaaccaaaaatggttcttttgttctcagtgcataggcattccacaacaacagccttttccttcctcttcctcttctctacatttaaggaaatttcaaatataaatccagctgactttggttctggacaaaactacaaatatggaaacctaaaaggggtcctataaacagaagggcttagggatttctctgtgtagaacaaaaataagaaaaacatgttactattttccattaacactggaagacagggcaccagttagcacatcagcattgtgcagctggaaccttcaggggaggggtgagaaggcctgccactcaagatgtaagcagctagatccaaatctgtcttgaagaagagtgacatgtatacagctccacatgaaagggagatagaataaattattcatgcccactaaaaagtaatattaggacatgaggcacatatttgtgatacatccaacagaccgctgattggaagagagatgctggaacacagctggaagacagctctcccatgagtcctccggaagcccacctgtcagcactttttgcccttctgcattcacgtggaattttgttcctgcttatgtccatttaagagcttatgggcttaaggcttcaagttgaaataaatactgagaatttatggcaccttgggcaagactatggaggaggaatatggacattagttgcccccagtgaaagcagtggacaaaattttctccaggttcctcctatgtgtaacaaattagaaactagtctgagaatccagaccactggtccctgtctcatgtgtgcctccaaccagatgtgggcaagtctattctctctgggtcctggtttgactggtggtagtaggatctctagagtctttttggctacagctcatctgcaattctaaggaaggcctaaagacccccatcctcttgtgatgtatacccaggagttcactgttcccaaggagctttagatgaaggacactgaggtccccaagctacccctatttgattttgtcaagctcagtactgtggtgagagaaagataacctggtagacattagaggcacccagatatagagccatagtcatttgaaagaggttgcctcctgccttgccttccctgtcctcctcactgagacacaaatgccttcactctgcagctcagccttagatagtccctaattataagttaacattttaacaaattattatttcatcaacagcattatcatcccattattgagagccagttttgtgcatgtctctgtgtcagatttacttggtcctttacaataattctgcaaggaaattatagccctgtgttatttgggactctgtggtagctttttattgtgtacatttagagaagcaaaattacatctttatatgatttgggattaggtttggccacaagagaaatttggatgagatttggaagacagaagtgaagcagccatttccacaccaggcttggtggcagcttgcacacatgtcactgatctgctggctcccttgtgggcacgggcagcatccaggctcccagctcctccatctcccactggattttctccttcagtttctctgaagcccgagccaggcatgtgtgcagcaccatggctaagggcattggcttctcccacaggtcactcatgctgccaaggttggaggtgttttgtcctgtattccagtttgtcctcatgagttcctgcatgggttctagtttgtcttactctttcctgtgcagctttccttcctgccagcctggctgatctatagtggcttaggtccaacgtcagaagcagaggcaacagccttttgtgtaagatctaaatcctataataaatcccttatcctacagcatcatagtagttctctgatcatatcctgactgatatttttgttgcaagtaataaaccaccttcacatagcctaagcctagaaagggtaattattttaaggatgtgggagtatctcatggaatccaagtgtggaaaggcaattggtcacagggaggggaagttcccatattaaaacaacaacacaaaaacacttcctttatcctctagtctctaccccatttatgagctccccttcaaatgcaaaaatggtcacacccttctgcattccctcaactcctgaaaaccgagttctaatgccaccaaactagattgtcaagagcaacgtatgtcttcacactctcaattacctgctcagcactgctgatgacttgctgtctggctggacctgtgactcaccacccagatcccccttaggtgaggggctgttgcctcagcgactgaggataaggagggctgtgcacctgtttagggatgaccccatccaatttcctccctgacaactgcactttgctaacttataagcatctcaaaattgatgtttctaaaactagacaatgttctttcttctgaatttgtttctccccctagaattgtccaattcagtcaactgtatccaccatggttcagtcagacacctggctccatctatggtcctccctgtctttcatcccctacgtatcattcagcatgtactgtcactcatgtctagccacagccatgcagctggtacaaagggtctgggatttctttaggtttccttcttcctgactccactataagaacagtagaagatctgtctggaggcaggcagatgttgaacaattagtcatgcatacatcttaggtcaactcccaaagtgccctttgtgggctgcttagtttcacaaaggcacagagagatgcaacaggtctgaggatcctgaattcattcttatcattttgataaagctatattctttattctagggcattgtccaggaattgttgccattaatcacttttattaactgaatatatccctatatgtgtgcactttgtattgatataaatagcgtacattcctgttgatgggggcaggtgttgctttgctttgtccacagttcttagtccatggcaatcaagaataggtaagtattacagatttgcaacaagaaagcaagaaaacaaagtagagtgcaaatttctataaagaacatagtcaatcaaatatttactgaatatccacttcataccaggtaccctgctaagtgctgtgaaagggctcactgataaataaggcatggatcctgtgattccacaaagccaatatgtgaacagtttataataaggaggaatttagtattatgactgtttatacaaagtaatacagagaaattgtatattggaattgtttctaaacaatgctttgcaaacaattataagagtaaggactgagaagtattaaaagatccttcaattgttttgggtccagtggagtatgtatctaggcgcacctcagcacctcagctattcatttccacagtaactgaaaagtctaagggagtaataccattaatacttatcaggggaattagaataataatagcatatgctgaggctaactatgtttcgggcacaggcaatgcactttgctcatattatatcatttaaatctcacaacaaccctataaaggagcaactattgaggctcagagagcatagacagtctcacatcctgtaagtggtaagagctacttgaacccaggtctcgcttctaaccagtaagtgagatgtcaggataggaactttcctcatccctgtagctccttctgagaagaaagaatgaagaaagacagtggaattatatggaggaacaggttggctggagacagggaagcagcataaattctctcaggtacattcctccaacaaacttaaggggagaaagaagatttgtaggtgggtgtttacacacagattgctagaaagtcaacttctcatttgaaaacactcctggatatagggctggagtgtgtcctagaatgtggataaagaaagaaacatggaggttgttattgaaccttgtagggagaatgtgtataagggtggtctaatttttaaagatgagctcacaaattcgagaaataatccagaaaactgaactttagaaaaatgagcaaactcttccataggactccattattcgtctagcagaaagtcttaggaatccctggaaactggggacttgcaccagcatggagacctaactgttaaattatctggaacttcccaaacagactgacatcttggtggttggaaatcaacacgttgggaatatttacactatgaaaatcagtaaatgtgtaaatcccccagagacattgttaagtatttaccagcacaccattgcctagaagcaacaggggttctttagtagaactaatgtggtgaaaactgggggaagagtaggtgtagagaaggaagatggggaatacagaaatggagttagagcaagtaataaatacagattatgattcccacatctgtgttattcttgttttttagaacctactgtttgaatgcaagaaaatgttgtaccacagatgttgaggacacaaattaaacccaaagcaaatcaaactcccaaccattaaaaacctttgagaccatgtggctgactgtatatattaaacatatataatactggaatttcctcaaattaattccatgttaaatataatgtttaataagtgataatgatatgcattaaaatgcatacgttacttaaactcaaagaatgtagactaagaaaagattttgaggggagaggtatttcatcactgatatcatttagtactgctagcacatagtggtaataaaaagagactgattggttctatgattgttctttaaattctcagagaatgcacccacttgcctgcacctgttgtactgctcttggcatgccatgcctggatcccatctcaaccaaaatattaagaatcttctcgggggtgaggcccggagtctattttcatgaaaagctcttgcatgtgattctaatgatcagccagttttgggagctgctgatatgggaacaaaagggacacaaatagctatgattcaaagtcaagggatatccaaggagctgctcaactcttcaaacccattctttcactggtctgggtagatggacgatcattgaaaacaaccctgttaaaggaacctccagggcaactataggaagttctcctattgtgtcagaaaaatacttagcatagatatttacgtgttaaaacaagaaaacacttatctgtaattaaaaaaaacatatcaatgagtcttcatccttattgattcctctgctccatcttcagaccatttcagaaaagtctatcctcaaaaaaggcagaatttttgcaaaacttagatgctattcctttgcctgggctttccgcctcactcagactaaagaccaactctggctatacatcctgccaccaccactctctctgtgactctcagtgtcttccagccacttggcctcttgctgttccgaactcattgagcactcccacctcagggcctttgcactgtcttgcctctgcctggaactcttccctcaggaattcacaattcaagtctgtgtccaaactcttgcctgatataaaagagcattatttttcttcattgcacgtaacaccatctaacatatcaatattttacttgttttttatttcctctagattattgagggcaggattttagatgtttatctagtgttgtatttctagaacctatcttctgccttgcccatagttgacacttaacaaatatttgttgaacaaataaatgaatcaaaatgcagatgaccaaacatcaaataataaagaaataataataataataataatgacttacaaaaaatctctttttccccaaaccttctaagtcatacttctggtaaccacttcttctgctagttgtcctgagatctctaaatgacatacttctacttacatttcttgatttatcccctgctatgccaatgtaatcactatgactatttttatactttgttatttgtaccttagttatattctctgtgtttattttcatgtctttgagacagtgtctactgtctcctcagttagataaggaccctctttctttccttccaccctcccagtgtgactaggatttcaagtctcatcactgaagtcttcaaaaagatctctaggagtctgtggctgttcctaacaacatttcactttcccccgatctccattttaaatttggaaaagaagtagtgtagagcaagatttggaatgttgtctggagcatctggagggcaaaaaattcttggaagcttgcctgtaaatgagcacccctatattcctttgggcatttcttctctgcccccttggaggctctagatgaatcatatccccagcccaagaagcaccctggagtgagggttggttctatgctaattgttcacgagcatctgactttttcccttttattaatagaaagtgaaggagaaataaggagcttagtgggcaaagatggagggagaaagctgcgaacctcattaacaattttatctcatcgcatgtaaattgaagtgtgtacattaaactttgtagagttcatctatggaatggcttcagttcccaaatagtcttactgtcactgttcttataaaaaattatactaaaggcttccctttaggtattaaggggatagcttaagcaagtgagtgcaaaaattttttttcttattttcctagtgccccgaccatatagagtaaataatgagggcttataaaattgcttcccttccagggtgtagggataacattttacatcaataaattgaaggaaaagacattgccccatataaggcacatagtagggctcaaggggaggaagaatctagatcagccttggctgcatggtggagcaccaggggtacttgaaaaaataattccctctagaaattctgatgtaattggttaggtgtggtctgagtgttgacatttttaaaagccagaaaaaaaaagaaacacaccctggtttagagggaaactctgggttcttctctacattttccttctgatctttacagggatgcatatcatctttaactcagccatgggcttggagggtcaagagctgattgtatagaaattttatattattctagatgtggtctactacatgttacaaagttgagtgtgtgtgccatatgcagcactttcttgtgagccctttgaagctccatgtccttagcagggttccctacacttagaaatttggaagccttcctttaagttttctgtccaaatccaataattagtagcataaatcaacaagcagaatgtagcaaccagtacagagaggctggcctgacagtcctcaacgatacttttatttttatatatccctatgctcaagattagtttattaattctgtagcaggttaagggagtggaggcagctggtaatcgttgattcatttatattttcattcaatagccatctgctgaatgtcttgttagggttggggactcaatattgaatagactgcacagcccctgttctcagcctagtgggagaaaaataagtaaatgagcaattgcactctgtgtgaatgtgcagtaatgcggggacacaggatgctgagagagacaagttgggacatccaacccagacatggttggtaggttagggggtggttatgggaaagtctcttgggaaaaaaataaaccacttggatttcccttcaagaaagaacgtgctgtgcttctagaaatgcagttatctgatagcatccagctgttaggctctcaggattagccttagctttcaagctgaccccacattcttataggcagggggaacaagcagagtggaatggatagtagaaggaaagggaggagagagaggtaagcagggagtttgtagcccagatcatgggggaccttgtaggccattgtaagaacactggcttctactcaaagtgatatgaggtgagaggaggggtgggcactgataagctaattcatgtttgatgttctcagcctttgactttgctccagcttcctagtcagtgatccaggggccctccagggtacttgcacctagcaaagcaatacaccagtgactcctagtctgacagattagagcagcttctgtgcagtgggagttctaaagaagctgaagatttcatttctaagttcaagagtgttatgtccttatagatgagtatgtgaatttttgtgcccaaaagttgtagaggtcaaggctaggacatgccagatgacatcccagaaactttttagggtgacaggtaaaataaaagaaccaacatctctaattgtcctccacgttagacttgccttccctgacttgtgaaacctggttatcagtgagcctggcaggggccgtgaaaaggtcctggttggtgtacaagcccaaatttggagcatgagtgtttattttctattctctgtcctctgaagatttccatgtagagagttgacctgggccttaaggtaacaatccaaaaaaaggaataatttagaagaaaattaattcctcccttgaggaacaatttctgtttataattttaaaatttgtgttttatttcactatttcctaggatttcattcctctagctgaacttaaagctgtcttcagaagacccatacttctcagcttctcatccagctgcagaatacacaccaggactgggtgtgcttggaagtccactttccaatagattttgtggcacaaaaacaactttttcaaaaccacctggtgccagggaatgccagtttcggggagagcaaatctgttcctgtctgcatcttttcttcatctgggtctctgagctacctggccttgataattcctggtcccaagtccacaatgaagacagtgacaccaccatcattataaaaataaataaaagagatctgatgattcacacatgttaatggtgaataaacacagaaaagtatttagcatcgcttcaaagaggaagagaagcaatgtagaggcaaaagcaataaagttaacaaccaaaatggcccccaacaggcaattggttaaatctggatactatggaatatctctgttaataaaagtattggcatcataaaatatcaagaaatcaggtcacaaatatgcaaatatgttcttagctttcaaaataaaagcatttaaaaagaatactctccaaatatcaacaatatttatttttggaagtataactatgaataattttaattcttccaaatatttttctgtaatttccaattttctagaataaacatgaaacaggaaagccaaatcaaaataaattataagaaaaataaaagagatattaaaatcaaatttttcctatagtataattttcacagaaaaaatgtttgagaaaatattttcaggaagtcatctaatattgctgttggcttttcctcacaagaatatgatgtgatggcaataaaaagttgttcataaagttatgatttaaatggttagactggtctcagtgaaattcagggtcagctctacagtttaatcatattgtttttactggctaccaaggctaaagaagtataagtctgctaaattccttaggggaagtggcacatttaaagggcgcaaatccattctcttgcagccctcttttcactgcaaagaatcatttagttaatatgatagaaaagaaggaagagggggcacctttggaatgcgacacttgcaagatcctatgaatgatgatgtaaatctttccctgtgttttgtaacctgaaatttccaagatattccactgtattcagatatcagctgcctattgtgggccattttggttgtttacaattgcctgccctacatccttcttcctctttgattattagtgatgctaaaatatttagatgtttattcactaatatttgtgaatcatcctttctctgagcctgggcccttctaagttcttctcccatataattcattctctctcatagaacatagaaatgggtatcatcttctccacttcacaggtgaagaaacgagactcagagaactaacttgcccacgattatacattctgtaaacaatgaagctaccttcataactcattctgaaattacatgtacatgtgtttgcatgtgagtgcccaatgaacatggctttgtcaaggatgtttcctcatctagtcaccgagaagtgagactcaatcgtggccatggagatgagaacacaatccaccaattaacagtctaaatgaataaagcgtaatttgtgttggagtttcacactggggcaacagctttgagatggagaggctttggttgctagcagccatttgggaaagagagaatcatgaaaaacagcagaaggtatggaagatgatgtctgtgagaagctagtctatgggaaaatttggggtgtgttgacaaggagtatcatcatccaattagcaactcctcatagtacccaaggttcccattttcctaggatctggggagtgttctgaagggcagatcagagagagccaagcttgacaggaaacaactggagaacatacagattgggtgcagaccctctttttctctggcttccttggtgatgaatgggagtgggatgttgtacataacatttgccaatttagcatgagctcagcctgtgctcagaggtgtccttgttctgcctgggtttctatgaaaaggagataatttaggtaaaagtgcctggtgcatagtaggaactcttaacatgagacttcttttcctcctaccttcttagttcattaaaacacttctgaaccactgctgcgcagcaagcatggcctaaaaaagaggttaaaatgttcgtaattaattaaactgaaagacagtaaactgggattagaaatgtacatatgtctatatgtgaaaaggagctaagaaattgtgagtggcagtagcctgtaattccctagagtggaggactctgaaagagggtgatttatctttgagcatcgctcaggtgccttttttatagagtcctgctgttgctatggaaatcgtgtctgtagaaatgtgtgcaaatctagaatggattgccagttgcatgaatagggaagtgtggggaaggggcaggcaggctgacacctgtcagtctccagcactggtcctgaagagaaagaattagaatggagagagtgctgtgggcaggcaccaccatagatagctagtgtctgagggactccaagactccaatctccactctaccacttgggtgacctattatgtctgttatcttagtgttttgataaataatcctttcactgtcagtgttacctggtttagttgaccgaatagtcacacgatcactacagaatatagtttctgtcataaaatgaaaaaagccaactgaaatgatcctgtacacctttgacgatattttgtgcacaggacttaagttattttccaggtaagaactatgtctccctttctctgcacttctcccacagcttcaattgaacagctatgcttctacccacccccaaaaaggaacaaatatttttatcatctcacaaggaaaaactaaagttgtactgagtcacggaactggagatctgctcggctctctcactagtgaagtgaccagctcttacatatcagccttccagactacatatttcaaatttgcaccacaaggaataaataaatgactgcctatagtgaaccccataagggtggaccattcaaagtttcttgctgtgagcaacttaaaaaaagtgatgtgttgaaaggatggagacatacagaatccttgcgggaggctggagaacaagatttagacagtgacaggcaaaaagtcctgggcagccagaccacagccaagatcaatgtcacagttgtcccattcagacactaatgcctctgccggtgggcattgaactcaatcgtataaagtgctggtgagcagggcaagtgctgtgctgacctgacaatggggaggacttgagcttgaggccctgtggcagggacaacaggcaccaaagccgcctaagagccaaggatgttggttgttgcttggaactgtgaaaatgtaaaggtagcttacctttcctgcctactggtccctcctagataacacatctaaggccacccaattcacatttgaaaaggaaaaaggtcagtctctgtttacatcccaagcaactgctgtgaggggatggaactgacttaatcatacaattgttttgatatcacacagacaaccacagtttttagtttggaaaaaatatgtgtgcattgacctgagctgatggcagcttcatctcccggtctcgctgggctttgctgaaaccagaccctaaaaagtatacttatgatattaaacttgtattttttagtgttggaagaattatcactttctaaacagagctggataattggaaaaggacagggggaagctcctgaaaaactgtagttgagtttatgcttcactgctcataagcagagactggcatccgcctcctaacttattgccactgaacattttatgtgaagttctgtttcagggactcaattaaatctatgtttcacccagctctctctatatgataagtcatggtctaccagccttcttccatggcaaaataaaactccatagtggattcaaccatcattgctacttgaaaatgcctcagagtcatctttttctatttctacttttacttaagtttcttctttttggacacattcttaatttttttaattaaaactctatccttttagggatatatgtcaacatatcaactgctttgtcatcagaatttaaaaagctgaagtgaggaattgcgttttggaatgaaagccacgtgagcagtaagggcagccagccagaaccttggggaagtgaatgaaagcagaactgtggtggaagagtttaaatcccctttacagtccgtcatcctcccccatttcccagcctctagggccgactggaacaaaggtggtgtcatattccctgggcacactgcctgccactgcacttttgttttttccagttttttttttttttttttttctttagagcagttttaggttcacagtaaaattgagaggaaggtacagagacttctttcctatatactcccgtttggacaaatatatgataacatgtatcctcctataatattacacagaatagcttcttccactgctcttaaaatcctctctggtctgcctattcaaccctctcttctgacctctgacctccattgatgttttctgtctccacagttttgccttttttggaatcatgcagtatggacccttagattggcctttcacttagtcatgagcatagtttccctgtgtcttttcatgacttgatagctcatttcctgccaccatacttttagtgtaagcaccaagttcattcttcctgcccccaccttgacaaagcatacatcttagaatcatttccttgccaccaacttcctgtgtgggtgagagaaaatcagattacacaccaaagtctaaacatctgcttctccactggtttcctggtacccagtgtcctcctgggcttcgagatttcctgtttgaaaaccaaatgtgtttgccaccttgtggtctgtatttttaaatttgtttttagccagatattgttaataaattcattgcctagggccgtaagtgtaagtgtgtgccatgtatttgtccttgtttaagatttccattccgtttaacaatgatggttaaagttacgaggctattcatattggacatttgtacatcagaaatacagaactgtctctcaaaaccctgcatatcagaatagttagcagcagcatgggaagaagctatttcatttccatggcagccaggacccagacgcgaaagctttatagattaaaatcaatgcctgaattgcattcagaaagcctgaattgcctgtcagacttcaggaccagcggctcaatgtgataaatgaggcaagggagtgcgtgccgcattagccagcactttggatcctttttcaaggcaaataccaaatgggatgaattactgggaaacagcaaatgaatgtcaagaatttgagatgtgggggttataatttactaattactgggaaaagatgaacacagtcaatgtgtgaaagaatgtggtagggggaaggctagatcagggagacaaagagagtgagctgcacagaaggctcctgttttctgttttgataaaaagagatggattttagaggcatagatacctggttgaactcccagcttggtcattaccagactgtgtcataaattgtaattagcatgaaaagagtatatgaacaccaagcctcatacctggcacatagtgggcagtcaataattaaatctgccttttcccagatccctttctattcttttcttatttagcactcagttgtacaatgattcagtgtgagaatgagcctaccttcctcccatggcaggtcaatattcaaagacagtgaagcaaagatgcagaattgagaaacttgcagaaatgctgtcacaaagactgggttcactataaaactgctacatattatgacgttttgatgcctgtacagatgtgtcacatggcaaaataatggaccatggttttattctcccgtgctcttgcctctcttttgtgacactaatgcattctctgagcccagtgggagctaggcagagtagtctcaatcacatgctcccaacagggctacagctccaagcagccaagacagggacttcctagggtctcctctctgccaccatggataggaaggatatcatctgaagaggaaccacggagtaattgagcaagatgtgaaagagcagccaagagacacagatgcttatgctggacacttgccaaccagttgttgaaggggtcagacccatcatttactagttctttaagagactgtgtaaggaaaatatacactcccctccctttctctagtgccctcgatttttggttgtggatatgggtactcagaatgccgaccacactgaccagtcttccttgcagctgtgccaagtgatgaggttctggacaatgatatgaaagcataggtagtatgtgcaactccttggaagtgtcattggaaaggagagtgcatatgtcctctgctgtccttccttgttgtgttatgcgtttgtagtggctgaaactggagcagccatcttggacatggatgtgaacaatgcaacagaaggagacagggttcctgaggctgtctggggccatgccaaccctagactatctagatttctttgtgtaataaaggaaaaaatgtcagctattctgaattttatgttactcaaaattacttaatcttaattaactaatagagattttaataaatgttctactaaaatgggtcccttgggcaaataaatcctggaaatgttgtgtattacagacactttttaaagatttgcagtgcaatttagcccattacaggctagtatccgtgggctcttgcaactcagagtataatccctggaccagcagctcaatatcacccaggagcttgttagaaatgcagactcaggcaacccaaacctactgggtcagaatctacatttttaacaagatcccaggttatttgtctgcaattcgtttgagaagcacaaccatagattacaatagcatcctatggtctttgagtctggaatagaagtagcagctgccagtgggtatgtctggagatcctggagcacaggaatttttcacagccatagaatggccgtgtgctcttcaagcctgaggttgctgctatcaacccatgaatcctccaaaaatttcagctctgagttttgagtcattttcaggaccccatcttggttgtcattgactgagctaggtttgtggcagacatcctcttcctcttctgtagggatctcggagtgtccccctggtgctgcacattttgtgtcctacaactgcttttcttggcaagttacatctagatctacgggaaaggtgagaaggggtcttaggaaacctacctggtgggctgcaaaacctctgtcagtcatttttattctcacacaactcacatatgggcacctgggctagctaaccagaacatcagttgaactaggttattttgtgtgtgaaacagtaaccaattagatcatttctttttgctttctggtttagctggtgatagtatgtcaaaggcattacttgctagtggtgagaatcttcttctccggagcctgacttcctgggtttggctccaggctcaccatgcgccagctgcaagacttcagtcaagcctcagttccttcatctgtaaagtggggattagggtggtatctacctcccaggttggttgtgcatgaaatatatatgaaggacttagacttcttccttgctaataggaaacactatataactacaataaaaatatatatatatatatatacacacacacacaagtaataagttgttaccactatgttataatttaaggggtcaaaatctaaatcatgtgaataaaaatctaaaaatgaggaatgaaaagaagtacaggtatgaattggcaaagttttcaatctgtctctccctttcattaacgcaaagcagacaatttgaacatccactgagtgccaaagtttgaggtccatgcagggcactgtggaaatgcagtgatgaccccagtgctgtgattcagtggaggagaaagagagatgcacacataccgtattaaaagacaaatgtagaaagtgctatacaatattctgtgaaagaagcaaagtacctgcagtataaatgactctcattttacaaatcaataggtggagcttcaaagatattaagaaaatagctaagctctcagacctgataaagaaagaggtgggattcaaactcaggtctgcctgatgtcagagtccttgctttctctgccacactagattgactacaaaacttaacaggcacttcatagagaaggtaacatttcaacttgacctcgaaagatgagtaaaaagccaaaccaaaccaaacaacccactgtacttataaacaagtctgcagtgattctggccctccaatcaccagttttcatattctgtccagcagtcgtgtccccatgaaacaggggcttaaagctggaggcaaggagaccaattaggaggctgtgtggggacaacaatgagggtcaggactaagagagtgacagtggggtcagttaagaaataaaaaaattctgtaaattactaaacagtggattttaaatctgctcatatcacttccttaattatcacttgccatgggggcaaagagacgcatcaactaagcattatccataggtctgcttctgaagcttctaaaattcaaatggatgaaattactttctgtaatccttgtgatttaactgcgtgcagcctcacagccagcaagggagggggcggggaggagtgaggatggttaaataaaaacagttctatttctggcatgctttaatgacaaccttgtcttagaaatatttttttttgcaatgtcttttcagtgtcattgaaagtgtttttaatttcaaagagaggctgtggcacgggatgttaatcaatgaaattctggcacgcttttctatgaacagggctcgttatttatatttaccagtgacagagatgagttcttcaacccaggcaatattatcattcatgcctggttaattaaggaagaaatgccttaggaaactcatcctgatattagaggggagaaggacctattttaggtcttgggatatttcatttccccaaagccctccctgtgagtcacaggacagaaattggaggcagaatagcatagtcgtccagactaaaaaatgttttggctgaaagactggataggcatttatgtattccttgcttgtaggttctgagtgctttctgttatgtatgcagggatgcaatgccagctttttgttcctctgaattcctgtcgttggacactgccaggatttagggtagggtcagccctgg

SEQ ID NO: 50 - DNA, 2563 bp, Homo sapiens
  source 1..2563: mol_type = genomic DNA
ttcagctttattttatagtattgaacattcttcaaaacagcccatctccataaagggaaactgggaaatgatgatatgagccgagttcttcccagttttgagaggcaagacaccgtgcaagtgcatgatgcctggctactggcacagtgacgtctcgtcgctaactctcgataatcccagatcatgattaaaaacagtccctcttggctcccagggaggctgataacacttgtcaagggtgggtgggctgattcaggggtactacattcgtgttgtcttgtctgtagtcccactcctaacctcagaaagtgagtaaatgaagaccctatacaccagagaaaacctgtcattcccgcaaccttccttttgccctgcaccctttcccatcccccaaagtacacttgacccctctctaaagtggctgaattctacagtactctggggagaaagtcaagttcttagctatgacgttggtcttttttccccttttgttaaaagaaaaaatgatgagaaatgttcctttcttcaccatctcctctttctcttcctcaatttcttaggaacgaaatcacccaagatataaataaaatgttctaaataaagctctgcctaactgcagcgtaagacatgtctgtgtacgtttgtgactgtcagcctccgatggaagtcattttcatgtaattaattattcttgtcgcagctgaaagagacaggtgcattattggtgtactttggcagacttctgggtgaacttaagaagtctgcatttcaaacacaccaaacaactggagtgcagctttggcttttgttctgatattgtttcttgacttggaacctttatttctcaattactgcaaactgaaaaaattggaaggtaatttactgataacttagatttttcaatcaaaaaccttaaattgcaaaattacatattttgggcatgtattaggtaacaaaagtaacaaagaatctttgaactgaacttcctgctctgaggcagaatgaggatgtgattttttttctcttaaaggcaattttgtttctccagcataaagtcgggtctgtaatccctggagccacgcagggttctgctacctgtttgtcctgattgttctcattcagtccaggtgatcagccaagatatgttttgatgaaaatagcaattttgctcacccccaacccacttgcttttgtgtcccccgtcactacctcaatttggccgtaattgtaccgctcccataacaacagccaaataactggatatttgtggagggcttataacagggttgtccacaaagccactggcaacgccaaacagaccctgtatatcagcatctcacaagagagacagctatccgggggagcaattcttggcagctcccaatcaaacttgctgctgattacctctgcgtttccaatctgaggccagcaccccatatttctcccacatttcctccgtacactcagtctcaggaccatcattccctcatggcctgcctgggacattaaaacaatgcacacacagaggaaaacacacaacaaaaactctttctctgcattgagagcaccaaattgcagcaaagatcagggaaccacgcggtctcagggctgtgcttcaggttgtgtgctagctatcagcaccaagaatcaaaacatctttatgggtatggatacagattccctttttgctgagccactgtgtgattttggggagcacgttcgctgatcagggtatgagagctggatggagaggaagtcgcctgttccggcctgcacgtcatgtcaagcgaagttagatatttccctaccgacagctattttgatctctcagtactaaagagaaccgctgcaggtaccctgaagttgaatcccacattctgtctgagtttaggactaggactttgcaaattgacagctcacatacttttgctggtgtgcggccaacacagagatgggcttcagtcaaattcagagggagggagggagaaggcaacaggagcaagggatggtgccccgtcttcaaacgcagactcacagggtttggaggtggccgcagtgtcacacctggagctacagatgtaacagcatcagagggaactgcaagcacagagcccagtcctcccactgaagaccacctctggtgtctctgtgggccagtggggtacctgacacctggcagcaaggtgggccctaaggcctgcaggcctcaggttgggtcaagaaatgggtgtcaaatgtgtccctttggatgaaaattaagtttttaacctaaacagggataacgatgtttcacatttgtaggaactactgccgtttccaagcaattgctttgttttactaatgatttgatcaactccatgcttttggctatatgtttgctgtctgagtgccaagtgtggacccaggccagcagctttggtgtcctccagagcttgggtagatttggactccaaggccctccccagccatcctgaatcagaacttgcgttttaataagatgcccggccgtggcagggatgaatcatttccctgtaagatgtgctttggaattttgtttaaaagc

SEQ ID NO: 51 - DNA, 8474 bp, Homo sapiens
  source 1..8474: mol_type = genomic DNA
tttaacgttcaaaaccagtgccaagctaaattagaaacaggagtctacatacctgtaacttacctcatcggtatataatttgtaacctgagatcccctcacagagaagtaaaaatagcaaggctctcaagcagacttctgctctgctgagtgcaagggcagggtgagggaggcagcactggggaaagataaaagggccaaggcagagaatgccaatcatgtggtcttggcaagaacaccttctctcctataccctgtgatggtatggagaatatagccaaggaggtgacatcaggaccttccaggcagaggagggtgtcactgaagttctgagcctgcctaatcccaagttaactagtccaggaaacgccagtactcaggcaggtgcagaacagatgagtctgttttcaagttttgtagctggctcccaacaccctggaagagatgatttgctgcatagttgtctcactctctggatagaggaaagctttgtccttccttatcctgctacatggaaaaagtgacaaagtagaaaggagaggaagcttattttttaattaatcacatcaaaaaaatgggcatgtctctgcattctttgtaatgattatatattgaaagctattaatataacaaatgcaatactgagaatttctaaatcaacaccatctttgtaaaggaaaccttacttgtattatttcaaatatgtttttgtgttaatttgtaaaactctgttcatgtaaatgggttttgttcattcgttttgcaaattcaaatattcttccagttaccatttgaagagaaggtatttttgttctgcctcctgttcataaataggttcatcaggtcttatttattacgaagaatcaagacagaactgcaagctcttcactttcctaaaagaaagcaaccaggaacaacacaacagagtagaaacaaagagggctatccaaacctggctttgaatcctggctttgctctataagaaatgtaacacctgagagacttaaataacttttctgagcctcactcaattgcctttcctataaatgggaacagtagtgcctaccatactgggttattttaatgatttggtaaaatggcatatgtcaaagcccctagaaagaaactgtccaatagaaatatcacgcaagccacatatgtcattttctagtagccacattttaaaaaggcaaaaagaaactgggaaaattaattataacaacatattttctttaatccaatatattcaaaatattatcatatcaaaacccaatcagtaagcttatgttaatgaaatattttacattcattttttttctatgaagtcttcaaaagccagtgtgtatttcacacatttggacactaaattttcataggaaatgcttgatctatatttagatttcttaagattttcagttgaaaaaacagattcacatacccaggttgttccaaaagtacttaaagttttccaaaaactgaaccaagtatcagtgttttaaatttaaaataatcaagattaaacaaaatttaaaagtcagttcctcagccacactagccgtgcatgaatcttggatgttgtttcaaaaggaaccatgagccatttattcttagcctgaaattggcaggctttacattttggagaatttttaataggactagcagaggaaccaacattagggattattttctcagtgtggggttagatgtgtaatgcatatgtttgccctgaggaccccaagccacatactgtaaaactctgttaaaatgcctttctctgctattaacattttcctgtgcacattgttcatttataatagtcccagccaaaagtttagaaagctatgttcccccatagaccattatttttatgctaattaattagcaaaaccaatatctcagctatgagatccaagctctagctactcagtttggctgaaatctgtttttctaattattgactactttctgctactttaagtccgccacctaagttcttggacagagctagcttctgggagcaggggatgtctccaatggcaacacagttccacactgagtggtggagtgggagggagggattgcagctgttggagtcactgtggatttgggaggaagtaagaggaagaaaggaggagatttgtaggacagacttaaaaaggaaaagactaagacttttgacaaaatttcagtgaggtgcaattctctaaagttgctaatacacttcaacactcttaaagggaacatagtattcatatacaacaaggagaaatgaggactcatctggaaaataattctgtgtatatattcttgtgttcttaggctcttgattctttgattttatttctgaattcctggaactatatttttatttccaatttgacaggaatttaggcagattgttttctcattatttcccatatttacaatttacattgcctgtctgattgcttacatttgggcatacagatatacagatatgaatatacacacatataaatagatacaaatagatataatctctcactatctgagtttttataagggtaagagggaacagagcagctactaatactctgagcataaaccatgcctcatgggctatgtgttccatctattcttaacaaccaaagaagaaagagtgattatccctaggtaacagatgagaataatgaagactagagaggttacatatgtgactaaggtcctagtattggtagagataagacttgaatacagatttgtctagaagaacactgaggctctttctcctaaaacatgtttcttggaaaaagacttgcacaacacgtgttgcctgcagtttgttcttaaaaggaaagttctccttttaattccttattatgaacatttattttaactcctaagacagtgtgtacaaatgtatcattgtcataagacatttttacacctgtcaaatgggaaaggtcatttcttaagagcagccagctgtaagtcaggggttttaaaattaaagaaggaattaggcgtgacaggcaataaagaaggggattgtgtgggagaaacaaaacagtttgaaaaagaacagagaccaaagttatgggatattttttgctaagggcatcaggaatctatcccacccttcaatatttggtgcctttctaggcacacattaagatcagcgattccctaagttcacaatatagctacagaactaattctccaataatctgataatttactctgttataataataaccacaaggactaaaagcttaactccttattttcctcacttccagttccttagctttcaatagaaccttgaatgcttttgtcaggaaaataattcaactggcaccccttgaggaatcgtggaggaaattttgccatcatttgcattttgagtatttcccgaattttaattatccttgaaccactatatgatttcaccatcttatattacaggtttagtatgtatactattttttatattgacccctacttttactgatatcattttccttagcccttttcatgtttggatctctacagttacaccattctagccatagcaaatggcacgtggggaataattaaggaacccagccaacaaacagaactgaggccccagacaagttgtcccagcatccccagccaagatccagtaattacggagcagaggcgagtgaccaatttaccattccttaattcccacaaacttgtaagtatagtaaaatggttcttggcactggatttggggagatttattatacaaaattagataccaaaaccaaaaattcatagggtgactatttacccatgttttcccagaacagtcccagtatacacctgtcgtcccaacataattatgattagtgctggtttcattcacccttaaggcatcccagtttggatgacagttcaccttctaatatgccatgttctgtactcaaattatggtcttgccaaatgtctagtcttctagtatcttaaagtttgcatacagagagccgaatgttgcttgcttctgaatttggaccacagttgtgactgagcatacataaaaattcagaattctttaagtttgcctgaaagaaaatagatgcttaaaatatatatttataaaaagatttctcaaccggtgacactgaaaagaccacataccaaaatgttatttacattgtggtacctagtaacagaagaaacaggtgtaaagagaaaacaaaattttgttgccatttcaattttcattgccacacattattgaaagaacccaaagcaatagtttcaagactatcaggcaaactagtgttcaaagacgaaggaaaatgttgttcacgtttactaaaagctgttccttcttcaatctatatagcaagcacaattacatgtgttatttaattttcatggcactgtgagataggtactactatcaacccattttacagatgaggaaatggaagcttgcagagttaagtagtctgcacaatcaatgctgataaatacaagaggcaggatttatacccaggcagtctgattccagatcctgttctcttagtctttagcacttactctctattcaaatataacatagagaaattaatttgtctgcatcaaatcaatttgtaagtatcataaatataattttgtgaagaaaatgattctgtattttcttccttctggtgaatctatttctcttctattacaatctgatcagtcttacaggaaagcaaaatcaaatatactgggtacattgtctgcaattttaagatgctaaacctaccagtatttagcaatattcacttagattatatactaattatacctacatattataggatgtatattatttataaaagccattgttgagtgattagttctacaagggctggtccttggcttgatatttgtcaatttttttttttttccttttacaacttaactgataggatattctcctcatattgtctatgaaagtgatacaaaagttatatttgagtaaaataacagagatagctcaagaaattgcatagttgaaagatatttgtccatatacaaattggttaagtatagcccacctcagtggaaggtggtatgtctagaattactggaaaaagtgcttattagtaatttacaggaagaaaaacagccttcgtgtctctcaaagaaagagttcatcttcaaaccaaatcagcactcttcccaagaaaaagaacaagtatacatcccttttcattgctgtaacatgggacaggactccttttggccagaacaaaaaggcagaacacagaggacttgcattaggagagataatgtcaggctgtgcagatagtagaagccagacattctgtagagtaacacgaataacaacagtgatatccttctatggccatgaacaggtgcttgctaggtgttaacgatcctttctgtccccattttatagattaaaaaaagatatgggaaagttaagtaaattgcctaaaatcagagagccaggatttggtctagatagtctgtgctcataaccacagcattatgctaaacaaagaacagagatttctctaggaaagaacaaagctattattttaccttatataattctatttaatccaaattcatgggatgaggcataaagaagatggagggggagattagaaataaatatcaggaccttagagaaccatatctgtaatgcttctgtagggttacttttacgtacacaatattttgtgtgatccatgatatggacccttctctctctgtgttctgtatttcagaagattcagaaagacctctaagtcataatgattccaagcttaattcaagccaaccatccagtatggtttgcttttaagttcagatgggcaaactgtattaagatgatggggaggagaggagaattattttattcattgtcgtgatagatataattaagttgtaaataactcagaagtaccaagggagaactgctgcaatcctaaataatgattaaaccaagcctaaaagagaccaaaaagaatcaggctttctgaacagaacagaaggctacacagaaactgtctttaaatgtgagagcaaacatacataatggtgacattcaacttcaggatgaattccttactccagattttgtaaagcacagaaacagtgacttcgtatttttaattttttaaagaaaaaaatacatagccggagtgattttaatgcattctttctctgagactgggacttgattagatggcaattcaaggtcctgcaaatgctgatcatgatggaacctaactcctctgttggcaggcactctgatggtaggcacttccattgctccatggccattattacaaccccaacagtgccaagagcagatgtttttttctgttgttgtcggtggcatggagtcctatatcccataaatgtaaactaggtttaatttggacttttagttagaaaagaaaaagttggttcttaaggggaaagatttctaaggacctcttcaatactgtatagcaaaaacacagaacatgccatatgaacctcacttgggaaatttaaataggagctttagaaactaacgtgatttggcaagaacgtaattgaatgtggttgtgaggtcccactagcaacatatgcttttactaagctgtactattcctttcagtctgagcttttaaaatgttactttatattttctatgattatcattcagaaccaaaaccaaagtagttaactctgaataaaactatcctattataaaattagagctcatcctgaggaagagatgtttaaatgtgacccctttctcttctctccaccccttccaaatacactcataaaaactaaagggggaggaggaaaagtctgtttatgattatgaattgaactttgaaccattcctattgttcctgccaaggatttttgaatgctaatgtaaaaagatatatataaagtaagatctgctaaggtattttaagagtctacagtaatgcaccaagcttttttccttgcttcattgttaaagataatatgcctttctaacaaaaatgctttcaagatgtaaccatattttgaaatgaatatcataatacatcctttgcaagcctggaatttggaaacaaagaacttgctttaatctgtttttcttcttacctgataagcaggatgacagtgctgctggaggaggtttagacctgtccccagtctcatttgcttttcttcctctttcttcatcgtgtgggccctgacaacctttctttctgaagctacataaattaaacagggctaagactatcaatttttgtaccagctccaaaacaagccaagacaagaaagaatcctccagttgatcgacagacttccagtatttctgctaaactggtagagcggtgaatctccatgctttcctctctaatgaaataacatcagaggttggatccttggctgtctttaattttttcccttttacgtagctaaaagatatttcaaatgatgtagaaagacaatatattctctttattgttcattccttctatttcacaaatgcttctagtcttggggtatcagctcccctgaaagtcagatctaagaacctcagcacacaagccctgaagtggcctgaagcacagcagtgaacatgcttcaaactgatgagttgatttttcctttatgaggtctgatcaacaaaataacttgtgatttttcagataggtacataaaggatggtttataataggtaatcaaaggaagtatgattcaatttgcaaagtaaatgtcagtagagccatctgattagaagacaaggcaataacatgaaggatctactagctccagaaaagactcagcagatagaagggtcccaatatattttagttgaaactggatcttagatctgactttagattgtggaatttcacaaaaccactgcattaccagacaattatgcatgcatattgtctaattggctaactccaggaacaaaagatggacaactgttacagttatattctgatgaattcaggctaaagtggcttgaatcaaggaatatggtgaccctaaaattataatgaaaaacaaagcaaaactatatatcttgttctttcctctcatatctgtggacattaaaaaaaaaagcctcagcttttcactagtctgaatggtataggttggagaagattttagacattttggacttaatagacaaaacaatgcagttaaatgtattaagcaatttacataaattaccaaaacattatcaaaactactctatgaagtaagtgcaaatggtatccccctttatgaatgagaacctgaggctttgaaaagttgagtgaaccaatcacttacagctagtaaatgacagaactgtctctgaatcaaggttatttttattgtgtttagacacagtgacttggaaagtgtgtttgtacaaatgaaaaagagagagggagagattgagagactcacccttgcccctctttgctttcttattaccatctataa

SEQ ID NO: 52 - DNA, 1938 bp, Homo sapiens
  source 1..1938: mol_type = genomic DNA
ggggctggtgaccagaggaggcctccaggcagaaagggggagtgtctgcagaagccagaggggatatcaggacagctgggagcgcctccaggacaggctcaggcctggcccctgcagggacagtgacagggagtgggtggggctgagctgaggaggctcagctagggggacagtgcagcaatgcagcccctataagaagacccaaggccccttcggcggcctgcacagaccacggcctgggcagagagttcctggctctggggaggcagcttaagcagcgactgcgggtgcccaagaacatggagcccaggcccaaaggaccttgtaccttccttgtccaggaggctgtaccaccactggcagggtagagaatcaatgccaaagtgagctcctcttacaggcgcacaccatcccattcaatcctatattcttatcccctaaggttccccaagcactcactaagtgctggcccctgtggagcacctgacagagaatcagactcctaggagcaagaactgggcacatctcgttcactgctgtgtctctggggcctagagcagaggctgacacagaggagaaatatctgttgagtgaagggatgtgaaaagaaatcacaaccacctgagaggcagaaagtattattcccattttctggatggggaaagtgaggctgagagaagggaagtgagtaacttacttccctggaactcaaactagagtctgggtttgctccttgcatagcagcggggcagggaaacccttcagcactgatttagtggcttttcctggaaggaagtggcatctgccaattagctccctgtctttcccggctagtcatggctgggatgcactcacagcccagagagggttcaggttaggagagtaaggctaagttgttcttaaaatgactggtaaagattcagtcacagttaacatttaccccaagtactgagcgagacacagtctcagttgttcaccaacctcaggaggaaggtggaattctcctctatctcacacatggacacagattctgtcaagtgaagtgatgttctcagtcacacaggtggctcttggacgggaacgaggcctgtggtcagctggatgtgaggagttggggaggaagccaatgagggaggaagctgtgggcaatgtgatgtctgggtttcactaggcacagggaggggctgggacctggcctcacaaccaccaccatgaagggacggctctgaagtgacagaccagacacctgggcaggcagaggcgttttcatccagcaggacacctgcagacacgtatcaaaacctgccatcactgtccttagtctacgacttagggaaagagtcatgacaaggaagggaagaggttgagggctgcatgctggtatccatggtggccaagaagcaacatgagtccacagccctggagctcagggtcaagtatgaaacagataagaaaacctaaaacatgggctttccaagaaacatgtaacacagcacatggccccggggggttcctggcacacctcgtgattgtcagatgaggagctgaggcctggaaaggaagagcgacatgtgaactaatgatgaaaccaagacgggtcgcgattgcctaccacccagagccccagcccttaggcacttgatgactccttcctcctgaacagtgatgtgtcagctacaacaccacagcagttcagatcatgttcaaaggtagagtctggacggcactgctcagcactggagccactacgtggagttcccgagcacctgaaacgtggctagtccaaatgagatgttctgtaagtataaaaacacaccggatttgaacattttgtatgaaaaaaggtttcaagtatctcatgaatgattttttgatactgattacatgctgaaataacattttctacatttggttaagtaaaatatttttaagttaatg

SEQ ID NO: 53 - DNA, 1638 bp, Homo sapiens
  source 1..1638: mol_type = genomic DNA
agcccagtcagaagagaattctacagtgacatgtttataattgtgccctcctcaagcagaaagcaatttaaacacaataaagtattgcataagaaactgtggtagacatatagaagcatacaatgtcagtgaatcctgaagatggatacaatgtttgatgactgtgttccaaaataccttcgagaatccagacatgttttataacttaaactgatagatatatattttaatatatttatgtctataaagccaagaagctaataatgccattttctcagtgttaatattagctgctttgtaaaactaagcaaaatattaaaataatgccatattttggataaacagtagactgattggaactggcatcttcaaagccaccaaagcattcattcacagctcaaactggagaatttactccctgtttttttttaatgaaacaaggtaaattttgagctgctttcttaattccattttctaagtgattattttaacacaaactaccctgagtctagctggaaagagaagagcaaagggagagaaagtccactatggaatggcagggctccaggtcacctgctcttctggaaaggtccccagaaattctggcattgtgttgactcagattctttaacacttgtattgtacacattaaaaaggaggaaacctttaaaacactagtaggaggggtcagttgcttacccagaattccttctccaggcaggtatagctggaggagctgttagagccattctcctgctatctgtcattttctcagatgtgctatcaacagtgcttgccactaaacctgctgagatagtcactggcccatgagagacaaagcttgtcacttaaccagagcactttggttgcaagatgtgaccaggtggttccatgaacacttaggtcctaacccccagattcagggaaatttgagcttcttctgagtgatatagaaatgctaagactgtgggactttagccagtgtttctttctcttggtggacactggccaacatccatcatcgaaagagccatctctgccctgagcttgccaaccacagaaaactctgagaagaataggcttctacaatctcatggctaatctgtgttttcccagacggtaaaagatctaaacataaacacaaattcaaaaaagcctaacaaaccaatcatgaagccaaacaaatgcaaacatggatatagtttcaggatgaatggatgaatcccatgtgctatttggtatgcagggaatctaagggaatgtatttatagtctatttcctgagagagtaatagcaaatgacatccacaggttcttaaattgcatcactgtcatcctggatgtccttatttcctctacttctgaatgccagatttgggactttaattatcaaatgggtgtccctgttcagatgtctatacataataaagtcagaagggagtttaatttgagactttgtgaaaatttacttaagaagcatttctatagctatatagaagggattcttctagattatttacagtatgagtttacttaatcttttgagagatttttaagagaaagtatttttatggtctcttacagtgaagaaactgcccaagtttccagaggatgacagagccggatgcaaacccgggca

SEQ ID NO: 54 - DNA, 9353 bp, Homo sapiens
  source 1..9353: mol_type = genomic DNA
aatgtgaaaggccctgagaaaccccaaaccacagctttcgatcaaccacagcatagtggccctctccagttctgtcagctcctcgccgactccagtttgtagaacagtgtggagaactccagcatgttaattccttatgtggattgagagcgttttgtggtcagcacagtctgaagctgccatgtacttcaccaagacaggtgttccggggtgtcaggggagagaagagcagcgtctgtgaacgagaacctgggggtctagagcactcaggtgctctttctaggggtgagtgggcaaacaccacccacactgtcatgtaagtttccacaatccagttgtcacttccagctgccaatgctgaacttcatgctcctgtcttccactttgggcttggtagagatagcagcccctggagcaggctgtcttggctgaatacatcttaccctctccatcactgccagtcgcgaatgaaaattatgtgaaatatctcacttgataaatctctaatttatattgtgtaaaaaagaccacagggaccagataaaacatgttaggacacattgctctgtggctactagttagtaacccctgaaaaagccttaggcagggcagggcagcgtagttgtggcacatggtttgtgttcccaggggcaacaagagtattccatttcagtttggagatccttgtcaaagccagtacattttgctcaattgatcattatttttctttggctgagttaagtagcttcgctctttctaaaggatcaattccttgtctcaggagcatttgaagcttctggatgaggattagaatgattgaaatctactgtgacctgtggagtgactaggcaagtctcagatgcctctaaccctctctgtttttctctctacagaaacttgcttcaagaaggagtctctactttaagtgccaggctaggagcctctttattggaccacattagttccctacagcccaattttgaattattattttattctttcttattctttaacactcaagaaacataaagcctgtgatgtagttcatgttgtgagttaagggaggtacaagaagtgtgtctttcaggttgagaaacacatgccaagatggaaggcagagcataaatgcacgggggagaatgtatgtgaaggataagggtggggagcaggagtggtgggcagcagtgcaggcctggcacttgctgaagggaaagagagttgggtaggaagaatctagactacagcatagctccaagatatgggaagcccaagaacagaggagttgccactggggcctgggttgtgcccagggaggggaggcctcagtgtgaaggctgcagggagcccaaaggatccatagttaaggcttcatcctgctactcctaaacactgcttctactgagggaccaaaaaagcttaaaaaccctcctctgtggctaaagtggcctcatggcactgatgtattcaatcaactggaagcagaggtcagctgggaactttggggattgatttacttttaaaaaaaaaaaaatcggaagttgctagcctcttctttctgccttgaatgtctagagctggacctgtcctcctgtgaccctgaagacagtagatgggttcttgatgacattctgagaacgtcatctatgcctattctgggctgcttgttgaatgagaaaaataaacctctctctacttcttatgtgagaaaaacaaaccactttgttgaagcctctgttagctgagttatgttattgcacccaaatgcccaaatatcagtgaaacttagtggtgtgtcagacagcccagatgttgatgatgtcatctggcttctgaatacaactgtgcctgagactaaacccaagccccggacttttcagctattagagaaagagagttctctttctgcttaagcagtttaggctggattttctgtggcctggatggaaagacccctgataccaaaaggttagaagcttttacagttccttgtttggaaaccgggtcctagtgtcccttgcgcaggatgcttttctgacttttgtggcctgccaccattcgcagtgggtagtgtgactcagcttcccattcccaaaactccagagccacattgctgggtcccttcactctgctttactctcctcccaagagtagcatgggccacgtgaattcatccatgtcctgaactaatctggttgttctaccaggttactcttaagttcatgggccatcacccccttgtacttactgagctccagggttttcatctcttctgagaatcgaccgtttagtccaatcttagtacagaccagcattaataggcaattaccacagggagataatacagagggaaaaatggctaaaaatatacagtgaatcacagaaaattacatctcttaaaacctacagtgtattcctattgccatagcaacccttggctagtaaagacagagatgatttatcacaggctttccccggagctcggttctgccggaagcacctccagcctgtggtgagttccagcgaggagggcaatgcagacacgggttttctctcctccatcccttccagtggcatttctgcggaagaagacttgagttccttctataaacccaggtcatctcccggcccctttctgagatgaaagtggctgtctctaacctctgacaacgcggcgtcctgccgagccagcggccttccaggagggggcagtggggcggggacatgaggagctgtaccagaccgtctgaacaaatgaggagaggagaggagttctcctcctttccttttcctccctacttctcctctcctctctaccctgctacttccttgtgaaatggactggagaggaagtcatgtcctcacttggaaatgagtctggtctttaggaagattataagtatgtaaataaacatcttgttatttcgttggaactctaaaattcactggcatagatctttctcataatgtaaaaattagattcttgatcaagggatgttaaaagtgggtaaaaatttgatgagaaacagtgtatttacacaatctcaaagtagatcctcataaaacacctattgatttcaaaggaaaaataatacagtagagaggactgataatccccacctgaatcaaatgatcaaaaacatcgcaagggacaaatgggacagattgatagcatatctcctgatgtgtgtgctaaggacatgatatcacctctgtggttttcctgccaaaaaggtacaaccggaatctaattgtgaggaaacatcagactaattcaaaaggagggcagtcgataaataaatggcttacattcttcaaaaatgtcagtgtcatgaaagacaaagctgaagaactattctaattgaagaaacctaaagtcgtgacattgttaagaggttggtgttggatcaaaacatttttttctctttacttataaagaacattattagaataactggtgaaattttagaaagatgtatagtttagataatagtgttgtgtcaatattaatttcccgagttagataatggagttatgaaagagaatattcatgttttttaggaaacatacactcaagtatttagggataaagggggatcttatctgcaccttactctcaaatggttcagaaaacagacatagggaaggaaaatcagaagcagacagggtaaaaggacacaggaattgtctgtactcttgtatttttctgtgaaaagtactttgtgcttttctgtaagtctgaaatggtttcaaaagaaaaagttcccatctgcagactcaaatccttttcagtgaattttcttcccagtctttctttccctccctctgatggtatgctccgtcctcacagctccctttacaaagcgaattctgtatgggggagtgtgaggaaggggtgtgtgtgagtgtgtgtgtgtgtgtgaatgtgtgggtgtgtggatgtgagagtgtgtgtgtatgtatgtgtgtgtgtaaatgtgtgagtgtgtgggggtgtgtgtgtgaatttgtgtgtatgtgtgcgtgtgtgtgtgtgtgtgtgtgattgtgtatatgtgtgtgtgaatgagtgtgtgtgtatgtgcaactactccgatgtcacagcaaaccataaagccgggataaagtcagttgttacgatgccgaataaggccaggaatgaagtcagaagccccttggtggttcttttcaggtttctgtggacaggagggcgtggtctcggcgggatgcactcccagctcagagagtgtgagaaaaagaccgagttcctagtctgcatgctcgcccgctctcactgtctccccctcccctgctctctttctcacacactcctctctctttcctcttctgtccgagagtgcctttgaatactctggaaccatttctctctgggacagacatctctccacagcagggttcttccctggggccagagagcaaggttcagtatgcagagcccgctaactttggtataatgatagctatgggtttaatgtatattgtgttgaggttcattccttctatacctaatttgttgagttttcatcatgaaaggatgttgaattttgtcaaattgttttccacacctattgagatgatcaaatggtttttatcttcattctgttaatgcgatatatcacatttattgatttgcatatgttgaactatcttgcatccaaatcccactgatcatggtgagtgattcttttaatgtgtggttgaatttggtttgctagttgttctgttaagggtttttgcatctatattcattaaggatattggcctgtaattttcttgttatgtccttgtctggctttgatatcagggtaatgctggcctcataaaaagagcttggaagtctcctcttctagtttttttggaagagtttgtgaactgcaccttagatattgacactgtcaacaactcttggttggcctttggtctttggcgttctagatttttctttacttctcccctcctgattaagggtcatgtgtctctaaaactccacaacggacacagtcccttcaggtgctccgccttctcaagctggctctccctaactggcctagacagcagcaaagcccagatatttccacgtgactcatgcaacccccttgcttccaatgttttgcctcctgctggtgctcagaagctcagagtggtattaaaggggcaacaggaaagcagcaagactgcccccaccccacttgctccatctacccactgtagggaagcaactccagctcctaattatttattttatgaagtgctgcccttggtgttaaaatcacctcagggcacagagcacacaattttaaaacaaaagatacaaggcagaggcttggcaatgacgggcactttttaaaaatcagcaataaaatgccagtttgaggctcaccccagggctctgaagattctccaagagggctctgtcgatgggaggggcagggagtgagtttcctacagctgtttgctcacgaagctgcctctctactcactttccttcacactctctctgagtttttatgactcagcttgaggccagaatcaactcatttattttcttcctgtacctggctcttggggctggtgggggtccgtggtgtctggcaagggcagagtggcaggcctgctttaagagcctggcgtgggggccagcatggcagctagtgcgtcactgctgggccttgccagtagttaaaaaccaaataataatattgataatcacaataataataataatatggtgagagctaatcttgactgaggcttctgtgaggtgggtatcttggctcatcattaagggttcttcagcttttaaagatgtgtttagaatcagaggggaaaagtccaaagcaaccatgttattggatgggaaaagaaaagtaccatgttgaagtcattttagcttgaaggaaggtcacagccactggtaacagagatactctcatggcagataggtttacctaaagcacgagcaatgaactaaagctggaatgatcccagctataattcccagcttcagtgcctgcatgactttataaatgaatttctaattctcagaactcaaacagtcattgccaaatatttattgaccatctctttctgccaggccctggagggatctctcaggagaagaaacattctctaatcaccacaggtttctctccagagacctcagcccagcacctctgcaaggggagagaggagaggcgtccttcagctcatttatgcctgtattgatcgctgctgcctcaccctcgatgtcggtggttaccatccagtcctgttcgatctgattcccatgcaaccatgttctggaattccctgagcactactccacctgaagagcagcccctgtctctcctggacagtaatgaggtttcctttgcagtctcacactggcttactatctcatcccgccaagcatccccctgctacattcatttctctctatgcccgggtttattcagctgtgttctcctctgctctgtgggcgacaggagactgtcacagcccagttggaattcactttatctttccctctgccctatacgctgagcaaacatgtgcagttggcatttcagcagtggaaattctctgaccccctttgctgttgcctaaaacagaaacctcagtggctttgcaggaaaattccacagtctcctgcaacctaaatttagctatttgttgaatatacatgatatgccttgctgttgtgccctcaagaggcttatgaagctgttggtgaaataaaacagacacatgtgaaacaatttgaaaacagcaaagcacggtataatcagtattatcattaggtgccaaaatgcgtgatactaaaataccatcaactagtcatagagctgacacacactgagtacccaggcacatctcatttcaacctcatgacaagcccatgaggtgggcttattgtccccattttatgcatcaacaaactgagtcacagagaatttaatagcctaaggtctcttggctggtaagacaacccagctctgttcaactccaaagtgtgtgcattctcattctactctatgctcaaccactgccctgcttagctccataaaggtggagtctaggtgttttgcttgtggctgtgttccagagcccagtgctaggaagcatgctgcacctcttacagaaagatctataggaaaagccagaccagctagatcgctttctcgagtcagatgtgggacaaatgttgaggggtttggaggatgtccaagtttggccatcccagtggtgtagggtttgcgtttgatcatgaagagtggagaaagagaaaaggaaactaacacatacagaaacacacatacaccgcatgaaacaacacaaaccccacataccccaacactgctcctctttttgaggttctaaatccagttcttcctggcctcttcctgcccctcacctgccttggtgggacctgcctgcttttatgcgcttaagtctgaatcacacctggagagcggggcaggcttaatcctattttttatcccctgagatgggttagtcctttgcaggctatatttgttaaaatgaattgaaatcaggagtgtaagactttttttttgaatttgtttctttttcttgtctctttttgggaagtagcaatggtagaaaggaagaacagtagagacactcctcgatagttacaaggggagatgaagccttgggtctgatctgtcccacattagattcccatttgaaaaaaagagaaaggaatgaagctacctccctcatcaagttagcctggggggtctgccgcaaagaggattgaaggggaaatgttcaattttaccgctttgttctgctgggttcttctccccaggacactgtttagtgcactgagtgaagtagaaaatgaagagcaggtgagcaatcagctgtgaaaagctttaatgggcccggcacccctttgggaatgaggctcggctcttgctagtgctggggaacaacatctctttcagttcagtaggctttgtttttgtaatgggaatttaactccctcaggtggtcccgggatggaggaataactgtgctcagcagcagtggcgctgcagcatcttctaaagggaaccaaggccactgtccccaaaggggcccctggctcactgggaggcctttggaaatctcagacttcctgggtgggagaggaggagatcatgtgaccccgtgaacagcatgatgctgtgattacctggggcttcctagcaagggcactgggacaggtcctggactgagagctgcaatggattcagacccatacattggatcccctttttatgtttttgagtagaaaactttataaattatgtttggttttagaactgtcctctaatgtaactctgtgcagagaggtttacgttatctcacataatccttacaattatacacggtaggtggcattattcttgccttacaaatgagaaattgaggttcagagaggttacataacttctcagggtcacacagtggagctggattccaaatgtaagttcatttgacctcgtgttctgcagccaaaagagcccacttcaaaccgaatttctacttcccaactaaatgccactggcatttttaaaagtacttgagatgcacctagaatagactctgatttttagtattcccttttcagttgaagaaacagattcgagaagataagggtcacacaggtaggaaggtagtggagagccaagactagaacccagacccatgcgaggaaaaatagggatggaaagcccaggggaaagctgcaaaaactggtcaagctgactgcccctttatctcttccttcctcaaattcacctccctaccctcttccacgtccctctctttccaagcattgggccagttccatttccacgtcctgagccagctgtcataatccctgcaaggtgttagctgataacagcctgtggaaggattgtaaatgttagcccagcggaggatatgaaaggctgtagaagtctaaaagcaaatgtgtgctaagacggtgacagagggcctggccaccaggcagccttgcaagatggcatgcaccttggagtattcactcagcgtctggtctgtccccagccttgctagtgcagacactatcttctcttgcctcaaatttgactggatgcagggatggtgatttgtagtaataagctcagagagaaatgcaaacaaaaaaaccaatttactaaagtataataagacataaaacagctaagctgactttagcttgaccagatctttcctctcacaggacaagaagatacctggggaatcaaggtctttcagggctctggaatcagtgcaggtggagttagataggaagaccagag

SEQ ID NO: 55 - DNA, 8756 bp, Homo sapiens
  source 1..8756: mol_type = genomic DNA
cagctcccccatcactgtccttacacgccaaatagctgtttctccccagctgaacagagcagtcctttggtgttcagaagacatctttctctatcatatattcagatgcctatgttgggaatgcagccaaagccctgagagagaacaaggtgctacagagtgcttgatcactcatcacattcccgccttgaccattggtccagagccctgaggctaccggaatgggtgaatcactttggaatcccagcataactctggggcaattctaacagagaagcagctatatatagggcccaatcttccagttttgtcttttacataaacattagcttgtttttcccccttcttaggaatcagcttttcctctgatggaggttgtttcactctgaccttatcctgagaaaactccctccttatctgtgggcatgtcttgactctagcagaagtagacataagtagagcgggtttgttcattgactgtggcctttataagttgcaccatgatagacgaggctgctatttctgggtgtccgcccccatcattaccatgcccggggctttacaaaatagctgggttgaatcacactgacagaatcttttagtcactttcaattctgccaaggaaatataggcatgagctttcagattctaaattaactttgattcctaccagatcccagtcccttttaggtcacaaagcatctgaaaactcagttgaacactttcatttaaaaagaacagataagggataaataattgaagccaataaaggattaagatctatgtcaaataaagattttatccaaaatgtaagaaaaacggagatcccaataaactattgagatctgtccaaactttccaaccctgaatggaagccaagacagaaatatccagttaaaggggtcactgaaatgtggttggctctttccccaagagcagctcatcctggcattagaagctgggctcaaggagccacatgtggatttgaatccaaagagccaaagaagaacatctgatgaggccacaactgagagaccaggagcaggtgtcaaaagctgaaatatgggagggatggagggaaaatattgcaatgtatagaaacattaggctcccctaagaaaacaagtgaaattatcacatatttttatcaaatggaaaagaaagtctccaaggcaaaggacagccccaatttgtgaagggtgaagctggaggacagacacggccaccagagagcaggaaatgcacttgatgccatagaaaaatctttgggaatcagctgtactggcagcccaggagaactgttgcactcagcagaaaccaagacagcagggttctctcttaatggctctgctcttttctgatatggccagctttccctgatgtggaagaaatacaagcaggctgtgtcacagacactctgccttcatggggctaaggcagacagagatttgggtgcaagttttttaacataaacttcgccttgtacaagatccctcctacccatagcatccttgaatgaggactaccagccctccagttaaagacttaaggattctagattcatccaagtaagacctaaggagaattcacttgccttaattttgggcaaatatggaccattcagatataacacatacacacacacacacacacacacacacacacacacacacacagagaggataaatgtaacactcagagatagaaaaaggttgggagagatgaaataagagcaaagagaaacaagaaatgaaagagagatggataaggtagttaatgaaaggaaggacagcactaattttgcagaattagatctgtaatgtggaggataagcctgaggcaatctcagaatgcatagcaaaaagacacagattaaagtgataaaagagaaagtggaggccagaaaatgaagaaatcatcatgtgagttggctgcatactagctctaaatgtttcttctactataaaatggggtgataatactactctatggggtgctataagggttaagaaaataagtaattagaaagtgcctagcatttagaaagtatcaaataaatggtagttgttactactattactactaatattattattaaataatgagcacagggtcacaagaaaaaatcaataccactgactgtggtcattttcctgagactaagttagtttgtcagcaagtagattttttttaagaacatagctgctatagttccttttttttttaaagaatgtctgctttttatcttctacataaattataatttaatgaatacaaaatgtttaggtaaaacttcctaaaagaattttgcatacagtttctcattgtcttctgagattaaatgttacaatggagaaatcagagacaagcttgaggttcttacatggaatctttatatggaataaaaattgcagatataacattgtcttcgaagcttagtaactccatatatctttctgtctttttttttcctttctggattcagttctcttaacctgccttctatgatatccttcaatgctgtcttatgtgtttgttcttttccagcaacattgctatgcttatgttgcctttatcttccatgaccatcatttttctttctaacaatttatatacatctttactctttaatttactcaagcctctccttctcttgaaattattgcactaaagtgacttctttttctatattgtagttttcagtttttttcctctcactttttctcctttggctatcttagaatgagtctttatctctctgtttttttttcaaaggtgccatgttctttttaacttctctgcatggctggggcaaagggatctgagtcctgggtcattctttctgaatatattcagagatagtcttttttctaattttcattgtttgcccgcagaccacccacacaggaggtatgtctggctcgcagaaaatcctaactcacaactggaggcccataaatctggaccatgcctgaaatctggagggttatgtatcatctctcagcattcttttcaaattgattccaaattcaccaatttggcagatattctctctagtctgattggtacttgtagctctagctatatttcttcagatgatttttcttttctgctttatgttccgtggattattttataggttacacactggagactatagtaaacagactttttaccagaagtccaatgtagtcttttaaaaataaaaatctgatgttaccccatcattctgcttccatcatctgctttccacccacatctccttttctccttcactcttctgcctctcctctgaaagctgtgccctctctcctggaaggctctgccgtttcttggttctacttgaagctcagtttaagcatcacttatcgaggatgctttttctgaacactctatatcctgttcccccacttactatgtcaaatctcccttcacatctgttagaaagtcccataagaacatctcgatttctctcaatgttgtaatcatgtggtttgggcaacaaatactgaattcaaattttaaattataatattcaaaggaagtttctctaatctacactggacatgttcgcagcaaagtacaaatgcattgtgaattcatttgaagtgttgcatgttagatattcacactagaacaagcaaatttaatgagagtgtttaataataatgggtgttccactgtttcataaagttcaccagggcctaggctactgcctggcacatagaagatgctcaataaatatttgttaaacaaatgaatgattgctatatccagatttatctctctacctacatagtccatctacatccaaataaaaggtaactaggaggtaggaagtaccattggggtggaaagagaatagtccctcaagataaatcagggtgttgactcaagttgttttgtcagcttgtctcattttgttttcctacttcttattcataggtaaccactttgtaaacctagatgcaaaatggaaagcctacagattccatgctaacgagctgtggagacattctcagcatctctccaggctgtcaaagggcttagatcaagcattcttacacattgcctaagctcttctcagacatatttgctccaggcttttatcaactgccaggaaatgagcagctgtgagttccaaacctaacaggtatgtggacggtcagcatgtcccattctgatcactcaggagagagacattgttttggggtcaactgtgtctgataccccttccctcccagtgaccagcagtttgtgtaagcttgggctcctgctaaactgggggatttctgccctcagcctggggacttctgccaagcagcacagcttcccagatgcacttagctcccagtgcccgtgttttttgaaatgtcacgtggattccaagggcagagatagctccggtaatgtctgtggctgtttaaaatgtggttcactgtgatgctgaatttgttttacctttagaaagctcagaatttaattatgttttcttttcagcccttaatagtattgaaagccaacggttttaacagctctcaggttgtactgggtcttagtttttgcatgtagtgcctttttccaactaattactttttaaatgtagtaagagttcttttttgctttaatctgtcttagattgatattactgtgttgtcttttaaatttaactattatttgggtggctttaggggttttctaatagaatcatgacttttagagctaggaaggctcctgacatattttctaaatctcttcttttagaagtgaagcaactgaggtaaatgtgttaagagactggctcaggttctccaactagtggtgaagttgggattaggaaaaccaggttctctggtgctcatttcaaaggtatttccactatagatgcttatctagatattttcactgtgcctcagaaccaccaccacctttaatgaaaatacatctgccaacttgtatggaaaagaaaaagcagtgaatgataggtacagtcaatataatattgttaccacccaagcaagaaatgccaatagctactaagtgtttctgtgtgttggtacagcaactgccgaaatgtagatgccctgtttcttatgatagggtcacaggagatatcagaaagtataataagcatattgatgatattgattatttagatttgcagtggatattgagggagttgcttggcatccagacattttcaaatgacacatgataggttaaaagtagcagaaaaagcctcatgttctttttcaattagtaatactgatagagtacaattaatttagtaaggctttatatttttggttataataataacaacaataatagttttcatggcatcttatggtttgcaaagctctttcatatttacaatctcatttaattgtcacaatcaatattttagataagtatctcccctttcaaaagatgacattgagactctaaatctcatagcttatctgggagaaattagattttaatccagcttctttctattcaaaatctcatgatctttccatcgtcatttctttcttcaaaattgattcaacaagaggaaaattattagcctaatctatcacattaatagacaaagcagcaaaaactgggttttgtggctgtattagaacagtggaaacagccttctttggatgagttcgtcctggttcacattttgccaatgagggaaacctgttccagaggtgggaatagctcgaacttcctggaacctgagtagctcagatatattagcagaccacagcgcttctcagcacaagcttggtctttttatacatttctgaagactcagaatggatacacacacacaaacacattataatctcaatggagaaaccatagaagggaaaaaacaggatattgtagtttttaatagagattagagatactctataattagagatgctcatgaattcccatagaaaaggaaaagagaagatcaccttctaaaaatctaagggaatcaatcccaaacttaagaaggtaaacagttgcagatgtcaaattgatacataataaacaattcctgcgaacctgagataaattgccagaaaatcacacaacatcaaacaatgtattcattcaacaaatgtttatgaagaggctactacatgccaaaccatgttctcagcactggggttagggccaagaacaaaatagacaattctttctaagggagggagacaggcaaagcataagtttgtgtcacaccttgaatctcagtgcttgggaacaacagagccaagcaatgagcatggatactaagacggtccagccattgagaaggtcgcatctgagtcaaggcctgaaggtcagtgaattcaccatgtggatatttgagaaaaaagtgttcaaaagacaattggccagtgcaaagctgtcaggggagcctccctgtggggttaaagaaacatcaaggcagggagagtccagtggcttctgtgtagatgttgggctggacgaaggaatatcacacacagtcttggagacctttgtatgataggaaggatttgggctttactctgagtgaggagtgaaaacaggtattcacaataggacttggatgatcattctagtggctgtgttgaagcacatctccactgccaccccccctcctctcctaacctccatcatcacttacctcaattattgtggataatagaggcttgggccaaggtcagggcgtggaggtgataaaagaggacaagttctggatgtattttgagggaagaggcaacaagatttcctggggtggcatgtgagaagaaaaggagaatgctcaggatataggcctgaccaactggacagatatttaataattaaataatagacacatgggaactactggaagaagatgctaaaagcttatgatatactactaaaatgaggactttaatggtcttgttcccctcagagcctagaacaatgcctcaacaggcactcaataaatacttgttgaataaattcataaattaatagtaattattaaaatctcctctctgagctaagtttaagttggcagttctcaaactctagcatgcaatgaaatcacctcattaaaaaacagattgcccatccccagagtttctgattcggtaggtctgggatgtgacccaagaatttgcatttgtaaaatttctgggccatgcagatgccactgcttcagttaactagaggccaaaatccaaatacccctatagattctgacaacaaacagagacatgagaattggaaaggaagaggtaagacaatatatgcagatggtctgattctatatgtagaaaaaatatcagacccaaaaaatgaggagactacactctattgagaggcatcaaccttgttacaaccaagaaatcctttgttgattttatgggggttttggacgccatttttagaaagcaaatgtctaccaaataaacagcatttattttttaaaaattcaacatatggaaatcaatagattatatggttcatttcaaaagcaagtaacaaaagtaattaggatatctctggaaaagaagcaagtggagaatggggctagcagatattaaaacatgttttaaaggttctatcattaaatgtgtcgtattggcatagcaatagaactgaatagaaactccagaaatcaacccagtttcataagaaaatttaatgtatgatgaaacaaatcagtagtgaaaaatgaggatagccacatcatgcactaggataaattctaattggatcagagatttaaacgtggagagagggagggaggggaggggagggcagggcaggggaggggaggggagttggggaatggggaaaggttttaccctatgactcaaaatccaaaagccataaaaaaagatcgatagatttgactatacaaaaattaaaggacacaccaaaaaatagtgaaaggcaaatgacaagctaagaaacatacttgtatcacagacaacagggcaatcctaatatataaagcacttctaaaaagagagactaaaaagagcaacaacctatagaaatgagcaagagctatgtacagactggcatagaaaaagaaatgcaaatagctcttgatcatatgaaaagatgctcaataacttacaataagggaaacacaaatgaataggacactgagatagcattccaaaagtttgacgactgacaagattagtgaaaattgctggtgggatataaaatgacataatatctacaaaagggaatggccatatctatcaaaatcatatacctttgacccagctatcccacttctaggaatctatcccaaagacatagtggagaagagacagggctactcactgcagtataattttctatatccttttgtccaataattgattgaataaactatgatgcatttacatagttgagacctaaaaagggataaatatgtatgtgtgtgtgtgtgtgtgtgcgcgtgtgtgtttcctaaaatatacacacactttatacatacacttcgtcttagctattctacttctaaatttatcccatacaaatgattgaaaataaattaaagtttgaccgcctggatgttttgtcatatggaacaattagaaaaaacttacatgtcccatagcagggggttggttaaagtgtttgtagtgaatacaaaataattggagagctccttctccaagaaggcttccctgatgccccacatttacacagctcactgtgagtgcctctctatggcactcatcacctcatgccttgcttttgaatgattt

SEQ ID NO: 56 - DNA, 4556 bp, Homo sapiens
  source 1..4556: mol_type = genomic DNA
tcagcaaaacctcctgccccctctccagctgcctggtaaagttaaccttggccctttgtcctaagggctatgtctttatccatctttctattatcagagtctaattcaatcctgacctgagggcagtcactcaacccacgcatgttctgggctcccatcgaaaagccccagtttccagcagtcctcagagtacatgtgtggtgaatccctactatgtggggatccaggaacaagagccaactggccctccaagactccacctcagttccgattcctgccatccgcaaactgccactgagatggcagctctgggggcagccttccccacccggcagacagaggctggcactcatctttgctctaatgaagcaatgacacagtgatatctgcctttcctacctggccaaagcctccctgggggcagaaaggaccatcctgctcttgctgtgtcctagggccacgcttgggggcgcccagaggccataaagaccatgtgccttagctaagaaacaatttgacacgtgccaacccagcctctggaccctgtcagggcctgctagcaagcttcctgagggcaaggctgtatctttcatatgcctgttattgccctaacatctggcctgggcctgctgcctggagcataacaggcctcgtacatatttgttagatgagtgaaggatgaggtaagttacaactgctttaagttcactgcctagtaaatggccttaatccaaagatcctttggatcttgagccaaaattatattgagccaaaatctacttccctccaatacagattatgtgtaccatgaaagccccatatgcatgtgaagatggacattatactcttccaaaagcctggatccaagctgaacatagttcccgtagcatttctcatcgcagatgcaagatccttcaccgtgtggccacctttatcaggacaaatgctttttttcaagtacatcttgaaatatggttcgtacctgtgaatataaccttatttgccggtctttgtagaatcaagtcaagatgaggttataaagtgcaatggctgatagctgtgtaagggagagatttggagatagacacatgggaagaaacctttgtgaagacagaggtagagactggagtgatgggcctgaagaatcacaggattgtcaaaccactggaagctaggagaggcatcgaacagattctccctcagagcctctagaaggagccaaccgctgacaccttgatttcaggccttcagaactgtaaaagaatacattttaatacatttctgttgtttttaagccactcagtttgtggtactttatgacagcagtcctagggcactaatatgttggaccaccaccccccttgatcaatatattattctttgcaatcacatggttgccttaaaaaattaaagactactaaattaataaaatgatttatggctggacacaagataaacaccccaaaatcaaggcattcatctttaccagtaagctattgaaaccataattcaatatcctattcacagtagcacccaaagccgtaacacatagcagtaaacctatgtgggatctgataaagaacactttttaaaggtggagaaaataacaaaatcctgaaagggaaggaaaggtccatccaacgagatattagaacatattaactcaagagtataaaacagaaaaacaccataaacaggcagatgctagttgacgtctgttacctcctccagaagcaagctcagagtgaggtgagatgtgttgagaattggggcacttaggtgcttacacagactaaaaatccttgttcaccatttatcattagcagcacagaacgtcatttctacccagagagatcctggcttaaaaccccatcaaccaaattgcttttggggtggaaaggaagcccctccaccttattttgcaatgatatgaaaagctgttctcgaggagaaagaaagggggagaggtaggaaagatagcagcttgtgctatctactgatcacgcctgcgtttctttggctagttctgccacttgccgttctctgagaggggtttggtaattaccaggacaacttttctcagtccagtcatttgaatttccagaacaaaattaggttggggaaaccccctgaatttgcagactcaatattctctgtttcatcagaagtttgctgagcacctgtggtgtgccaagctctgtgccagcccccaaaggttcctgagggttcccaatacacagttatttcaacaggatggatttgcacgaagctggtacacagacctgctgaggaaggggccagccaagcagtcatgtgccactggatccgcttggttttctccacctcattacatgagaatgaccagatggtttataaatacaatcatttttattgagtaggaagtaattaaaatgtttatacctgaggccattttaggaagaacttttttgccatggtatagtcacaggggccagatcccagatgctggtgtggggtgaagcgaggatgaggaccagggagatgatgcccaagtgagaggatattgtgcaaggagctggcaaatgtggctcaattttacagagcagtggagggagctgcattaggggctggcctagttcagtggatggtgacctggacacacattataatcagctggagagctttaaaaaaaaaaaagatctgtgctcagatcccaggccagcccagttaaatccgaaccttgggggagacactcaggcattcaggtatctgttgttgtggatgaatctctacaggtgacatgtgaaagctctgagaacagagctccggccactgcttagttatacctgacctgggcaaataaaatcacacacacacttacatgcgtatacacatatacaagtgtgtaaaatctggttaatgttctctgtcatttcctttcctttgactaaagtaattgtattgaagatctctcaggttgctgagggcattatacttgtgcctttttaagggtttgcctacagagtgtggagatggggagcccacaaggatttttctagaagtttctatcctcagagatttgctctaacagagggtggtcagttctgaattaggcatcctcttgctcatgcttctgatacatttggacttcagagtcctatccttatgccgctagtcacccagtgattcccaaggaagaaaagcatcataagatttagagatggggcttccctctctgtgattaacctaggtccagttcagaatgaagaaactgaaaagagaaaatccaacccttttcacatccaaccccaccctgtccctgggtggcccttaccactggaaaagcctgcccaaaaactttggtcctaagaaaaaaagaaactggtgcagctgcttttatggaaggagctagaccagagccaacaagtcaggcctctgtaagaatcttccaaggaggtaaggcggggagaaaaaggattcctcatttacattgagagtaaacttcctgtcctattaaacaatgaggcattcaaaatccgcctgagtaaaagtcccctgatgcaatgagagggtgagcaatttcatgtcaggctgttgttatgggatttatacatgtcctagctagggcttgaggtgaaagtcagagccaaaaacaatagccctaaaccccacatgcacagaaacctcattccagtggagtttccttaagagagcacagcagacttttcacaaacaaagtgacttcaaaggtagccttgcatcactgcctcctcccttcccttcagcccagacctggcccctctgtttcttggtttaatttatgaggttctcacacacattgcaaagcaattatctgagctgctggccaacaggaaggtaattgtaactgagtacttcctttgtcaaacgctgacgctggccaccgcgaggtgataggaggcgtgccggcagctcagcacccagtgacaggatacctttctcccccacccacacaatgctgtctgactgaattctccagggaggaggaagccagtgggtgaggaatgggggaattttcttacatcacttgacgtgcttgctttcactcgcaaaatcctgagtcatgactgaagctgtattgcctcaagctccggctgaaaccaggggttataggtttggaaacctgattccacggctgcctcttgtccaattcagaggaaatgtgtattatttcagttgatcctcttcaagttgaagtgcctttggattgaggggattacgtgagggaagtggctctgtagtggcttagtaagccaatgccatgcgagtgaacaatatttgacattcggtgagtttacagaaagggcagtctctctgtttgtttaaaccagattaagtgaaaggaaaagtttttctttcccatcccaagacaaatgtgaaacacatgttttcccttttgactttgtaagtacaaggctcaaacagttctccccaccccctcggccatgagtttttaaggattgcagaatgtttcctctctttggtggccaaagccaatgtgagcctttatatttccagacagtggagctgatgatgat

SEQ ID NO: 57 - DNA, 3748 bp, Homo sapiens
  source 1..3748: mol_type = genomic DNA
gtatgttcaagggctaaattgcttagtagttgtgcagctacagggagagaccagttatagccacaaagtccttggaggataaaaaaatggtggtggtggcgctgagaatccttgtgaggagggctaggctttgttctgggcagtaagagagcccagggcacagaaaacatgattaaatgtgtgttgaataaatgaatgaataaacacttcattggagggaaaacaccagtaagacattggctgtgtcctcaaggtgctcattccccgttggaagacacaggcccattgcgctaactagaacacaagcgagaagacagtaaatggaaatgttgttgagccttggtagggcttggcaaggtctcttatggctctgctcctgtatgctgtctctccattctggaagccctttttgctgcagtgactggctccatccacctgccagacagacgctttcgttccctcttgaaaaggcttttgcttgatggaggaggaaaccagggaggaaaagagacgcatcttattctcctgagaagagtcatcctgtttgtctccattggcggctggctttatttggcttctatttggatggtgaacccattgccttgaggtggggagaggaggagattctggaaggatttcagtattgaggcgcccctaatacctcccagatgaaaaactaggagtgatcctcccaagtgtgcgtgctgacatttgctccccaatgcactaagcataagcttgttttattgtgtttacaattttgtattcagaccgtaatattgcaaacatgaatgtcctagacagtagagatgtccaggcttttctctaaagccagagctaatcctgcaccagagatccagtttgatgaactcttcagtgttcccaaacctcatgtgcttatgagagatcagatatctgtcttaagcagctctaccctactgcctacccaggagaggatcttggcatgtgcctgcctgagattgacctcctgccctgtgaccctgagtatagagcagaggacaggcagtgttgcaaatatgctcctcacaaatccagataggagatatgatttaacactatcttctccttgaaccacttgataatttttttttcaatcgcaagaaaacttaattcctgttccactgtctctagctctctgagggataatgctctgacaatgtagacaaggaccagatgtgcagtggatgggtcggctgctccaatctggctgtggagacgtgagggaggattggggagacagttgtatgtagtccatagtcaggataaaaggcctttgcgagttcctaagtatggtttcttgagtctgcgcagaagggcgtatgtgggaaacataaacaacacaaaataaaacggtttgatcagaaattatagttattaccaattccacaaatcattctcctcctttatcctagactacaaagaagcatcacaaattattgacaaagatattctggaaggttaactctggattaaatcagacttctctctcagcagctggtgcctctcatctaagatcaaaaggatcttgaaccttccaagtgaactgctgtcaagcaaacagcaggagaacctctaatattatttacatagcattttctttttccaattatgtattggtttcttcttggtacaagtctagcttataaatgtggaaaccaagacacagaaaagaaaacaatgttaaagttaggtagagagtcagtaagacattaacccacagataaatgtgtctagatatcacctctacttattcaacaatccttttcagttgcctaccaaaaatatgaagtcatttttgctgagggtgcagtaaagcatgccaacttcattttccaacctggagtttcttgggcctataggaaacggattagagtctttgaggctttggggaccttgtttgcaacgttcctctaaagtagaccttataatgaatggaagcaagagaagagtgccagattattaatgctgtcaatatcctgtgtctctgagatgttgcccttatgtcaggtaccagtgaggaggaaaaagcagaaatgtgaactctcagtcacctactcaggccaaggtctctgtctaggaactgctatgtgtgagtcaatttcctgatgacagtttttccataccatgctgagtggaatgtcttgtttatcagccctcacacctggtactgtggcatcatgacgattgtcatagtctatgggaaggtgacaatactgtgtgactcccagtttcagactcagtcattaagcagaagaaagaatgggaaggcacagagatcagagggagaaggacaaagagatacaaggagagtgacaggaaagaagaaaaaatgactgagaaactcctaatgcaaagtgggtaatgggttttgaaaataacagggtgagccaaagagaaagggacaaatgatttagcagagagtggaacatatactggtacctccacctactgtaccccaagccttgtgttgaacctggacaaacagtctggcccttccagggactcccagcacaacgaaactcaaaggaaagaagagcaggaagttggtgggtttcaggtgcttggaaagaagtaggctgacatctgactcacctattcaactgcaagcaccaacacacacagaagctttcagatgagatcatctggtcgtgacttgcaaaagatgatttggtggcatatggggctaggcacttatcaacatttttcttccagcacagcaaaaggtgggtagaattgcagctcttttataataataaataatgttatggaaaggaaaaatgaagtatagagaagggacagaccttaaaaaggatgctcaatataagaaaaacaaacacacattgttctccttgatccattgagaggatatggtaggacagaaaacacacagaaagacgtaggcctaagcctgcttttgccacttactgaccttggtatcttacaatgggaaaataatacctactattcagggttgttgtgacggctgatgatgagatgtgcatttgttaggagatcagtaactgtcagccattattctactatctgttagcttcaagcagcagtgtgaggactaacttctccaggcagctcctccatctccccagttattgttgttttatggtactttggaaaggagtttcaagttcatttggttcaagtcgttccttttgtggtccagaaaggtgggtggctgattcgacatcaattagtggcaaagctgggagcagaggatcctaaggccacaagggagtgctgagcacaagattcagtagaaaacacatccctcatagcagctctctgggttcagggctcctggaagcctttggagggagacaaatgtttttcatttaagaaagtgattatttagtgactcctgcacaacctcttctgcagcccctggatctgattacacagcctgccccacgaggtgggaggagaagctggcatgtggcatgtggtttaaacgtagttgatctttttaaaatcccacattttagccagtctctcagctccacatgatctattctcagaaaatgctaaccggccagaaggaaagagcaagagaagaaaaggaaggagaaagagtaaaagagaaagaaaagagtgacaatgaaaagcaagtaagaaaagtgaagagaggaaagagaaagatggaagaagagcgagctggagcaaaagggagagaagtagcgaccctgaaacagtgtgatgagatatggccatagta

SEQ ID NO: 58 - DNA, 5013 bp, Homo sapiens
  source 1..5013: mol_type = genomic DNA
aaatagtgtcttctggttttgtctctttgctgagacagttgcttgggatggctttggaaaaacttgttcctgaggccaggaagggctctgggaagttttcctcagctctcgaataccccacttatgccctcaaacaaaaaaatgagttgcctttaggatgattcatttcaattatttcttctcccttaaagctgtttggctctgcaggggtttggggaggcaaggaggccagctataggtgcaaaacagcttcatcgctggagggacaccatgcccaaggtgggaatggtaataacgatgaagacccgagaaacatccattccttggaaaaaccccatgtggtgatggtcaagtgggcactaccctctagacagtgtcaggaatgagaacttaagacagcctaatgaaaatcagcctaaggatttttgccagaacaattgagaatgagactctttagagtcactaaaacatggataagaatacttgctgccttaggtaggtactcaaggtcacagagctaaggtgcagacagcaagcttcaactcaggcagtctgattgtaatgcctatgtgctccctgtgaccttactaagccatttaatgtcatctccatgagggagtagagaggagcctataagcgttaaggtgggagacaaaaggaagaagtcagatgtacatctggctatggtggactagaaatagatcaacttacattagggaacagggtcaagaacctataaaaatttatgttgttgaaaatgaccataaaattgatttctgaatgacagtgaaggagctttgtgggcctactacctagtgaaaccagtgaaaattatttaaagcaaacatttacagccttgggcatataacaaatgaagaaaacctattcatgaaaattcagtaagagggtaagtggaaattaaaaaatatactcctacaaccagtggattaaaaaagaaaaaggaaaatcagaaaatacttcaagatgaatgagattaagacaaaacatactaaaacttatggggcagctaaagcaatgttcagaggaaaacttatagttgtaaaagagaaaaaaaatcttaaatctataacctatcactttaagacactaagaaaaagaagtacaaactaaacctacagaaaatagcagaaagataataataaaaatcacagttgaaattaataaagtaggaacagaaaaacaatggagaaaaagtatgatgccaatctagttgtcttaaaatgtcaattgacatacttttagctaggttgaccaagaaaacaagaagactcagtactagaatcagaaatgaaaggatatcaatactcaccttggaaaacaaaaaagattataaagaaatatataaatcattgtatgccaataaattagataacttagattaaatagagaaatttctagaaagactgaaataactgaaacttactcaagaattagtctgactagacctataataataaaaaagatcaaattaaaaataataaaaactaccacaaagttaagcccaggtccaggtggctttcctactgaattctactaaacatttaaaggagaattagtactatctctttaaaagtttccaaaaagaaatttaagtatttatttaatgattttaaagatcagtattaccctaatatcaaacatacaaagacatcacaacaaagaaaacaagagatgaatattcatgaataggggtgtaaaattctcaactaaatactagcaacctgaatctaataacataaaatccaggaaatctaattttcagtcaaaacaaggctattttcaatgcagttctgcctccatttctgattcacaaacacacttcccaggaaagaagcctcttgtatacatctcatgctagtgtgcatgcagcttgagaagtcagctgcatggagaggagctggcagaaataaaaatttttctgaaaaattcagagaaaacaacaactgtaatgtacatatcattttgcacaatggcccgaaaattatagggaaagaacatagatgatgtgaagaaaaataatatgaagaaaattaatttttgttggtttttcagactttcttggaagaaacagtatttctttcctcatatccattcatgttaagaattcctaggatgtctcaagcacaccctcaataatttacaattgtcaagccacaatttgtagacactcacaactttaaattaaaaggaattcctagagaaaagttataataattagtttgtgttgtttcagttaggatgtttttgtccataaataatagaatatcagatgaacagtggctgagacaataaagtcatttattactcacctagcaagaagcctgcatgtaggcagttcctgggctgtttcatcagctcaacaatggcactaaagatttaacatatttctacttatttcgtcatccttaactgttggagttttgcctcatgctcattgccttgttatcataaaacggaagctacagccctagacatcacatctgtgttcaaggcaggaagaaagaagagataacacaaataaactcttcattcattggtatcttttataaaaaacacaacaaatttttttccccaaagactcttggcagacttcataacgactagactgaactgggtctcacagccatccctaaatatgagtaaagctggagaaaaacctgattagtaaaagtgaaaggctgctaagattggcttaaaacaaccatgatctagtaaagtacttccttgaacaaaatcaggattctattagggaggaagttgagaatggctgttggttgggctcctcacaggtatccacaaatgccatgtaatgttcttttccctgagctattttcccttctatccaatcccattcttagagtctcccaaggagttagataggttctgtgtgtcacagatgtaaagttagactctagtaagtgagcggctcagatgctctccaaagctttctaaaatatttctcagaatgaaagtaattctgtggttaaattcttttgaggaaatactatctttaaaacatagagcacttaagcactcttataagacagaggtgtgctagatgccttagatgtattatccaacttaaaagcctacatttgtgaaccctgggagagcgggagctgggtcttgtttatagctatagctgttttctaccagagtgcctggcacatggtaagtaatcagtacatatttaataaataaataaatacaacttgaagacaacaaaaaatactttttgatggcaacatggataatgaaagtacaagtttcctcctactaaggtaatcgatgggatagaaataatcgcatgtctttttctgatgtttactaatcctcacttaagttcaaaaatacttttccttggatcccaaatactagtgagagcacagctggggttctagacctcacttctgattctaactccttgcattcaatttaccgtgctacattacatagaggataagcgaagggggccagttgtatctggaattagaatgaggttcttctcattctaaagaaatgtgatattgtataattacctaagtcataaatgtgtttatgcgtagctaagtttattacattagcttcaaagcctaagcagctccacacaagacaccagacattcacattcatagggctggagggcataattcaacacaccttgtctgccaaatctttcctgtgcttgaactagagacaaaatgaggagctcctaaatagttgacaatttctaattcattcttttatttccagtgcctttgtccaaattgtaatagcataattctgataaacaagtacaggtatcctttccaggtatctaggtgccaagtgcccatctgcctaatgggcacagggtggattgggtagtattacatgtacttttccattctgcccttgcatgtcctcattcatcctaggatagagtgttcttctttgctacgttatggggctacaaaatccaaattgaagctgagcaagaggtttaggaaactcaatttgagaaaagccctttggtaataataagttttaattactattttcaaccttattttttatttaaggaaacttcttccatcctctctcagccccactacagaaaagacctgtgacccagactggccaatctgtgtactggaccacagtggcttgttcaaattttagcgcagaattagagtcttgtaggattaatatttgaacacaaagagaaaaattctctttcctttgaggatactttgaagctgcagatagatgactcctagtaaatattttgccatttgttcagatcttatctgcaaaatgaggttaacaaacacaagtggtggtggtgatgacacaagacatttattgagtgttacttatataccaataactaccctaagtgcattacacatattaactcatttaatcttcacaaacttataattatgattactgttatattattgctataggcacaatttgaacccaggcagcctggctcttgagcctctgctgcctctaacagtacaggaggaggaggaggacaaagagaagaacaggtttccaggatactgtgaagcccttggatccagccgtgtctgaacctagggcacctggattcttggttaaatgacccagtacatcacaattgttatttagccagtttgagctgagttcctattccttgaaacttgcctaaaatactctaccaccatctctcattctgattgtgccttatcatactgctttaccataaaaacattgtagaaatttggaaaatgcaaaaggataccataaatgaaaagaaattgcatatgatacagccaacttgagattattatttttgttttcttccagtctgtttttaaatcatgtacatatacacctgtaaagttgtatatgtttcttgtctttcctttttctttttatattaaataatatgtgttcccctatattaaatattttgagaccataattaatggcaatataatttgtgtgcacacgtgtgtgtgt

SEQ ID NO: 59 - DNA, 1153 bp, Homo sapiens
  source 1..1153: mol_type = genomic DNA
gggtcttctcaccctagagaatctaccattgctcagctgcagggcgacacccaacagtacagctgggtaacatagaaacacacactgacttcatttacacagtccctgcccagagtcaatagaaaagtccagtccagattcgtcctttgcatcataagaacaggagcctggctggatttggaccccaattagtttgctaatccctggaataaactaaaggggcccctgaggcaccttccagcagaccctttttcttaccattagtgaaaaaggtctgtgcggtgggggcagtgtgtctatgacctcagcttgaccacacctagacaggggggaaaaggtggaattagtccgtctttcctatatcaggcccaagcctgtctagggcaggcttctgttctgagcgtattttccattcttccctccacctttccactcactcaacaaataccctttccccttaaggtgatcaaaataagcttctgttgctggcacccaaacctccctgactgacaattacactggtagtctcacttgtccagcccagtggatcttctgagcatgcattcggggtcaggggcccccaaagtgctcaggattctgggtccccatcaccatcatccctgggatgggtgatgtggtcccagtccctgaccatactcagtttgaccctggagtctctgagtccccaggtggacccaggcaactcacgcctctccttggagtaacatggcatcggtgcatcaagcaccttaggcgtgggtcaaccaaacagtaaggggttgcatttgtcagatgtttctccagaggcttaagcagtgcccggtagagacccaagtcaaatgtgtctctgtgaaaggggattcactggctcatgtaactgaggaagacacaggtgtgactgggagctccttcacctcccatcccagttcaccacaggtgcacactgctacccaattcctgccactagtggtgggtcctcattgtcatctggtcagtggtacctctccagcatcagagcctgcctcccagacccattcccagcacccactgtcttcgatctggataggagcagagcataggcatgctgattccatgatttattgagggagtgatcttgggagactatgagggaggcaggcagaacaaggaaggg

SEQ ID NO: 60 - DNA, 2938 bp, Homo sapiens
  source 1..2938: mol_type = genomic DNA
gcactggttctcattgctccatcaaatactaaactctaattagtgacaccgtctccagccattctagtgtctaaaatatcacaagtcacagtatgaacgatcgaagttgagccccaagttagcagaggtatttaagttacatagttgatgaaaatgcaaatggagacataacaagtatgaaattcagtcaccaccaaggaactagtctgtgaacacttaggattttcaccaagatatatgcattgttaatgctttttccttctaattaaaaaagaaaatggataacagatgaggaaaatttgatgtgatggtttttaggaaatgcacaaatgactcatgcactcgatttgacaagggtactggtttctatataaagaaacaacttaggcccaaaatagaagttgtgcacagcagattgtaactgatagaggaactgcacattccaagtagtgtgaagtggtcgcaagagcagtggctctcctgctctggggctgagaacccctctcctttgtttagcctggagaaagccccagtgaagtgaaagctgtgagcacaaagggctgagaggcaagcaggacattgtgttcagagtcaggatttcccagctggaaaggtctttacagtgcatcagttaaggttagatgcagcagcttggaaccgaagtcttagaggagcactctcaggctgcaggctgggtatgcacaatgttgtcagggtcctggcttcattttcctgcaccaccttacctgtggccccctttctagtgatgttatttggggcaagttgcttaatctttctgagactcagtttcctcatctgaagaagaagagtaattgcaatacccatttcacaaggataatgagcagattaattaaaataatacctgtctttgcaacagtgatacagcaagccttggctgtttttatttgttttgttcaggacttgtacaatgccaggcatatagttggagctcaataaatatttgtgaattagttattatcattattattgttgccacaaaggacccacaagcgacatcaagagcccactccatatagcacctaggaagagcctaatttaacccaactttatttccaaccagccaagctggagtgcaattgtcaaaaggcagctgatggacagcccagcccacgtcggaccggtggagcccaggactcccaaatgctgtgcctccgccagaccagacgctgctaatggcttttgttcgtctgccaattctactgcccatcctcctctgggaagaaagagcagaccggacagagtccttcagaggccgacgcatgccagaggctgggagacttggaaggggagtgaggagacagtcactggggaagacagttctcagggattccgcaaggccctggaggtgtttctgtctttgtaccttggttgcctctgcgcaggcagccttgcttcatatcacctgcacttccttgcttctagagggattgtggcaagaaagctcgcagaagtcaaaacaagcctggtgatatgaaaaccatatgaaaacttggcagggtcccatttcagactaatgggcctgccagacatcaccggcccacagacactggccctttgtgaggtcggggactggctccctcactcccattctactaagacagaattaaaaaggaaacaagcaaggcagccagtctcttttctctcatccacgtgcttccaaggttagaaacagagtaacgtcaggtactttgagtgatgaacacaggatgttgttggccaggcccattcacagtgcttttcagtcctcacttgatcttcatggtagacccatttcaaaaatgaggtcgctgaggtttctggggctcccaaggttccactaaaaaatgatggcccagccagctggggttcagatgcagacccatctggccaaaaaccttgacctttgttgttacagagctacctcccagatgaccttttgattatttcagacaaatttaccaacattcaaaattttgcttttagctttctgtttcctgttctagaaagaaatcgagacataacatttttttttcatgacgttattgaatctttggacaatactgtaacatgccattgttatttatacctgaaattcctgtatttcaaagcctgttgggtgtggagggagactattttccagagccatcattattgcagtactaaaataagtgctcttttaaagcaacttaaaaaaatcaaaaactgagataagtggctgtgcaagtataatgacttgttctttgttcccaaagactggaaaaacaaggtcggtatgcaaatacacctgcagattataatctgtatgtaatttacaattcacatctcttcgtgacatggcttctgaaaaaatctgaagatattttaaagacattttaaaacttgataaacttttgtaagtctggcaagacccaggtgctctgaattcctctcttatggattacggggctggaaattgaaccctctgctagaagctgcccggtcacttctgtaaaggccctgatcattttagaagacacgaggatctgcgccctcatgaagagactcatgaaggcctctcccacttagctgcaatgaaggaaaggaagggggaaggagagtgatcaaaggaaagggagtaaaaccccctaattgcccatggcaggggtcaatgaatcactccacgtgcacatccaacagctggtgagaagagctgaatctgtagggactgataaggaacaatctttttattatgttttatttacagcacatttttttaatgcaggccagggtataatatttacttaagaggtaggaactatttagatgagaaaactgcttatacttctagcatctgaaccgaagtagagaggttccaaaaatccaaaccatatgcatgtactgcttttaaagaatgcactattttttctcaattgccaag

SEQ ID NO: 61 - DNA, 2081 bp, Homo sapiens
  source 1..2081: mol_type = genomic DNA
ctcctctaaaacacttatcacttggttctgtaatgcttacagtgtgttacagtgaaaagaacatgggctttggaatcaaacagcctgtgtgaaagctggctctgccattcatctgcctgagcttaggttaggtacttcctctctctaagtcagagtgcggctactgatgactcccctggttgttgtaagaattagagaatggaatgcatatgaaagtaccaaacactgtcagaagcagtaagtgctcactaaagattggatcttctcttccctagtgacagtgcgtgacttaaagcctggcccactttatttattttgtgtatctccagagcttagcacaagatttggtaaatgggcattatttaaaaacaagaaagtggctaagtgctctatcaagggtgaaagtggaatttctggtaccttatctgaagtcccccctgttgcaggttctcaacatctgttgtcaatcttcctattagttaattaaactcctccaaacaccatccttgactgttgttgcttctggctttttcccaatgagtcaactcttcaaggaaatgaaagagcctttgcacagaaaggccagaaaaccctagttggccagcatcctttgggtctaagctatagcagcgcaaattagcccagccatttatatgcaaaagccttgaagattgtttgaaaattgcctacctaaggagtgtaagttaaaattgattcaaaaactgtttggttgcatctaggaaagtgaatgccaaattggtcttttcaaattcaaatctaagttaaccttcaacttaaaacctacagcgcctcttcattatcacttagattcaggggcagaatctcttccggcctgagtccctaagtggaccagcctggctcccacattgcatcttagcacttcagctcctttgttttactaatttccgtgtgtccaggatctggtgctgcactgttgagttgtgctgaatcactgttatttctgggtttaatttctgcagttaggctcagaaatggggtaactctgtgtctcatgggcatggtgaagcaggaggagcaccccttcctcaagtgataaattgcaatgaagcaaccatgtcagaaaaaaattagggaaccattgatcctcatacaataatacacttatgtattttcattggaaagagagttatctgtgctctgtgaacattattggctagccctggataaccaattttatgttttcacaataaagttgagccatgttcagatttttatcccagggagtttccctggatattgacgttgaaagagcccatgggggacatttgcaattggaactatatccctagatgcctccacaagacacatacaccagagctgtcaaaagcagagttctttataacctatcacatgatagctactggcagcctcgagctaaccccccatctcagttaaaccttcctaacagcatccttacagatgaaaaactgaagttggtgtgacttaagtatctcgccttaaggcatccatccattgaagttgccttcttggtctaaacttgagcctgcccagttcaaaaggctttctttcagcctagatctaccactacggaggtttttatatataacgtattttaattacttgtgttcttgttatcatagatgtattttaagagaatagaacctgcttttctaaagctcaagtacttttttataacagccacttaattggacacaatactcagacatgtcagtttttatatagctaaaaactgaaataaaactgcacattaaaaatgtattttgccaattttctgcaatgaaggtgtatcatttttaaaattagagaaaggaaagcattgtttcaaaataactttggattcaatcaatatggtaacattatcagtgcattctgggtgctgggctgatcatgcgtgtgtattaatctggtttattttaaccgcaactctgaggtagacgcatttgcctgatattttaaataggctgtggctggaaatgttaagtaacatgttcaaacaggcactatgtgctggagccgggacttaaaccaggccttctctctaagtgcagccccctttc

SEQ ID NO: 62 - DNA, 3911 bp, Homo sapiens
  source 1..3911: mol_type = genomic DNA
cataacacagtcttcagcccctgccccagcacctggcacagataaggagtgggaatgtttgcagaagaaacaaacttcagccaattcactctgagttcccctggacctgctcctccgtttgaacccttcttgtccctcaagtttcctgccccttttctcaccagcagtcccaccttccactgtttttctggaaggaggtcagacacaggaccatttgaaattcttgccagattggattcacctgtagtcaaaatcccgagaatgaatgtcaggcttcctgtcccaaacctccttgggccagggccctcaggactttcccagggacagaggtaggcagacaggccagagaagagtcaagcagagaggatggtgaggaacttagttcagaaatgatacttaaaggctggcagttgccaagctccatccgagggcatcccagagaccccagggcccttcaggaagcgccatcctcatggtatcccagccagcctgagtggcaggggtcggggccaggccagcctgagcccagcacgtgtaatgaccatcctgacaagcacttcctcccatgccagctcacggagctggcatcccgggcagctgccaggcacctgctgccggagagaaaagccccatcatcccagcctctgtggacagacaggccggctcggattccagaaaggcatgcaaggtcagagtggagccccttccccggatgtgggctgagcagccagcacagggagaagaggagccaagcggccagggtgcagaagggtgaggaagatgagaggcaagggacaacctctggtcctccccccaggccttcacttccactaagcatactcttcatactcattgtcaccctcttgattgcgggcagccctgcccagccttcccagctaccagcccccaccaggactctcttggcaccccagccacccagtgtcctcctcccttccaggccagccaccacctccatgctgccctcaccacccactgctgcaaggcccccatcacagagcctcaaagccaatttctctcccagctgaaacccacccagccccaagcatagctataaagccacacatctgccttctgccagccctacctgccaccctctctcctgtggacacactgacatttgaaggatcaatgaatactgagcagcagggccagatgagaggctccagaaccagagggtccccttcctcactcaactcccatcaagtgagatcagccacatgggaccagctggggaaaccgaggcgtctgtccactctttgtttacaaccagagcatcttatagataaaaccagacaaggtccctgtcctcatgtagctcacaatcaagttaggaaaacaggttctaagaatgaatcccaggtcccatccaccagatcctgcctcctacagcaggagcctaaacaaggcaagttcctgttgaggcagcacccacacgtcgctggtgatggtgaagacactggcgtctctggtactgtgaatgcagctgtttgaactttcccttgggcacagagcacaaaggtgccaatttcttctgttaaccaatcaagaatttacatgagggctccctctgtcaactgatgcgcttcctccagggaccagctttgcataccagagatctcagggagaggaggctttggatctagtctcaaaacaataaaccaggtgggggaccaaggccactcattcaccctacatttacataactaagaaagctatttaagtggctttcattgaactgttatttcattttaaactgtctctgtgacttgtggacacagagagtggacggcacttaaatcctcctcgttggtccccagagccttgaggcttatctggagaacagacctcccacagcaccctatcccagcctccacacaggctctgctctgagtgcttttcggcagggaaaaggaacacgtccaggtggaaatcttccaaatgtgtcttccccatcaccctgtcaccagattgttctcagcaaaaactttctcataaattgtctttgcctttggcttttcacacacaaaggtcacatagttgtctgcctctggtatcacacgtgtagctgcagaagtggtttcagaggttcccacactggaatctccagaggaattttgatcaagaaaagggaaagttcagcaatgctcaacccgggtttcaagacccgccacatacaggacccagaccctcccctgcccaagtgaaatcattaggactcaacaggataagctccacgtttctgacttgctgcataactccaaaacatctcttgtcactatttgcaaaaccctgcaggggcctcagaaaagaatcaactccattattcctgcctctaaagagttagcagggcactgggtaaatgcatgaatgaccgaatagaaagcaaatgaaaataacaacaaccttggggagttgagggtaaacattagattggaaactggaaaatcgtaagttcctagagtttgttgcttcatcgcgccgattaaatggtggactgcataagctctaactggaaagcgctctgcaaactgagcacggatatggctggttgcaacacatcacagtcttagaaccacttccaccacctcaggttaacaccataccccggctctgtccttcactgactttcatcagacaccaataatctcacaacacgaaacttgcgtatgtgaagtgggttgccatgtaaagtgagttttctaccagagggatcttccattctgtcttgttcaatgcttttaacaaacgcattgaaggcttgtttattcaatctgcatctcaaagctcggatgcatagctaaccatgtcagatggccaaatcgggatcatcaagaatgtgaactccttgaagatgggtagaaactgtaacacgaatcctaacagttggcacacataaaactcacatttgtgctccaaaaattggcctgtaagtgcataacgggaaagtgatttgacagcagatcgatcaaaagacctgaggcagttagttggaccgagttccacaggaacaatggtatatagtgtgaaaagtggagcgtccctgagagagcggtcaatccctggactcagttcagcacgtcctgaatgctttgctgtgaagggcctggcacctgagcgtgacatgagatgccacatgtctttataagctgcatatctcaaaaggacttcaggtgaaaggaggtgccagccagttctgtatactgattccaacgtcacaccaaatctacagaatcaaaacctcaagaagagttagagaatctattttttctttgagtgaaaatcaaattctggtaattttcttttttaaaattataataaaatttaaaattatataagaatgttcttattcttaggtaatataagctgaagtattagaaaggtgaagtgtcatgccttaatttacttttttaaaaaaacagcaaattgcttttaataatttggaaaaaagagaaagcaaacgagacaaaatttggggggagggaatcattgcactcttcaacttttctgtaggcttgaaatttctcaaaacaaagtttaggggattaagctgttagctattctgatgtgaaaacatgagacccactggacagataacctctgctattccatccagcctgagtgcctggctgaggctgactgagttctttgtggcttacagagctgaggccacagtgacccctagaggtacttggccccttgaccacagtaagcccatccccccactggcatggggcctgtgtcaatgcattggccctaatgatgctcccggaaaaaggaacagatccccgatagacaatctccagaaggatgaccatcccagatggaggctaggcgcaggagtttcacacctgatgtgagcccagcctgaccaaatccatcagaatgaacta

SEQ ID NO: 63 - DNA, 1009 bp, Homo sapiens
  source 1..1009: mol_type = genomic DNA
aactgaaagattttcaatgaatggaaatggaaatgacactgcaaaatttctaagactaggtcatatatgattaactgacttctacctggctttctcggggtcttttgtccttggaacccagccaccatgttctgaggaagcccaagccaaacagagtgtcctggccaacagacagctaaggcctcaccagagaccaacatcaactgatgttgaatgaccctcatgtgaatccagcccctgacattgtgggcccagataagccatactgcttaagccgtctgaatttctgatctgtaaaaaatggttgtttcatcccactaagtttttggttttaatttttcatgtggtcaggcccaacaatcttttgtaatttcttatttcagttgtaaacagaaattcatcttgtttctacagaggtgataaatgatctatcctattttttgtcaactcaattctaactgaagcattctctggacactcggaactaaaaattcacgttggctcagcaaaatgcaaatgaagagctctggtgcaaatgctattatggagtgggtaaattgagaatgtgtgataatcgagatcacatgtccccattctcacttccagttgtcagtttatgctgcctcagcaaaacgagttctggggcaaatgctgtataagcaaatgttataattacccaaagatagctacgatgaaaaataagaagaagtcaagttgaaaacgccagaacgcatgaagaactcagaataagacaagagagatgggtttggagacaaaactgtcagaatagagatgagtaaaaggatagtaataatcgttcctgattattggaaattattgccgtatttctccctgtacagaaaattatgcaaagtttttcacctacatacacacatagaaagaaatctctctactttcgggcagaaaaaaatttgttgaggcactttggcctagcatatcactgattaacataaatgttacatatgtgcttagaaatgtgttctccaag

SEQ ID NO: 64 - DNA, 1804 bp, Homo sapiens
  source 1..1804: mol_type = genomic DNA
tggaagctcacaatggcatggtgtcaaaaggaatgagcctctaatctgacctgaaagcatagtgcagaccacagatttctgcctctgaatcggaatattcctatgtgatcctggctgaactgttagacattctagagcttcggtcctcagcagtcaatagaaaaagaaatctatcctcttacccttacagtattgttctgaggatcaaatgagctctattcctcttacccagatcagtgttcttattaccacctgatgcatcatattgatttacctgattgtttgactttaaaatgtcagttccataagtgcgctttgttttgctgtaatctcagtgcttagaacttggcacacacacgccccaaaaaaaaaaaagcgaggggaggagacaagaaaaaaataatcagcagacagtgatagcagagtatgggacagtgaaagagatttcagtgggccaaccatacaaaacaaaggacaggagaccccatgttccctgctgcccatcctcatctcctggcttggatgcattagagcaaagcctcagtgtaatcatgggcatttggattgtttactcctcgttaaccaccctctttgctcacttaattggtccaaagcctgtggcatgagtaactaactgtgcagcgatttggcacgtggtcagcagcagtagcagccgtaatgtactgcactcatactctgggtgcagttcccacacggtaatggagctcagacaggctgccgctgcctgcagcacctttgatgtcctggcaaacacacccatgggacttgctcaaccacaggatcttcactcttggcttcttatgggtgcccaactagttcccaggtccctgctgtctgagcatcttacacaagtgggcagagaaattcacacccctggtttaagggaactaaatccaagttgcgtgcctggccacctcccctcacacctctcctccttggattgctctgttacactcacctcctgcctgctttcccgtaaaggtcgtgcctcaggccctttgcactagctcttcccccacatcagtactcttaataaagaatggttaaagaagatttggagcttgactgcaagcgtttgcaccctagttctgactctagaaagtttttaactgctttcatttttaaagtggagatacagtggagtcacactaagaattattgcattaagctgtgttattgcatgtaatcccaacaacgtgtgagggctcagtgtttattttcattatcactctatgtattacctccttgtgaggaagcccaccttctggttgaaaagagttggggccccagttctatccctgtctatcttctgctgccccttcccaaggcaacccttctttgggatctagggcttcttgaaacagtttgaaatcactttagcccattctccttcattcattgaagagagggaaatatactgcttaaggtcacagtcagtcagaggcagagacaggactagaaccttgggttgtttctgccacagttggggtccccaggtctccctgctgcctcaggagattgtgctgctgccagaactcccagattctgctccctcccttctcccagtctcagggataatcctttaatgaattaagtggagcaattatggggggtggggaggctttggtactttgtggtaaccaactggtatctcatttactgtcagttgtgtacttagggataaacccacttactttcctctccccttcaagttagcagtccctaccaggcaaaggggataaagtgtgaacatgaactttctctttttgcctcctctccagag

SEQ ID NO: 65 - DNA, 14186 bp, Homo sapiens
  source 1..14186: mol_type = genomic DNA
ttctctttcaagagcttataattttctgtaaaaaccaaacattatcctttgggcacgtgtgttttattctttctcttgattctgtgttataaagtttgattaaaaaggaaagatactttttgtctcccacacaaagaatgttcataattgtaatttttgggacttgttgccatacccttgagcctccctttgtttaaagagtatttttaatctataaagaagtatctgtgtgtgtgtgtgtataaaatcaaaacggcatacctttttgagaatatttagtgattcctgaagactatattatagttgtataaagaattatttagcaatctaatgtggtaggtgatctggtattatctgtctctttggtcacatgaggataaggctgcccatctcggttatccatgtcctgggccttaggaagaaggcagccagaatcttagaatttgaactgagaaaattgaacacaagttagcagcctaaaatataatgaataaaaattgtatgattttctcatctttattttgtgcagacaagaaactgaactttgcaagtattttctgtaaagggtgaataagctatccaaagtgaggattttatgtagaaaacatctccagctgtgttaaatataaagctgtcagttctttgaatatctccttgcaataattaatcatccttctcagttaacagcacagacagtacagcacaggtaagagagtaggctctgaagccagtctttgtgactctgggtacattgctttcatttacaaaaagaggatgatatagtacctaccatagtgttggggattaaattagttaatatatataaaacacttagaacatagatagtacatgtatttactgtttgttctgtataatacattttaagtggctggttttagttaagttctctcagagagtttggcatttgaactggtccttgaaggattttgtaaaagtgaaacataaagaattaaaaatggtaaaagctatgtgaattaagtagaggaagatctttgtttccatttattgcatactagctatgtgcctgcctctgagctttacattatcttattcaatcctcataataaccccaagataggtattttaagtcatttaacagatggcaaatagccacagaaaagtagccactttttcaaactcacaaagtcaaatgtttggcctagttctgtcagactgtaaagcccatgcttttaattggtatctaatactgctttcctatagtagactatcagattatcattccgagagactctggatgtgtgtttgctggtcatttcaaaggctgaagagaaacagacatgcctgtccttgccaaatacattgtttccctatcattctgtcacttggaagtacttgtttctgacctggaataatggtattgtggttattggcgtttttgttacctttcttactagaaattatgcagattccccaattatatccagatgcacaccttatattccaaattttgttgcctgggattgactgagtacccttttgagttcctgcatcagagacgtgtttacagttgtgttcccagtcttcagcatttatggtttcattttgtcaaatttgtttgaagtataaaaacgataatcttaattctgttactgatcattccttatttgtcacatcatctttttttgaaacgtttagggaattaattaataattttatactcattctttttccactcacatatctcactggctttgttcagaattaaatataaacccagtaaaactctcttacaacaatactcttgtattcgttctggattgtcacataggagatgtgtttaggtatctaacttggtttcagcattgtacgacagaatctgtggcaaatgtggcatggcatacattatatatggatattcataaactgcttttacaaacatttccagttactgaaccagtagcactgttccagtcagtatttttgtctgtacatgtatgcatgtgtgttttcccatttttataaaaatgtgttaaaagtaaaatgctaatgctgattatacaaagatagcatttgacaaaattccttcataactctgaagaaaatattctatgataatgggtagagttatttacaacattaaacaaaaagtatttgagagcaaccgttaaatattatcacagggtaaattcagcaactgaaatccttttctcatagtttgtattaggaaattaatagtctctcagatgtatattggttttttcagtagttattcttgtgtgatatgcatgctcatgtatgtatgtatttcttaaaaatcactggacttttgtggacttaattgttttcatagttttttcaattttacatattttaaattaaagcaaatctatggaatgcagatttacttatatcacttagtctttttcctttctttcagaaaagtggtgtggaaacaaaatggttttagtattgccttataaacaaatttaagatcatgagtttcttttattatcctctttgttcatttccttgaactacatccctaacctaaatcagctggagacagggaagaaagtgttagattcttactcagtttagatcctctgccaggaaaacttgtaactaaattaacgcttttttaaataaaaggagtcatttttaacaataacgtcagggcagcaggcatccatgaaatgtatggtcactgatcattagattctaatgtgtttactattttgaaactctagggagaagaaaacttatttttgcatatgttggaaaaatgtatcctcatggggtaatatagataaatttgtaatgcgtattttctaattagattatttaggagaaattaccactttctctttaattattagcattaactttatcatttttgtttcaagatgtcacagctattgttcagcactgaaattatataaattattagctgaagtgatgactggccatttattctgttatttctttcagaaagtctgttgttgtaaacatccccgactggaagctgtaagacacagctaagctttcagtcagatgtttgctgctaccggctattcacagacatcctcttgatattctgtcccggagtggagttgaggaggctataaaatgtgtgggaaacctcagaaatctttagctgcattcttttactgcttgctcctaataacaggatatgccgtctcgagctgacttagcgtgctgctgctggccttagagaatttagttaaaaaaagtgttattcctgtttctgccctgtcaatgaaattttagtctttataacctttccaagtaggctggagtttaacatcccaacaattcaggagactgattttctccattgtaaattctgtgggttcttcatcactaccacccagcagtatgcaatctagaaatttcagctttccctatacttaatgtgtgtgcaacatttttctccgtcctcttgtgatattacagctcctaatcagaactctcctctaagactgcaagactcaagccactaagcctgtttacatttgctaatgggtatgcatgtctttttatattaatatttttcctaatattttcagattgttgctttgcttccagctgccctgaaagcagctgtattgtatctattttttgtgtttccccacagatctgaataatccagggcttttctcacagatgacactcagtgtggactagtataattttatagctttctgagttttaaaaaattgttattttatttctgttagtctttacatttctattttattggcatttttcttttcaataaagcattgttttttaaatcatttatttttattaaactgctaaacatactatgttaggttccatacatgattaaaccaaaagataaaggtcttgccaatctaatgttttacatgcttttctttgattagattataagctttctgaggtcagagacattaaatcttgataattctattttttctgttataaacagcaagtaagcaccttgatgtatagtactttagtaatgattttataaatgctcttttgttagagtagtagtttataagacttatttggggatgggggtcagttgcttattttcaggctgagttattgctgagctcagaagtcttccctgccaaggtagaatgtaatgtgttctgtggtaataggattattttgtttgaccaggtgacactgtttatttcttataaaattttgatatatgctttaaaatttagtaagatggatgcataatttatgcagtgaaatttgaaaggtttttcaaaatgataagccaaacctaagctaaatgagggggattctttataataaagctgttatactctgcccagctaggccagttatccctagatgctacagattaatggcaatctgttatgtaacatttgttctatattattctgccctaatgaggtgaatcaaagacgatgtcagttttaattctggatgcccttgcttcagtatagttaggtacagcctttttgtagttttcagatggcttctttatgtttttatattgaagaaccactaacttcccattggcctataaggttagcaattgattatttaggcttgaaagttagatcatcagtatttgtcaataagcctgtaataacttgtttccctagcccttaacttagtgttgaagaaaaaaattgtggtatggacttagaaagcacagaaattacttattatttaagatttgttctaccaattgatatttaaagtaaaacatttttagccattctgtaaatgacagtattcctgagtatatggtttagggacaactttttaagcaaataactattacaaaattgggtctaatttatttcattgtacaaatattctttagaaatagtcatataaatacactttggacttgttctcattgtctccacacctcagttccttctacatgagtcatgggcttgaggttacccagaaaggactgtgaacctataacccagcatgcatgtggtattgcgtcagcatgttccccttctactcttgttacagctttattcattggatcaatgtgcattatttgctgtgtgtgctcagtttttgtttcttttttttagtttgaatatctgggcttagttactttttaaaataaggctgtttcagatagtttgcctagcctggttaatgtgggtggacccatgctacatgttttggcattgcctttttttatctgtaccatattacaaacttcctagtaatttgacattcactgctgtctttgctttagctttccaacaggtttggtgaaaattgctgttcaatatcagcttgcatcagaattacggagggcttattaaaacacagattgctgggccccaaaaaaagtttctgattcagtaggtctggggtggaacataagaatgtgctttagttcccaagttgatgctgctcctctaggcccaagaatcacagagctcatcttacattcttttgatattatgccaaatcctgaagtttgattataaaaaggcccagagaattgaatgtgttttcagagaaaagcagaagaaaaaaatacggttgtaatagtagttaaaacagaaaagcagtatggttgtagagtggggtggcggggaggtatatgttaatcttgagaacaatgcttttggaaaacaggaggttcctgaagtcctgggttacctgtgaaatgccaaagatttttagtttgctggcagtgttatatgtagttttacttccaaatttgaagacctcttctttgatgcaagtgtgttggtaggaactgataatagttagtggagatatttgtgggaatatatttttaatcagaaagaatgcaaaaagagggggagacatttctttgagacaccttaatgacactgggaacagtagaaggtggaaatgtgggaaagatgtgcctgagatgaaattatctgaatgttgagggggaggagagggttgatgcatcatactggtcatcaagtaagttcttataaatgcaagtttgattgttttgaagggaaggatataggaaggctggataatcagctacatttagatatggaacactacaggatgtcggcatttcaactcacttctgccttttccttggattctacactttctctcaggatatctttccgattgagtcttgcctcatttagaatgctgtgcctgttctttttttcaacagagtcttacgtaaagaaccgtacaaacttagtaaagagtttaagtcctgctttaaaccaagtttcagttcatgtaaacatcctacactcagctgtaatacatggattggctgggaggtggatgtttacttcagctgacttggaatgtcaaccaattaacattgataaaagattttaaatgctagttattattaatgctgtgttgtaggtgttcagtaaatagttgccataatgcttaaagcagacacttgtttttattaataaagatgatgccaactttttggtttagaaatgctttgaatctttgatattgttgtaatatagtggcaaagaattttaatcccagcttctggcgctatgttacttggggaagttattaatctctctgtgcctcagttctcagtcgactaaattgaattataatagtacctgataggattgttgagttaatttgaaacttgccaaatttttacattattttataatttatagtactttaacatattgcaatgcattacaaagcatataatgtaaaggtagttttatgaagtgtcaaattaagcatatttgggaattgcccatcaatttgtttttgaacctcagtggaatttctttcatatgctagatgtgaaaaaagcatatgcatgtggtttaggctacaaaatagcctgtaggttaaattcctaattctagtgtttaattctttttcttcttgtttaatggccatatgtttatatctttatattacatacattcctgattatgtaataaggtctttgatcatgtgggctatcagatttgcctcactttaagaagctatacatgaaactatgttgtacttaagtgaaaatttgattcttgtttgaatgcagtgaaacattctacttacagttagctgtctcccacccttacaggaaggtacatgccttcattttttacagaattttttttctgattaatgtgtttatcagttttatgttatgaaatctggatatatgttaaaatgtctcagttttccccttgtttaccatattgccattatagataacataatgtaaaattggatgtctttgataattttgggatatgttctgaaattaaatttgaagatttggtatcttataattatttggtgagcatttggcatgaattatttagcttctcccctttatgatttatttctctcatcgatttctatggttctaatgtctcctgtagacttcagtaagggaatctgtaatatataatagttatcttttagttatatttccactttttttaatgtgttttttttttagtaaatgtcaaaagtgttactcagtaacatattatgcaatattataatcaactaacctcttcattgataatgacttcatattgagaggttttttaaatctagaaattgttttaaattccaggttaaaatgtaaaggtatttttattttttaattcttctcaaagctactagtcattataaaacaacaatttcacatttagaatttttatatgtatataaattagattatttttatattagccttaactttctttaattttatgaaaaacaactagtcccaagggtatttttactatttaatatacattattcctgtgattcaaaatcttagcagtagaacctgttgagctacttaacttttcttgggactatccaggtgcagttataactggtttcaaagtttttccttgcttgtttcaaaatcattccttaaaaaatgttcagtatttttcttttgtcaaaaaatatgtgggttgttctgttttttaaaatagtttgagaaacttcctttgccacttgttttctgtgcaaattgtaagagctcaaattccctgggggtagggaggacttttataaggtttttttggttgaagggtataaaaggcacttaaaaggtatacagttacttttatttatcccagtcattttgacattatgataagctttccccactttataggttatctgaaaatttggtggggggctttatttatacttaatataacaaaattattttcctatgtgtagtcaatagcacatacaaaaatgtttgtagtcagtagcacagtattcaccaaaatactgagttattttcaagtaaaggtataagtatgaatgagttgtcagtccttctttttcagataactttctaaattaaacttttttaatgttataagtgaccagactatgtcactgatacaatgtcatgattagatgtgcagaagactttacatggatgataaatgaaacatatagaaattaatatcagattaaactaaaacattaagcaaagtatgcttgcatacatatatatttaatgatatgtggagaatctttgtgcttcatagtcactagacctaaagtagaagcagttgtcaagaatgtccaactgctattcatctttgtggccaaagtaaatttcagattttttttttgaatggttttctctttattaacccatcatcatgacaagtctgtcaagtgacacatgtgaaaagcagatgtccttttaaggaggcagtgcaatgcaggagaaggaacattattagagataaaggaaaaaaccagaattccactcaacacttcctcaccaaatgtgtgtggggatttctaccaacaaccaagttctttactgcagattctccagtggataccagccaggtgtcctctaattcagttcaattctattatctactgggagatagtgtcagatcccgcaagtggagggctcagtcctacaagactacccctacttcagatgtcagtcacaagcagttagcggtttgtgattctgaccaactggctagaggtccccctcctcaagtgtgattaatttgctagagcagctcacagaattcagggaaacactttacatttacccatttattataaaggatttatgaaggatacacatgaactgccagaaagacaaggagcataggcaagatacaagggtcacggagcttccaggtgcaccaccctctgggaatattcatgtgttcagctgttcgaagctcattcgaaccctgtccttctgagtttctggaggcttcattacggcatgattgattacatcattggtcgccggtcctgtcgtgtccctggaggttggagggtggactgaaagcttctcatcatgctttgatctttccgatgaccgaccccaccctgaagctatctacgggtccccagcaacattcatttcgttaacattcttacaaagagattccaagggctttaggaaatggagatgaagagcaaatatatcaatatcacaaacatacccttgggaattagacctctgatctaatctaggctccctcacttgccatttttgtgaccttgaacaagttgttcatcttttctagttctctgttaaaaaaattaaaataaaaaaagctatcaggttgttaggaggactagataagagaactatgtgaaggacccgcacagtgctagcaacaaagttgtttagtaagtgctatttgcttccagtgagctccctcagtgtgtaccaccaaatcacatgtatcccagtcaaatcttaaagtccaccttgaatctttaaggtgtgtttttaaaactgcttttgttttgatccttggaagttaaattttttacacaagcattcaactgttagcttcaaactgaacagaaaatatctgtattatttcattatgtataaaaaatgattaagcctgcctcaggcagttcagagcaggttcaaggagtaacattagactaatcagagtgcctaaaaagctttatgcggtccaggttgtacagggatgttggaacagtaaatatgaaggttcgagataggtagaatagagcaggttatattagagaagtctttctaaaattgtgttggctagcctgggatttgaacttataccgtgtatgtatgtccatgtgtaacaggttgcatgcaagaagcagtgttcataaaaggtggcaaacttgttttacttgttcagtcttttcagatcagtacttataaaacatatttacagtaaatatcttatattaaatattagctaaccctattaaattgtgtgtccttgtgctgcatcatttaatgactaagttggcatgtgatttgagatagctctaagtgctaatggatttgaaggagttaaaggcaaaaccaggaagagaaaactatttttaggaaaaagcgatcatacaacaggccttaaatagaagaaatgagatatcttatctttactatgtaacctttaattccttacacattttgacttcatattttttgtatgtaaatgagaatgttatagtcttgtaaattccttttcctctgtgactacattcagttattttatgtgtgtttgtgcatgtcggtaagcacagtatttagtagaatgagttattggttaaaccttgtaagggccacctgtctcttaaattgtaatgaggatttgattagataatctagataaatcactaagcactcagaaaatatcccattcaaaactcagttccagtgtcaagtctttgatttggaactccaaggtagttggtcctctctttgtgccataaatgtgcctgtactcagtagtgctaaattgctatcaactagtctaaatgtacactaattcatctgtgtgtccacttcccttctttcccctccctgatggaagatggtctgggagcaaaggtgacaggtaatctttttttaaacagctaaaggattggatcaatttttcacttccctttttcacacttttttccttttactaagaaaaagtgcaaatggagaaaactgtccactaaatatgaattaaagtgctattatcagaaattgtgtaaaagcaacataatggcactataaaaaggtttatcttagaatcaaaaaccttgggtttatattatagcctgctactttctagtcatatgacagtcaagctatattacctctttggcttacaaaataggtcatctgcttgttatgattgatgtgaaatattcatacaaactaccactgtgcctggtaagtagaagacactcaaatatgtcttggagttacagttaatgtttaaatttgagaaaaaccaactttatcacatagatacagttgtttactgctagaaattttatttaaattagttgatactcttggttaggtaatgaaaaacaatggttttatctccattatgctgtgatagggaagagtaacacagttcaaatggcatacctgacaattaagcatagttaatcacacaagaaacaagtctctacttgatacctctccatcattgatactttgtgccatacagtgaaaacagaaccatctaggtgttcacggatttatttttccttatgaaaggtagagagaaaattattcccaatacaaaattggttattaggtgccttttaattaggcgtaagggcatatgaacattacctatgagaattttgtcaaaattatttcccagttactgttttaaaacagcctctcttccccacttctaatttctcctttctattctaccaattttactccatacctttttgctaaataaacattttcttccaactctgtttggtgttttttttttgatctgctcttcaaagtatcagctagtgattaggctatagaactctaagacagtagcttaaacaagattttttttttaacccctgacacaaagacaaagctttttcagaaactcttatgtttttagctgtaagagatcatattgtcactgagcaaagtacttacaattcgtagtcatttgtcaggaagggtgaactgactaaataaattatcctaccaccacaatcccataactctaaacttagatgtcaaacaaatatacacacagctaaccacaaaaaaaaggaaatcccagctgccagaagtgaaaaaacagccagaatagtgaactcagcagctgaaggtacactgacctaggtggatgatagtgttggctgtgggtctcaccaatggaccctgagactttaacagcaagagatatggcctgactatacagggaagctggactgacaaagccaagaccttagaaggtgcagcctcagaaggaggatgagcaaacttctacaacccagggaagctacaaagttagttattttaattaatattttcatgtatttccttaaatcacttagcttagaactatggaaagtcagaaacaggccttggtaggtaaattgcccaagtcgtggtgaattcttaaagacagcattctataggaaatattttagtgctggcagtttagttatttaatggtggaaacttgaaatttgatctttgaaatataaaggaataaagagtacattttagagacatcaagctctacgttgagaagacattgagagattacatgagtaacaagaaagagaaactttctgagaatgcacagctcttttacaaataacaaagcatttagttttctttaggcttggggcaagacattaaatccacccctggtttataaacagatcacaccaagtttagtgtccactgatatctggaagactttaagggtattacatttttgctattaggatcttgttgcctctagcaacatttggtcccaggacaagaaataggggcgggtgaatgggagagtcattaagaaagcatgtagaattgaagaaacctgaaaataatggaagattctttcagaactcatcccaaggtctgggtaaagcctgtaatgtaacatacataattttgtaaacacaccacatcagataactacagatgttttggacagcagctgattgtttaaaactcatttcagagtaccaggtattggagggagggagatgggggttgaaacagtggggtagaattcagtggaccaggattctataccttttaaggcaattcctgattatatgtatttataagaatgtgagcctcagtcaccacattccttcagggttgggtataaaacgaaatacttggaagatataaagattgtagatgacaggttttatgttcaagcagcattgacattgctactgtaaaagtataatagggagtttatcaatgctgtaatatggttttaaaggcttttttctaactatagatacttatacattttttcttttggcaatgatttttagggagaactaaatcattcttgtagccagactttgcaaatgaaagaaaacaagccaagggaacctcacttaggatataagacactcacaagcatgagtgttgcaactccagttgtcgtgtttttaataacacatttaaaaagtatagacggtgaattcccactaagtgtgtattctttgctgtttaccagggtcagcatgagctgcgaatctgcatgcgtgtctgtcccacattgtggttacctgtttggcagttttgcaacattttaaccccagattttattatttttcaaactcattctcctcctcctgttgagactctgtcctgcggggaatgaaacaagactctgtcactcaatctgttgaacatctatttgagaattaggttcagattccctggcttaagccacttaattatctagccaggagttttcaaatatttttatagtggaacctgctctttaaaaagaaatcattcaccaaagcccaccatgtaaagcacatgaacgtgaacccacctgctaactgattccctggttaccccttttagcacctgaggcacacaataagaccactgagttaacccaccttctgttatccatctctggaacagactctccaatatagccattgtaaaccatcctgtttgtaatcctgtgccttcgtatatccagttcttccattgtgaatgcttcatcatccttgatatttagttgtccttgatataatcttttcctagaaatctgccctgatacccttgtctgtgttcctttgcattcattcctctatctttgctacactgtatttattagtcagactctcccacactaatagtgttctttttggaacatgtcttattcgatattctgcctccagggcca

SEQ ID NO: 66 - DNA, 9518 bp, Homo sapiens
  source 1..9518: mol_type = genomic DNA
tcctgtccaacccagtaaaattagaggatattgtgtattaaatgtacattttgcattaattcttgtaattaactcatgtaaattaattcagtgccaggcatatatgaagccataattaatgattgtgttgttttattattgttaacaaggttcctattgcattgcattcaaatgatgaagcaatttcctaggtaggaacaacacttcttgaaacaattaaaatgcagtcacagagtttctttaaaatggggtttaaaaattccatttctaggcacctagagaatacttctttaagagcacaagggatatatataattacattatacatattacttaccaatacgtaagcattctaaaatatgtgtagtgatacctatccatttcaggatagttattacctctggacaaggaaaataaagggaagaaagatggaacttcagctgtaactatggatacatgaaagtctattagagtattttttctgtgcttaattttttggtctataacattaaagtgttgacattaacttttacatgcagttttattacttagtaaaactcaaatatcacctcccttggaaaccttgctggaatcttctggaaatctccctccttgggttcccacaggtcctgttcagttcagtacccttttgtggcccacatctttatgactcaggtgtaaagtgttaggatctcaattgcagtgactggtgctaggatggctgttattatatgcctaaaaaatacttataagtaatgaagccaacttacatattctgtaattgctgatgaggtgtgctgatggttgctgataaaaatgaggaaggaatatgaagaaaggtacagtcatgaaacaatacccctgataaacccagttgttaaatcctcaggctgccacaacaaaataccataggctgggtggcttatggaacagtttctcacagttcatgagcctttgagtccaagattgaggtgtcagctgattcagttcctggtgggcccttcctggcttgcagatggctgcggtctctctgtatcttcacatggcacagagagagctctggtgtctcttcctcttcttatgaggactctgatcccatcatgagggccctacccatgttctaatctaaccctaattaccacccaaaggacccacctccaaataccatcatattggaggttaagacttcaacatatgaatttgggggagacatgaacattcagtctataacatcagtccaaagttaattctctacctgtgatcattttatagaatgagcaggcagggtttctgctctaggggatgctacccctggtcagagactcgtgtggatttggcagggaggaaactgcagaattgtatggcctctttcatcggcatgtttctataatctcttcacatacctgaatgcctgaagagtaagtgaatttcaacactatgtgctttgtaattctcatcagcttgtcattgattgctctcccgtcaagatgatggagaattttagcttttgcaaacaattatagaatcttctgatcaattcacagtctttgaatggtctgcatatccttatgcaaaagtcatttcaatttttaattattttagaaagtgatttgatgcattcattctttaaggtaaaataaaacataagcataagcagttagaattagataaattattgtaaaagtcaattcaataatcaattattacaaatctaaataactatgcacatgacggttgggtatgttagcataaccagataattggttattgatagaccgtagggtacataattattcattacctggatgcaatgctgatttctcgctgtgctgatgtatgtcattagctacattttaaagaagaccttattcacttgcttaaggctccatagaaggtagatcttgttttttatctctaaacaaaaaagatttcttttacattttttatcatgacataaaattgtgtactttcctcatttagaacttcaagctcctgggtttgtaagaccaactgccaagcctcctaaagaagtttctgggagtccctcctgcttattccatctccataaacctcaggattcaaactgatgactcaccatattaggtttctggcaatagactgatgtttctcgccattgctctctagagcactgtaattttagccttaagatatcatgataatttctcctacctgcaaattcacactcattaaactgggtgataattatgcaaggactcactaacaaaaggccaggcaacaattccataattaatgtctaggttcttgtttttccagtgatagatttttttttctcttttcagcttaaataaagatgacattgtcgtgatcattgtaaaactagccagtaaaaaatacattagactacagaaaagaaagtcgggttctgtttcaggactaccttgtctgttctccacaactttttagtctctgtaggccaattgcactaggaaccactctcccatctactattattactacaaaacacacagcaaactgtgtgcaagcacatacatgtttattcacctgatgagcttttactttaactatatggctgacataatttgctgctccgtagaattcaaaaaaacagtcactttttaaaggatgcataggtataatatgactcagtaaatcacagaagaaatacaatgaccaatacatatttgaaacactgttcacatctaccagtaatcataaaatataaacaaaagaaagaaatccaatcaaagccagagtaggactccctctatggagggctgcattttgtgtttgacattggaagacgtctgacacgaataacttacagaaagccctgaagaaaacaagacaaatggcacccagatgaaaggcactgggttgtcagagcctgggtgatgaggacaaggatgaaggagaacagtaaagccacctttcgcacaataaattatgagataaccctggaacgacagcaaacagctgtcggtttcctcagagctaacagcagccacctctgctgctttttccaccaccagctcctttgacaaccagcctaacagaattgatgatcaaagctctccagtgtggcagcaaagcctttgccgggggaaaggtctttaaatgttactggagcgagaagggaaagtgccaggtaggaataaccttgacagaataaagtgaacaacaaagtgctggaagcaaactgttaagagggtgtggatacagctcgtgaggtggcggctttgtggaggttttcactggtccccaacaaaaaaggacaatgtagcaagtttcccttatacctttcacttaaaagactgtcctttattccaagatttcttctttcacagttttgatattaaatctccttttgtgaaaagacgatagaagatggtgttcttcctctctcttatattcatggtgaaataaaatgttggcaacaaaagtatatctcaataaagctgttattttaaaatatagaaaaaaatctacagagaagattacagatggtttttagttcttctttgtataggcatcctaaattctctattaccatataatgttatgttatcagagagttataaatatttcaaaataattatttacgacatgaggtttagagagtgctgtaacacagttgcatacaaggtacagggtggatgaaaagttctggtgctatttggatgtagggggaaaaaaagcttgagttgagtcttgaaggatacgtaggggttctccaggtggatgaggtagaagggcattctgggagagaacagtatgttcagaggctctgatgtaaagtatcctgacctgcttggtaattgtgttcgttaggtatgtctggattttaaagcctgttgggaggttgcagtgggggtagagggaggccagcaagataaggctgggtgagctggaggactgaggttcttatggctgcattcagcagagaccagacattaccttaagaagccttcaaggagtacattgttaagagaaacggcatctgtgggataaaaagaggcagaaagatctaagcaatgatctcaggctggaaagaaccaaacctttggcagagtctaaaacccaggcgctgaggatttgccgtggtccaccttgcatggttactgagcacccagtatttgcaggcttaaaataacaagatagagtatcaccactacctcagttagtctcattattgagggatttcagatacaggagcaactgaaggtagcaaagcaaacaacacaatgtgctaagagagcagtttcccttttacaaaatagtaatgatgaattattttcaccaagtaaaattcaagctttgaagggctggagtggacttggttaaagactaagtaactgtacctttatttcaattgcatgaactaaaactacaaaacaagtgaagacatgcactgagaaagtggagtatactatgacggtaaatagttttttagcattttaatgacagagagagattgtgtgtgtatgacacatcaacaacaaataaataaaagaactggtgaggatagatgagtcactgagaatgtttaaaactatttggctcccccaggagatgaccagatagtcagtgaatgattcagcatgggaaataaccagtaagagcaaatgcgaatgctaaagggatatgaaaatctcagagatggggaaaacagggtcttcacttgccactgagagttttaaaaagattatctcaggggatagtaaaaggcagggcttcaacctgcttgttctaaaggtagctcataaggtatagtcgagaaaacaggaagaagcattagtcactatatttagctattaacccaagattccagcaatttctgatgcctgcagcattcatctctgttctgctcttataaaacccttccagagtgacttgggtgagagtattgtcttagtctgtttgtgctgctataacaaaacacctaagaataattaacacataatagaatttttcatagttctggaagctgggaagtccaggtgagggcctagtctttgctttcaagatggaccttgactcctcatgtggtggaagggatggaaggataacaagggaactagtttcctccatcccattcatgaggctttcactctcatgacttaattacctcctaaagaccccacctatgattaagaccaggctcatgattaaatgtcaacttgtgaattttagaggatgcatttagaccacagctagtacctagagggtctctatacacattttaaatggcatcttacatgttcacccagagacaccataaagttgctgtcagataaggattttacaagaagagccagaaatcaagatttttgtataaaactccagagttttaaatgttggctcaaactaaagaaaattgtgcaggccaaacaaaacatatctgcaggccaattttcagcctcaatctaagtaagagttgttgtaagaacaaattaaatgagtttaaaataagatcaggcaagtactcactctgctcgcttcaccactttggtgttcatatgttaactgatttaagtctcagaacttatgagggaggtgtaattactaccttatgtgataaagaaagtaaggaagagaagttaagaaccttgttctgtcatacagtcagaagaagtggggcgggatttgaaccagacccaggcaatcggattcagatttcacctctcttaatcaatgcattatgcttgcagtacaagcttctgatccatgatagacagtctgtgtaatttcccttccacatcccctcaggtgctttgaggcaacttcagggggattttctgatgctaagaaagacagtttggtattttaaatacaacaaaagttgtcatttcaattaggtatctcaaaaactactgtgatgttcagtataatgatggaatctatatagtcttgactaggagttggaacagtcacgaggacgtatatgctcagctcttggtcgccaaaggacaaagttgcactgaagatggtgaaatgttacatcttcagagaagaccacctctcctcacctgggctcccaagtagtgcagacttggatagatggatatggctggacgaatggatagatgaatgagtgggcttcccaaaggtatccatctatctgcatataccaggagacacttttgactcttccattacaagatgaattacaaaatagagtgtcaaagttaaacttatctggtgtagttacaattgtagtgtttaatcatcttcactaagcattccctaaaaaagagcttagtgaaggaattccataggaataaatgtcgctccaatgtagctccggacaacattgatattgaggcgatgggggatgccatccacccgaatgtccacaatagaaaatgatttaaattttggtacagccatagaattactattagactaccaaaaacattgtttacaaattatgtgtaataaatacttatgatccaatattaaatgagaaaatgatacaagtttatacagtatatgtaaaaatgtatcatttaagtgattttttcttaaacaaaaaaagacatcaggtaagtaaatttattgattacctgcaataaggatacaaagtaatagacaaaagaaaaacaaaaccaagatatttcaaatagttgaggagatgagaagctctcaaataattagattatgaagaaatagatggcaattgaaacgtgaagatatataacagttgctataatatccagaagagagaaattacctttggctgtggatccagggcaggctaggaggagcatgagctgaaccagaagttaactgggatttaggcaggctatagagcattccaggcaggaatgggagggatgcaggttccaggctgatgcaagtatggaaacatgcaatatatgtttagtgtaatgcaagggaaaaaacttggaacctcagttggaatcctggctccatgatttctctttaatagggtatggtaacctctaaggtctcaggatcatcagccatatacagggacagtaaggtatctcataggattcctgtgtggattaaacaagattaaattcctgtggatatcccttctttctcttcagctttctttgttgttcatttattcattaccattcatttagcatgtgatgtagcaagcaatggttggctaggcttgtttgctccgatcctgccctgtgccccacccttcctgccctgttgggccccagagtctgacccctatggactgtgtaaggctctcttgctctctggcttccagttgggcttggccaggaaataattgggaggaggaagaagcggggcaacttacccagtcaccgcttgtcccctgcgttgccatggctgtggtgggtagctcctctcccatagcctccgctcttaccatctccaaacacagctctgcactcttgccccttcagttctaccggtggggatagctttccacagtggtaggccctgggtgccgcatcatgtgttctcttaaccctgcccacgtttctgtacctggttcctttctcaaattcccttcagttaaactcctttaagaatgccatctcttgctggtaccctgatacaaccataaacaaggtagacaacatccttgccttatggggcttatgttctggtaaacaaccatattaacataatagctttggatacagtgaaaaataaagtaaagtagtgagacaataatggctgagaggggctactcgagggaatgatatttgagctgggacctaaggaatcagagggagccagcaatccaaagatctgggagacttctattttaggcagtagaggcatagtaaaaaaggctaggacaggatgtggtgttgggagaatgaaaagcagtggggctgagaaagggtggtgggagctggtcagagaggcagtggggactggtcaaggatggacacagtgagccatggagaaggatttggactttaagagtgtcaggaagtcttcgaagggcttgggtggggagtaatttgagccatatttagtagacaaaggattacacatcaggcagagtggagggtactgctcctgtccaggccggagttactggcatgatctagagtgatggcagtggaagtggagagatgtaaatggatgaggtcaacttgcactgtgattctgaactataacaatacttcagtctttcccaaccagtatcctaggctcctggaagattgtgaaaactctattttttttttttacatttatatgttcattttgattattacatatttaaatataaccccatttaattttatttctaagacctccacagtgccaaataattgcctacaataacagcatttaacatactgtggtgtttttattcagacatacttagaggaaatacaacattcataggtgcatagttcaataaattttcacaaactgaacatctccatagaagcagcacagtttaggaactcaacatcaccaagactccaggaaccctctcctgtcctctccaagtcactactcccacaccctagtttagtttagctggttgttgaaatttatcagtacctgagaagagggggaaatcaggagaaagcagattgctaaaaatcccaccctgggatgtggttagtatgggattcccaccaggaacaggcccacaatttctgttcctgcttattgcacctggtaatgaagaactgagcttgcaaccaggttatccccaaagcataagcatatcacagtgatataattgtttgcctgatccccatcaatggacacggccctttgcataatgtttgttgatggctgtgactcttctgcatgccttaggtgagtcatgagaggtcaataaaacagtgatgccttgacttttggttttgccaggtccagaccaaaaggttttattaatcgagaagaaacacagctgaatcagcttctagtcacatgtacttccctattcattagactgccagggttaaaagggaagcagccaaagtttgttttgatgtaaccctttcattaccccatggcctcatgcctcccacttgtccccggagcacgtcactggatgcacacatacccccgcccatcattccaattctgctctctgttatgtgacagtgggtggtattgggttaatcagaggaatgcccgatgtggtttggtgggcatttgaaagttaagaagcctgcttctaggtcaattctttaggctaactagtggaaagtgacagaaacttgactgagcaagaaataaacagaaaccccagtaattaattagctcctacagagatcagaggtcaggcatggttgtatgatgtcattatgagagacttctctccatctcttggtactgcttcccttggctctgcctctggtgacaagatggctgtcagtctcaagcttgaatctcacctctcagcaaccccaatggagcacataaacttctctttccaatgtcctgaaatcaggctttgtctgattgaagaggcctagttctcatgctcatcctgggagtaaagtcagcaccagccaaaatgctaggaccaa

SEQ ID NO: 67 - DNA, 3021 bp, Homo sapiens
  source 1..3021: mol_type = genomic DNA
cggggtgccggcctctccccggctggacccaacgctgcccggggctgctgcaagagcggccccgcccgtccgccgcccaacacccgctcttcctccgcaccgaacacttcgggcttgcaggagacaactgtcacgccctcttcccttttctctaagacgctgcacattcttgcccacatctgctttcttgtgtgcaggcctcccagggtatgtccgtgggagtgaatcactggctcatagggtgtttaaatgttcacttttggtgatattgccaaattgagttccaagtggtcataccagtttacacacccaccagcactggatgagtgttattttcccacatgctcaccaacattttgcagggtgacacagcagttgcctcctgctgacatcccagatatgcaaatgctggggctaggagcatctgtctcagctggactatcacctcctccttcaggaaggccccccagtctaccttagctgactggcagcttcccataacctcgcgtgctttcccatcacagcactgggaacttgaggggtgactgtctcacagcggactgtgagctccccaggaagaggtcaatttggggccattgaacaggctgaaaggaacacaaagttgctacaaatagtactcatctacttgaagtaaggagctttctctaatcaaagtacgcccttgggatggcagtagccacctacccaccacacctctgtgtcctcagcatctggatacagcagtagcttccttttaaacagcacatcgttgcttttgcatcagccagaaaaaaaaaatactttttagaagaaaagtacattatcatgagtcatagtatttgagtacagctggcctgaggtgaggctggaggcgccgcccccggtgactgggtactcagggttgggttgagccaggagggtggtggttgcctcaggctctgagggatgggcagggagctgggcggggtcctgagacattcaacctcagctggttcctgggttccgggccctggagcagtgagggaagccagttatatgggaggcaggtgagtcaaaggagggtcaggctgggtgccagagaggagggccactgcttctgtgagggggcctctggggccttccgtccatccccagtgggcatatggggacaccaccgaggcccagagacctcttgcccagtcccccaccctctgctgagctttgcccagggtgtagccagttggcctcagggccaggctgccagggtcaggtcagctcccacgcctccaccctgggcagggcccaggcctgcattcatgggtgcgagaggcctgagccctggatgggtgtggttgtgtgtccttggggctggtgttttggcgtgagtaatactgtatagatgtaagtgtgtgtagtgtttgcaatgttgtagtgtgggtgcatgtgtctgtgtgtcctgtttgttgagaggattcattgcatgtggttctgttaggagtgataatggggtgttttgagacatttcctccttggtctcttttttggtgtgttggttatgttggtgtttcggtatgtgcgctcagtgtgttggtgtgcatttggcaggttgtgtctattggtgtgtggctgttgggtttgttgtgtcagtcgtacatccacgttgggctgggtgtgtgcatttgtatattgctctgtgggtccctgcaggtttgggagtagatgggagcggccctttgtccagccacaggaaaaccccacccccctcatcggcaggggacttgggaggggcccagactggtaggctccagcctcccgtccattgttctggggcctctctggaaaacaggatgggaggtgaaaggtggacacaggcctggacccccacactccctgaaacaaccaggggcacagcacagaatccttaggagggagggcctggggcccattttgtggatgagaaaactgaggggtttcattcctattaaagggagagagcctgaagccccacaccctttatgtgcccacccacagcctcctgccgggtttgggggatggaaaaatgacaccccagaaggaaacaaaacggtctttgtggtttccatttctgaatgcttaccatgtgctaagtgcagatcataactctgggttaagttcatattttgtacccattttgcacaggagtacactgaaccataaggcaacaatttattcttcaaaggacactatctaaacattactgtatttcaagtagtacgctgtgtgaccttgagcaagttgcttaccttctctgggccccctagttcagctccacagcatgaccaaaggagccgtggttcctgctctcttagagcccatagcctgggcagggaggaggaggcaaggaatggcaaatacacaagaaaacaaagatcatttctgatggtaaaagaaagtagcctagggggatctgagaaggcctctggggaggcgacatttgacctgagacttaagaatgaggagctggcaaagacctggctgatcagtgttctaggaacaagtaacagcaagggcaagggccctgaggcaggacagagctattaataggaggcctctgggctggtgagagaaagggaaggggcccagttgggtagtgggcaggggaggagtttggatcttattctccgtgagatgtgctggttctggggtagtgggggggcctgttctcctgttacaattgggggtggggacagccagagccaaaggtcttcccccacagctccggcagtcccagcccagaggcgccggcctggcggctggaagattgcactgtgggcacatctggggagcagctgccgagccagggccagagtgggtcacgtggcacgggcaggaccgtcctggagcctacaaggccaagactcctaggaggggcagtggagaacactctggacgagaacacgtcaacgaggaggaactgtgcactgtagacgtccaggcaacggccccctgcaccccacacagggacagaatgccacctcccagctttgtgatctgaaacaagtc

SEQ ID NO: 68 - DNA, 2909 bp, Homo sapiens
  source 1..2909: mol_type = genomic DNA
acctttctgggccctgtttgttcctctggatacaatacagatgtaatcaggcttcctgggccccatgtctgccctggaggtcagttgcctgcattgtcacatgctaaaatcccctatgattccaaaggctttgaataacagtgttggtttttactgaagattcgggggggaggttgggttatatcaccagcaaaacatccttaaagatttcttgaaaacttccccgcaaataccctttttttttttagtgttatatagagagagatgagtacttttttgctctcaaagggcctctcagctctatttttaataattgtatgattttataaatttaattcatagcaatgtctccttccaagacactgataatcattataataagcatttattatactcacagtgcctcttaaatcctcacaatccagtaaactataggtactatttcccttttctctataaaacatttagatctgtgacttgctcaagtcaaacagctgccaggtggcagatccaacattgagacctagtcctgcctgactttggatgctgtgctttttagcccctgtgctcactgcaccgccccgtccctcctgtcggcccttacccaaaagtgtcgtatctctgggaatattaacaaagttagaccatttttgctctgaagatttttgccctctgctgaagtacaattggaatcagtttgaacaaatcatgtttccaccaaccaacatctatggtgcacttggcactataacaatgctgttgattcattatttaaaatttttaaggttgagataatagaaacccaacttaaaaaaaataattttgattcagataaacaaagtctaggtctcaacccaattcaaagcctgtagattcagctcaaataataactcctattcctcctcttttttcctggccctgctcttctcttcctgtcttcattctgtatgcatcaccaaaagcctcaggttcacatctcccaactcagcaaccccaaagaacagagaatgaatgcctctctcgatactcatttatctttgattagctctgcccaagtcccatggaccaatcattatggactgtgattgatagccccatcaatgccatatggggtaggggagaaattacccaaagaacccaaaagagaagaataggaaagtgtgtgggagaagccaaagaatagatgcaaccatctaccacctggtctctgccatcaaattagttgaggacatggatacatgaaccaaaagataatgtgaagaagcatacacttgcccaatggttggcaccctgtaatgagaagccttctgaagatcgcccccagaggccttctagcctgcagttcttgtttggcttccttctcatccccacgccaccactcaccagtttcctgtgagcgtttcttagtgtcaaccccagtaatggtggcaggaacaggagagaggcccagcagacaaaatggtaaagctgggagattcccactcgcatggcagcaaatgatcttttcatcagagcaagcagagtaaacaggagctggccatttggtgaaacacgtagcgtaagacccaagaaaggaataagaaagagccaagaggaaagtagagaaaaaattaatttgaaatgaaactagtttttgctggacatgtagagtaatagtacagcctttctttaatggaaaaactccattcattatgcttagagagtcacttggcctctcaacatgctttaaagtcatgggcttaataaagtccagactcggagtctcttagttttctttttatgtatttcatgtatatttcatgcctcctgtcagttataatttttttaaacaaagttttcttaaaagactgcctttagtgagaactcctagaaggatttataaaccaccaggttttctgtttctgtcctaaaaacactggagtaagtggcaacaggtctttggggagctgcccatacattcttcatatcaacgtggctagagtgatctgatttgagcaacagaagtcattgaattcttagaagcatcagtgatttttgtggaggctctaacttggtatcaatcttgacaattgtgatttagaatgtcacggtttttatgaatttgttgacataattaaagtatccaagggctaccacatgaatccttgttttcttagggactgaaagaaagagtcgtgattatataagatatattccagttctgaaaaggaaacgagtcagtaacaatggaaggatttccttacttttctgctactgttctggagagcggcacagtccagatttgtttaaccaaacctgttaactttttcataaatttactggattgtaaataggagttaatttactccctatccaaattatatattaaatattttggaatactaaggctctggctttaccattgataattttgattaaaaaaaaaaagacacaccttgattttgtttgatgtgctgtgcttctttgacactagcagtggactatttgcttatcaatggaattcaaaagaaaaatgacataattttatttttggggaaactcagtcttatttcctttcttgtgtgattctccaaggaaaaatggagaccagaaatttggttaaaatataacttataactggcaacccacaactcattattttccatacttcaggtatgttttgttctactcttaaggggaaaaaaacaacctatgtaaactaaaattcagtaacccagaattcatcaagttctctgttatctagaggcacttagcagaatggtttaatacctggaccctggggtcaggctgcctgggttcaaattctgtcttgccaggccactgcacttcactgtgcctcagttgtttcatttgtaaggttaggattatagttgtaacatctttatagggttgttttgaagatgaaagag

SEQ ID NO: 69 - DNA, 4306 bp, Homo sapiens
  source 1..4306: mol_type = genomic DNA
ggatgggggtcatgttgcttcacgcaggccaggatggccctgagatgcctctatgggcaccgcctccactctccagccagtctgcccgctgcactcctactccgaggtgaggtcccccagtggcatccctcctgctacacctgtgactcctcagtgtccacgtggccctgaagcctttgcggaggaaacgtgaattgcatcaggaatgaaagaaacgttgtcacccacttcacgaatctcccgagccagcgttggggccacagggccactgactccaggagaacaagggtctctgccttgtgtaggggggtcatgctgtcattccccaacccgtggggcaaatagggactctcaatggggagacagcacccccccttgcaaagtggcccaggcctggtgagggccgtggctgtcccaggtggacaagtcagggagaaacacagcactccaggcacccgagggtgcttctggggcctcaggaggagctgggctgtgggtggccgggccccaggcccatcccgctgagggcagcattccgggtaggcccagcaggtgcggccacagacagctgagcttcccgggatgcttgtggcgtgaagtcagctgcccaacccggccactggccgcaccccggctatttttggatcctgcagctcagagcctgccttgctgctcccgcccctggagctcctggaagtctggtcagagtgtggacaatgcccctcttgtcatacatctgccccttccccacagcccccacgggccaccctcctcccccagacacagctggtcctggctgggctggggtgggctgcctggctggaggaggctggccttggaccgggccctggcatcacggcaaagctgggactcagggagtcggaggcgagaggctgccagggttgtcctgggccgtgtctgtgtcagctcgtgtccctccaggataagtggcaaggcacacctctctctggcagagagggctctggaaggttttcccagggtcctcctccctcaagtcctcccatgccctgaggctggtccctgagggttggtcggggactccagtccttgctgctcagaaaagcatgcccagtgtccctcgccctgcccagtgtacgtccaaggctgctgaaaatggagaattcctttacttctgccttcattccttggacagatattaaaacctggtgtccaggggtaaatcaggtctggttccaacttcaaacaggcagccctgaccgtgcagcccgaccttggactgggtaccgtgaccttggagcaggcagccctgacctggaggcatgcagctcctgcagaaccagatgtgggtgggggggcccatagtcacctctttgttgggctggggaggtgggttcccagcttcggaccaggtgaatgggggatgcaagcgtgggtggggcaggtgaagcaggaagggcagggacaggtcagatgactttagcatcacggcggtgggacccgctgtggtgttctgagtcaggaagtttggttgcagacaagggtcaggagagaggtgccgagggcatgaaaggaggtgtgggaggttattagggagacatttaagtcgatcaatcgtgagccacctgtgcctggatcctacctgtcagaacgcctgagtggccccacgttccagcctagagacaatgctgaagactctgcccgtggagtggcctgaccctgggcacaaggtgatgggcagagcagccagggttgcctgtgcccagcctgttgtgcagcagtgggggacagagaatgctcgctgcaggggaaacaggcctgcccccaggctctgccctggggcccctgagcttactctgtccccaggctctagctcacccggctctgcctgtcttggctgttataacaaaaccatagacctggtggtgtagaaacaatgtatctctctttggtcgcgaagtccaagatgaaggtgctggactgtgtctggtgagggccgctttccatagacagtgcggcctcttttctaagggcaccagtcccattcacgggctccactctcacaacctaatcacctcctaaaggctcacaatattactgcactgggggacaggtcaacatgtgcattttgggggaacacacatttcgaccatcacacctccgtaacagccaaccccgaggtccagcctaggtggctctgggagttggtggccggaggtccacagagcagcttggggctgtgcccagctgggccccttccaggcctccaggtgcggccacacccatgtttcggaggctggggacacagcggtggctctccactctagcaagcgtgcagcacccgccagcaggatcctctggctgcagaaatgcgtaagaagaggcgtccgcgggacccaggccggatgcccgtggagagggaggagccaagggggctccgcagtggaaagcagtgggagcctcccatgggttctctgctgggctgcatcaccccaaccctctcaggaggggaagccctgagccccccttggggttgattgtggcagatgtcaggggctgtgaccaaaaggatgcctgggccaggtagagaaacctgggagatggccccgtctgaggcacagcgcacggtgtcttcatcctcctgtgtggactctctgggttcatggaggagccctgacttagggctggggttaaatgtgccccccaacccctcccttcacccctgacccaccttggccttggggaggagaagaaacaccctacaggaagatgaggaaaacctgtccccacaggctgctggctgatacccccgacttaggacgagcctgggttggggagccgtcctgcccttcccggctgtcatggaatgtcaggtttctgggagcctctgcttcctgctgcaggctctgcctctgagttcaggaaggagacaggccccctggtcatgctgccaggaccacatggctcagcttgctgggcccggggagggtagaggctggccatctttgctgtttggtacctccttgtcagcccgtggtgggggcccagggtatggcgagggtgggaggcttctaggtcctgggggtcaggatgaagggagcccggggagcagctcttggccctggtgggtctgggctccaagttgtggtcagaggtgacagggtgtcctcgggcctgtggtcagtgccgctgggcctcatggaggtggcgtcttggatgacaggagagagctttttagggctcagattagcctgtgagaccaaaaccaggttcagtttccaaataacactcaagagacctgggcttagcattggccccccagctttgcaggagaggggtgggtgcccacggggaatcaaggccctgcgacccaagcccgccaggctcagcactgaggagggaggcgggccccaagactgtgcagtctcgaggcgtccaagccacagggcggagcggcgggagcgcagccgagagtgcctcctttatgcttgggccaggcggggccacgggcctgcagccataaagttgggctgtggatgtgccccagatcctgctggtggagcccaagacgccgctgcccccacactccggagcttccgcccggcccagacatccgtgtgcagtgtgggagctgaggaggggccccccaggatgctgtgtggggtacacacagcaggagggggcagggcaggacgcagtttcccttcaggttggggtcctgaccaggggacagagccgcactgtctccccaggcctgcttggggacaaaggccatggatgggtcttaggaagtggcttcagggctggactcctcttttcccgttttcccaaataaggtgttcccagttccctgggagaatgagtgagggctggggatggctgactttgtggtgaaggtgcagagagtagattggggttctctttgatcccctacaaagaagggacgcccgtggtgtgcttgtagtgagatttggccctccgccccaaggcagcagcgcgggagtgcggggctgggtcctacccaattacccaccaccaggctggcgagcccaggagcctccagagagcggcgaggtggttctgtttcgaccgcaggccagcttctcctggctttgctttggaggcgcccagaggggtggctgaaggggggctggcacagcattcttggggtgcctgggggcttggaggttctgagcctgggccgggccggagactcaagtccggctgaggttatcagcaaggtctctgaaagctgacagagcagagcaaggatacgcctgacgctgtgcaaggtttgcagagtcgctcagcctctctgggcccactt

SEQ ID NO: 70 - DNA, 3237 bp, Homo sapiens
  source 1..3237: mol_type = genomic DNA
gatccaaaccgaggaagtgggaggccgcgcacagcctgtggtttgtgcagcaccctctgccctgttcttaaagtaagaaactgatgtctctaggagccctggtcaactggaattggggacttggggtttggttgagcctgctaggccatctgaacccacagcaagctgatgatagactgcaggaaaagctccgtccttcaccagccccattccatgacgcactcaacgaccctgcctccaaggaacaagcttgcagatactaatcagggctgaggccctgccactcaataattacccactgtctcatcataatcccggaactaagcccctgcccaatccatctgggacccttttccatctattgcacagctcaagaatacgtgtgtaagctattacctctggttgtgctatctgtcccctgagtatagattcatggcagtgaccaattaggttgccgtgttctttttccccctttgcttccaattcatcctgtacttctcagcactggtaatgaagcagattcatcgggtgcagaaatccactcacctctaaaagctgtctcatatgatgcctaatattgtaggatactttaaaaagaggcaagatcatacctctgtattctgaggcttctttaagccactctcgacctgccactgccttctgccaggaacaaatcacacatggaggaaaatattcctgaccctaatccagaaatcaaacagctgagaagtgaagagggcaaatgtggggataattgaatcctgcatccctgaatgagaacccagtatcatgccaagggtgaaatccaagacacgtatgtggaaactcaggttctgctgttggccggccagtccctcaacctcttaaagattccatttttgccctcgctaaaacagcgtctgcttcagttgctctgaaagttaactgagacattccatgtaaagcactcaactttacaaatgataaaaaaagttagctacccctaccattttctgaattcctatagccaaaaccaaaaaagttagctcttgatcaaattgtgctgactattttgtgtgtatacaattcatcttccccaaaaacatatacattcctgaagggtaaaattcacatcttgttcttttgtatttcctacaggtgaccatttataaatgcttactcattcctgtatttgaacataagaacacatcataaacaattttgaaggtgacagggacaagtagttagttataattctcaaataaagaaattagataaggatgggtttcgttacagttagttgtagaattgcagaaactgcatggggcccactggtcaagaaaacctacctgaaacggcaggggctgttttcagtgtacactgtcagggcacaggcccagctcttaattagcaggaccccatttcagatctttgctgatactgacagggttttaattagttaaatctacaagttggctcttgttctctggcagagaggaaaatatgctaacatgttccaaaacaccccttccaggctttgaggaagtaccctgaagaaggaccaccctttggaatttataccagcagccctgacagctgttttgtatttatttctacggttgggccatttcaatttggggcttttattcattatataagcaaatatttgttaaaggatcaggcacggatgaggtatgtttgtcaggattgtaattaaggtggtgtgtataccagggttgcaagaggacagaggggttgggtcttcaacccactgtgtgttactttgaaagactcacagacctctctgggccacatctggcccacatgctgatgcatgtacagtcctcattcctactgtcctaaaaggtagagagaaataattaatttggataaaacattttaaggccctaatacattagcttttggcaaattatttgcctgcagtgtatactaacgcccttttatttttcagattgtccttttcaaagtgaatgatgttatcaatgagtactttgctgaagacataattatttctctgaacagtttcctgatttgccagttgttgccccctgaggaaaaccagcctcagaagtgtggcactcacttctcagctcccctgggcatcctgcatcccccagcattacttgtaaatatttcaaattcttgccacacgagaacacgtagcatattcacacatacagtattgactctcttataggtcaactccttcatttcgtttaatagctttcatcttggaaactgtaaaattcatcagttggctcctctcctccaagtaggatgtgtctaccttgataaggtggggaaactgaggctgtgtagttacaagtctttccagggagtcagcctctttcttatgaggagttggaatgcctgaatcccagccaggcccccacactagagttctccatgacctttgcgctctacatcaaacacacctgtggcttctgtaatgtgaccttccccctcctcctggacctccgactccttaatcctccatcagtttctttccaaatttaagccctccagaacttgctctttctagtgtccttagtatcatccctgtatagaacttggaagctccttctccagtccggcccatgccctggctctgattcttagatgtctgtgcctggaaaaagcctatctccttaagttccatatggccccaaataagcccattaatgcctctccccaggaacgtatttcctcttccaaacaacagtattttacttccagcagtggcgccacacaatttgacacttcccctttctctaattgcttactttgtgtgtctgttttgcctcctgactagactgcaaatctttgagggaggaggttcctggagcaggaggcccccggagagtccctaataagtgccaaatgcaaaaaggttgaagtcaccagttgagaaaatccacataaaatacacttttggcctgtataaaatcagcatgatgttttctcctgagaggttcacagcatataattttagttcctattctccccaatgctcaggttactccaggaagcaatttggggtgagagaaaagcaaggccctgctgaattagggaaaaaatgctctaaaatatctccacaggaaaacttagttccacctgactgaacattcccaacaggcaaggtggaaatacctcgttaagggactgcaaatcaagcagtgggttcaaatccaacattagctgcttatggtctgtgcacccttgagaaaatcacttaac

SEQ ID NO: 71 - DNA, 2130 bp, Homo sapiens
  source 1..2130: mol_type = genomic DNA
cgcaacagcaggtcctctctctgtgagacagctatgtcctcctgaagtatagctttgacagctgatacctggcatttgcaaggtaaagccagtgttaattaatgaggcatattttcaactctctgttaactaaaagtcatcatattcaaaaatggtcatgatgactcgccaggaggagagccagaaggtaagtttgggcattttctcccttgaccctgctcctctcatgacaaacagcctcaagtttgagaaagctggtgaaggttgggagaattatccacttggtatggatggaattgtttttctgggcttagggaatggtgccttcattagcaccacattcttgtcaaaagagctaactctctaccatctatattaattttgattttacagagatactcagaaaagatttctgtaaatagcctaatcataatatcaacaaacatgcttttaaaaaaaatcttagaaacaatagtccattcgcctagtaattagttaacacccaagcatgccttcaaaggaaagcatgacaaaatgactactttctagaaaacctatcagccttgtaatttcagttgttcatttgctttcaggttgaaatatatatatcttaaatgtcagtgatatcttccatagatgcttacccagttgaccagatatgcagccatgaatgtggaattcactaagcttgaaatcctgttttcaggctacacactagattttctaggtcttgacactgtttcctttcatctcttttaatcatgccctaaattcctccagcaccatgggaccagccaaaatacaggtatctatccaaaaattttaactttgtgcaaagggctctggaaatttaagttaacccactgcctccacttttctctccaacggaaaccagaaccaaatactaacattctctccagaaaaaataaggaaataaatgtgcaggaagcttaggataatattgtcacattgagaatctgtcccattctagtggggtactcagaaaataaacaaaatgcatctcatgaagggaataaaatctgaatcagaacagagacatatgatggtgctaaagaccagagccagcataacccatgctttacgggatctagcattcaaatcattttggttagccctgtggactgtgcctcccctcccccaaaattcatatgttgaagccctgatcctcaatgtggttgtatttggatggtttctcttctggtgaacttcaaacaccttctccttggccctttccctaaacctttctctgctggaccaccagagcaagctttctgagttcatactctatcgcattgcaggcttctattctgtttacagtgaatgctccttgcatgtattaaatcattgaaacatctcaacaaaggccatattcttatcgatttgttaaggtgagccaaaggaagcctccttatttacctctgtgtttcttcttcacccctctcccaccttcttctccacctatcacaaccaccaccacgagccttaaaacgttcaggattcccaccctgggcctgacaccacacgcccgacaggccttccatggctggcaggcaggccccaaagagaacagatctccagctgcatccaattgcttgcagcttccccaacaagctgcattcttctgccttttcacatgtaaattcctcttaaatgtctgtgcctaaacctcatcgccgcttggcacctggtgaattccgctccttcttagaaattcggcataagcattgctccctccaaaagccttctctgacgcctgtaggcctcaccacattcatatccatgttacggaacttagcacactgcaacactacccttgaatatctttctccaaaggggaagggctacagttctatttatctttagattcccagtgcctgacacatactaggtttttgaaagcaataaataaatgaagaacatgaagggcaaacacaaggggacagctgtggagtgtataatacccatattaattgggttttattaactacagaaatgattctaacttttgtaactccagaacctctttagttatcacatttcttggttatacatgtagacatgcatgtgaaatttttattccgtaaaccatacttgattagacaaataatg

SEQ ID NO: 72 - DNA, 4564 bp, Homo sapiens
  source 1..4564: mol_type = genomic DNA
tggaggtcatccaggcctgtgcctggtaggaccccaacatttaccactggtctgggcaaattgctgcttcccagaagtggctttaccaagacaagacctcacttaggtcccaagtgaggttagccctctttttatcatgacccaagcccaggaagggaggacacagggcctattacattatgtctggaaagttggagggtatccagtcgtgttgcataacctgtgagaggccacggccacatcccaagaaggatcccaaagatgagcttcacccaccaatgctcctgagatggccaaaggcaggctctttactccagaaaccagacaggaggcatagggagagagagatttgagtattaaatcctgggctgagacggtgggacctagtgccagctctcccatggagtgaccttaacaccccgtttcatgcaccaagcctcagcttcctcatggacaatggacttgcaaaccctgtaaagtttttgttaagctccagcaagacaggtatgagaagtgtgcagtgggaagacacaatgcgagctattgttagtgtctgggaggtggcatgaggagtatgccaggctggcaggaaatgtgggggacctggcacctatgattccagagagtgtgcccaggagctaggtgtctaaataagcaaatgcaggcctccctgatggacagtcatgcaccccgaaggtctgagtgggtagttatgggcaccccaggtttctggaccagcagcccttggagctggaagcagaatagagacagagtctggtcagggttggggtgggctgttgtgaagctgcattgtgcatttgctgtgggaatgctggcagtcacatgccttctattgcctgcccggctgggtaaaactgagcttctgtgcctttaaaattgtttgacatctttgagattggaaacagagctccccagtggaaggccagcagtagctatggaatgctattctaatacaaaacttactctcggtgtttaaactgataatcccactggcaggctttggagcgaaatcacctattatggaaaaggccccgattctctgggctgatctttgcccatgtgggctagtgagaatagagctgttttccctctcaggctaagccccatctcccaccctcagctgacgagacaaccctgagattctagtccggatacaggactgagagctcagggctggcggggagtccccatggtgttttgctctaagacactcgctagcccaaccccttaggggtgcctgggctggcggacaggtctccattccaggctgcctccctccccagaccataatgtcaggatgcgggtttcgcagagcctgggtactgcagtttagtcagctcccggttccttcctcccactgcggggacccttgtgaccgcctggggactctccagctaatccctcctgggcggccctcccatggtcaagtcctctgttccaacggagctctggaggcagagagagcagggttaaatcctggtctgcaagattacttagggcaagttcttaggcctttctgatctttagtttcttcccctgtcaaatgggtatagtaatcacacacatttcaggttaagagaatgaaatgagctagactagaatcaacagcctagcacggtggctctcaaagtgtggtccaggagtccgcaagaccctttcagggtagctttgaggtcaacactgttttcatgaaaataaagatgttatttgtctttttaaatcataaaaacatgttactgaaaaacattttccaaaattacattgccttgagtcaaaaggttagtgatatcatcaaaaagtgaacaactacttagtaatgtaaatgaacgttgacttgtgcctaagacagtccttggaacgtagaaaatgccatatgagttttgggaaccatttgaaattggcatgaagtctttattcacctaaacctctgaaaagcactgctctaggagcagagaaaaagggggattgtacacatgactgggaaatgcctgcaatatggtcactgcaaaggctgaatattaaattgtgattatataaaagtataggatctggaaataaaacctggaagtgaaatattgaccaaaagaattctaatgagggtttctagatttaaaagctaagcatcaatctaacttagctgttatttactaataaatccctactttactccaagaatctaagaggcttagaagctggtttgtgcacagctgaggccaatctgagaggctacgggcctttgttcttccatcgcctcaagggccagacctggaaaaagagagagggtttctccagtgctggcctgcctcggaactacctggtgcttaggaacaagagtggtgactgggcccactctggcacctgctgagtcatagtctctgtggttgaggtgccagattcatatttttctgaaactccttgggattcatcagcaggttacattttgccagggttaacacccatttcctcgggtgaggatgtggttggggaggagggcatgggtgctcatcacagccctgtacctgccgtgggaagatgtgattagggacaacctcctgtccatccaaagcttcattgtcaaaaacacagcagttctgagtcattaatgtgacttaggtcttcaaagtttcgtaatcattttgctggcttggcccgaaagtggcacatgttcctctggtgccagtgtttgctagaaagaaagctgagggcttttacatttttcacctgccacgcctccaggggctgagggttcccttatatcagaagccagagcaatcactgacccagtcctattatgtccactcttggtgatggcaaaaattctgtgaggttgaagagagagaattgttctcccaattaaaaaacagaacccctaacaccctaaatgctctgctctgttcagtagcccaggctacctttgagaatctactccagccacctcctcctcagtcctaccctccgacccgccctgaggctggagtcctgctggcacttggcattgagacgggcttctccagggtctgagatcttggagggaagaaccgttgcccatagatcagggagctgagaggacaccagccccccgtctcccttcctgcttttccaaatgcctctgcaacaccagggtgctgggcgcatgggactgagggcctccctctgggccccttctcaccctgggctacccaagtcttggacccctgtggaacatggtcagccttctgatgagtccagaagcatttctccaacatagttgtgagcttaagggaattaaacttctctttctaagctgcagatttcctgaggcccaaacaggcaggaggctctgaatatggccgcaagagctcaggctgttactgcccctcctaggtgccagccccaggcagggttttccgcccaccccccaggtcagcctggcatagagggtctgggggcttctccccttggggaaggcagcagaagcaattgtgctctgcttggggtgaaggtgttcagataaaaatgcctggtggagtatctcggaaccacagaactacctgtgaacgctgtggtgctgtggtctgaaaacagggttttggagacatcagtgggaataaatctgtgactgcatgatacagaacaagctcctcactcagtggtctcagaatgtcttcttcttcccccagacatgtgcagacttggcctctcctccccaccagcccctatgtccctactcgcccaggagcccccagcacatcccaggttctgccatatgcacacactgcccccagctcctctccctgtgtctgcctgcttaatatctatcccactagccaggtctgactgaggttctccctatgctcttgatttaaaaaaaaaaaaaaagctttggaaacaaaaatttcaatccctttgccactcactagctgatcttaacatagtggttgggagatccccgttctgctagctgtgtggccttgggcaagttacctacctctctgtgcctcagccttgctggctgtgaggattaaataaaggtgacgtgtctgtagctaacttagagatgcgcggctcccccatcctatctggcagcagcccctcccactacagcatctcccactgggcagctctgggaaccgaacatcatgcattttcctgatctcctccctccctgacactcctggctccggcctcagggaattgttcacctccttaagcaactgcctcttcaaacccaggccagtgcggttcctgcagcctggagtgccaccccttctctgctggggaactcctatttataggagtcgggcaaatgtcctcctcttggaaatccttcccagcttcagggctctgccctttactcccacacccttgggtcaatgtttgccctccctggactgtgacctcctccaggaattaggctcgggccccagagtcctacacacagcaggggctggggctctaagttaaggtcgtagttgatgggtcagcctgattgagctgcgtgtgcagaggatctgaactcctgcagacaccacttctgcatgagtcttggtttgccgctctgcaa

SEQ ID NO: 73 - DNA, 3037 bp, Homo sapiens
  source 1..3037: mol_type = genomic DNA
atcatcaagtctccacaataccagttccttaaaggagaatgtttttgttccaactgaaaccaaaatattttagtgaattttgtcttcagtattacttccttcctattctaatactgtggtcaaaagtataacttttggcaaactctttcatagcggaatctgcttagtttgcgaatttgaagttagctgttgtctttactctttatgtaggttggatgtgcctggctaggttattgctgaggataagaagctggcactggaatggtaaagtgccactggccctttttgggtatgtggtgcccagacctgagctgctatttagtctgagaggaaggcacaaagagatcagtgatttatagaccctctgtaggaaaagaaaaaaaagcaaaacaaaaaagtccaataaacttaagatacttttccatcagcagctggttttgccggaaatatttcttgtattgaatccagctttagaaattcctcctgtagtctgaagacctgtcatcaggtcaatggatactataccctttctaggtactagcatctctagggttggtactccccaaactgcctttactggtgcaaggtcattctgtgggcaggcttacccaaaggagttcatgaagtagatagagctccacaggacgccacggactgggagtatagagtgtcctttcttcacagcttcactgtctaattgattatttctttatgtactctctatttccagtggggtacttgggattcttgttctttgtgatcccagacttactgacttttctagatgagccagattatattgacatgctgacaaggacagctcacagaatgatgggtgaatatatttcccttcccacagagtaaattttctagatttgcccaaatatattgtaaaaatttgttctttctctggtaaagattctaccacagtctcaaacaatgcatcaaataaacacatattagtattgtctatagcatatataaagtataaatcttgtgagaaagttatcagagaaatcgacattttatttgtctccatagagtctgtgttttaatgggggagtcagggaaagtctaaatgaaggtttttttttcaatgtatatgatcctgttttgaatgttctactgttgtggggagtagaaagtctctaccaaacttcacagagatctgcaatacttttactatttcatatttctagggcagcatctacctcatgtgattttattctatttaatataagtgatttttagaatctataccaatgcaacttggttacgtagtttttcatgcaaataaaaaataaaacaacaacacacctttgatggaagttctggtatttggttctgaaaaaatgatcttgtggattcagatccctttgagtttcatgggtttttgaaaagtattactgagacaaggaagccagaatagtttgggcatctgacagcctctgaggtggtgaggacaatgctttcctatttgcagtttgtgacctctgtaccacaaatagtgggcctgtcagcctttcatctgtcagagtggatggaggacaacgttccagtgaccctgcaattgtttatccccggcccctgcttcctctcctctctgactggttaactttctcacctgtgacactgaaagctgggtagtacagggatggagcaaggcgttattgaattccccgccagtcaaagggagatgcaagaaagcctctgctctattttcttttaaaggagattgaagagaacgtttgtgtcatgtgtcatgatgtgaatttggcgtctaaactgagaagcagagagttcattgttcatctttgtgcttcgggctgacaattctttaggtggttttgccctttgtctagaagagtaatgttacacgtggagctcaaaggaattttcctgcttactatttaatagtacaaataataactttaatgtcatacagaaaagacacttccctggagtgggagaagagaggatggggcagactgagtgtttgaatcgctctagatcagagttgcagttttgtggtttctcctttggttcataagctgtatttgctacaaaacattttagaaatcttagcataaaacatcctctattgcctcagagtcgtctcgaaataatttcccttaattaggtctaatggactgtctcatgaatctccatttggtctgacagaggtaattagccatgaacaattgtacttggccgtttttaattagaccagttttatttttacagctgccaattgaagaaacctaaagactttactgatcgtatctatttttttttttttaaaacatctatttaaatattttatctctgtagcagacaattttgcttatctaattccagattcctacagcttcttcttcctgattgccacctccctctccatggaagattctgacacgtctaagccaattttaatagtttcatttgcttgttgctgattggtttaggaatggaaatgtgacatagttccagccagtgggatctgaggagagctcacctgggaagctcctgagatggtttctttgctgataaaaatgagttacaggaaaaatcatccagtaatgtctgcgtctgtgacagaccatgtgacagacttaaactcagagtaaccagctgggcaccccagggggcagagctgaaagaagaaaataacctggctcattgataattgttgagccccggtttacccacttgaagctggtctgcttcttgcaatctgagatacatgttctttgtggtctggccccactaagtcaggattttctgttacttggatctggaaatgaataccatctggattatgttccttagaaaggtaactactgccactgtatatccttggactgtattgagctttaccatggggtttccagcctgagagccagctgtgagttagcagattctgcctcatcctcttctaaatcatttgcctcatgttctttggagtaaattcctatctcaggccagtcatcttctacagggattattggtgttggcatgtgggggaaacagactctta

SEQ ID NO: 74 - DNA, 2495 bp, Homo sapiens
  source 1..2495: mol_type = genomic DNA
agtgagctagccagcccagggataaaagccatgaattaatgaggcttctgagaatcctcagcaattggactgaccccctgcctagcttcccagggtggttccaagcaagtgtggcctaagtagctgacagcaggaggtgctgataggagtagtggagtttgccagaagagaagctagaagaaagccttgctaccctaaatagtgacagctggaaatcacgagggagcattctgtttgggagtcatgacccttcccgtcacatttctgcatctggcaggctgttggagagatctgtgtgacagcctcacctttcagcacctcagtcacaccagggagacagcaggagtgaggaggaaagagagaagagagcttgcgcctgcttcctaaactccttctctctgggtcaggaccaggttacagaggtcaccagtcatgtggtaacatcctgatgataagctgtgtccatgcatgtgcatatgggatgctgccaactaatttcccaagagccaggcagataacataaacgtgtcaaatcagctggatataaggtgaattggagaatgatgagtattatgctaacctggagagcttatactttgcctaaagtatctcaaagtttcagtaatatgtggccaaatataacaatgtaggcaggatttagcttgtgggccaccagctgatttcacaggagaaattgacaattacttctaaattgcaaaataaactgaaccccaggtctctaggctgtcgaatgggtgaaaagcaaatcaggagaactattagagtagcaatcagagtgaaattaacatgaaccacaagagaaagccaggcaggggaacaggaaaacatcaagcattttgcttaggaatcagatgaccagtgctgttcaatatgccagtgtttgtatttctttccttcaaggctcctgcccagcagatatgaagacttgcagttcattgtttaagaactactgtatgataacaatatctcttgtttttaaatttatacatccctttttttttttgtctcactgtgaaatcttttcaacatacagagaagtatagatgagataacgaatacttcatcatttggtcatgtttgcttcagctatttataaaactcttcatcttgacataattttagattttgaaaatcaaatattaacaccttatgtaaccacagcacaatgatcaaaacgaggacattaatttgatacaatactcttaactaacctgcagaccttactcatgtgtcactaattgtccctctttttttctgggccaagatcctaccttgcacctgggacagttcctcaatcttcttttattttgtatgacgggatatttttgaaaaggactggcccgtgattttgtggaatgtctctcaatttggatttgtctagtattttctcatgtttaaattcaagttatgcatttttagcaataccacagaagtgattttgtgccttttcccatatcaggaagtactcaatgtcaacatgttccattacaaagtgatgttcacttggatcacttggttaaggtttctccaatgtaaagttactcttttcctctttgtagttagtaagtattaataatttatcattcagaaatacttcgagaatgcaagtatactgttttttattataattttatgtactaattttaatatccattcttgcctgcaacaatctttggtgtttgccaaaaggtgatttttctatgccttctctctgatgtattcatttagccagctatttatttatagctttatgggctaatggaatttgctttataagctataacccattgctatcactgttttgttatttaaggtgtttcagattgggccaatgtcctgtgttctttctagatgctcctgtgaacacttcttattcaaatttttttgtttcccacggtggatgtttttactgtcctgatgtgggacctatcaaccctatatatatttccatccatgacttcatacattgattacatgtctatgtatccataaattacagtagggatttttgcatattttatgactctgggtaaatgaaatattataaacacatgtaactagactttcacaaattaatatcactcaaaagctcattcattttaactgctgtgtcacataccgttgtaggattaaacaaatgtatttattttttcatcacttgaaacattttgattgcacccaatttttgcccttataaacatgttcttgtaagctatctcctgaactattatgttacagtttcattgggttatatacttagaagtataattactaggccttagtatgtatgtcttcaatttcattagatctcgtctccaaaatggttttatttgtactcctaccagtgatacgtgaatttttgtaggagtgaaatggtatttgttgtttttatctgcatttccttaattattggtgaagctgagcatctttcatcctttgtgcatttctcctctgtaagctgttcataatctttt

SEQ ID NO: 75 - DNA, 2248 bp, Homo sapiens
  source 1..2248: mol_type = genomic DNA
gtaaaaatttcagaattgccttacaacaattttcaaataacacaccctggccactccacacaagagtgcaaaggacagagcccttacctagtggctggcatccttgttgctgattgctaacctgtatctcggctccgtctctgccacaggatccttcatacatgtgtatggacatcacaaagaaatgcctcaggggggtacagtgggcggcccctggtgagggctttggtactacatgctgatggttttctgagttgaggctgcaggtaagggatggagcggatcatggtctttagagagatgcgaagtgacgacagagacttcgaagggagtggataaataagaaataatctggaccggatacgtcagaggtgggtgggctctggtgggagctggcagagagaaatcctatctgttgggacatgtgtgctcaggactgcaggaagatctctgaggcaggggagttcagaggaagcttccagagctttctctgactttcacgtagtcactccggcttgttccatgttttgttagttttcatttcatgaacattttgtaaatagtgtaatatggtaaatattgtgaacatagacataggtcttaactccttacccacacgcctattataatactaaagaaagcatttctctggcttattcaaggccagcagttgtgcttggcataacacacaaaaaatctcattgttgcttccatgaaaatggagatttattagatagaggaatgtgtaaaagtatcagcacttaaatgcatggacagtgtggtgccattgaagcgattgtgaaacatctgcatggaaatttttggaaaacagacttggcttataaaatccttgaattcaatgtacaaattcatttttctctagacatatctagattcccccagtattccagaaaaagcccatctcttgaatgggataacatatgcaagtgttttgtaaacttaaagcactgtaaatagataaatcattgttattagtaataaaattccctaatatcatgcaataatctaagtgagaagaatagggggatgtggatgagaagcagaggaaaggaattgcctctcgacttgcactttgttggatgcttgccaggccggataggccaccccagagacagaagttggcttgtatttatttgtcccttaattctgaaaacataaaccatcacatcaatccatattttctttgtgaattttctattattttaccgtttttacattcattcattgcacaaattcattggggtaaaaagactttattccctcttatggaatttatagcccaaagatgtaaaaaaaaaagtcttatttttaaaaattggcttatttatttcttatttactcactggattatttttatccatctggaatttattttgttccatcatgtgccaataacctatgtcaggttttttaaacactattttccgaacactttttgctaactatggaaaaatacaagaacttaagactatattagattttcttgtgttgagtaatttctttgggaaacgagcctggactatccaggtggtgtaatcacaggatacttgaaagggaagagggaggcaaagaggcctcagagacatgccacgtgggaggactcagctcagcactctggctttaaagatggaggaagagactaggagtcaagtcacacaggaggtaaaggcagggacggattctctcctgcagcctcgagcaacgcagccctgccaacgcctggatgttagcctggtgagagccatgccggatttccagtctgtaaaccatacggtaataagcgtgttgtttttggctagtttgtgtaacttagtacagcagcaataggaaaccgatatgtaagtgaacgtgcagaggagctgggttggcgttgagtttgtccactctcactcccacgtgggaaagtactggttcttccctgggctggcctggactgtgagtgaattatctcctcatatgctgtctccttccttctaaccctgagttcagtccaagctcagcagctcaacccacagccacacacttacaaacttctcaatccttctggcccacttctctgtggttcccaattaattttctgaaaagaaaaaatcagttctaatacgtagaaatgagcatcccatccaccctctaagccatttaggacatttagtaaaagtgattcttacaaaatttactcctaaaattctcgtgtagttccagtctcctgcctagcgcttcagtcgat

SEQ ID NO: 76 - DNA, 3309 bp, Homo sapiens
  source 1..3309: mol_type = genomic DNA
cgcccccggtccacgctgctgcgggggctgacgtgtctgtccacccccgggaatcccccaccctcttcctcggaccttacacccgccaggggagtagcggtggaaggagagagagagcgaggttgcggggggagtacgtgtgagactgaattctcccttcctctccctcccggctcgtccttttagcaagtaagctagttatggacaccttctctgcccatgacacccactcccctaggctcaggtctagctagaatttagggggtcctaaattcacacaaaatcctttccactccccatcctggagttgaagattctcccaatgagcctaaccttcagtgtgagggttggtgtcacccatgagcccctgggggccctgagagggctgttggggtgacagagttgcaggatgcctgcttgctggggaggataccccagcccagaagagggaagggattaaactcagtccccatcccacctcccagcacaccaggcagctgccagcaactgctaaaaatacccagccctggccctggccattctgctcctggactggaagttgcccagagttggggaaatccaggccaatggtgttgaatgccagcaggagccagccacttcgagtcgtcaaggtctcccttctctctagctgtaggctgagccagccacccagggcacatccagggcctggaggttcaggcccccacccaaaaggggcataaacacagggtgtggtcaaggcatgaggagtgtcttagctccctgctcatctctcaggggtctgagggttgcaggtcaggctgtgaggtattttgtgacagggacctgctatcagacgattgtgacaagtgtggtgggggtgggagggcatccatttggggctcaggtgacatcacagatggatggaccctggtgggctgtggtaggttgtcatggagacctgggccaggctctttggtgctgcatcagtttccttggcaacgcctaagaaggccttggattgaggaggctggctcggtaagattagggccctcttgtccttgaggtcttcaaataggcaaaagctgggctgaggggcctgcccatggtcctaggggatcttggaggcctgagcatcccctgctggttctggcttcctgaacgaactccagtgggaagaggaagttatgggcagagcctcccgttagcaggcaagtctaaagagccaccacacacttcccatgaggaatcctggctggctgcctgtgccccctccaaccccatgactgtgccactggaaagagcctcctctgggtgtctggcagagggcgggtggggggtagctgtggcccttcccagagggggccatgcaaagaaggagggctgggtttcccctctggcaatctgagctcttgacctcagcagtgtttcttcattgccaagggaactgcaaccacaaagggccactcaagccccaggcattccgtggctgctccaacaggccagagacactcaccacaagctagatatggccaggggccccagggattaacaccaggacacaaccttgagacccactcccaccacctggttctcccagctctcttggctgcccaggaggaataaacagattataagacaaaacaaacatgaaatgcagaggcagttggctcaagtcgggcacacagtaatatctcactaagagcacgtttatcatgatcatccttcgttggatggggcaggttaggtgaatttgtggtccctctcctgtttctgctccactctctccaggatggtggtctgtccaggagtggggcagaggtctcagagcatctggggttccagaaagttccctgggcactggcccaggatgcacagatggtgccctgcaggcagccaggggccaggcactgatgagtcaagagtgacaagagaacatcatgtcagcagtttggtgtccctcacccccatggcctggctgccaggaaggccagccaaatgactcaatcacacctgcctcccttcatccctcaacacagacaccatggccacctcagacagggactgtccggcagaggtacacacgtggggaggccttcagctcgggaaggagccctcagcttcctcatctgtgcgggatacagtgcctagatcatcagagctaggcctagacccatttaaatctcccaagggcagtcccaagtggttgccatgactatccccctcacagatgaagaagctctgggcaataggggctgatggcttcacctctctgatcttaggtagccagatccagcactcctcttggtcattaggtggcgaggccactcctcctaagagtgagctgagaataggagttggggttcaggatgggcttagccccttctgtaggcaacggcgccccctgccactgaggcttctctgcccaatcatatctgggcctcagtagggatgagtattgccatcagagaatgagactaaagcaggaaggatgctccccctgggaaagtgatcctaaccagctaagccaaagtctgtccaggtgaccaaatgcaatctgggcatagggccaaggggaggcagaggccttagacccgagcacgtcacttcacagtgctgagcttccctactcaacactgggggtgacacatgcctttgggagggctgattgagagggtgcctcaggttcccttcaacagagaagtctgatgtcactgcccttatcagggaccagacggccatcactacagctggaaaagctggcatcacacctactagtgggcttataaaatatataccactcctgggccaaaaagcgcaaacctggactcactgaggcctttagaccaatttccagttggcaaaggaacaaatcagactgcagaacttgacacattagggaaatccagatatgcctgggtattagatggtgttgaggaactgttcattttctgatcagataaatattgtggctatacaggcttatattgacttaggatgcagtacttcaaagtgaagagtcaagtctctgcagcttacttactgatatttatatatgcacacataaatatggcccaatatgtcaactgactctactggtggtggtgagcatacaggcacattcgtacactactctttccattttgattttattttacgaaaagttgcaagtacaccaccctttagaggcacttactaccaccatccccccctcaaactccaaatgactccaccatgaaagacaccaaggcatgctgggaactgaatataaattaactttattacaaaaagcaaaatgttagtttctcattgtgagtgattcaagaaaac

SEQ ID NO: 77 - DNA, 2385 bp, Homo sapiens
  source 1..2385: mol_type = genomic DNA
agaagccttccctgctccataagagcagcctgccgccccggcgatgcctgtgcctctcccggcgcgcagcagctggagtggtttattacgttgcaggcaccaagtccatcctctcctgtaaggacagtgcttagtctctaacatgggggtcctcttgcctcggagaccagcgtacacacagcccgttgcccagggagcggtccggccagagtgggccagggggtgattgttaaatgtggctgcaacaggccctaacaggaaagctctgtgtcccctactcagcttggcttaacactatggagaaagttcttaattccctaattgcctagttcttgggagaggcttggctgtgcagttctcaggtccagataacacacactataatcatctgctgagccctgaggaggggctctgtcctggggcagcagttgcttttggattggtataaagaccatgatattccttttgccacggggtggagcaaaatatagctgggaaatactgttttgcaaagattttctcagtgatgggaccttggatcggagacagaaaaatttgtgtccaggtctcacctgagaactgaaaaacgcgaaatttggggattctagtcctgacaattgttgcaacttggaggagctttgagaggcccaagcctctcaaagagcacagacaacatctgaggatagcaatgcaataaaagaactactcagggagcgtacaaggtgctaagtatgtctttcattgtctgaaaattgctgagggcaacagaaaatgtgtatgtattatcactgttaattagagtacagcctcacttcttctgttctctttatctgcttctcttaccttctttcatgagcgggagatgggttcacaattaaagatgttctctttagaatataagacttgcagaaaattattttttctcctatttatagaagttatatttgcattaggagcctggtaatgtctcataaatatttccttttagataaaatattaattaaaggagtttgttgagttatactctgaaatcctaactaaatagcttctaaaacaaatattttaagcattttttctcatgtcaggagaattcttcatacgtccataaacagcagatatctatcagtaaggtattttgcaatatttaacataatactgtagctgctacccagtttaaaaaaacaaaaccaggcattgccaacactgccaaatccatgtgagattacctcctcttccctcaagaaccagccacctgagttttttctctttcccatggcttttaaatgctaaacagacacacacacaaacacacacacacacctactatgtttgagacactttgataggtgcttgagagagaccaaaaggtataatggtgggctttgatctcaaatgtctcacatctaacagggaaggtggcatgtctgcgggggaagtcaaattaacagcataggttttaattcctaggtgttaattgggagctggtgtggaattgacaggaatgctaacacagctcggagaagaggagctgggactgacttctgacggcaaggctacgcagagtctgaaactaaggttttgatgagagggagaggactggaaatcaaggcttgggcagcctcgggagaacaggctcagacaagtcaagtcccctcacactggcaggaatttcagacctgctagcagaacagtttttccaagatcatatttctaattgttatgagatgaacggtgtcccaaattcagatgttgaagccctgattcccagtgcttccggtatcctcataagaagaggaactactttgaggacactaggagaaggcagctgtctcccagccgagcagaggcctcaggagaaaccaactttgttgaaccttgatcttggactcctaacttccagggctgtgggaaatcaatttctcttgagcaaaagagatcgacgatagagtaacatcaaaggttctgtgaactgcaggtgaacagacgatcatcttcacctacggggctcatcgccgtttcattcctgtgcttctcgggaggaaagttacccggagcagcctggtggaaacccaagcgtcctggctgcagtctccaactggagtgtgaatgctttccttctgacggcagacagctgagaacctgcagggttaagtgcacagtccgggaaggttctgttcccaggacagcgctatcgagtccactgggcgctccacgcggtctacctaaagggaacagcctgcggcgcggcactattcaaagggtggggagatgaaaaataccgtgctgcttagcattgatgacgcttgcttcagggaaactgactagaatctatttagtctcgagcatttctgagaggcaagcaggcctcagggcactgcagaggga

SEQ ID NO: 78 - DNA, 2732 bp, Homo sapiens
  source 1..2732: mol_type = genomic DNA
tgcctgcctggccaactgtgggggtcacaccccgtgtgtccctggtctcaattgcaaagcgctgtggaggtgaagtggcactgcccacttctgaaggtcatccaccaatgctcctgcctcctgtagatccagtgaagttctgggctggttctgagccgagggtgtcttgggaggtcaagcccatcatccaaggagccctgatgggggggtcctcagggcctagccgaagagtcagttccgctccatagcagcctgatggttcaggtcagtcccaggcccatgagaaaaactccagttctgcaccaaaacacagtggcctttggagtccatcagactccaagcattaggaagggccaagctcttctcccattggtagttctcacacacctgtgggtccagagggggctacccgccatgatcctggagctgtggtctaagggatctctgtatacctcagctgcggtttggtgttttttattagtgaatggcttagctccaacttctcaagggaacatgctgtgcccttatctttgtgtgcctgcatgacagcacacagccagcatgcaaaccatgacctcatatgcacttaacacttgactatatctctggcgactagactctctttggacagggacttcttaattctgtatttctgaatggcctagtgctggctgtgacacagggtagttatataattgatggtgttgatttgaattttgaataaattttctaaatgagtcgacattatgacacaatggatatagatgagaccattaatgcatttgaaatttacactagtggacacatgatggtagatgctatgatacaggaacccatgcaaattccaactttgagaaactagtaacctatttcgaaattatacctttcttcatgggcaaagatgatccaagcatggaggacatgacatttaagccttcttcccagtgtaagggattgggagtgagaagggatgaattcttcctggggcaggaagggaggatgaaatcaggaaaggcctcccagaggagatgtttgaaggatggacaaatccagatggacaatagagtattctaggctggagggtattaaccaaagcatttctttgggaggagaacagtctcctctgctctcactgtagctggtggcttgattgctgaccaaggtcagggtgacacctttggccagggcatttgcttctaagtcagccaagggctctttctgtctgaaaatagctgcctgaattcatttccccattagtcccagaggccaaagataggagcaggctttctccaccagcagggcagaaactgcagctagaaagccggccctgcagcatgaggaacccctggggagctggcaggagaccttgaggtctgaccggctgtgtttggatggaccctctctagctctttcccccatgtgactgaaaaccttatcataatgaaagctgtaaaaatgccaaactttaaccttattaaagtcacactgttctggtcttggcaaccataataggattttacaactcaacctctactagagtccttgaacagcccagggtctgtcagggtccgtgatgggtgacttatgccttcagggactggtgcccacccacattcccaccctcccgtgtccatccaccacccgtgtcaccgtggccttccatatggcctcctgttttgttactcatagaaatggagaaatttccactaccatggaccccggaaaaacatatgagcatccctacagcattaccagctgacggtattttcctccggattaatgaagcacagtggaattttagcagttctgtaatgattaatatgtcaaatccatattgcaggagagggaaacaggaaagcgatgtgagaacctaggaggggggatagcaaagccaaagaaggaagggaaggaagagaagaacaagggaaggtggggagagaggaacacgccacaggggttaggctcgagctaggaaggtgggtcaagtcagttcctgaggagaggtctccggagcagtgcataaggttctacatgtcgggaggtcatggaccttaaaggtatggggtttatccctagaaaaaaaatacacataaaggggtttactaagcccctaaacctccatgagaaaacacaaaactgggttcagctgacggtgtttggactgtgtcatccctgtcagcatcccctctctgatgcctttcagcaagtttggaaagatgacaatggcgtcctctgtcttgccctgtgagaccctctcctcaggctgcagaggatgaggtcccttcctccaactctaggcagtccagtgccacatgaagtggtgtccatgctgtgcgtgaacatgctccgcagtcttgagacacggctggacaaaggaactggccaagccaccaatgggaaatactttgcaatgcaaatgagtcctccaaagccaagcattcctctcctgctaattacttttacctggttgcattacatattctgtatagtcaggagctggctcaatgttaatgtcattgtctcctgaaatccaagagacctgaatttcctttctttcttgatctaagcatgtagtcatgggccagaatatcacatactatcctctgaacatatgcttctttaacaggaaagaaaaaatcaggttctatatggccttaaatcgtctgtgatgggaactgtacaaaggtcaacaacaaacactagtttacatactcagtaggtgaaagagtgag

SEQ ID NO: 79 - DNA, 2973 bp, Homo sapiens
  source 1..2973: mol_type = genomic DNA
aatttgaagacactgagagtgttgcaaaaaaaaaaaaaaaaaacagagagagagggtgtttcagttgcctgcaccatacatctcgctggttccgaataatgcaagtctctcttctgttcttgagtttcctgccccttaaatatcgctttaccagaaaatgagaaagtcttcacccaaaaaaacagcacagaagtgaagccaagtgactgtaggtttcatagcctggtcaacagggaggcagctggcgatcacttcctgcctcaccctggaggaaggagctactaaggaaagggacgtctaagctgaaacaacttcaggtggttcctgtccctctctagagactaaagtctcactgcatggaatgcccttgatatccagactgggaaaatctaagcctgtggacaggaagaaaagtcattcatttacattcgtttatcactcctactactcatgagagtcaagtgagggtgtctctgactgcttcaccccttggttcagcccagtgaaagctggtcaactcttaatgctcagatccgtaatagcacaaccattttagtatctttctctgaagactgcaacacgctgtcatgatattagtcaactgacattattatagtggaggctgctccttgaaagcccttcactgcctccctggggtgtgggtggaggatgagataagcagctcacaagtgggacagaaccctggctctgcggaaagcccgggcttcctgtgtgagcgcatagacagccctgtagtccctcagccctgcctgccagaaacccaagcccgaggatgcccctccagatgtgtgctggaactgagctttctgaaatccttctcctggggccagacactcatcaaaggccaactctatcgcagcatttgagtcacctgttatgtttgagatccgccctgtgttgggagctgaaacctgttggctggtcttgctggggtgaagtctggaagaggactcctgctgtacttctgaggtccatgctgcagctcacagccagtgtggtcagccgaatcagctctgggcggcttattgtgaacaaaggagctcagttgctggggttcactcctatcccatcagaatacaagctgcactggggctagatgtccacggctgtatcctcagcacctagaagagagtctgggaccaagtgtgaacatctataaaccgttggcaggatatttagtatagcaatatctttggataaacaaacaaaattatgttgttggaaaccaaacgacaccactgatgaatgtacttaggctacttaatcaagaaatagtcgcaatgattctacttctgtcttcttttttaaaaaaaatttaattagttgttttattttagaatagcattactgattacctttcttccctggaaattaacacatttcgtagagacttaaggatgcatcaatgaaatacttagaattgaaagaggaaaattacttgatttctcaggatgtgtgtgtctattggtgctctagcacagttatgccactgaaaactgatgcaacttttgtgaagcaacagctgtaggcctctgtgtaacacttccttgtgtagaacttaggttttagaaaatatcagaaaagacctctttataggcttaccacataagtacattgttttcatcccccagtagactgcgattggctttgactggggagttcctcatttgtctccggggttggggcccacttctgttttttgacactgaccagctatgctgtcttcatagaaatcaggcagtgggcagtgacctggttcttactaactcttgtgatgcctcacgcgatgttatatctgtcaccagcacgctctcctctcctgagagagtgttagaaaattgcgtggtctgggagtgcaggggttaatgaacagttcggagatgcttcctctccttgtggcccagatgtcttcgggaatgcttgaatcctctccctttgcatcgtagggttacctgtcacatccctttcacttgcagggaccatgtctggacaaattagccaaagactatacctatgtttgaagcaaaatttaatattcctccaaaattatggctttccaattctccatggctcctcttagacaacaaatatttctgagcactgccgtgttccaggcagctgctgagatctggatctgatgggtaaatacacacagaccccacctctgctctttcaaagcacgccttacactcaatgagaacaaagaggaatcacagttattggcatagctcattttttttgtcagatccagttttgccatatgacagggccaaatctataaaacattgggagctttataggagataaaatacctctaaagagaatttttatagcccaaattcctatccagagtctccagcccctttgggtagacagccccctcaagattctcagggagctcatcacattccccattgggcagtgtgactagataagggccaagggcatgagtcagtgtgggaagtggcccagctcagctcagccactgtggcaggcaagaaggatgccacgaacacgccaatggcctcagggaggtgggtgggtgaagtgtggtgggggtcatgtttggttatgtaaatatgactccaggcagagccagtgctgagtttgatgatggaggaggattggaggtaaagctagagctctcagagccttccctgggatagagattcctgctgacactgctggccatgatagtctgtctggggagccatctcaccttcagcttgtgcagagaagggtggctggggaggtttgcatagctggtaggtgcattgatgccacttgtttctccacttggcttcagtccagagccccactctgggctggtggctgggtaagaccataggtaaacagtagacttatagtatggtttgggggctattttgctatgacacgggcttaatggccctggtgtcttcatgctactctgtatgctttgaaacaatggct

SEQ ID NO: 80 - DNA, 2353 bp, Homo sapiens
  source 1..2353: mol_type = genomic DNA
attgtcttgcaggaggcccgcgccaggctggtcaccgggggccctgcttaatcgcaaatggtgcaacccagtttttgatgttctaaactattaccatacaagagaaaagaaatttagaaacaaacagacggaatacattagccacatcttttttcttagacccttctgatattggttaaagctgctttgctctttacttggaagtagttttgactacttttaaagtaacatttgccaagagaatgcatgctttgtcatgattcctcatgtttggggaatttgagatttgagttctgggctgagtggatcaggagcagctactcagcctttccagttggagtagaatggggacaaggaatgaccaatgaggtcaaaggaagaactaagcactgtccccaaaatgcattctggaaccattgggctgggtgctttacttctctggatctcccctggggtccccagagccttactaaagttagggtttctctgtagagtgttctgaaaagcagagaaagtccttgtccaaatggaatccccagtggttaagattgcttagactcatacccatgaatcaaggcaatggatgatgtcttctaggacagatgtcaaaaccagctctgaggaaggtgcggtgtgagcagggagagggtaggctagatgtctcctagcattttcaatcctctaaactgaggtcagaaaggaggaaaacactgtcttcactgttcattgcctgcacggcagcaggagaaataagggaataacaccaatttgaaaccatgtgtatgcccatcccgtactccatatctctcctgcatcccctttgttcctggacatttttataactgtttccaaccggaagaagtgcatgaattgatactttggcatcaggctagatgtgccactcgggtttcttctctccccccctcctgctgtgtgtgtgtgatgtctaaaggcctccccatctggacactcacccacggcattggacattttcctttcaggtttatcccacgcagtcctattccaggaagcctcctcttccccacttagtgtgtagacaactcactctgccaacaataagtagtatgacaataaactagtttctaactactttatgtggattaactcactccccatccagttctctgaagaaggtgttattgtctctgtttacgttgaggaaactgaggcacagagtggcatggcatttctcttctgcctctccactctgtgagcgatctgcgtttagcagcgcctgccacaggggtctggctcttagtgaatgattgctcctggatctcccaaactcttcatatactttcacatccccaagattcctcctgccagctccatctgtgaaacttgtgggccggtatttgaagaatgactaaagggggaagaggttttatgaacttcagactgtgaactcaaggaagagagtgagtcaggcctccacattcctgaaatactttcaggaggctcagtgacctcagacaataagaacctgaggcagaatgtccttgggaggccccccaccaggaagaacgtgagggcagatgctctgctgtgctgccttctgtggttcctgcgcagtgtgtgggttaggaaaccactagccagaggcccaggcctaccgagagcgctgccttggttttgccaggctgactcctctccctggaatgcttgaccccaaatgcttgtgagcttgtcatcccagactcagctcatgtgtcacctcctctgtctgctacaggtagaagaggctttgtggttgggacaccacagggaattgcgcttgcttctctcactcctaaacgccctagaaaaacattgctttcaacatgtgtgcccaatacattcttcaacctactgaggttggcaacagctccaaggttgaggagtgagccctgtgccggagatgtgggtgctgccttgcttctgaggtacagagacttttaacccaggcgctattttcagagcggagacatagctattcaataagtttaaataattacaaatctttctccccagcttctatctccgtccatctctttcctcaccaagcttaaacccaggtatgaggtgcaaccatccacaaagggaaagacagagcaaacgagaggctgacaggtaaagaatgagtctcacccaagatgaaaatgttagaaaagctgagggagcacattcagtcaagctcctccaagctgcaccactttccaacagaaacaagctaactaggctgtgggttactgcaagtgaattgtaggctcgtctgcctgtagtgccatctagtgcctctggtctaaggaagttggtgggcttggccgtgtcgtataacctcccgtgtcacttc

SEQ ID NO: 81 - DNA, 2251 bp, Homo sapiens
  source 1..2251: mol_type = genomic DNA
gacccaaatcagatactcgtgagccaggcagtgacttgggctcgctttgattggcacaaataaacagaaaatgtctataaacttctttagttggaatcagaagtgggctttacttggattactcctccccagggaggggcatgacaacctatttttaaaggctcaagtggaaaagtccttttgcagctgtgaaaacaccctcagggtcagcccccccacctccctgccccccctgttaaccgcaatggggatgtggtctccttgatacggagggagggagggaggccgtcccctgggtgctgggggagggagttgtggccggcatgatcctattttacacaggaagaaacagacacccagccaagaccctcatttctgcaagtcaaatttagatacaaagaggcacctcatggtgacgaaaagcatctaaagagaatttcccttgttcctgggcgtgagtgtgaaaatgggttctgctagcttttgtgcctgtggttattttaatgaatagctgtgtaaacattattctttttgcaattttcttcagcaagttgtgactgcaggctccctcttccctgatgcccagagggagcacagttcgatttaaggaccgggcaaatggatgtcatccgtgtactccacccattcctgccccaaaaggccaactttccgttgaccattattttggatttctgagttttggcgcttctctggcaagtcactgtgtatctctgagcctcaggctcccctctgtgacagggagagtgccaggttgcccccagggcttaaatgagctactgacacgcagtgctcacctcagcaggagacaggcacagctgcctgagtgaggctaaacatgagccccggggcagcgtggcagagagagcacgagacaggacgcatacccaaatcaaggctccaaagctttgggtcagggcatcttgccctggagccagaggtctggcaaatgtttatcatgggggtggggtgtagcatctgccctgtctctggtgtaaatattcccattgtagcagatgtctgaaagagatgggaagatgcatttcagcttttgggaacccaagtgactccatgacagcactgaaaaggcatctgacaccagagctgggcccagggtagagtccactaagccttcaggaagtcaaggacgtgagttttggagtcagatgggttggtcctaacctggctcctccacctaccatttgcgtgtttagaccttgtcagctataaaaagaacacattgagagaaatgtagagtcatggattaggtggaagaaccacccggtcagaaacagaacaaagccaagccccaggcgatgacctaagcggataaaacacaagggagggtgagggaattggagatctgccacgtttgacagcgaagctgggttccagccaggccagtccacggggcgctgcttcattctgctgaccatccatcaggagctggtggcaatcactacccacttcatccttgggtgaatcacttaaagcaaaagaaatacagaaccccggaatcttccatagtactatggacccctttggctaaagtagcagacagatacattttagattctactctctagatcacaagcctcctagtttcttgtttcgggagccctcgatttccttgaaggatgctggcgtttcataacagtcagacttctcgctttgtgaaatcccgtcctaatctccaggtcaataaagtgactcatctttgggcttaacatccttttgtttagggagaaaatagtttctactgagtgataatctatttataaaacgttgcatctgattagccactattttatcttccaacaatgcttaggctttcgtgttttaaggaggaaaatttagtgggttttcttggccaacctcaaaggtgtctgctgggattggttcattttccggaatgcctaatggattgtgaaacatgcccgcctcccagcaaggtacctcacttggcctattgtgtacgacaaacatcttctgccctcatgagctgggctgctgttgatcccttccagccgaaagcatgcaccagagaagagcaattagtttgtattcctacaaaaattttcatggattcaaattcttcatagaaagaatgaaccagagagtaaactgaaccacagattaaagggctcatgtcaggcaattctttgaacagtttttttggttttgaaccatcattatttgtaatattctctgaaaagccagcaaataattgttt

SEQ ID NO: 82 - DNA, 4498 bp, Homo sapiens
  source 1..4498: mol_type = genomic DNA
ccagggaggggtgggggatctggggaaacggtcttgaaagagctgtatttccaaaatgtctagaagttggtccggtgggacacccaaagcagcagaataaacaactcccattgaagttacaataataaaatgtttcatgataagttaaaatattctttatattattttaatttcttaaaattaagaattattaataatcattaagcttataataaaaaattaaaaaccagactaggaaatgaaccacatgagggtgtgaactggtaaatgggaaaattagccccttaagtatctgagacaatagaacaatttgaaaaagagtatcaaatagttatggtcaaaatacagagataaaatgataggacataggactatgaaaaaagaacaggtaaatttgaaaaagtacagctataatatgtagaaataaaaagtttagtcatttacatataaaaaacctcaatgattgaagtcaaaaatctaataaaaatagctcattgaaaaggggaaagaaaagtaatatattattgtttataaatatagaaatttaaaataatatattaacaaatagaattccagcagcttacatcattaatagatcaaaagagaaaaatggaaagatcatcaccatatatgctgaaaagccatcagaatttggcatcgacatataaacaaaccctctgtaacatgaaacaaagggtaaagttcagcccccaaaccagtgttagaaatactagttctaaatgcaagatatgaatgtcctccttcacccctattcttttctgaacagaactggccacctttatgtgtttatgtgtgaagactctgttacaacatcccccagttttctagtgtatacatgtgtgtatgttcttcatgatcttaagtgctagatgaggcttcacattccatagtgaaaggtgacttgtgacaaagtctgaaccaacttcacaccacatctcacggttaggagcactggaaattttttaaagattaaatacacagggacagatttcctgttcaccccatctgtggttattccagtatatggaataaaatgcatgacaatgggagagacaggaagcctatactagtcattaagcatctatggaggggaaagggcacttccctttcaatttcccactaccctgatttcaacttcacatggaatttcacaagataatagcaagttagtttagaggatgtttcacagaacaggctgctttgctgagacctcagaggtgtcagttgaacaagacgaatgatttgcatttggctatttcattcctcaaggtcagtgctggcacgtccctcctctggctttagagattatttactgacaaaaataagtccagagaagttgtcaatactcaaggtcacattgctggttagtgactgaataatttgtgccatctgctaggtattactctttatacctaccaactcatttaatcctcatatcaactcacgtggtaggaatcattagattttatttatttattattagtgggaataggttgtatggagccaatctggtccaccgattgacttgtgcacgtccttaccagtcctgtaggacatcaaactgcaaccttggtgtcttgggcaagaagcaccaactgaatgatctagcctaaccagtcatacccaaaagtaatctgtgtgggagaagacagctgcttttaagccaataaaattcaaggcaggtaagacgacttagttatgacactgctgtctccttctcctggtaaaaatgcttacccgctgtggctgctccctcagtgcttctacctggtgcctgacctttcagtcttcctaataaatctgcctgagcttcggtgcctcctcaggtgtcacgaggggagtttgtagaccctttgggagctgatgtcaaatgctcaacaaatgttctgttaagttagtgctgcagtagtctggaaaggagaagattaaaggaataaaatatgcccccaacagttttggagaacactagccagtggggactctgctctgtcatctaaggtgaccatatggttggctgataaatgataacaattttctgtaacacgtgggcttttcatcttcccaacagtctggaggaaaaatattagatatgatgacaggagcagtttttaccatgtcgttaagggcccaaatgtagttttagattccatagacactaaatccagcaatgccacaagaatgcctgtgcaaacaggatctgtgttgcagttaaaattttttttttatttctgatatatacaaagaaaactcacttcatctagcttaaacaaataaggttattttgttaggaggtacaaagtatcagcagaatccaagagagcaggtgttgctgggtctctcagggacctgccaccaggttcccacagggcatcggccaccaaggaagaagctccctctcccatctttcgcttcctgcatttatttgctttattcttccctccatacagcctgcctttctctgcctctcctgggcttcacatgctcagttccagacctgagcaatgattgacttgctcttggtccctgttataaattccaagggaaaataatagaactggcctggggaggtatgtagggtggcctagagcatctggcttcagagagggacgtaattctgggtgggagttaataatctagaggagattgtggatgatacagacaccaatagcctctgggacctctctgatctatgacagagcgatttctgtcagggcacaattgctttgcattacaggctaggaatctgcaagtcagtgtttctgcttttttggaggtttacttcatatcttcactgggtactattgaagaggtgaacactgggtataaatactgttcttattactgtgttacgcacgcacctgagaagttctaagaggcgattctatccaggtttaaataaggagctttgcagaggtagtatatctcatgatcatatttgagatatctggcagaaaaaatgggtgtgttcttgactgtccctagtccttgagtttggaggcacttctcttctatccttccaaggctctctgctattcacccatgccctcttgtcaactttagtgtccacaagtacctgatagggacctcaccctgcctaaagcaggatttttggatttgccttggtttctgcccatgctgttccccatacttgagattcttttaattccttccgtgtctctaactcatacatatcatttaaggtcaagttctgtaccttttgtcatggttcactgggcttccttactttagaactcattcagtagtgattcactgaacagttacttatacagcagagtgctggctggttatgttgttcgctagggctaaacaaaaaaattgatttcctcacagttatggaggttagaagtccaagatcaaagtgtcagcaggtttgaattcttctgaagcccctcttgtcattcctctctgtgcactcgtgtctctttgtgtgaccaattttcctcacctctttaaaggctctatctccaaataccatcacattctgaagtagggggcatctggacattaacttatgaattttggagggacacaattcagccccaacaccagtggagacaggggtgaagaagacgtgcatcatcttagagctcctgtagtttgacggtgtggtagggaacacaggtattaactatttacatgactaggccattagttaccactgtgggatgatgcaagaagtagaatacaaatgctggaaaaggtagtctggggaagtgagactgaaagtgtgagatctgaaagaagattggatgggagccagccagctggaaagagagagagtcaagtattccagacaggaagatctgtgaattccatacccagggaattgaaagaaccaaagtcaaagggccagattacacaggaccttatttactgacatcctcatttcatgcttacctcggactgttgtcaaaaatcttttaaggggagtgttggcgtgcacaatttgcttctcctgattcaagggagaagacttcaactccttttgtattctctctccacatatgcaacatagggctggacatatagtaggtgctcaagaaataaatgtggatatgagaaatgagatgaatagaagcactcttgggtgtgtttctccaggctctgaaaggtgaggactttgatactataatgccacctagtgttcttgttattacaacaacacaggggagcagggtggtgctagtcttcaatccagaaaattggaaatcaatttgtttcatcagcataaattgatatgtccgaccacccatcctttatccattcatccatctgtgttgatgccccctgcatctcttctatgagctgaacatgtcacgcgcctttctactccttgtggttctttgattgctcagttttgggtctttttgtcctcctataagtaaggtctcagataccagtatctagattctctgagctcccattgtgctttctgttaaaatggcatatactaaat

SEQ ID NO: 83 - DNA, 1706 bp, Homo sapiens
  source 1..1706: mol_type = genomic DNA
ggctaattaactcttagccggcgggaccctcctcctccgaggttggccaggagcagcggcatcccaggcgttcctgtctgatgtcataggctgccggcgattgcggagaatcgccaccacgcctttatgaaggtcccaactttgccatctgataccctttactactgacaggcgctcagccaatcaggagcggcgagcggggtctggggacccggagccgccgaagccgtctcgggaaccggctcttaactctttgcggcgcagccgccgaggcagcagggccaggcgggaatctgggaaggggcgcctaaagagcggatgcccgagggctgcgacgccgcggggcttgcaggtggcgcggggctgcggcaggggagctcctcctggggatggtctcccatattgaaggagagaagattatccaaggggatagctcattgagaggtcacctttcaattctgccttccatagctgtggtcctcagccttcagccacaatggaagtgattatgtccataggtgtgagttcaaagtacagtgcagagtcaccaccttaaatcttgacccttacacatctcctttcctaggagcaccatgaggtcagctgctgggtcacagacaggggtagtgtggtcagctcacctatgttgttccaggacctgtggtcctctcttagcaaatcccctcccaccaaggtcattattgtgtgagtggatctcatggtcttcttgaagagttcttgtctctagtatcacctcttgtccattttctctttttattctctcacacatcggagcatttattcatctttagaaaatgcatttagggatgtgagcaagtcactgtgaccttgaagtctctcaactcccagttactcccagcacccacaaggcccatacaagtgtgtgtgggcactatgaggaagatggtttttacactttgggctcacatcacgagattcattttaaattgtgggaagacatgagttgacccagtaactcccactgtgtgaacaccactgtgtctgtgaggaaggcccttgcaaatcctcaagtctctcagactcgcagaaatgcatttggaaatatggccctcgagagtgggtggcccgtgggaaagggctgtgagccgctgagtctccagggcttccgaagttgcttggcataggcattgcctaatatccatgagcttaaatgctactgggaactcaatcataatgtatttacattcttataaatccaaattactgcttggaaaccatccagcaaattcatttctgagtaagacaaatacatgaaaatcttgcattatgtggatatgtgtccaaaatattacaataaatatcagtgaatttgcattcaaatattaggctatttttcccaaatatggcagtgctgtgattatgtgataaacatgaaaaaggcaaaatgcttgtttacattttgtcttcaattttctattagcagggtttaagtgtagtgagtttgtattttctcttatttaactggccattattctctctgaaaatcatattataaataacctacacagcagaaagaatgcttgatactgttaaattattctttttctttttaattaataccttcacccttgtgtcttgaatagaataatctaagcacagacaagatgtgttggatcatagttaaaacatttaaatttattactggaataaagagccagagtccc

SEQ ID NO: 84 - DNA, 2000 bp, Homo sapiens
  source 1..2000: mol_type = genomic DNA
cgtcacgtggccgtctagacaccctgtcgctttaaaaaaaaaaaaaagcgattgtgtttcgcaaacaacagatcgggtttctaaaagctatttctccccccccccccgccaccgccaccccctcccgggtctgtagaggggtaccgatggaggggagaggtggggggcagagaagctcccagaatggattgagccccggccggagcatggagaaattggaaaagcagggagcaccgagcgggcgagtttggagctgagcgagcgggagatcctatattgtagtaggatgccaacagtgcagaaccctactggacggacagaaaaggccacataagctgcagttgtataacatcataattttgttgtaaattaaaatatggcttattgaaaccttgtgtgaacattctaaagcaacctgtgaagacagcagctgagaaagcacaggagagaatggagaaggagtgatcccagcactttgggtaggcaagggaagacgaaagagacagaagtgggattgattccaggctggggcgttgccgcggttaagaactagggccaacagcaatccccacgttaggcgagcacttggcagagtttataagattttctgggcaagtttccgctctctagggtcccattcctctccctaaatgagagtggtcagaccaggcttcaagcaacagaaaggattaaataagcagaaagtagaggtcataattctaattcagtttcttggctaaatggagaatctgaagaattttggagatctgagtagtgctaaggctctgtaatgagaggccggagcacacacagggcaggtaggtttcatgggtgtttcaagagtggagtctgacgtcacggtttgtctgggtggaacatgcatgtacttgcagagatatgttcccgtatagtgtaattgtaggtgtattaattcaggtgtcacccctctctacacggctttcctcttccattttgctaccctaggtagaagtgggtgcggtgctgagcaagtagctacaatgattggtgctttatagctccctggcatcccctgaatcagttaacatcccagctttcctgatagccccaccccatcacattcttatccctctcctctccaagaaagaagccagaagcctggcagtagagtcccagcaggctggtgtgcctagaaaggtagtttctttctttcccttcacaggcacaaaggaggccaacaaacaccagtttcagactattctgacccttaagaaaaaagtttaagagtccaaactatttcgctttggtgatagtaatcaaaccctaatcatggtgccgccacaagtcacagagaccaaaatagcagagttatgacaaacaaggatggaatcagctaaagacctggctattaatcaccggccactctcttgaccggctgctggaccataaaggaaaaatctggagaatgcagtaaaatgaggttcaagccagaagggattataaacgttacatagcccagagagtctccatcttcagtccatcttatttggtactttttctttgctttttctattttattgatttttggcaatgcatgcacaatggtacacaattcaagaatacaaaaggggaggaagaaaaagatagcttcctggctgccagcacccatgcctctggcagccaccattacagccaaagaggcagaacatgaacattaggtctctgctttgctctccagtcccacttgcctggtcaatatctcaaaggcaccctagctcttaggtcccagatagccaaacaggcttttcctccagagctactcctcaccatatcacaaccagaaaccaaggacaacctctcccttaccccttatcctaccacccaaagcctatcatctccaaaatgcctctccacttatctgcttttctccagtctcacccaccacaaccacccagcatctgtccgggactggtccacaccttacctctggtcttcctcagacccacccagtccctctcccc

SEQ ID NO: 85 - DNA, 4772 bp, Homo sapiens
  source 1..4772: mol_type = genomic DNA
cattcctttatgagcctgccaaatcaaagaagcctttacaactggctgcctaagaataccagggcacaacaggaaacatgttgaaatattattgaaaacaatattggaattaaaattaaaaggtatcctttgttataattgggacccatagcattttttcattatgcttgctgccttaccggctttatgaagtttggaaaactagttactgaattttaatatgtgaaatccacacctaaaaagagcagaccatgaccccatagaagtctaaaaataccctctgatgccttcagaccttgattctgttataatattcttaggttgccaaattaagaattcctgctaatgcagaccataatgtaaaggaggttcggtttgtccacccatgaaccttatgtaaaacttatcacatttctgcttcctttctgctgaatgagagcatctatttataggctacataacatgaaaagcatgggagggttttctttcaggcaataaaaatgtgatctaagtcttcccttgtaatgaaaaacaaacagtttgacgtgtaattctgtaactgcttaaaagagtcacttatcaaaacagatcttttagtcttctaacagagtgtgtaaatttgggtaatgagttaccaagacttgaaaggtgtgttgtaacatattcaataaatggttagtgctagtgtgagaggaaggaagaaagggtcaaacttccagaaaacattaggtgggtggcagggtgactctcccaggaccctgaattcctgatttcagcagtgggcacatgattctgactccacaaatcataatgagctatctctatgggaacagtgatcgtacttccttgagttggaagttgagaagcctgaagctcctgggctgatcatgcatttgcaagattaagtatttccaatatttattattataaaccacatattatttgttactatcgctgtagtatatgacctggaacgctagactaaggtatatgcatatttttaaaaaacaacttactgagatgtaattctatatcatacggttaatctacttaaagtacacattcaatggttgttagtatatttatagttgtgcaactatcactacagaatagtttagaactttttatcaccccaaaaaatattctatgcccattagcagtcactccccattccaaccccagtccttggcaaccactaatttctttgtcactgtagatttgtctattctggacatttcatataaacgaaatcataatgtgtttttttttgtggtggtttctttcacttagcgcaatgctatcaaggttgatctatgttggagaatttcatcctttcttattgctaagtttattttaatagtgtaacgaataagataaagtggctgtgatgaaggtctggaaagcatctgcctaaaggcaaatatagtgattactctttacatataacctgaaatccagaacaatatagcatctgaggataaaataatttattgtagggcctaaggtttgcatcctgatgtatctgaaatgtgttccatattaccttggctttcaatttctctaattttctatttgattagaagttattcattttgggggcaatgatgaaattccaattcgaactgtcttatatcttttaatcagaattttcccaaagcacaaaagtgaaaatgtagaagttgctgtgtttcattagtttcattcttttgaatgttgctacactgaaagcgctgtgtgaaattattacaagttttcaaatggttgtaaaagaagatgcatatatggggactcactcaaagggcattatttaaaaaagaaatttccagccacccagatctcagggtagctatgtccattcaaggatagagaatccaccatctgtatgattatttttacgtctgaacttttttatacaccagagagactcaaaactttcagggtcatttgctcagcatgtagtggaatgtcctgcgcaatagattccaattaatatgaccatgttcaatggccctttaccaccaatgatatctaactgctttccacttggctttatatggtacttctttgttacagctgcaaccaggtctatcgcccctttgataaaaatttcagttaccccagggagaagctttcaactttattaagtattgaagtctgtacatttgttcttctttggaagagcagatcaaaagcacgctttctaagtaagtaatctgataaattaggatagcatttgcagacttgggcaatatgtttttccgtaattgaaacctgagaacctgtgtcaactgactatactattataaacttcttagcatttaacaagagtttcttttattttttgaaagtaaatagaattccttgattcgatttaaccaattctttagcatgctgtttcctttttgttgtggttaggaaaacctcaatgtaggtttagagatagaaaactgacaatcaagaaaaaaagcaattagatgatcaagttgtgcagacaaaagcccgtgttgtcgtcttatctctggcattaattactgtaaaaacttgagcaagccacttaatttttctcatctttgaaaagggataattggctaaattatgtgtaagacttctgctgattctgaaattctctgattctaagtcttaggcagaatgaggctagacacagaatacccggccatttttactttggcctgcttcaattccaggtttggtttggcaatggaataatatggtgctatttggtaggaatgagatatatctggatcacttaaaactggagtagtaataccttccaaaggagactagacggttgtatttctcccacagtcccaagctactagaatgttgaaaacgcaccagatttgaaaattggctcttcttcgcatacatttacaataaaaatacaaataaattcagatttctccatagctgatgggacaaattttgccttctgtgttcgtctgaatctgagggttacctctgtattttcaaagatttagattgcgacctttgaattccaattcgtgggatttggatgcttaaaaaggaaactatataaataaattttaggtatgtggttgtgttaaaatcctcttatttgttttgaacttagaatgggttttttatcatgcatgattttgtaacatcattcaatgataaaaatattagttcactgacttactcagattttctgaatcttgacccatttccttatacaattgttaatatcaccactaatctcatcagaaaagaagtgttgaaaaactatcaagttcatagcagtagattcaagatttctaaaattttaattttctcttgaaagctcacattttgatcataggcaataaataccatctttgtttttcttgaaacgacaggcttactttgtccattaaaattaaagtctgcaatctgaaaattcatcatttgtcagtcttttttttcaagtaaaaatgatgttttatgaaaatgtatgctggtatgagtttcttttcctagagacaactatcatatttcaagaaatgctatatgcacttccattttgtaatacggttttattgtgagtgcgtgatggtgaagaatgactagggttcaatattgttacagtcttgaatttccccttcgccagcttcttactgctccatttcccatgaagttaaaacaccaaaagtaagaaactattaacactgaggcccactcattagcagggacccgcttcttaatgactagattttcttgtcttgtgtctcccattttctaatattgtcataaaggttgtaaaaattcaagccaaaacactttatatggtatataattttttaataaagttaaaacataagctaatattacattgataattacaaatatgtcctttgacaggaatatctgtaatccttcaccatgtgtgatgcacttggtgcttgaagattctttaggaactgcatatatgcagatgaaatgaagttctttactattatccttatcaacataaatttcttgagaatgtaaagattccctgtgagaacccaagaacataaatgctaacaatttattcactaaaactgaaactaagatttgaaaatttaaagaagataatctttgagtgctaggaaaagctaatcattttctttaatcgctcttaagcttcattcttcctgaaaatttaaagtcttcctaatcagcaaatactacaacgtgtaaaaatcatacaggtgtacagcaaatgtgtctttaaaattttatgtcctactgatttgaaaattaaatcatattttaaatgtattaagagaaatcccatataaaagaatgctaggatgaatctttcagtgaggaattctattgttagaaaattctctctaattatattttgagtgtctggatttgtgtatttcatgtattttaagtcaagatatgttttagtagaagctattacaaaagcatttcttttcatttacaagttagaataataccaagtcattctagtaaagtatcctgaaggaaaaaaacaactatgagtcatggctgagtttgctaccagtaatagaattaattttagtatgcattttccttgacctcacaccaatagagctatttaacaatcctgggctgttctgttttacacagttagacttcatggatccaaatcctggttacagggcttgaacaggcaagccttaaactctgtgtgtctacttcctaaaatataaaagagaataatgtcactacttcatgggataattgtgaggattaaatgaatacatttaaagaattta

SEQ ID NO: 86 - DNA, 3625 bp, Homo sapiens
  source 1..3625: mol_type = genomic DNA
ataagctaggattcgctcctgagaacagcacatcaggcttgggtgtgtgcgatgccactggagacgccacaggagaggctgctgggcagctctgactgtggccgaccacatcaaagctctttgtaaagcaccaccccgtgttgagtagtccctccaggttctgcaggggcttgctcaacccgtgtttatccagcagagacaccattaacattcacatacagaaaacaagctgtgagcgcttctgcattggctggggaaatggccatgtacatttcatcctgacagatattgtagtaaagagtgcccccatctagcagaaagttggtcgaggcaacacagcatttcctgcatcatcaccccacgtgcaaccctgtgctcagccgtggaacatcaatcataacaagttgctgttcagatcatttcctgtgggactaaccattaaggacaccttttcttcaacaaaaccacatctgttgcatgattaataaccacagctaacaattatttagtcttattttgtgtcaggcactggcttaggtactttataaacgtgccttcttcacagccacggtatgagatgtaggtactgctagctttcccttacatgtgagatagcaccccacattcacaggccaaatgccagatggcatggggtgctagctctgagaacacccaaacctggggcctctggcaccaccccagccaggatatggggctgagaattgaactagaaagctcttcccagcagcacaccaggagttcgacctgcagggtgtcggccctgtggagctgtccgggcacactttgaaccctggttctgctgttaactagctgtgtggccccgctcatttgttttcactggtaaatgggagtaaattaataacccacccacagaactgttattagggacagttgaaaaaatgcatctaaagcagaatagacaaaaatggatgcaatttttagtgctaggcctttaagaagtggccctgtcctagaagcacattaatctcgggctgaaaatgatattttaactgaaaattatgtaatcttcatgaattttttaaaaaagactataaagctcctcacaatgacttttccagcattgtgctttgtgggattggggaggaaacccaactccagcctcttcgattttcatgtggctttgatgaacccatgtgcttttgcttggctttagtgtgtgctcctgagggcctgcttagcgccaggtggcagtgagccctgggctgaagcagctcacgacaccctcctcccctgagcagctgcccagacagctgtccaagatggccccacgagactcaactttgccaaaagagcaagtcaggtagctcagcatactcaataaaacctgaggggctaatcccattctacagcagtttccctggtgactcacagtcccccgaccttgcctatgcaatgagcacgtaggagccaagcgtaggagctggaaacactttttcctgcttccatgcaaacccggaggtggctgccatcatcgtgttgtgagcgtattccccaaaatagtaaccacactattctacaatatcccatatggcgtggtgcatgcctgctgagacacgttttacagcacctgtcagggaggggagtgggtgggcaacagttagaaagccaggtcagggagagggattcctgagaactgcagacccatgaggatggaggggaagcagaaactaggtttaaccccaaggtcatcatttgaagaccacagttaatggcagccaaggctcaggaatagggaagatgcagtaatcaagacaggctctctggcagccttgcaccttggggtataggtgaggaggctggcaagggcaagggcagggacacatttccattacagaaacaaaattgctggttggagctttcaagggcaccagaacaaggtgcacagggacccaggagctgagggtgggacccagtgggagttgcacacagccctgaaggatttgtgcccccaggggctgagatggggacctcccaggagcatcgtctgagccccatttgtgcagcatggggagcaagtggagccagagaccagggtgagacagcctcctccaatgcctcgaggctgggatgtgcctccctgcacctgatgccatttgggacagagaaatgagccggctgctcggaagacactctgatagtagagtgtctgcagtctgtgccttaaagagcctggtaaaacctaaaggagaggttcaaactgagcacagaaactggggagcttcatggtaaatttgatttcctatcaagacaagattagtcagaaaataaaatcctgtgggagttccaggttcctttggtctgttctctggggaaccccatttatgaacgggaacttcccaggatgcaatttgaatcagccctggctactgaggtctctggctcctgggaacaaacagcttttgccaaacaagcactgccaggaatcgtgagttcataaatgaatccttaggagcaccattaattgcccaatgagcgatttcaggcaaagaggtctctgatgtctcctagatggcaagtgagtgttggaacttgcccccaagatttggagctctcgtgcagctagtcaaggggatgatggtggtgaatttccagcgacactgccaaggagtgtttgcatccacccctgactccattgcttttgttgcagagctttgttctggcctgcagaggcacagtgggtcttcttgcttggggagggacactgctactgtcacatgagaacacgtccaggccaaatacatatgaggtacagccaactgcgtgctgaaaggtaggagagcaatgccttgggtgcagcaagacacatcagcccagcgtttataaatggttttttggaaacatttataaagtcaccacacccttttcatgtaaaactgtaatttgtgtattatttaaccattatttaagaatgtctgctcatggtgaaatgtttgtctttccaatatttaattaaatttcatttcatggtttaaagattcctttgctttgacttgtctttatgcaaaaacgatcagcctcatttttgtgttctaactgagtggaactatgcaaatgttttgattaaatgataaactattttgccagaagcagctatttccacaggaggctatgctactgaattatccaagcaaaatggaatgatgccagtcgctggcagatgtagggaaactgatagaagcttctaaatgtgtctattatcaagcaatcaaaataaatttaaaacaaaaacaaacaaaaaaactcaaaaccttaagataaagttggtagacatggtcacacgtagtggtgggccagtttgcttggtatcttccacctaaactttttggcactcaatttacacattctgaaactttccaagtttgttccttttcatttggaattcccttcctatagaaacagtgttaaagttggttgtgtctcaggccagcccatgagaaacttttcaatacatagtatattaaaacacgcatctgcaatgaaaagtgtggaaaacaaaacgcaacaatatcaatcattcaaataggaaaacagtaaaaaaaatttaaaaaattatcttatttctgaggttggttctgtgaaaatc

SEQ ID NO: 87 - DNA, 2068 bp, Homo sapiens
  source 1..2068: mol_type = genomic DNA
agtggctataggcacggagggatcagggagcatttcaaatgctgtcacatcagcagcacgcttcccacacaaaccccaggctcctgcagactggccagggcaccagatgctaccagttcactggaaagtacactgaccttggagtcgggttggagtctcccctctgccattgcccatcttgagacaggtagttaataaatatatagaattccagagtccaccctgtatccccagagcaggtgtatgtccccccggagcagatggaaaccgtttgtatgctgagagagtctgcagatttaagatgacagttgctcttagcctgcacctgagtatattccagctctgcaattcggagttgggcattgaaaagctaggcctgcccttgctgaggaaggggaaagccatatccaacattgctgccactgtataccgagccctgtgccccagcaaactcttccagtgtacttcctaactcagtgtgacctcaggcaagtcgcttgccctctctgagcctccatctcctcatttccacccgttgcaattccccaaaccacactgccttccattattatgaagctttgttattaagacatgagcgagagagaaacattcctagttgctgaggaagacattagtaaaaataaaccaggaagcagaaaaattcaaaatccccccacgaaaacctgcacctgctttgtagggaaggaattggctacaggaaaagaagggtgaaaaagccaacaacccacttagcaactaccactaaagatcaatactggaccaaaatagaatcccatgtaggaagcgatcttgcttcacagccccagaaagaaaaaaaaaaagttaacatagagaagccatttaaagcagaaaaaatatttgtatgtaaaaggaaatattttaggtaggagttgaataccaaatgattttgggtctctgtggggagtaatgaggcgcaaacacacaattctatatgcatgtaatattatccacgctcggaaagcttttaaatcacagaaatgcagttagggaccattgtacaaaataaacatcccacttggtgtcttgcttgccctaagttccttgatctagagcggcactgccagatttttacagccccgtaaattaacgtggtgctgatttgattgagctaaattaataagacttgtgctttgttgcaaaataactattacagtatgttgtggggacgcactgccgataaggaaattggtgcctttaacaatcccactgaagtttcatggaggatttcaaaaccaattttaattaaaacaaaagccaatgtgtggcaataatgtgggtgttaaggagagaaaatgcaaaatgtctttttaaatccattttaatatgtcttaaggactggatgatcgatgtggtgagagccagcggctgacactgagctgggaggctgcctgtgcgcctctctccagtgacaatggcaccaggctcacccggtgggcctctgcgctggctctcgatttccagacctggggtcatctcccaggttgaagctccggggaaggaccccttttctcagtccaggggactgtggcacttcgtcctccacacaggctggagcagaggtagacagagcctcagcctccaggcggctccagcctcaaagcaggttctccccagctcccttaaggcacctctgggaagcctggggatggggattgaataagaaacatctggagtgcactagcaagaggaaggtggtgccctgaggcagagcagtgaggatggaaaacctgcagaccaggcagaggcctagacctgcagtgaggagggccccgaggctcgaagctggagccgctgggggcctggggccagagaagtccccagagccggggcaggagtgtgagactcactctaagtctcaacctccctcacctcggtttcctcagctgtaaaatggaaacagtgatagaatttacatcacatagctgctgtgaggattaaatgaggttggtacctgacacacaaatgcctaagatagttattgatataaaaagaaccataatatgaccttctttttacctgtcatgtt

SEQ ID NO: 88 - DNA, 1279 bp, Homo sapiens
  source 1..1279: mol_type = genomic DNA
atgcctactgataaggccccgtgccactctctgtatctggggctggacctctctggagccaagagacattggtgagctcacggtgctgccctggtttggacagagagaagtgtcagtgctccacgggagggctttgccgctggggtttctcagaggccagcggtttgaccacggcagcttccaggaactgggatggcctgtctggggcaggagcagggagaatggagccgacaggcaggagcaactagaatcccttgaaagaattcaagttcctttaatgtagactgttcctaatacacaacggggtgatcagagccagtgaaacagtaagcagaggaactcgaagttccgcccagtgctgagattctatggtttgcaggatattacagctgtaatattttaaatactttaaaatcatgaatacgtatcagttttaattgatccaggcctaatcttagctagaaggaagaaggcccggccactatctgagctctgttatttgacaggcttgcctgtcctcttggcactgcttctttctttcattgtttaatttattgctgccacaagatccttaaagcaactcattgagtgtccaggattaaattagctcttggatgggtaactatatcggcctttagggtgaccaacatcctggtttgcatgggctgagggatttcttgagtgtggagcttctaggggtaaaactgggaaacctaaataaatcagtcaacccaaccacttcagcaattgggcaactgggttttttccattcaaatgcctaaaatttttgagactgcacctctggtgtacttactaagaaactgttgtgcctaacgtctccagatcttggttgttgatgcaacaagcacacaacccctgtcacagacatgccattttaaggccctggtgttgcatccaagaaccactcagagccttacataaacatatcttctaatgtccccaaagcactttaatgatgagtgtgcctcttgccagcaagcacattacatgctggctgtggagtattcttaggattctcactgctggctgccacacgaccctgcttagtgcttgggagccacagctgtgtatctctgaacccttctttctcaaatgctagtgctctgtgacttccagttttctggcaaattcaccgttgcccggcatgagccctccaagtgcccttcttgtggttagctccaaagctgaatgtctgtgtgcaagccccttaatagaatctagacaagatcaggcctcccctctgccaaacccctttctg

SEQ ID NO: 89 - DNA, 3490 bp, Homo sapiens
  source 1..3490: mol_type = genomic DNA
aaggttttactttatcacattaccaccaaaacatctatcaaactatagttacttagaaatagatatattttaagaacccacacaacacacccaaggacatcagaaagagcgttccccatatattatctctgggctatgccttcccatgcagcgaaaatgccactcaaattaggctgttcagaaaacagaagtgtaagagtttaaattcccttcttcttttacagaccaactgtaaaacaaacaacctgtagtatcaacaataaaaaagtaattgcttgagcttctcattctagaaagacagagaaagagggaaggaaggaaagaaaaggaaggagggagggaagaaggaaggaaggaaggaaagaaagaaaaaagaaactctcaatggctccccactgcccacagaaaaaggccacgctctttggcatagcacatgttcctccataatcttgagaaccctcattcctagaattgccttctcctttatctgtcctctgacctcttatcccttaggattctgttcaaatattatctcttttctaataatttatcatgctgcatatgtgtctcatgatgcatttaatgatttgtttgcctgtccctgccaatatgttgtgatcttaacaaccaagagcttggtgtttgctaatacctctatctagcattggatattgaaaggacagtttaagcaccacttttctgtaaaatgggaaaaaatactacccgtcagcattttcatgagattaatattatacgcagaagggcctagcatactgcctagaatgtgtacctatccattagcatcataataccaacaatatttaagtgaatgcttaaatcttgctttataaaattatagtcacagttatgcaaaatatacttcattagatttgggcaaagctggcaaggtaaaaggcagtttggaggctgagctgcgggtgacttctggaatgctcattgctgactcacttagccgtctatggctcagcttccaagactcgccagtgagtattaaacaccagccagcccacaaggtaatcagtccaggagagcagctgataacattcctggagttctatcaaaacactgaaggcttactatgaggcaatgacagtggtggatacaaaataaatcaggtgttttcctggctctcagctcaagcttcatgcgggcagcagagacatatacaagggacaacaagacaagctggaattggcagatgttatagtagagatacaagcaaatacattgatggtgcaaaagagagatgaattttatttgagggaatgggaagcaggaacgcaggtttattctgtacccatgctgggccgggggtagctcatttggtcttctaatatggtgagatagagaatgttagatgagcaccctaaggcccagagaggtgagataacttagctaaatcacactgatagtacactggcctttctggttttgactgcaccagctaatccccaagggagctgaaatggccacatggaagcagagcgccaagtagggagaattgagtgccccagatagaagggtgtagacaaaaccatagaaagcaagggaactgtatatttaaacgtttcaatacacatacacattgtgaaatgatcaccgcaatcaagctaacatattcacctcacatgctttttatttttattttttgtggtgagaacctactctctcagaatccagtctttacctatagtcactgtattgtatcttagatctccattacttattcatctgcttagctgaaactttgtgccctttgaccaacacccatggcaaccaccattctactctgctgattctacctacaagtgagatcatacggtatttgtctttgtgtctggcctaagtcacctcacatactgtcctcaaggcacaccaatgttattacaatataatgcccttctttgaccaacttaaggtatttcgtatctatgatgcccatttcctgccgacatttgccccagtttatggatcggatcactgagacaagcacttgtttcagtaacttgtgcaaagtcatgacacaagacagagcagacccttaaacacctggtgctaacaatctactgtgctctagacaaagtgaagaagatgtggaccctgagcctcagtttcctcatctgcaagatggacataataccttgtctcaaagagattttgcaacttcacaagaccgcacggcgggggtatctactgatagggagataagcaaatatttcctgtaaccctggaaatgtcattagggcccttagcaaaagagttctaatgttcagagagactatctggaagagagaaatctacaactgaaagagatgggcactcaagaggaaaccaccacctttgcacatctgagccactaacaatgacttgttgctgaatatgcttcttagctcctactagtaaagtcttgtcaaacaggaaacaggtttttaaggcaacggggtccctgtgagttcaaacttctgctttcaaatgttcttacaagatgcacaattggcactgacttacaggaaatatttaattggaatcggaaggcgctggctccacgggcctggttttatattgtctacaggcttctgctgaaatgattttggtgtgtaagcaccagtccgtcacttatttattttgccaagggggagtaaaaccaaaacatagcccaaggggtttctggggcacaagtgcaatctgcacagggttaggaaaagagtgggggagaaaacatgcaaaaattcctcttaaaaaggaaagggctgggacttttatgagctcagtatcagttgcttgagctgataagcactggtcattaaaggaaggaaaagtcacctttgtgtatataaaatatacaagattcgtagtctgaaaagactgaacaggaaagcattgggtaaatagcggatatcccttaaagtataatgtgaaaatcaaataacttcaagaaggttttaacaacaatatgacagtctagcgcaccttggtaggagggtggggtgcggacagaagaagagcatttcctggaaatcctataatgatttcattttctaatccccaaatgaccttggtccatggggaggccctccctccctcaccccaggccttggttttttgagtcttcatctgtgtgtcatcatggcccttagattaggcctcccagggtagacaacaatcctcctgtctctcagagaccactaactcttccttatcccttatttctacttcctctcggaacctgcagattccggtctgataagcatatctgcattagaagccagatttgcatatctcaggactcacccttggaatattcaactaactggcctgtccccattcggtcacaattgtaggttcctgcaaaatgatggtttatgacaagtgttcttccctcaggctaggttagaaaagattttctccagtagaaaacagacagaaaaataccttatttttgtagaatttctaccatgcaggataaact

SEQ ID NO: 90 - DNA, 674 bp, Homo sapiens
  source 1..674: mol_type = genomic DNA
tggacaagagacgcttctgccttaggtggtgagagaggagcgctggcggggtggaggaggcctctaatagagagcgggaagagttatgttgctttcccagttagaatcacaggataataaagccagcagactccaagcccttagcacagagcgtccttaatgaacagtcccctcattctgggggagcacagccctgagcacagcctcagacgtcaacatcccgaaagagtgagagtcccagtgccgtgagtcagtgtctcactgagctttccccacatgttcctccttaaaatgaatcaagaaaaagaaagagacaaaccagagaacagcctgatgtctttggccagatctcagcatactctggacagcaaagcaagaggagtctcaggttcatcgtggaaaacgtgtggtttctggcttctggaatttaaatgattgtgtatcttcttgtcttatcaattatcccctttacgtaaaaccatctcagactatgtgactgaacgctaaaccatataatctctaattcagtattcctctttgtagacccagaagaagatacgagacaatgtggaaatgcaagaacaagaaggattcttaacttttacttgtttctaaattgttagttgtattgaaactagtcagttgaatgtgtcccattcacactcttacctcac

SEQ ID NO: 91 - DNA, 4676 bp, Homo sapiens
  source 1..4676: mol_type = genomic DNA
aaaatttatgttagcatgaacactgactccttggaaattataccaatgctcaacaagcaaatttttagaaagaaaaatagaggaggacaaagaaggaagaaggcaagctgcaactataaatttctaaagagcagcttgttaaataaatggtttctctttggaaggcagatgtttatcagtaaatgctcctctgaagtgcagcttgttaatctgcagggagatgtctgggcacttttgttccaaaggtcgggatgagataaagggggaaagcttgacagctgctctgggatctgtattcatgaagcaacccggaatcaggtttatgctcacagctaatcacagactttccttctaacatacagaatggtgtgcccacatttgtgagtacacaaacgtgcgtgcacgcacgcgcacacacacacacaccttacctgattctggcatagcaataaggagattaatattcagaaagagttctgtaccagaactttgtaccagtgtttccaacagtggtgtacagcacactggtttgaaaccaggcttgaagtctaaaacaagagagctacctactgtttaaaatcttgggaaagacttttaaagaatgttattctccatgagggcggggaattttactttcttagcattgtatgtcagccctggcacaggatctagcccaatacatatttgatgaatgaacagataaatgaatgaatgcacaaatgccatattacagcaaatatttcacctcttggggatcttagaggaaatttgtccagccagcatttcctctctctaatatcataccactatttgagctcattggaagagaagcttaaaaggcagacataaatgtttattcctctatcgctcagccttgataaccaaataccaatcaactctacaaaaaagcacacccatccctctggatggaatgggacatccatttccctacagaaggcatgttttatccctaatgttggtaggaaaatggttgggtaaagtcctttccctctccccattttgggggcagtcatgtcctcattactgtttcctctctctcccaaatatatccacaaatgtttgctacttatattacaaaacctttagaaaattctctcttcccaccttccagagttctcaactttcccgtcttcaaccctaagtaaaatttcaaaggtggcatccaaagccaaaccgcacttttcacttgacaaaacccaataattactctttcttaagtcagatgctcctgctaatctgatacaaaccatccaaaattagcgccttccttccccttcaatgccaatggtattaaatgactcaactctgcctcacaccagccccttgtcaggacacactctcttgttctgtagcatcttttccattcaccatgataaaccatcttcttcacttcagcacccctctctatagcttaaataaagtcaggcttggctctttcgacacagggttatagcagcagcagatgctctgctatctggcatagttgggaaaaatgttgcttaatctatattccagttaaaggacaggggtcacatttatcaaacttgggtggtcaaccttcaaatacagtgattcacatggtaaagtgtacttagtactgaagtgacactgaattttgatgagtctttgatgattcagaaggtactgcaggaatattgcaacacttatgagtcatcattattatcagcgatcactggattgtaggttttgaaaggccgctgatggatggaccagatttgcttctgtgcactgggacacagcacatcctgaacttgcctggaaaatctggagggcactagctggcatcttggcgtctgttcaacaaatatttaagttcctactatgtgcctggcagaattgctagatttttggttctgacatatctactgcatatgatgatcttccttgagtgccttagcaaatagaatgggtaccatgcctccctgtctcttttaggaaaaggtttgttctatttttcccagtgttaattttaacaaatggagaaatttcacagggtctttctcttgattttatctgatctgttgggaatatggagtgtaaagcataactaggtttctgaattattttcagttgacaattttctaactgcaaaagagaacaaactcatttctatgccaaacaggactaaaaaacattcatggatgtgtccacttccttgtaactcaccaatttattttggttggggggggggcggtaagtccactgattgatatttgatccctaactttattcaagacaagatttaatttgaaggcccagtggagagaaatggtcctgcagaccaggaagcagtcctggctctgctggggaccgctgtctgtacttcagcattcccaatggttttaaactggatgttagtacttgcccatagcaatgggaaatatctgatggtgactggaaggctttatatgagccattgtctgaaaagagctgcattaatctatcatatcaggtatttttaattaatggtatttggcactatataccaactcgattttatatattttattgaaaatatgcacaaaataaataaaacaatgtcacatgattaaaggtaagcattgaaattctttcagataacctggagtttctctttgccgagtttctctctgggtgaagaaacaagattgtgagagttcagagttccttcctcacctgctgcaagttcacactcagcaggtcgccagactataataatacttaaaagcaagggatgaggcaaaaggcatctttgaaaattacatttgaagaatgcagtgtaaatggaattagatattccagtctagtcacatcgaatctttccaaaactcatcatttctcccatctcttagcaaatggtccagccatccctcattccccaaactggtatctgggcactgctagcagcaaggatgcaccactcttgcccctcacctttctagctgctgtttaccctaagatgttcctttccttaaaaactgtatttgacatatacaaacattacctacaaaatcaccataccagattttaggttatcattccactttgcctttattttttataatatattaagtaaacatatttaatatgtaaaaaactgttgcatatagtaaacacttgtaagcctaccaggaggaaccagaacagctacttggcatctacctatgtgctcctgtcccttccacttcctccttctgtgggtaattactatgctcaattttggtttatcagttaccttctttttttttagtatacgtatgcttagactacatattctttggttttgcttgtttttgggctcaataaaaatagtacgccccatgtggtctcttcaaacttgcttttttcctattttctgagattcattcttattgtgaggtatagctgtagttcactcatttccactactgaataatatcccgtagaactattttcctgtagacagaatttcactttttccagttattaacttaaatatgtaactatgaacaacatgcaagagtttgctggacatactgggagttctgcaaatgttcaagtctgtaagggataattgttttttagcagtggtatgttgtacaagtccctacagatctaacactgtagacccaacctttgcaaatcttatggatataaaatggaatctcaccctgaccaatgctacagaacatctatttatttaaaaggtagtcttcactatttttctatttttttaaataggaattctctatatttgtgattatgaatccttcatcagttatttgtattataaatgtcttctcatgtggatcactcacttgcttaatataagccttgagttttaacagaattgacttaaccatcttgtcctttatgattactactttatgtctcaagagattctttcctgccctaaggcttttttcatccaaacactgacaatttttgctttttgtattaagaaatgagtgactttatgctgcccacttgtccaactccgcacgggatatcagagcccagctgagaacctgctcaatcctggaaacaatagatgttaacacccttaacagtaactttctctcctcatttcctttttggggtacggaaatatgaaattatgggagacgaaaatgcccagaaagcatggctgcttgaccacactggggcaaatggcttcagtgtttctgacaagcaagcagtgagcttttgccaaagagttctgcagccatttagtccagccttgaaaacgaggtcttccgaagagaacagagctacaccttccccgtcagaatccactgttataaccacttagattaaaatgccccatttctcacagtatcattttgtcacagggtcacacttctctattcaactcagtttcaggcttttcttctaagaggaaaaaaagacgactaagtggcctcctagattccactgtaaaaacagaaaaaagcaggttgacttgctttgaaatcaaacgatgtcttcagtctgtccccatctcgagggaccagggaaaccacgaaccctgctcactgctcttcacgcaaggtcccaaaaaaagcgcctgttttccaggcactgcaggcctgctcctctacaaggtccactaaatagtcttcgaaaaccagaagcaggtaagtgtcagccagatgcactgttgttgttatttttaa

SEQ ID NO: 92 - DNA, 11241 bp, Homo sapiens
  source 1..11241: mol_type = genomic DNA
ttagattgtgaccatggaaatgattgttagaactttaaaggtcatttcctctttgcagaaagaatggctgggattagacctttatgttctctttttggtgacccagtccctgagccacatgggatttggaacatttgaagagaatactcaatgaacacagaagcagtttcctaatgctgagaccactgaagagcattgtgtctcagtgctaagaaaaaaaaattaatcaaagtggacacagttttctagatagctatgtgatctttgacaagttacattatttctctgaacctattttctcatcataagactggcaaaatgtctcaagacatcatctgctcactaaagtgtatgcatccttcttcctgggtacgccactttcgggaagtggtatttccttccacagtttatccagtcctttctacaggatgatgcacatagtgacctgacagttcccatcggtatcccaccatatctcttccttgcaaaagggcagctacactttctgctgtccaacttactcaccaataaagactgccagatgtagaaaaaaagaaccgaggaaaatgggcagaaaagaaacctcttgacgtttgtttgaaattaacccattgggtgaccacagtaatgaactacttctcccactttcactggattggcagctttgatccggaaatcccaggtgcctgagtgaggacagagctatgaaaattcaagcgggatgacattttccaaggagagttggattagtgcaggggaaaatgctgccaagctgaaaaccttttagtatggggaacgagaaactgcataagcaacatcatacctgcaacatgagtttattcacagtgaagagctgtgacctttgtgtgaaggaaacttgggctccgcagccgacgtttttctaatgctaatgttccccttcccagctgcaaaactcatctgcactcttctcatgcttttcttctccttcctgctctggttatttttccgggcacacttcttacactgtcagaacttaatattattttacaaccctgagccaaaccttgggagcttgaagaatagctgagaggcagacacaagcatgaactgaccccctttacacacacgtggctcttaagataggggaacccaggactctgctggtgttgtgggaaatggtttgtccttatctgaactcagtttgtgttgggggggcttcttaaattctgaggatgagaaagtgggttcagaaagttttactgctctagtgtcacagacttagcaatgataaagtcaggatttgtatctagatttacttacaccatgatggcaataatggccatttactgagcatcgaacatactctaaggacttcatgtctatttctctcattattctttgtaattctatcatcataccaattttaaggttaaaaaccttagtgaggttttattacttgttcatgtctactgggatgagactttatgataaagaaaaagcgcaaagcaaatgagagctaaatgttcctcttgcccatgccacaggtgttccaatagcacatcttcaattactagtttgatcttaagaatttcataataagaacctaataattaggatgcaattgaggtgcaattctaagcactttcagatcatcagcaatccttctgccaacaaaagattgtggtgtggggctaacgacactttacagttatcaaaggatcacacccaagacagacggatttttctaattgtaattgtccctcttgtcacacctcactcagcttcaaggagtgaatgcatgaaagctgctgagaaggtgaataattattccaggggacatgtatgcatttcatacgtttgagttcagctcacagagagtgaatcaccgaatactggaccataggcattgtgtctatgtttccaaaaatatcacaattatttatggtatattattgttcccctcttttctgataagaaaacctttcctctagtgtatttttatcttctctcaggattgtccacagaacttggactgcagattcgttagcagggactggggctgctttaagtcacataaaattctgtcaattgtctgctcattacgtcttctataacatattgtagtcttttggttttcaaatatactaccatgctatttttcagcaggcaagcctgggcagatattttctccattggtctacagataatgctattttccattttctgccttggaaaaaaccaacatttttacaaatgtctattgttttcccgattcaaagagctatcagcatttatttgatataacttccaaaagagatcattttaggagtacttactacatatgagaattgtcttcacacaccctgatttccatgagcatgagaccagagaggtacttttcccctacagtggttacaggaggttgtaccatatgaaaatgttttaaaccatctaatgtcaacaatttcgtacggttcgatctaataccacatgtggaacatagcaagtaaataaatatttgttgattggatggtatgcatttagccaactaaaacttaaaggaggtgaaactatttacccagcagagaaaatcactctcttcatatcggcagcaagtgggctcttgtctttgagacaaaaacaaatctttttgctgcttcaattcattcacttgttcattcattgacctttcagtaattatttagcatcttatgtgacggcaccatgccagctgagaaaatgcaagcccactgtcaaatgttgcaaacacctagaggtgccacttcctggtgataagactgaggaggcaggagccaggccaggaaactcttttaaggagtttgtattttatcttgagggaaatggggaggcgtaaaaggtattaaacaaggcagtgacctattcagctttatgtattagtacagatcatctgaaaaatgatttgaggatgtttttcaactaaaaagtgaactaatgggagtttttgcttatggaatcaaatttgtagttttggtatctttccatctttattatattggtcttttcattatgtcaattatatctctaatcattttggagtggctttatgttcctaaggatggattgtcctgggtggaggagtgagttcagatcacctataattgctgaaaatagaaattccctttaccaggacttcagcaatagacagacatgagcacaggacctaggaagatgtatcttgagttgcttacaatttgcactgaaaataaaaaaatcttcccttccctccaacaagagcctgaacaatgctctagaagagctattttccagcacacactcatgtctcttctccaacactcaaagatttaaaagagtcgctgaaaagcttttaaactgaaagacagaaaataatactttcattgcattattttaagcatttatttttttcttgttgtatcatttaatttttgtctttgttttttatgaaatatttacatctgtctcattataaaagtgaattaagttagcatatttagtaaatacctactaggtagacagaataatgcagacatgttaggatggcctggagtagagaagttggaagggaagtatgtattttgctgacttgaatgagttacaataaaatcagtgagtaagcaggaaaacaattccttcactaagaattggcaggtcaaccattacttccagataagtacacagtcttgtgaagatttgttcaacaagtaattactgatcatttgccatgtgcaaggtattgtgccatgtgctggggggttagaaaagaatcagaaatgaatatactcaaggaacaatgccatttctatgaagactataaactgtgaaatgatctttatcagtcaagaataaacaaaacaaaacattgactacaatcacatcgctccagcagcgtcatcatttcaacatctatttggcttctttgcttctgtaactgtgtggctacaatttatggttgtaaatatggatgaaaaatttaaaatgttccttctggaaatgtctggtccaaggacttaggatccaacctgagttctttgcatgggagatttgggggaatttgcaatcgtgactcaaggaaagggctcaatttttttcctgaaatcaaacttctcttacaagatgcatttgagtacattaaaggtaaatagcacggaaacatatggtttgaatttcccatgaaatgtgaaagttttgataagcacatacttcagaaagataatttggcctctgtaaaaccagaacatttaagaaatccttctagagactttgccaaaacccagtggactgtttttgcctacagcttccacttcagccagacccctagtgacccggccaagtgaaacagagaactcagcatcttctgaggggatgtctgccaatctcaaccctggtatatgttcaagggcttccagctcatcctgtgagccaagagaacaatttgaaaacgctggactcttagcatatctgcttcgggttactttgagagacagtgtgtgtgagaacaggatccatgctggaaactatttgcaaaagccccacttccttctgccaggccagcttctccgcaacccagacagtccgagaacaaaaaaacaaggactattgtcaaattaatagcactccctaaaaaagtatggcaggtggtgagattcttggccattgagttccagccaaaacatttcacatttttttctcctgagggaggcactggagcttgctgtgagtcagaagtggggggctgcagcagagagggccgcttgagcatcttgcctgttcctgatgagaattagtcaattcttggaaagagggaagagctcgccttgctctgctacccacttccagattctctgctcagccttgggttggagagtgagtaatctaatgtgattctgtttattccactcaatacatatacattactgagtttttattcagcatcaggccttggctctgtctctagcttattttctcacatggctattacccacagcaactttagaaataacagcccagtacaaagagccaaacttatcctttcgggagacaaagacgatatttaaagataaccacaaagtatggagttagcacagaggacagtggaatcagaagactttgaagagatggattctaagtaggttttgatagaagaatgaagttttgaaaacacagggatgacccttccaggcaaagagaaacatgtgcaaggacatgttgttttcccgaatgaagctctctcccatttgtaggaaagtggatgggaggacagcagaggatgccgagaaaggctagaacatctcaagcttgttccactgttttcagctgtaggtaaatattaaaatatggaaaacatctttctcctagttcaagtacctggtgtttaacccaatgtaccttcttcatgcattgtgtgatgtgctcttttctactgcattcaatcaaagccatcaagcccaattttctataaaatgaaggcctctgaagaaagggaagaatggctctgctgttcgggagcactcacccaggaggtacctaggtgcctgggactgtgcaaacaaacttgagagtatattatgatgggaatgctgaggaatgaggcagattggggttgagattccaatgggtgagctggaaattgtctgggaaggaatgaaatcctgaactaagcaaaatttggtgctgtttgccctatggcacaaggtcagaaagtgcaggagtcagggagactggggtctcgttccagctgaacacaacttgctgtgcaatcatggcagatggctttccctgaaagtgaaatggattagaccatgaaactcaaagaacatttcagctccaatatactataatctatccattgtcagcaaatgctcaacattaagttaaaattaaaacaaattatgttcaaatgtatacattcgaaagatcagtgaaaaagggaatattagtacaatgtgctagacacatgctatgttcttaataactgtgtaaaagtttctttcatccctattttgcatatgagtggatgaaggctcagaaaagttttccgaagttctctcaactcccaaatgagagaggcagggtttggtagcaagtcagtgtgggtgcaaagcccctgctccttttttttgcaccatcatcctgaaacaaagtttattaagcactttgagtggacgagcagagtggtctgccttccaaagtcaagaggatgctgttaatggaacagggaaatgccaaggctgtttcctaatcttggcatagaagtgtggcatcatacagaggctgctctgcacaaagtcttgtggagtccgccactggggcaaagttacaggggtaaccaggcaaactggagcccactcactggaaacagcttccccactcccccgcctctttatccaggtcagtggggctagctttgcttcaacatggccaataaaacctcaccccagaaaccttccccagtgtaaccccaattggagaaaagccatgtgaattgaagcccagtggccaattcggattcagtctgagcaaggtcatgaaaacactgcattgctggctggttggttgcaggcctcctgtaaccaaataatcacaaaccttgtggttaacacagaaattgatttctcacaaatatgaaaaagttactttcacatgtggaagctagatgtctaagatcaaggcatcagcaggttgaggttgttcgaggcctctgccctttctccctctcttcttctaaggacaccaattgcattggactagggtccatctgtgacttcatttaaccttagtcatctccaaggccttaactccaaatacacactggggatgaagatgccaacatacacattttgagagtgcacaggtcaggaaagaaggtgttctagtgaaattttcattgcaaatatattcatggaaaaacatgagggcagttagcaatgggatttagaaacacttagaatagtagttgcaaatagatgaccacattgctcatggagaaagtgattcttttcaagtgactcattcaaaatggctgctgcatagcacatgatgaaatgctgtgacagatgaggcagaagaatgacctctttagcgatggttacttgaactcaggagaaccaggaacactttgtttggtggtgacttcatcttgaaatctttggcctagcagcctacagatgctgagtgattgttaaatggacaagtgtatttgcaggaaattcaattcctgttttaaaattacttattcctccttccaaaggttttgtcaattaatgtgacacttaaaagccctttgatgtcttagactcaggaagctgagtgtgcaccagaatgcacaatcttctccagaaacgctggaaagcatctttcaaagcctctgcccttaaccccaaccctggggatctctttctctttttgtgtaggtgacggagctgagggctttcttgctgaggtcctcagcgttcttccctcatcagtccaatcctgctctgaaggggccagtgcctctcaaggtgcttcccaacatgaaggacacttgttatcctgtctctggcgatcctagctcctgcccagtctgctcactggaatgattcgccaaggaccatagatagtcacactggaggatctttgaaaacactcatttcaaccccttcattgataaaataagcaaataagaccctagaggaagggactgttttaggagaatcagcaatttagaacccaggcttcctgattaccatccaatgtcttttgaatacgccacatgtgtttctctgagtttaatttcaaggcactctgttaaacctccttgctaagtgttctgattttttcaccacgtgctcatttataattattactccctcagcattgtagtaaactgtatctctgttccctgaatgcactgtgcattttgcaatgaacctgttcatcttgttctttcaggacttatcattcatactctcttcttctcaagtgttactattctcttctcctagggcacaaatcaactcaagcattgaattgctttatcattccaggtactaaaagtttaatttcagaagtttcaaggcatgggcatttcccacttctggcagcatctgctgcatcgttcctttcccatcattctttccccccttcatcctttcccgtctcaacttagagccacattcaactaaaaattttcaagcagatgccatgtcagatgcttaatatgtgtgaggtcatttcattctgacaataattcagggtaaacattattatccccacttttcaagggagaaaactgaggctcaggagggtgacgaactgggaccaggtagtgaagaaataggtggtccacatgtgaacaaaggttctttcctgctcactgggctttaggctacattgttacttaggtttgatgggagttctacttgatagtaaaatcaggtgaaattatcgctgtattccatttctctggcaatagatgtggtgacgtgctgtctgccagcattatgcatcgagttacatctcttcccggttcccacatcctgtggataactcaagcgccagccagccgttaaaatcatttcttccaaatgagggtggtgggacctgacatgtggatgtttcctgcttccaaatccaagaaatgaaggtaactgactccaagctaaaagagaatcatcctcatctgtggcatcacttagcagtgttctagctgcggctctgcagtcagcaaggataagaaagctgtggggtcagagctgtggaaggaggggagcttgaatgggaggaggtaggagcaccctgcctggagatgcagcccagaggaataatatgtgaatctcacatgactcagcccctgtgcaggtaataaaatctcaacgagacatgagcttatttaagaaaacacgcaggaagaattcatcattgatgtggctattttgggagatgaaagggagcgtgaaacggaggaacatgtgaaaagcattcaagctgagtgagtgtgatagaaaactaagatttcccttcataattgaacagtaaaaatcaagtgtgtccccatctttcaagtcttcggattggaagagtgaggtttgggtgcagatgttggcagttctttactccctggataaacagattgtggttttgatttggaagggattgcttttttattttattgttgctaagagaaaccaggtctcaaatgcaatctcttgggctgggaatggagattccccacgcccccttgaacgtgccagcatccctgtgaaaagaggcactgctgttagatgctgctattacctcatagtgagagcagaaatcctttttctatcacttgcaggagactctcagttaaacacgatcagctacgaagatgacgggacacaggggagagtcttctgcctttgatgagatgtgccagatgtggatgttcccagctactctattctctccggggttctcacagcagccttgtttatcttccagacatccatccagccttatatcccaagtaggaaaggggtctccaaccttgattcctttcattgtttttcctatctctgattctgtgtcttcctacttctgacttctgacatcaattgttcattcagtcccacatatttatgaagtcctaggtctctgttctcccgtctaagccccctcccttcactttttctgatgctcagcccatggaaacacttgttcaccatttggattttcttcctggttaagccacccagggcaaacatccctcaaaccctactgccctgtgactctggcatccgaaatactctcagggttgccagacccctttagaaaacctacacattcaccctgcagtctaccttacgaatgccacagttgctctgatattgtctctcttggcctagaaaacatcaatgactttctactggtccaaaccaatcccaaaagtgtctcctgaggatgaatcagtggagattgattctaatacaagattcaaagcagtgtctttctgctctttgtaaatatttattgaatccctctctgtgtaggaccttaatagattctgtggatacagtgatgacaaattagatgtgtgtgcctcgtgatagttaccatttgtcatggaagacatcaaacaactaacactgtgaagatagtttgaagaaaggcaaagggtgttatataaatgcaaaagggggaatgggtctggcaaacatagtctgaaaaatcagagaagattgagacctagagtgtgaggagggattcattaggccaggtccagggacaatagtggcaggtggcaggaaccatatatgtcaagaccatattgctggaaacagcagatgtgttcaggacccagaaggaaggtggaatggcttgaggacatgtgatgctttcaagcaatgattcttgagggactttgtataatcatctggaacacacactaaatatctgattctcatccccaattgttgaataagagtctctgtggatgaaactctggtagataggatacaaaaatggttgtgtttttcactctctcctaagcccatgacatgtgtgatgtaacttcgcagctcttggaggtggggtctctttctttgatctgggctggtggccaacagaataaaaagaagtattgccaactatgaccaagactcaagaagtagtgtgtgcttctgaccctctctcagaacctgcccccctccccaaaacaagagcagactagcctggtgcagaatgagagaccacatggaggaccagcctattatccctctgagaccatcctagatcagcctatagcaagccagtccctgacaagcaagtccacccagatcagcagagccgcttaccctaggacatcagtaaatccagagaccaatggcttaaacaataatcattgcaacaataataattaagtcactgagtggttatttattctgttactagagacaacaattagtgagataggcagaggaggtcagcttgagtaggactttgtaggcatgatgtgaatttggtgttttgtcccaggagttatgtgaaattactgaaatgttttgagcatgaagtggcacaattaggtttgtgttatagaaaaatgaacttggctattctgtggagtataaatggtgggagcagggatttagaatgaatcataggaatcaacttggtatagtttagttttcctttaaatatctatattgggctttagcaatgtatgcgattacaaggaggaaggctcatatcctgtctttaaatctagtgagaacagaaaga

SEQ ID NO: 93 - DNA, 1397 bp, Homo sapiens
  source 1..1397: mol_type = genomic DNA
cttagtcgcaggtccccactagtgataaaggaagcgatgctgctgcataatagagaatttgtctgactttgcccagggtttctggcacagaccttatattgttatccatgagacccttagaccacacttgagtttatgaactcacgatcagctcctagatagcttcaggatgagagctgctcaccagaaagaccagctgcatcattcgagggctgggactttgaaccaggctgacctccagaagaggctggagagtgagactttatatgtcaaatgagaaaattggtatgatttatacaagcattttgcccagaagagcatgccatgctgagtaatcccaagtgaggacataataaacttttggaaagtgttttaggtccttctcttaacctgcctctgtactttttgagtgaagaggttactacagattccatcacactgttgtcttctgacattttgttggtttcagtaaggtcaggatatttttcatcctggatttccttgaaggcagagtccaagaatagggttcgtgttcaggtagttttataagtgataataggaagcaggagtgtaaatctgagctttctaagaagaaaacagactatgcctcagcattgtccaccagaaggtaaaatggagaagcaattcactattgtctccagcccccaagggctattgattgcccacatttatttgttacttgaatgagtgctgagcagagtgtggagtataaaagagggaagagcagcagacacgtgagttgaggcactgccagcctgaaacaggccaaaacctacatagaattgttcagtaaggacaagggtacatgagttggtgtacaacaggttgcatatactgtgtatttattcccttgagtccctccttgcaaggccaccgcaggtgatttatatcaccctacctaaggctacagctcctgttgggtggcccatgaacctcggatctgagccaaccaacttctggctccgacaagagacccaggtaccaaaatgaaacctggttctgaattggagaatccaatgctctaggggatagcaatgtaaatctgccaaacccaaaaggctaagcatcatgagattgggaaactgtaagaaacatctactcaggtgaatctaaaaagaaaaattacaaagtatgtggtcaaaatatttatcagaaaatattttgtacaaacaaacaggagaatttattactgaggaactgtaagttttagaaccatattttaaagtagactttacaccatgtggtaaaatccccaaagaatataagaaaatgtagtatccatgaagaaagaacaagaaattataaaataaaaacaggtggatatgagtcaagaacagaaagatttttggagggagttgtctttttaaaacaagctt

SEQ ID NO: 94 - DNA, 5008 bp, Homo sapiens
  source 1..5008: mol_type = genomic DNA
gtggcagttttgctaagaagaaaagcaggtgaaagtgggagattccaaaagagaccagagagaaggcagtgtgtcctgcccaatctgccgctagggtcagagaccagctccctagtcacactcggccaaggcaggctgccggctgactccaactctaaacaaagccaggcagccgcccaggtgggtgaaggctgcagattgcgctcaggcgtgacgccggggtgcaggtgggagcgacgcccgggtgcgtgggtgcagcgctcgcccgcccgcggttaggcgcaccgcgggaaccggtcaggtgcgccgtgtggggcggggcgggcgcccccaggctggtgcctggggcagaggattcgcctgcttgacgcgctgatgccttgatttggtctgacaagtggaggctgctttgagaagcggcctgtatttttgacattcaggaagctagaaataaccagctgggctcctggcgctctggccgcaaatgtcttgcgctttccaggcaactgccagattgaaggggcttttccccatgtagacctgtgctctgtgtgctaggaggccgaggttccagtccaggctcggcttctgactccctactttaggctctcccacaagcctcggggggggtaaacaaaagtgagaggtgaagctgctgcagcgaagtaccccttcaaaatcttcagtacccttcagacagcgctctatcacataaactgtacaactccagtcaaccagcagccgcctggctggcacaggaagagccaatgcaattttgcacagtcttgcttgaaggtattgcctggcaaatgggcaatggggaaccacagggagggcctcgagctcgctttggcactctcacgaagtccactcttcatttaaaacgttgctcagccttaagggggcctgaaaaccacagcccccaagggagccaggtctgttcaaggcattgcttccaagtgagactcttcacaagccttttttcttggagaccaatggagcagcgttaatctggcacagatgagcactcccataatgctcaacatctctttccaagagtggagagagatgatgcagagatagtcaatcatcccaatataaaaattagtggacaattggcaaataggaaggaaatatttaataattgtgttgttcatgcatctatcccagaaatgaattcaatcaaacactgcattatgagcatgaatttgtgttacatgatagactcagggatggcaacaaaaatatttgagtgctactctttgctcgaaactctgctttacttttgctcattcaccatgtctcatgaattcccctgtcctttgatttctggttctttaatattctgggattcaatctttctccccatccccactgccttgattcacaacctccatcatctgtcagtagatttattataaagctctctgttatggacctcccacacctcagaatgtgactgttttgggagacagggcctttaaatgggtaactaaggcaaaattaggtcatatggctgggccctaatctgatgtccttataagaagaaagtaggacacagatgcacagagagggaagaccatgagacagggagaaaccggccatctgcaaacctaggaaggagtcctatcctacgttgatctcacaagtccagattccaggattgtgagacaatccactgttgttcaagctccagtctgtggcacttagtcatggcagccctcttaaaataacacagccttctgcctgggctctgtgcttgttccccagttaaattcagaaaagtctaaaaattattgttgtttatctgaaattcggttcaactgggtgtcctgttttttatctggcaaccctgtacaggaccctttgtgactgctagtgaacagactgaaccaaaatgaatttgataagaattgtttcccccagttaacacaatagaaaaagatagtcaagattgattcttggctttcaatattctttcaatttaccaccaacccttgttcacagtttagaattatctgaggagcatttaaaaatatccataccccatccctaaccaattagaatgggagttactaaatggggagttagaaaggcagggcagggagagaggccaggcaagtgtatggccactctcaggagccaaccctgcacttgcacagccctgagagtgagaacctccttaaatcttatgccctatacacctgttgtagtcaggccctatttaaagataaagaaaaagaagggttgtgcagaagagcttccaggggatttctgagcagtcctgggtcccagctctgtgtttcattgctttagaaggaacatcgacctttccgagcaatgaacatcagctccagctcataggcatgagtcagggcaggcttcaccaggccaggttcctgaaatgtaaactcacagcaggctctggtttcctctagaagaccactcctaaaaggctttaaatgcttaggttccttgttctttttcagttcaggctggtgattgagtagctttagtcattggcatcctaaagattctgtttcacattcaagcatggttttaaattactacaagatgtaatgaaagtataggtttcactcttctctccaaggttttaaagactctcacccctgttctccttcctccctctctgccttctgcccttttcttgctggtgattttctgccccatcactggcagcctggtcaggagcacctcattaatccacccactggtacagtgtgcccagaacagccaaggccaaccttggaaggccaactctctgaacagtcccttcctttacctctaagttccgtgatgtcatcagcattctgccctggctggcggccagatccttaactactgattcccagatctctccacgttttaaaattctaggaaaagaaactgtacttttccagtgctaaaaaaggatgggaatggttggtggaagctcccagcacagctaaaattaacttctttcttttgtgataattgaatgattttcaatgatgttcttcctataataggattcaaaaagttttcctcaggggaaaatttggaagagaaaaatacaagggaaagggttctctcagtagcaatttccttgcaatatgaagctcatgaaattacatgggtgtatctcacttagcttaatagacatgtccctgtaaatttcatataaatggaatgtttaaaaaattaaatcactgtaagtgcagctaaataaaggaagcccggggtgactcactttataaagtgaattatctttatgtaatgtacatcatcctcccccacacactgatttatattataattttgaatttttcagaggctggattctcataaggtaagggtgcctcttacgatgacaactggactgaggtcacagctgttctcatactctgaagctgaacttttttaacgttctctggggagtgtgtagatgccagggttctgcggggatttaagctcttacactgacccagactcctcctggcctatccctggagtgatgtgggaaattgtcaatgactggacagtggaagaagtcatggtagacaagctgaggctagaagcatgatctgtggccagaagactacggccctgcgtctgtcctctagctggggattctctgagcctcggtatcaccccagccagttgttaggatttagatatgaaaactgtgctgttgaattcttaagcaaaaaaatgactaactttgaggaagacttcaaaagagaggtgacatttgaaagttacaagtattatcttatttaattccttagtattggtgagatgtccagctggttgcccacctagtatttaaaaggcactttttaatttacatggccaggcagtgtggctggctaaaagtctctattttccagtctccctggcaggcagctatggcccataagatggaaacagatcattgggtcagacttaaaagcttcttaaaaggggtataggctatttacttggagtttctaagctctttcctcttcttcctggttcttgcctgaaatgaagacaagacggctaaggctctagcaccaacttgtgaccttgagaaaggcaaggggagtctggtccctggtgaccgtgactgcttagcaaccttggactgccgagctccagacttgcttgtttaaatgaaagaaaaataaactatcttacttaagcctgtgttatttttggcaattgttattagcatctgacccaatccctaactggcataagctcctgaggccattaggcagaggtgcgttcccactaccctacactgttggggcaagcctgtggctccagggcacccgaagagcctagagtgtttcctttaaattgcaattccttttttgtcaggccctggcatcaagcacacagcctggtcagtagcactcaatatatgttgaaagaatacaatgaataactgccgctcgaccatttcccaaggagctgtttttctgaggggttttcacttctcggatttccacaggacagcatcagtacgaagcttgtgggacatccaaagtgctttgagatttaaaaaaaagaataaatcggaaatgataaacatttaaacaatgaagatatacataaagcctcatttaagtagtatgttttaaaaggtgaagcaaaattgtgtattttttgtagattttaaaaatataattaaatgggatgaaactgctatcccaaatcccttttaaaaaattatttttgagtgattgcaaaataacaaatataactttgtaaaagaatcgaaattatttagaaaggttaaatattgaaagccctttcatagaacagtttggtaaatatccttttaactcttctgtacataaaaatcatgcaaatagatgcgtataaataagctcatgatgtgtatattattttgcaacctttgcttttttcacttaacatcctatcttgggcatttccccacgtctgtgcatttagattaacctggttctctgtaaggtaatagagcatcctttatcccaat

SEQ ID NO: 95 - DNA, 3639 bp, Homo sapiens
  source 1..3639: mol_type = genomic DNA
cacagaggaagattcaactatcccttcttgaagaagttgagaagaaacaactatgttcaccagtctaccctaacacaaaactgagatttcactgtttcaagaaaaaggctgaaatacaggctagggaaaaaaagctcttttggttaactttcagtaaggtagaactttccaacactagaacttaggcagcagctgcctttgaggcttctctgcaaagatgggttctgaattcaaaatgatgaacttgagatcatcggcccttcatctttgactccaataacaggacagaattcacctgagtcaggtgaataactgcagactatggacatcttggtatggaagggcttgaaagggatccactacaggcacagaatcacagtcctggaggccaccccatcagacagccctcatcttccactccgtactctcctgaggggacaggactcatccaggctctcatcaagtctgcaacttgtgatttgtctcttccaagcagagtttggtggtgagaattctgactgattttgtgtctattgctacttcaacagttattgatcatggatacattgtttaaattgtctaaaccttggattcctcttctgcaaaatgaggaaatgataacttcagaaagcagagagcccctttttgggtgtcttggtcttggataagcctttgatggacttttgggttatacaaaagtgatgcccagctcctgttttcaaatagtgaggttgagatgatgagaagataaactgctttgcctgggaaatgcttcctacatgtgttaagaggaagcatggtgttggaaggagggagaacatacctaccttataaatgatgagaggcagatgtgggttaaaacataaaccagcctgaagttgattcctgactgctacttaccagttgtatgtccttgagcatcttaatatttctggtacctggtgtccccatctgtgaatggggggaataacaatattaactttgttttttatttgactgaatctcattgttgtaagctcactgttttatctttggagggaaaattatcactagatatcctgaaagtgaatgggagttgtcagtgggaggagagttggttctgggcatgggctacttttcagagatgatataaatttagagggaaaaatggggccagtgatttactctccggttgagaatataagttgctaatgtagagtgtgtgaccctagctcttctaaactttcagtgtcgttttagcaatcctcggcctctcctgaatgtctggagacagggcctgtctctcttgcccccactccgcccccccttatgaaatggcagcccctggtgcagagcctttgccaatgggaggctgggctcaggttgcccgcttacttagcacaggacaaggcaagaggccaaggcgatttcctctcttttactgcatcataaaaaagcatctcattcttcagacacctcttagtgctcatgtccaggccatgattagcagataatctcctaggtctgtggtccatgcacgcctgcatcccagctgccaagacccacctgagaggaaagaaaaggtctgtgttgaagaccccttcccacggccttgagggcccacctccttgaagttgctcctttctgtctggggggtaatcccagccctctcctccttgctgacttggctccccggctgctatataccttctcaaagagaaccccgccaccaaccacccagacttcctgagggtcacaccacaaagagacggcagaagacggatggaatccagatctctatggtctacactcacctggccattttgggactatttctgggtgtttcctagtaaatgcttgctgagatggggcctgagatcccaggggactggccctggatctaccctgggaaatcatcagagctgccatgttgcctgttcagctcagcagggacatctaggaggggacaggaagaggtggtcagcatggccactcctcttccagcctggtgagagcatagtagagagagcagcatgggcttgagtctgagacagacctgagttctagtcctggcatgccactttctagctgtgggacatttgacaatataagtcatctctctggaccacattttaaaaataaataaaaatgacaggcattacactgtaatggaccaagagagattctccatagagtactcactacattattattattatcatcacaaaactaggcaaatcacaacctatgtgagtctcaaatttgtcctctaaaatagtgcctagtgggttctccagttagcgttagttttcttcacttcatattgcaagtccaagagggagttttggtagcagaaagaaatgcaagttaaccgaaggttaaggctcaagttctatcacttccaacatgtgcctaacacatctatatctacatcaatatcttgacatatatgttcccagtcattttttttcctttttttaaaacaaagactgaccattctactatatttgaaaaaagtatgctcatgaaaaataattaatagaaaaaagttgaaacatgaatgtagtaaaatcagaaatagctctccttaagaataaatacatcatcctattgtttttgtattcatacacatacgtatctaaaaacactttaataaaatgagatcataactgtaaatgctatcttaaaatatgaagtattaactttaaatttactggaatacaacagaaaaaaatataccaaagtaaaactatagaaattgttgaataaatttttctttactgtagcatagtattttttgaaaaatcttctcaggttaattaaatgattatctaaaacatcaaaaggtggcagtcatcatttcttctgaaactacatggttcaattttagtatcagttgtgtttgggctgtgaaagataagggcattagtgctcagatttcttccagctcccttcccaacaaaacttaagtcttgttagttaccttgttatttttacactgttcagatgtatggctctgactagaatagttttatttatgcattaatcccatcccccgattaacttctatggggcagaagcagatggagcagcttcatagagaggtattgtttaattcaggaaatagatttgctaccactaatgatatttcattcctaagtttatttcttttctttatttcttaactggctgaatattattgtcacatagttttgttcaagaagggctcatgggtgctgtattcctggtttttcatgtctgagaaagtattgcttttttataccattcttgtatgccataatatcctgggtgcactttctttcccttagacattgtgaacactgtcttttggcatgaaatgttgctgtgggagaggctagggcaagcctgatttgaacccctttgatgtgcttttcctgactgtatgaatgttcaaagtgccattatttaactaacttgtatcttgaactgtctgcattttcctggaacatggtatgtctttcaacttgcatattcaatttttttatttagatgattgtaaaattttacttttggatttgcatacttctttattaggtgttttaaaatctattatctttgtactttctctaatgtttttgttttaatctacctttactttatctcagactctcatccatg

SEQ ID NO: 96 - DNA, 1146 bp, Homo sapiens
  source 1..1146: mol_type = genomic DNA
acttctactgagctgggttgaattttcttagccaacattctgctctaattgactagtgagagagaaatgagcatgcaaataaagccttcatagaaacagctacacagctccaggctccttctcctttttgaataactaagattgtttctggcattggggacagctcctccaggcatttcaaaaccattaggcacagaggagggagagattaatgtcaacaggaggtactaggaaggcctcctgcaggatcctgcaggaaaacacactcctctgctaaaaccatggctccaaagtgcaagtcttccaaaggcagaaatccctcttccctttagcacccacatctgagtcaccagcaagtcctataagttataactacaaattttattccaatatatacatttttctgcctatttctactactaccaccctaatccaaggtacatcatccagtgctggtttttcctactaatgctccttggacctcttcttgccaacccaaaccattccctacacagcagcatgagtaagcttaaaatgcaaaccagaccatggcactctcttactttttaaataattatcaattttgtttaaaattaaatttaaactccttaccatgggtctctaaatatgcatgatttggtccctgcttatcttttcaacttcatcccatcgatttcttcttcattattctgcattcataaagacctcctttattttgtccaacacaacacatttttcccagctcatggctttgtacttgctgttttctctgccaaggatgcctgatatgaaagaagaaaaaaagaaagaggaaaggaaagaaagaaaagaaaggagaagagggaaagcctgagctaaggcacaaaggccagaagtagaattagaaaaagataagcactttggtggtgcttaagtataaacacaatgatgataaaaggatgaagaaacaacagaggctaagaccagattaagtaaatatgtattagctgttattgctgcacccagaatttagcagtttaaaacaacagttaatctcacactttcaatgtcagcaacttgggagtaacttctggttcagaactttttatgaggtttcaattaagacacagcctggactttagcatttaaagtcttgaatgggactag

SEQ ID NO: 97 - DNA, 1820 bp, Homo sapiens
  source 1..1820: mol_type = genomic DNA
atggatagttaccagtctggtatgtattctcacttttcttcacaactcagtgaaggtactgttattcttattcccattttacagatcgggaaattgatgtataaggacaacaagctccaggagggcaagaccagcatctctttcactcacccctgtgcctggaacatagtaggctcataaatacctatgaaatgaaattcagtatgactcttagattggattctagtttcaagacaactccccaaacacttcaccacagaagagaacattagaattatgagactggagtcaagaagatcagacaggaggttgttgtaattcaagcaagagatatgaatttcagctaaggcaacattatgaaaggatctggtaacttttacttattattaaagctctatttaatccgcttgctataacctagttaataaacttggcagcattagtgcacttctcagatcatcatctgagtgcctgcctcacactgtgcaaagagctttacacatatgctaattcttactgcagcactggaaggtagatatcattatctccattttacagatagaaacaccaaatcaaagctcagggaaattaagcagatttgccaaggtcacacagatagaaagcaggtaagtgcatagccagtctatttagccctagctagttcctattaatagtctattgaatgaatctacaacaatatctgttgatgattatttaatggaatttctggatcctaaggtagagttatgttataggtatgtgttttatcatattacagaagtatccatcaattcattttactgattgttcttgacaccaatgtcctttgatgttgccttttctgcatctgtggaaataatcaaaattttgtagtgttgctttattaacaggatgaactcttgatgttttttctaatatgaaacccacttgttcatacttctttatgtaccaatggagtctgttcattagtatttagttttatttaagactattatccaaaattcatatgactagtctgttgtttcttttctggtatagcatggtcagctttggagatcaatgtaatacctacttcataaaaataaattctctttcatgatttgaaataatttaagtagcattataattatgttattattaaagacttggttcaatctggttctctttatgggggtgtattcagttactttctctatttaagatcatttgtctctggggtcaattttagtcacctgcatttttttctaaaaactcatctacattttcaaagttatttccatagagctattgtaagtagtctttaagttattttaattttttctgtgtccatgattatttctcttttgtcctaattgtgtatatttaccatttctccctcattttttaaaattaggttggttagtagcttatatataatacagtgttggatgttgttttgtttaccaatcaaaaaaattaatagataataatcccattttatttttttaaatgattgtttgccttcaacacttatatattaatttcttatattttgtaacagatatagcactttccccttttttggttattttttatttggttcttactatgagccatattaagaggtaataccctcttttgttcttttccctgcatcactctcaaataatacagtaagcttttcctacttttcgtgttttccctactttcccctaatattttgatgtactatccattacatattaatataattaccatctagttgaaattctacatgtaaatattcagtacttaccatgtaaatttttcttcattctttattgttgaagaattagcctgaa

SEQ ID NO: 98 - DNA, 5410 bp, Homo sapiens
  source 1..5410: mol_type = genomic DNA
gtagagaaaggacaagaagagacaggaaaagaactagatgcatagcaaatcttcagggtgagttgtcttgtgacacttttgtttcaggtacttgatacacacatacaagtatcttggggaatccaattgattgtataaattacatagttttgtgagaagttgagcccctggatggagctaggagaggggatcggagaacagccttggtttaatctttcatgtaacttgtgtgcatggcaagtctttttccgtctcagtcctcatgttttcttcctatgataggagaatatttgaataaattaggagcccttgtattggtttccatcacttgtgtgttcatgaactcagcagttgtaattactttcataagataattgcttaaactcaagtatttctaacttgcagtttctcacaattgcatctaacctgtaaattactatatattttaagctgaaatattttaaagtgaattgcaggcccccctaaatatttccaaatgtatctctaaaaaccattttcttaaacagtcctaacctctattttgtgttcagtttctcctgacatccccaccggaacccaatctgtgatcgtgcgttgcacctggcctggatttttctggtcacttccctgacagtgtctcctggcttgccgctgtgtcccctgtttcttccagtgcagtgtatgggtctttggggtgcactgaggtgatggacgcttagtccagtctggggccagaatttccaggagatgctccctgagattgtcttgaggggcagatgagactcatgcagccatagagggagggaagggcattccaggcagggggaacacccaggcaggcattggggcagggagcaacatgggtctcagctctacaagcagctatgggttcccctggagtgtgactcggcatgaggctggccatggaggagcagaatgttccaccgaggaactgggactttacgttgtgagtgatgggggctcactgagaggtgtcaagaaggggcaggcaagtcaggtctgtatgtcaggagctcaggtgagtggagtgcccgagggaggtggggtgaagggaggtgggcacccaggtgagagctagtgcagggctggggaggccaaggcctgggcagtgggagaggaggggcagagatggacagggtagaactgagggcacctgcgattggtaggggtgatgagggagggagaaaaggccattgccaggtttctggcctggttgccactgcccattaacacagggaggtgaaactgcctaaggaagaaccctggaggatcaggattggttctggaaagtgttggagttaaggtgcctggagtgcctggatcgtgttccacttattaggggtgggccgacccaggcggcgatgctaagcaggtgctctggcgtgcttgtctaagctctgaggagaggtggtgggtatggtgttgtcagcagatgcacagcagctggcatggtgagaatggatgataccaccaggtcatagacagaaaattgcagtgggccgaggtggccgaatggtcggagaagggggaggagaaacgaggagcatgtggatgatgccaaggagggttcgaagaacagcagagaaatgtatagcgttccacaccccaggtccagagagacaaatggaaaaagtgccgtgcgctgggaaacagggcggacacggtgtcccttgccagtgcagtttggagtaggaggaggagaggaacaatacagtggcttaaccttccagtacaggcatttggccccaggaggaaagaggagggaggatgagataaaaatgacagtcgaagtactggagtcaaacaggtgccgcatcacctctgttaacccagccacatgttcatcctggaacccaagttcctcatccaggcctcggtggtcccaggaactggccaagcctgcattctttctcccataagcctccacagattagagcatggccactcccctgccttgcacttctgctattcctcatcctggcccccttcccagacttctttgtcccgccaagtcccgtcctttcaaactgagcgtttgggaggtcttctctgtctaccagcagaaaccatctcacctcagtaatggtgagcccagcccacttttttctcccacacaattgagatgtctccgaccagcctcttcagtgctggatacttcttgcattctatgaatttaggatctctacgtgtttctcaacacacatgcatacacaaggacacacgcagacacacacattagcacacagacatacacacacagtgacacatgcatacagggacacacacatggacacatgcacatatgtgcacatacactgacatgctctgacacacacttgacacacacgtgtgcccacacgcgcgcacacacacacgcacaaacacacacacacaggtaacattgacagctgtggctgtagaagtgcttccaagggcctttcttttttgtccttgtctggagtttacagcacttggaaaatgtcaggcgtgggaggccttttggtcttgggttagggtaaactccctccacatatttggagagcctccaggccctgcaaaccgctcacgaagcagataacagtgaccttgctgtgagggaggacggcccgccgaggccagggagctgtgctggcagctgaggttacctctcctccccgctgcacctgggtcggctctgatgtcaagacctcccccgctccatccccggttgggctcccggccctctttgtgccggaaacaaggcccagctgggaggaggggagggcagagcactgctctgtagttccaggtggagaacagcccagctctcccctaggggctgcaggctgagtgtccgactccaacccccttgcctttttgcaggcccaaagcggcttctccaggatggcgacagattttccaagactcatcccaggctggagggggcccagctgcaacttcactggtgtgtgtatgtgtgggtgagctgaccctgagagtctggtgctagagggctggaggtcatgtcatcacagaaccctctctgctggccccttcaagcatcctgttcagggagctcactgctaccaggggctggagcctctccaggtttctgtctctgtcacttccttaactcttgagctactcagattaggactagcccttcttccacatatgtgaaaacagctgcattcatacattcagctacatatagctttgaatgcaaggcctggctgggagctggggatagacacatggtttgctacatagttggtggcctagtggaggaggcagacggcaggccctggtcagcgtgctgagtctgggggaggagcaagcctgcaggaggaagttactctgctggggagcggggccttaaggtgctccagacagaggggacaggatgcgcaaagctgtgtgggtctttgccttaggcaggggtgggagtcagcatttagttggagaaggggttcaggggcgtccagtggtgtgggcagagcacagggggctgcagtggctctggcgagaggtggtggtacctaaagttgtggcagtggccacggggctgggagctgtaggtggagttgggcagaccaagctctcctctgcatggagctccagagtccacacagatgtctacggatagctcgaagccctccctgatcagccagctccctctctttggcccccatttgcaaggaaagttcaaatgcggtgctcaggatggaagcagacttctaatgtgacttaccgtagttccatctgcttataaaaacataagtcatgccctttttcacgccgaaaagtgtgaaaaaagaattttatatttgcaaaagttttccctagaagttgctgcctgggctccagtgttccatggtcaggtcctctggagccgctgccgcccgggcctgagggcccaggcctgtgacttcctcctcctgtctgtctgcccgggagccggcaccggcacccaaagccgaagccgcctccctcttatcctctgcgagatcagcccagccaggaaggggccccagccgtctggctgtctgagccgggaccggttggcgggtgctctggccttcctcattagcggggtctccactgcccttcctctcgcaggctaggctgtggctcctattatagctactggagggttttttagtcaaagagcaccatgcatcatttattattttaccattcactcagacgttccctgagcacttcctatgctgctgctggacccagggatcagccaactagccccaaacggcagatcctctgtggagagtgaggaggttgctgggctgaatcaaagtgctctcacgcacggtggaagttgctacgtgcaaagagatgcgggcaaccaagtgctctgaggtgcagaggaggcagaggccatgccaaggagagcttacggctctcagcccttctccgtgccaggcgctgggctgagcacttcacatgctaatcctggaggtaatccatggggtaggtacgatgactatttccagatgaagagactgaagcttagagttaagtgatcccagtgagtacaatgcctggattcatagccagaggctgtgctttgaagccccagttcagttgccctcctaccatctctacccactcccccagccccttggggctgcagagcgacagcaagctacttcgtcatattgcatctcagttttcctgtctcctaagcttgttgtgggttttaaaggaacaaagcctctagtgtgctcagcagtgactggcacagaatacatgctccataaacgttagccatgtttatttttgtaattgtcactactaggtgctttgtaggaggcccctctcgggctggtgcatcctctgccatttctgtctcagggtcagcccgtctccccagtgctcacaatctgttcattaagatcatctggagtctgagtcccttcttattaaaattgtaaaaatatacagaaccacaagttaccattgtaactatttttaaggattcagttgagtggcattaagaataactgttatgcaaccatcaccaccaagaattttttgctatcctcgactaaaactctatccataaaacacggagtcccatttctcctccccccagcccctggcaacctctacttcgcatctctatgtatttgactcctctgggtgccccatataaacgaatcatacaatgtttgtctctctgtgactggcttatttcacttagcatgatgtcctcaaggttcacccatgtgtcagaatttccttgtttttgaggctgaataataatccatttgatggttttatttatccatatggccacctgggttgcttacacattttggctattgagaataatgctatatatatatacatacccaaatgtacccaatatatacttcaacgtccacaacta

SEQ ID NO: 99 - DNA, 2690 bp, Homo sapiens
  source 1..2690: mol_type = genomic DNA
cagtgtgtagaagactggagtgggagttgaggtggtatctgattacatcttgatatccacactaatgagcatacatcactttgtaacaaacgggctgtttctgagccaagaccatgagctgatagtgtggatggtggctgggacaggggagagagcccatcctgagggacggtcaaatcgtaataaagtagcaacaaatagctaaagctggagtgtaacagacaggtagaactgattgaggttcaaatgaagacagaggagaaggaactaaagatgatttgggggttctctgcttgatggaagtggggtagaggtgtcatgacttgcaggacacagaagggttgaggattagatagcagtaaatacatgcatgagggaggaagggcatggtttgggacatgttgagatcctatgttgaagatcaccatccagtggggacacaggcaatttcagtgcagtgtgattagtgactggatgaggaagcttagagaaggttccaaaatggaggacacacagaatttggttctagactacaggttcttaatcattttttttccagtaaatggcagtgtggtttaaaaaatcagattcaagtttgacaacttgttcaaatgaagctgtattggttctaaatagaatttatatgaaagcacaataacatattaccttgctgaagtacacacacacacacacacacacacacacacacacacacacactactctcacctctaaatttctttctcccgccagatggcactatgcttcaatatttaaagctacagatatggtgaactcagttgtctttattcttctcgctatgtggaccttaataaatgggcttaatgacatatgctgaactaacatcctaactgctactcctgtaaaataaaaagcagccgagctatattatgctttatgaaatatttataactgccaaacacaaaggcagtaagaacattggtttatacagcaaaagccgaaactctatttaagtgaagattaatgtggaaatgttatcacattctcacactgcaaaactaaattgatatagaggtgataaatgtggatagacgtggaatatacatataggcacacattatacattttacctgataaaatataccagctgaatttgcctaaggaagctgctaaagtcagatttgatgacgatttgtggaagttaccttgtggggcagcccaactgctagaacatttcaggaagttctatattgatggaagagaaattttaattatttctcctaaataaggagagagccattaggtgtgggattttgtgttctctttcaaatttgaatagacagaaggtatgtagaaatcaacatgatcttttcctttattcacatataattgtaaataacatgggtaatacaatttccaaatctatttactatgttgttaaagtagttggaatggttcctagatttataacatattccacaggcagcaatttgaaaatctttgagaagtttaaaaaatatctggcttacaatcatctcattaaggtataaaatcagggcttatattttatctgattgttattctcagatctacagttttttaaaagacaagtcttggttccttatagatctatcagcttccctccatgcacatttggctcatgaatgtatgaaatactttggacgtatttctattaaattgctttcctggggaaatacattatagttctcttttatttagattaatgagttttaggttaatctgtcgtgaatcctctgtaaatcagattcttgatagttaatatttcatctttgtatatttaaaatcagttcacatgttaagttaaagatgaaaaagagaactaaatcagcaatattgttatgtgcataaattaatgtgcatgtatgtctgctttaagtttagtttatagaagttttaaaaaatgaaaattggcatttttctacaaagaatgatgccttttgtcttcctacctgcctaatttcccaagaagagatctagatgtcagaacatgctgattgactatattttctaagaaaaattttagaattattaatatatctatactaactaaacacatccacttacctgtttttctggtactcttggatatagtcttttataggatttgagctgtgaatagacaaagaagatcatttgtccagctttggttttagtttttctaggactgcattgcacaagccttttcccagggtgcatcctagcagcttagtcttcaaatctcagcattggtatgtggggctgcgttcagcaaataataaaaatttagttgttttgtcatctggacctgcattatgtttgtgcatgcacccatatatatatatatttattaaatatacatatatattatatacacattatatccatttatataaatatacagatgtttatatacaaagaaaagttcaaaacattgatcattgtaatctatggtaataacattatagatatcttggtatactgttttatgtttcctaaatttcaatgatatattttattttgaatcagaaaaaagttaaaattatagaaatttaaataatacataatttaaaattctgaatactatagggaaagagtaaacttaacatttttctaggtagttatctaatttctcagaattatttttgaaaaaagccatcatttctctatataaaatttattatagactaa

SEQ ID NO: 100 - DNA, 579 bp, Homo sapiens
  source 1..579: mol_type = genomic DNA
gtgtgtttctcggcgcgcaaagctataaataatggcacagatcattaaaccccgagagcaggcccagccgccccgcaaacaagatgaagtacaaaccaccacgtgaggaggagagagggaggtacaaggcagagccccagacccagcctgtacctctcagctgggtcccagtgaacttgggcctccaaggcaccaccagcaggacctggtgtgactgcacatgatcagcagacccttcgttttggggccagttgtcaggtagcaacccctgaaaaaatcatacagcagctgaagtgaaatacaacaatgtcttcagagcacttttacccacttccttctaaaaagatctgagatggcctaccgtataagacacacccatgaaagaacaagaacagataaagaagtggattttaaaatgtcctgggaaaagaaacccaagaatggacagttactgcagaggaacccaaaacttagccctcagcttcctagatgaaaccaggtagtagcttaaaagcctcttcgcctacaccagaaataaactgagcacctactattagtcatgcatcaaagacatgatca

SEQ ID NO: 101 - DNA, 3793 bp, Homo sapiens
  source 1..3793: mol_type = genomic DNA
agcaatgaaaaggggacggagtgctgtggggttcagccagtgaaatgtgaagaaactccaggtcctagaatgtggaatccaggagagccacaggcttgcagtcacgagggccaggccgactaagaactggagaggctgggattcaccgggaggcatgggatggaaggggttgtcaccacacaggagccaggaacccagagacaggcagctgcaatgcagaaagaggaaaagacatcttttgaggaatgttcgcagcgtgaagcatttggcgtgatcttcaggacccggggctcagcctgggtggtgctgaaagtgacccggggctcagcgttgttgcctttgcttctttaactcctgagctgcctctgtggtccccttgggctcccagagctgctttgcctggagggtcagagagaagaagcgccttggactcacttgtgtgtcaaaaaggagtgtttgcaggccctagcagttttttatgattggccatatggctctattaagttcagaatcaaagatatattttttgacttatccataagaagcataagagaaatttaaataatcttaggtgtttttctgtccattttgcataaaatcacctgagtgttttatcaaaatgaagatgaggaaacggcctgcatttctaggggtttatttgctttttttttaaatcattgctggttctagttggaattgccctttgggactctcaggtctggatagttctctgaggctgatgagcgtggcagcgtgcccctggtgcaggcagccaaagaactggaatctgactcaggggcagagaaatgggttaggggtgaacccaacatctctgctcacttggtccttgaagcttttcttggcaaggatggtggtgggggacctgagtgccatcgtgttcaaacacccacagtggtgacatcatctctgcatctgtacagtgcacagggtgtggttagagcaggagttggaaggagaacagtcatagcttccagtcccatctgcttcttgtctctatgcccagaacctgaccctggaacaggactggtgcacagaagaccttgtgtgagtcctttggtaccacgtttgctgcacgaatgtctgagacacctcacagtcgcttgttcagcctctccctcctctctccgctggggaacatcatccctcatcactggaagaaattacaaggattgtgtaaacaattacaagtattctacaaacagtgagcatgctggtttggcccatctatttgtaagaaaaaacatggggttttatggcaatgagttacagctctaactcctggcacaatcacaaatatcagcacaaaacaccaagttatgtctcccttctgtcctaaaagggtaactacttcattcctaatattactgcacaccaagtctctcaatccgttaccaattttatccttgactcctgatgaaacacaaatctctttgatcaggttatcacatggcataggatggtggttgcttcagggaaataatttaaagtactttgcagtttgctaggtgccctggggcatagtattctttgggcctcctaaggttctgatggtgatactgatttaacgtatagaatagtttttaaatgtgaagtgctacagggaccactatgcagggagctgaagggctattgagctggacacttgagcattggctgcattgaatgataaatattgttctcttcctcaaaatagaaaatgacaatggaaaacttttaataatgtcaacattggtgactagaaaacatatgtgcctccttatgaaagcaggggtggtgactgaatagaattgttttgtttttagagaacgattttctttttgttttttcttcttttatagtattttacttaaaaatccaagtaatagtattctactgtattgcgaaaagtcagaacacttatgtatttttatttaaaaacgaacgttttagtcagcacccaatgcaacaaaagcaaactagaaattgctcagcaataaaacaggttgattctcacctttgctgatttagccgcatggaacctaattaatgggtagattatacatttgctcaacaagtattaatgggttctactcccgcgagaccctcatgaagtcctgaggttgcctctcgttcacagggagctccatgaggctggaggacttcatcagtcccaagaaacacgtgggcctctcagtaggatgctgcagggcagagacctggttctgtagctgactccacctggctaaggagcctctgggctgtggatacaccacctacagctgctcatcaggtgcagggcaccagattcccaaattctgctgaggtttttactctggtctgtcactgactggtcctgaacaaagtcactgttttactttgtcaaggatattttcatgcgaaatgagctcaggtgcacttcaaatcttctcccacagccactgtccccaggacacggggctggatacccaggttgagagaggctccagcacgcccacaccacggacccaaagccatcctacttcaactacaatactcattggaggagatctttcagatctgacacagccactaggtcaggctttttgaaaaactgaactggggaacacactggattggaacccagcactgtatgactagaaactatagcatgttttccttgtgagcataaattctaggattacaggcacaattttaaaagacatctatcaatattggactggcatgatcctccatgtttcagtttactccatatcctgttattagtaatggtatatatcaaaattacagtggcctcataggtatccagccattttcagaagtctttggaaattcttggatgatttctaagatttaatttatttttaaatgctttttccttctatgcatataaatatatataaatatttacatatacatgcgtgtatatatgtgtgcatatttgtttttatgtacaaacttatagacacacacatataccttaacttgagagaatggagatttcagcagagaaaattgtagagacatggccatgagacgtgtttatttgtaattgatcccttcaaatgtagagaaacaaggacatttgatggccatagacttggaaagattttttttaaatacaatcatcttgctatattttgaaagtcaattttagcacaatggagaaacatatttggaagacatttaggtgtgaaagctgagttttgttgatatttatgggcagagaaaacttcctttttgactattctgcagggccagagacagattgatcagttctagtgtttttgaagaaaaattactctctgtggctgacgagcttctgggaggcaggaattgtcccagtcaccagcacccaaaggaatgcccaccgtttaataaacatcccatatcttgaatgaataaaaaaatgttaccgatatatggtactatcagtttctaggcatggttcatgccccaattaaatcagtttcaggaagtagccttttttgaaacacacaaaatcttgaaaaatgctggtgaaaacataaaaattatctgggaaagtcagagaaatggctcagagtccgtgattttatggagagcataggctaccaggagatgacagtgctcttcatgtaatgagtgtggctctgccatatgagccaggctctgtgactggaccaaccagtgaaattataaggattataatttagatataaacagaccctgatatttattttccaacattagaacaggctatcacgtgagataggaaacattacggaagcattctaagaaagttagataaaaattt

SEQ ID NO: 102 - DNA, 4515 bp, Homo sapiens
  source 1..4515: mol_type = genomic DNA
cactagaagtttagaaatttgaaagagagaatctatttttggataggctttacccaccaaatgatatcaggtttcctgaagatttttaaattactgaagaaacaagttactaccagtaaagggtttagatactcacaggaatcccatttggtaagcttgctgcaagcccactatgtgagaggtgctagtgctagaagtagctcaagggtctaaaactctccacactaatggattgagctcccagtgtattgctacattattttatttttggtccagatatatctgccctaattgtagctacaaatacctttttacgtacaggctttttcattatctctctcatttctacttctgagtctgccctgaggtactgttctttcaccatcgcatctttcctctgatggacacttattatgccaccttgtgggaacctagaacttattctcccagatgctgtgagtgctgctggcagacagcccttagatcttagctccattcagaaatccttagctgaagaaagaaaaaatccacactcaataatggaagtgaagacataagggtctggccttcggcccagttggaacatctgtaaaggcctgctccatttgcagtgcatctcatagatttacagtgagactgtcttacctctcagcttggcttcgcctcctacccacttttgtttccttttcttctttcttccagtattgattccaaaaacatatcatagtaaacttcctgtatctccttctcagttctacatcctaggaaatccaactcacagcctttctcttttctgctcctctaaaaaactgccacttatgcctgttctctgagaaactcctatcaagactgggtgaaacaccatttctcacacaaagccctctctgctacttcaggtgacaccccaaactgagttttagtgtctttttcctatgatgataatagggaaaagaggaggtaattacaattattagtattatataacttagtggctatttgatccattgatattaacatttaaggttttatctttcaaaatacatgtagatggttaacacatatttctgtgaagccacagaaaagcacggaatattacttttttcaaagtaaaaaatacagtgctatctacattgtaggaaaggcacttgctgtttaatagggaactaaaaaaccactggctattgagtttcaaatctgaaacatccttcaggcttctccgcatgtgaaactagatcaagaagagaaagtctttgctcttggtgaactctgaatcccattgcttcttctttgtccttaatctctcccttcctgaactttgttcctccagccttgctctaattctctattctaaatccctcataattggcatttctaaagtgcttctgagtttgtttttactgaatcctaactaataaccagtgtacactacactcaccatgatcatctttaccacacagaaagtttgtaatcccatttgtttccttttaaatctgctactatttcaacattggcaaacatttcttatcaactataaaaaaaaaaatggcactccacagttcaacacatatgaaatagaatttgaaaaaatgcattaatggctacaaagcaaaaaattaaaggcaatggttttaaatgttgagatctacttcatgtagatacagaacaaagaataacctacattataactgctcataaaaaatgccgtgcagcaacatgggtttccaacacatttgataacctgtttctgtctcccccttcctccattaaattgaactctctgaattattcatgaaggctattttaatgggataattttttctggcgaacgctgacactgataccaaagaatatctctaggtattcatcttggataaataaggctcctggtaactagttttcttgatgcttcaccttattcaaatgaacatatatggaagtaactaatgtaaaccattttttaacccttaatgttttcaatatgtttctctccatgtatgtatagtcaaacatatgtttacacaggaattagccagggtgttgaattaaatgaaagtaaagcagaaagttaagcaattctcaggtgaacttgggcaggacattaagaagaaatttaaagtccacagtttccagctgtgatgacagttcggcattgctgtgggaatagcccatctctccacagtactttggccatagtcagaaccaatcattcacttgaatgtgtacattaagggttcttagcccaggatagtaggattttagggagttgatgaatcctttgaaatggaatatcaaattttctgtgtattatttttcttctgaaaataaagtttatgacattcatcagcttctcagaggtgttcttgactaccaccatacttctaaatggcaagaattcttatttcacacagaaaagaccttcggcagccaggattctctggatttccctgttcatgtgtgtgtcagaggtatttgtccctcattaaatttagtattataaaaattcacatgcaaacctttcaacttggaaaaaattgtactttgggttaattatatatcaacttttacagtttaatttttacaaatatctttgagtttttcctaggtatctacaagtctgctcatattttttaaatattaccgcaataatttttgatttttatactaatgcacaataagaacttaagttagatactggaatttattttagtcacaatgtaaaataaggatttatattacctttaaagaagcaaaattctcagaaatacagctttccaacatttctaatcattctgtaataaatcttttaagacactgaggtgactgtcactgctggatgtgtcctgggggcttttctgtcagaggagatagtgactcttcccatgcagattaagctggtgtttacagatttgccctgggtgtgcatttttatcccgctgtgatcaagtaacaccacctgacgagtgccagacagatctctggataagaaagttcactatgccctcactttgcacaagatgcagccctaatctggagaagagtctgtttcttgttcttttctcagtgatactgttatgtacactttacctttaggtgaagcattaggaatgaatctgtctgtgctgaaacttagatcatgaattgaaatcaatgtacaaaccaacattagggtttctggagacatgagaggaggcctgtgtgacaagtggaaaacatctaaaaatatttgagtccttagtaatttcaaatttgtgtggagagttttcacagacaaaactagtgacataagaaatcttattcgcaaaagctaatcaggctccagatgctgtgagtgcaaaccactcagccccaggtccttttaccttttcaccatttggtcttaaagcatttttaatatccatgaaacaaaactggctgttttcttcagatactgaaactcagctctccatatttcgatttcttacttcattagccaagttatgaggtaaatgattagctaactagagtttgtcacaattaggaagtgagaatgaaattcaatttgtgtttagaagtttattttgcattaagctcttctatctagggttttaaaccacacagaaaaagcttacacttgttaggtggtagagttattttttagttgcatacattgatataagtaattcatctttgaataagctcaaaacttgggttcacattttccttccttatatctcttatttgagacttacctaatgaggaacctgacatttagagggtattaattcaccaaaagccactttcatgcactgttttgatcagtaatggaaaagtcacactccatctatttggatagtgttttggagtatacagatcaatatgattcaaaaagtaatgtataactaacaacaaaatatcagtgaatgcactaaatatttgacatgagaattcaaatccaatctcacaggtgttttttaagccttggcttgagtcaggtctgctaatatcccattggtcaaagcaagaatgagccaagccaagaatcaaaagaagggagaagtgagggtgatgactatattgaacaaaatcccacacaggaaatgcagaagaaatatgtaactgttttcttgtttccttttaaaataaattttgtctattaagggaatatatatgaattgtcattaattttatttataattaactaattattaatttaaatccttttccactgcaaggtctactataaaaagaaattcagagagacctgggattctagtatgtatttgttatccttgttctccaaaaggttaaaggggggtggtagtgtataatcataactaataataattagaacataagaatctgaaaatcaatattgagggggatctaattatgcccatggcatttattttcttagtttgagaatggtgtgacaggtacggatgaatccaaacattccataagctcctcaacatttcagcttccctgcagttaagctatgacttactctagccaatgaaatgactgtagaagtggtat

SEQ ID NO: 103 - DNA, 470 bp, Homo sapiens
  source 1..470: mol_type = genomic DNA
tgagtcacaagaggctgaaagtgacctctgggtgtctataattaggccctgcctgacgtcagggcaacggattttctggcttggccaagttcaccgtgccaggcactgagttaagtccattatgcattccctgacacttgcagcaaccccactaggcccccattctccaaggagaaaactgaggcccaggagggtcatgccctgctgaggttactcagatcaaggaggtttcaactgagccagcaggacagaggtgtgctgaggcttggggccattcttctgaccacttctgctcctttttccctttctctaattttttccccacatggccctttgcctgaaatgcccttcatatctatcaggtttctgtgtaattgtcacctcctccaggaagtcttccctgaccttttcttctttgaactgatctctgaaatgatctctacctgtttattgcttgttgatgtgaactc

SEQ ID NO: 104 - DNA, 1086 bp, Homo sapiens
  source 1..1086: mol_type = genomic DNA
gaatcatctcagactgatgagactcttttcacatcagaatataaaccagccaaggtactctatttgaaaatgcagagcctgggtcttgtaatgaagtgcctgggtaaaaagagtctaaatgtcattgactttgagttctggagactggaagttcaagatgtcagcatagttggtttctggtgagggctctctccttgggttgcagatgactgccttctgctgtgccctcacaggacagagagagatagcaaactctgttgtttcttcttataagggcactaattccatcataaggccacctaacctcatctaacaaattgcctaccaaagcccatcctcaaataccatcaaattagagttatgtaattataaaattctgtgctcaataataagaaaaacaattaaaaaaatcaaaagaatcaaacaaacatgggtgacaaataagcaaagaagaagatgctcagcatcattagtcattaggaaaatgcaaattaaaaccataactactgaatggctaaaattaaaaagactgaccataccaggtgttggtgaggaggtagaggaacttgaactcttataccttgctggggaaagtataaaacagtacaaccactctggaaaacagtttgtcagtttcttagaaagttaaacataaacctaccattttatccagccattcactcttaggtataagagaaaaaacatgcatctatatagaattatgcataaatgttcatagaatcttcattggtaatagccccaaactgaaacaacccaaatgtccatgtacaggtgaacaaaccggtatatcagaaaaagagtatgcagtatgagttcatttatataacatgctaggaaacgcaaactaacgtatagtgttagtggtttctggtggatgaggagatgtgaaagaacaggaagaaagtgttacaaaagcaaagaaactttttgaggtattttcactatattaattgtggttatggtttcatagttgcatatttatgtcaaaacctatcgagttgtacatttcaactatatgttatttattgtaagtcagttataccccaataaagctgtaaatattag

SEQ ID NO: 105 - DNA, 15 bp, Homo sapiens
  source 1..15: mol_type = genomic DNA
tacaacctgcttact

SEQ ID NO: 106 - DNA, 16 bp, Homo sapiens
  source 1..16: mol_type = genomic DNA
tcatactatatgacag

SEQ ID NO: 107 - DNA, 20 bp, Homo sapiens
  source 1..20: mol_type = genomic DNA
gtggggaggtcagctacaaa

SEQ ID NO: 108 - DNA, 20 bp, Homo sapiens
  source 1..20: mol_type = genomic DNA
cggaaatggtttgaaatgct

SEQ ID NO: 109 - DNA, 20 bp, Homo sapiens
  source 1..20: mol_type = genomic DNA
acagacctgcagcagtgaga

SEQ ID NO: 110 - DNA, 20 bp, Homo sapiens
  source 1..20: mol_type = genomic DNA
gctagggaacgcagaacaag

SEQ ID NO: 111 - DNA, 20 bp, Homo sapiens
  source 1..20: mol_type = genomic DNA
aaggcttcccagagaaggag

SEQ ID NO: 112 - DNA, 20 bp, Homo sapiens
  source 1..20: mol_type = genomic DNA
actgggtgagtctcgctgtt

SEQ ID NO: 113 - DNA, 20 bp, Homo sapiens
  source 1..20: mol_type = genomic DNA
tgggacagcagagctaaggt

SEQ ID NO: 114 - DNA, 20 bp, Homo sapiens
  source 1..20: mol_type = genomic DNA
agattccagcacgcacttct

SEQ ID NO: 115 - DNA, 20 bp, Homo sapiens
  source 1..20: mol_type = genomic DNA
aaagggaagagggaaaacga

SEQ ID NO: 116 - DNA, 20 bp, Homo sapiens
  source 1..20: mol_type = genomic DNA
cgtctagaaccagcccagag

SEQ ID NO: 117 - DNA, 20 bp, Homo sapiens
  source 1..20: mol_type = genomic DNA
cagccctgctttagttcctg

SEQ ID NO: 118 - DNA, 20 bp, Homo sapiens
  source 1..20: mol_type = genomic DNA
ttcgttggggattttactgc

SEQ ID NO: 119 - DNA, 20 bp, Homo sapiens
  source 1..20: mol_type = genomic DNA
tttggagatggaacctggag

SEQ ID NO: 120 - DNA, 20 bp, Homo sapiens
  source 1..20: mol_type = genomic DNA
tctggtatgggggagacttg

SEQ ID NO: 121 - DNA, 20 bp, Homo sapiens
  source 1..20: mol_type = genomic DNA
agctctgggttggactgaga

SEQ ID NO: 122 - DNA, 20 bp, Homo sapiens
  source 1..20: mol_type = genomic DNA
tgcatacattctggcagagc

SEQ ID NO: 123 - DNA, 20 bp, Homo sapiens
  source 1..20: mol_type = genomic DNA
ggttgggtgcctattaaacg

SEQ ID NO: 124 - DNA, 20 bp, Homo sapiens
  source 1..20: mol_type = genomic DNA
ggttcatgagcctttggaag

SEQ ID NO: 125 - DNA, 20 bp, Homo sapiens
  source 1..20: mol_type = genomic DNA
tgttaattcaggggcacaca

SEQ ID NO: 126 - DNA, 20 bp, Homo sapiens
  source 1..20: mol_type = genomic DNA
ggtggagagccactgaagag

SEQ ID NO: 127 - DNA, 20 bp, Homo sapiens
  source 1..20: mol_type = genomic DNA
tctctgtcccttgtgtgtgc

SEQ ID NO: 128 - DNA, 20 bp, Homo sapiens
  source 1..20: mol_type = genomic DNA
cttggaggtgtgggcatagt

SEQ ID NO: 129 - DNA, 20 bp, Homo sapiens
  source 1..20: mol_type = genomic DNA
gagccaagtgcacacagaaa

SEQ ID NO: 130 - DNA, 20 bp, Homo sapiens
  source 1..20: mol_type = genomic DNA
tggtctgttcctggccttag

SEQ ID NO: 131 - DNA, 20 bp, Homo sapiens
  source 1..20: mol_type = genomic DNA
gtggacgacaagggaggtta

SEQ ID NO: 132 - DNA, 20 bp, Homo sapiens
  source 1..20: mol_type = genomic DNA
cggaatggctcctacaacat

SEQ ID NO: 133 - DNA, 20 bp, Homo sapiens
  source 1..20: mol_type = genomic DNA
gaggctcctggatctctgtg

SEQ ID NO: 134 - DNA, 20 bp, Homo sapiens
  source 1..20: mol_type = genomic DNA
ttgggaggcaaaggtagatg

SEQ ID NO: 135 - DNA, 20 bp, Homo sapiens
  source 1..20: mol_type = genomic DNA
gaaatgagtggtggcagtga

SEQ ID NO: 136 - DNA, 20 bp, Homo sapiens
  source 1..20: mol_type = genomic DNA
cttaggtctgcgcctaatgg

SEQ ID NO: 137 - DNA, 20 bp, Homo sapiens
  source 1..20: mol_type = genomic DNA
gcacagatgcatagcctcaa

SEQ ID NO: 138 - DNA, 20 bp, Homo sapiens
  source 1..20: mol_type = genomic DNA
gcagcctggacttttctcac

SEQ ID NO: 139 - DNA, 20 bp, Homo sapiens
  source 1..20: mol_type = genomic DNA
tcacctccaagtgggtcttc

SEQ ID NO: 140 - DNA, 20 bp, Homo sapiens
  source 1..20: mol_type = genomic DNA
agctcggtctgtcgtgagtt

SEQ ID NO: 141 - DNA, 21 bp, Homo sapiens
  source 1..21: mol_type = genomic DNA
caaggcttaataccgccactg

SEQ ID NO: 142 - DNA, 22 bp, Homo sapiens
  source 1..22: mol_type = genomic DNA
aatgtgcatagtaaccaggctg

SEQ ID NO: 143 - DNA, 19 bp, Homo sapiens
  source 1..19: mol_type = genomic DNA
atggggtctctggttctgc

SEQ ID NO: 144 - DNA, 22 bp, Homo sapiens
  source 1..22: mol_type = genomic DNA
caataccatcttgctccgtgaa

SEQ ID NO: 145 - DNA, 22 bp, Homo sapiens
  source 1..22: mol_type = genomic DNA
aagagataccgctatgcctacc

SEQ ID NO: 146 - DNA, 19 bp, Homo sapiens
  source 1..19: mol_type = genomic DNA
gctgctcgccagtaaaggg

SEQ ID NO: 147 - DNA, 16 bp, Homo sapiens
  source 1..16: mol_type = genomic DNA
tgcttgctagtgtggt

SEQ ID NO: 148 - DNA, 15 bp, Homo sapiens
  source 1..15: mol_type = genomic DNA
aggtgtgcgatagag